(19) United States
(12) Patent Application Publication
(10) Pub. No.: US 2019/0032064 A1
(43) Pub. Date: Jan. 31, 2019

(54) Personalized Delivery Vector-Based Immunotherapy and Uses Thereof

(71) Applicant: ADVAXIS, INC., Princeton, NJ (US)

(72) Inventors: Robert PETIT, Newtown, PA (US); Kyle PERRY, Lawrenceville, NJ (US); Michael F. PRINCIOTTA, Hightstown, NJ (US)

(73) Assignee: Advaxis, Inc., Princeton, NJ (US)

(21) Appl. No.: 16/073,194

(22) PCT Filed: Jan. 27, 2017
(86) PCT No.: PCT/US2017/015403

§ 371 Date: Jul. 26, 2018

Related U.S. Application Data
(60) Provisional application No. 62/287,871, filed on Jan. 27, 2016.

(51) Int. Cl. C12N 15/74; A61P 35/00; C12R 1/01

(52) U.S. Cl. CPC C12N 15/74; A61K 39/0011; C12R 1/01; A61P 35/00

(57) ABSTRACT

Disclosed herein is a personalized immunotherapy composition for a subject having a disease or condition, including therapeutic vaccine delivery vectors and methods of making the same comprising gene expression constructs expressing frameshift-mutation-derived peptides associated with one or more neo-epitopes encoded by nucleic acid sequences comprising at least one frameshift mutation, wherein the frameshift mutation is specific to a subject's cancer or unhealthy tissue. A delivery vector of this disclosure includes bacterial vectors; or viral vectors, or peptide vaccine vectors; or DNA vaccine vectors including Listeria bacterial vectors comprising one or more fusion proteins comprising one or more frameshift-mutation-derived peptides comprising one or more neo-epitopes present in disease-bearing biological samples obtained from the subject. Disclosed are also methods of using these compositions for inducing an immune response against a disease or condition, including a tumor or cancer, or an infection in the subject.

### CROSS REFERENCE TO RELATED APPLICATIONS

This application claims the benefit of U.S. Application No. 62/287,871, filed Jan. 27, 2016, which is herein incorporated by reference in its entirety for all purposes.

### REFERENCE TO A SEQUENCE LISTING SUBMITTED AS A TEXT FILE VIA EFS WEB

The Sequence Listing written in file 490970SEQLIST.txt is 180 kb, was created on Jan. 27, 2017, and is hereby incorporated by reference.

### BACKGROUND

Before personalized medicine, most patients with a specific type and stage of cancer received the same treatment. However, it has become clear to doctors and patients that some treatments work well for some patients and not as well for others. Thus, there is a need to develop effective, personalized cancer vaccines effective for a particular tumor. Personalized treatment strategies may be more effective and cause fewer side effects than would be expected with standard treatments.

Tumors develop due to mutations in a person's DNA, which can cause the production of mutated or abnormal proteins, comprising neo-epitopes not present within the corresponding normal protein produced by the host. Many of these neo-epitopes stimulate T-cell responses and result in the destruction of early-stage cancerous cells by the immune system. In cases of established cancer, however, the immune response is insufficient. In other instances, development of effective, long term vaccines that target tumor antigens in cancer, but not specifically targeting the neo-epitopes thereof, have proven difficult. A major reason for this is that T cells specific for tumor self-antigens are eliminated or inactivated through mechanisms of tolerance.

Neo-epitopes are epitopes present within a protein associated with a disease, for example cancer, wherein the specific “neo-epitope” is not present within the corresponding normal protein associated with a subject not having a disease or a disease-bearing tissue therein. Neo-epitopes may be challenging to identify, but doing so and developing treatments that target them would be advantageous for use within a personalized treatment strategy because they are rare and can vary from person to person. Some neo-epitopes are a result of mutations such as frameshift mutations, which may lead to the expression of nonsensical peptides. Nonsensical peptides potentially possess expressed immunogenic neo-epitopes and therefore may be useful in designing vaccines for personalized treatment.

Listeria monocytogenes (Lm) is a gram-positive facultative intracellular pathogen that causes listeriosis. In its intercellular lifecycle, Lm enters host cells by phagocytosis or by active invasion of non-phagocytic cells. Following internalization, Lm may mediate its escape from the membrane bound phagosome/vacuole by secretion of several bacterial virulence factors, primarily the pore-forming protein listeriolysin O (LLO), enabling the bacteria to enter the host cell cytoplasm. In the cytoplasm, Lm replicates and spreads to adjacent cells based on the mobility facilitated by the bacterial actin-polymerizing protein (ActA). In the cytoplasm, Lm-secreted proteins are degraded by the proteasome and processed into peptides that associate with MHC class I molecules in the endoplasmic reticulum. This unique characteristic makes it a very attractive cancer vaccine vector in that tumor antigen can be presented with MHC class I molecules to activate tumor-specific cytotoxic T lymphocytes (CTLs). While residing in the cytosol, the bacteria can be recognized by various intercellular receptors, for example by recognition of peptidoglycan by nuclear oligomerization domain-like receptors and Lm DNA by DNA sensor, AIM2, and activate inflammatory and immune-modulatory cascades.

In addition, once internalized, Lm may then be processed in the phagolysosomal compartment and peptides presented on MHC Class II for activation of Lm-specific CD4-T cell responses. This combination of inflammatory responses and efficient delivery of antigens to the MHC I and MHC II pathways makes Lm a powerful vaccine vector in treating, protecting against, and inducing an immune response against a tumor.

Targeting neo-epitopes specific to a subject's cancer as a component of a Listeria-based vaccine that additionally stimulates T-cell response or is used in combination with other therapies may provide a vaccine that is both personalized to a subject's cancer and effective in the treatment of the cancer. Antigen fusion strategies, which increase the immunogenicity of an antigen or the ability of vaccines to stimulate T cells that have escaped tolerance mechanisms, may have a particular potential as immunotherapies.

### SUMMARY

The present disclosure provides personalized immunotherapy compositions and uses thereof for targeting potential neo-epitopes within abnormal or unhealthy tissue of a subject, wherein the immunotherapy comprises the use of a recombinant Listeria vaccine or another immunotherapy delivery vector as a delivery and immunotherapeutic vector for expressing peptides and/or fusion polypeptides comprising these neo-epitopes in order to enhance an immune response targeting these neo-epitopes. The personalized immunotherapies created may effectively treat, prevent, or reduce the incidence of a disease, for example cancer in a subject. Further, the immunotherapy delivery vectors and recombinant Listeria of the present disclosure may effectively be used in combination with other anti-disease or anti-cancer therapies.

In one aspect, disclosed herein is immunotherapy delivery vector comprising a nucleic acid comprising an open reading frame encoding a recombinant polypeptide comprising a PEST-containing peptide fused to one or more heterologous peptides, wherein the one or more heterologous peptides comprise one or more frameshift-mutation-derived peptides comprising one or more immunogenic neo-epitopes. Such immunotherapy delivery vectors can be, for example, a recombinant Listeria strain. The frameshift-mutation-derived peptides can be, for example, disease-specific or condition-specific.

In another aspect, disclosed herein is an immunogenic composition comprising at least one immunotherapy delivery vector disclosed herein. Such immunogenic compositions can further comprise, for example, an adjuvant.

In another aspect, disclosed herein is a method of treating, suppressing, preventing, or inhibiting a disease or a condition in a subject, comprising administering to the subject an immunotherapy delivery vector disclosed herein or an immunogenic composition disclosed herein, wherein the one or more frameshift-mutation-derived peptides are encoded by a source nucleic acid sequence from a disease-bearing or condition-bearing biological sample from the subject. Such methods can, for example, elicit a personalized anti-disease or anti-condition immune response in the subject, wherein the personalized immune response is targeted to the one or more frameshift-mutation-derived peptides.

In another aspect, disclosed herein is a process for creating a personalized immunotherapy for a subject having a disease or condition, comprising: (a) comparing one or more open reading frames (ORFs) in nucleic acid sequences extracted from a disease-bearing or condition-bearing biological sample from the subject with one or more ORFs in nucleic acid sequences extracted from a healthy biological sample, wherein the comparing identifies one or more nucleic acid sequences encoding one or more peptides comprising one or more immunogenic neo-epitopes encoded within the one or more ORFs from the disease-bearing or condition-bearing biological sample, wherein at least one of the one or more nucleic acid sequences comprises one or more frameshift mutations and encodes one or more frameshift-mutation-derived peptides comprising one or more immunogenic neo-epitopes; and (b) generating an immunotherapy delivery vector comprising a nucleic acid comprising an open reading frame encoding a recombinant polypeptide comprising the one or more peptides comprising the one or more immunogenic neo-epitopes identified in step (a). Optionally, such processes can further comprise storing the immunotherapy delivery vector or the DNA immunotherapy or the peptide immunotherapy for administering to the subject within a predetermined period of time. Optionally, such processes can further comprise administering a composition comprising the immunotherapy vector to the subject, wherein the administering results in the generation of a personalized T-cell immune response against the disease or condition.

In one aspect, the present disclosure relates to a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein the one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease-bearing or condition-bearing biological sample of a subject.

In another related aspect, said recombinant Listeria further comprises at least one nucleic acid sequence encoding one or more recombinant polypeptides comprising one or more peptides fused to an immunogenic polypeptide, wherein said one or more peptides comprise one or more immunogenic neo-epitopes. In another aspect, said one or more peptides are sensical peptides.

In another aspect, the disclosure relates to an immunotherapy delivery vector comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein said source is obtained from a disease-bearing or condition-bearing biological sample of a subject.

In another related aspect, said recombinant Listeria further comprises at least one nucleic acid sequence encoding one or more recombinant polypeptides comprising one or more peptides fused to an immunogenic polypeptide, wherein said one or more peptides comprise one or more immunogenic neo-epitopes. In another aspect, said one or more peptides are sensical peptides.

In a related aspect, the frameshift mutation is in comparison to a source nucleic acid sequence of a healthy biological sample.

In another related aspect, the at least one frameshift mutation comprises multiple frameshift mutations, and the multiple frameshift mutations are present within the same gene. In another related aspect, the at least one frameshift mutation comprises multiple frameshift mutations, and the multiple frameshift mutations are not present within the same gene.

In another related aspect, at least one frameshift mutation is within an exon encoding region of a gene. In another related aspect, the exon is the last exon of the gene. In a related aspect, each of the one or more nonsensical peptides can range from very short (e.g. about 10 amino acid sequences) to very long (e.g. over 100 amino acid sequences). In a related aspect, each of the one or more nonsensical peptides is about 60-100 amino acids in length. In a related aspect, each of the one or more nonsensical peptides is about 8-10, 11-20, 21-40, 41-60, 61-80, 81-100, 101-150, 151-200, 201-250, 251-300, 301-350, 351-400, 401-450, 451-500, or 8-500 or more amino acids in length. In another related aspect, the one or more nonsensical peptide is expressed in the disease-bearing or condition-bearing biological sample.

In another related aspect, the one or more nonsensical peptide does not encode a post-translational cleavage site. In another related aspect, the source nucleic acid sequence comprises one or more regions of microsatellite instability. In another related aspect, the one or more neo-epitopes comprises a T-cell epitope.

In a related aspect, the one or more neo-epitopes comprises a self-antigen associated with the disease or condition, wherein the self-antigen comprises a cancer or tumor-associated neo-epitope, or a cancer-specific or tumor-specific neo-epitope. In another related aspect, the one or more nonsensical peptides comprising one or more neo-epitopes comprise an infectious disease-associated or disease specific neo-epitope. In another related aspect, the recombinant Listeria expresses and secretes the one or more recombinant polypeptides. In another related aspect, each of the recombinant polypeptides comprising about 1-20 the neo-epitopes.

In a related aspect, the one or more nonsensical peptides or fragments thereof are each fused to an immunogenic polypeptide. In another related aspect, the one or more nonsensical peptides or fragments thereof comprise multiple operably linked nonsensical peptides or fragments thereof from N-terminal to C-terminal, wherein the immunogenic polypeptide is fused to one of the multiple nonsensical peptides or fragments thereof. In another related aspect, the immunogenic polypeptide is operably linked to the N-terminal nonsensical peptide. In another related aspect, the immunogenic polypeptide is a mutated Listeriolysin O (LLO) protein, a truncated LLO (tLLO) protein, a truncated ActA protein, or a PEST amino acid sequence.

In a related aspect, the one or more recombinant polypeptide is operably linked to a tag at the C-terminal, optionally via a linker sequence. In another related aspect, the linker sequence encodes a 4× glycine linker. In another related aspect, the tag is selected from a group comprising a 6× Histidine tag, SIINFEKL peptide, 6× Histidine tag operably linked to 6× histidine, and any combination thereof. In another related aspect, the nucleic acid sequence encoding the recombinant polypeptide comprises 2 stop codons following the sequence encoding the tag.

In a related aspect, the nucleic acid sequence encoding the recombinant polypeptide encodes components comprising: pHly-tLLO-[nonsensical peptide or fragment thereof-glycine linker₍₄ₓ₎-nonsensical peptide or fragment thereof—glycine linker_{(4X)}]ₙ-SIINFEKL-6× His tag-2× stop codon, wherein the nonsensical peptide or fragment thereof is twenty-one amino acids long, and wherein n=1-20. In another related aspect, the nonsensical peptide or fragment thereof may be the same or different.

In a related aspect, at least one nucleic acid sequence encoding the recombinant polypeptide is integrated into the Listeria genome. In another related aspect, at least one nucleic acid sequence encoding the recombinant polypeptide is in a plasmid. In another related aspect, the plasmid is stably maintained in the Listeria strain in the absence of antibiotic selection.

In a related aspect, the Listeria strain is an attenuated Listeria strain. In another related aspect, attenuated Listeria comprises a mutation in one or more endogenous genes. In a related aspect, the endogenous gene mutation is selected from an actA gene mutation, a prfA mutation, an actA and inlB double mutation, a dal/dal gene double mutation, or a dal/dat/actA gene triple mutation, or a combination thereof. In another related aspect, the mutation comprises an inactivation, truncation, deletion, replacement or disruption of the gene or genes. In another related aspect, at least one nucleic acid sequence encoding the recombinant polypeptide further comprises a second open reading frame encoding a metabolic enzyme, or wherein the Listeria strain comprises a second nucleic acid sequence comprising an open reading frame encoding a metabolic enzyme. In another related aspect, the metabolic enzyme is an alanine racemase enzyme or a D-amino acid transferase enzyme.

In a related aspect, the Listeria is Listeria monocytogenes.

In a related aspect, the nonsensical peptide is acquired from the comparison of one or more open reading frames (ORFs) in nucleic acid sequences extracted from the disease-bearing biological sample with one or more ORFs in nucleic acid sequences extracted from a healthy biological sample, wherein the comparison identifies one or more frameshift mutations within the nucleic acid sequences, wherein the nucleic acid sequence comprising the mutations encodes one or more nonsensical peptides comprising one or more immunogenic neo-epitopes encoded within the one or more ORFs from the disease-bearing biological sample.

In a related aspect, the comparison comprises a use of a screening assay or screening tool and associated digital software for comparing one or more ORFs in nucleic acid sequences extracted from the disease-bearing biological sample with one or more ORFs in nucleic acid sequences extracted from the healthy biological sample.

In a related aspect, the comparison comprises comparing open reading frame exome of a predefined gene-set selected from a group comprising: nucleic acid sequences encoding known and predicted cancer or tumor antigens, nucleic acid sequences encoding tumor or cancer-associated antigens, nucleic acid sequences encoding known or predicted tumor or cancer protein markers, nucleic acid sequences encoding known and predicted infectious disease or condition associated genes, nucleic acid sequences encoding genes expressed in the disease-bearing biological sample, nucleic acid sequences comprising regions of microsatellite instability, and any combination thereof.

In a related aspect, the disease-bearing biological sample is obtained from the subject having the disease or condition. In another related aspect, the healthy biological sample is obtained from the subject having the disease or condition. In another related aspect, the biological sample comprises a tissue, a cell, a blood sample, or a serum sample.

In a related aspect, the nonsensical peptide is characterized for neo-epitopes by: (i) generating one or more different peptide sequences from the nonsensical peptide; and optionally, (ii) screening each the peptides generated in (i) and selecting for binding by MHC Class I or MHC Class II to which a T-cell receptor binds to.

In one aspect, the present disclosure relates to an immunogenic composition comprising at least one of any one of the Listeria strains of the present disclosure. In another related aspect, the immunogenic composition further comprising an additional adjuvant. In another related aspect, the additional adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

In one aspect, the present disclosure relates to a method of eliciting a personalized targeted immune response in a subject having a disease or condition, said method comprising administering to the subject the immunogenic composition of the present disclosure, wherein the personalized immune response is targeted to one or more nonsensical peptides or fragments thereof comprising one or more neo-epitopes present within a disease or condition bearing biological sample of a subject.

In one aspect, the present disclosure relates to a method of treating, suppressing, preventing or inhibiting a disease or a condition in a subject, comprising administering to the subject the immunogenic composition of the present disclosure.

In one aspect, the present disclosure relates to a method of increasing the ratio of T effector cells to regulatory T cells (Tregs) in the spleen and tumor of a subject, said method comprising the step of administering to the subject the immunogenic composition of the present disclosure, wherein the T effector cells are targeted to one or more nonsensical peptides comprising one or more neo-epitopes present within a disease or condition bearing biological sample of a subject.

In one aspect, the present disclosure relates to a method for increasing neo-epitope-specific T-cells in a subject, the method comprising the step of administering to the subject the immunogenic composition of the present disclosure.

In one aspect, the present disclosure relates to a method for increasing survival time of a subject having a tumor or suffering from cancer, or suffering from an infectious disease, the method comprising the step of administering to the subject the immunogenic composition of the present disclosure.

In one aspect, the present disclosure relates to a method of reducing tumor or metastases size in a subject, the method comprising the step of administering to the subject the immunogenic composition of the present disclosure.

In a related aspect, the methods of this disclosure further comprising administering a booster treatment.

In a related aspect, administering a recombinant Listeria or composition thereof of this disclosure, elicits a personalized enhanced anti-infectious disease immune response in the subject. In another related aspect, the method elicits a personalized anti-cancer or anti-tumor immune response.

Other features and advantages of the present disclosure will become apparent from the following detailed description examples and figures. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The subject matter regarded as the disclosure is particularly pointed out and distinctly claimed in the concluding portion of the specification. The disclosure, however, both as to organization and method of operation, together with objects, features, and advantages thereof, may best be understood by reference to the following detailed description when read with the accompanying drawings.

FIG. 1A shows a schematic representation of the chromosomal region of the Lmdd-143 and LmddA-143 after klk3 integration and actA deletion.

FIG. 1B shows the klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIG. 2A shows a map of the pADV134 plasmid.

FIG. 2B shows proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3).

FIG. 2C shows a map of the pADV142 plasmid.

FIG. 2D shows a Western blot showed the expression of LLO-PSA fusion protein using anti-PSA and anti-LLO antibody.

FIG. 3A shows plasmid stability in vitro of LmddA-LLO-PSA if cultured with and without selection pressure (D-alanine). Strain and culture conditions are listed first and plates used for CFU determination are listed after.

FIG. 3B shows clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIG. 4A shows in vivo clearance of the strain LmddA-LLO-PSA after administration of 10⁸ CFU in C57BL/6 mice. The number of CFU were determined by plating on BHI/str plates. The limit of detection of this method was 100 CFU.

FIG. 4B shows a cell infection assay of J774 cells with 10403S, LmddA-LLO-PSA and XFL7 strains.

FIG. 5A shows PSA tetramer-specific cells in the splenocytes of naïve and LmddA-LLO-PSA immunized mice on day 6 after the booster dose.

FIG. 5B shows intracellular cytokine staining for IFN-γ in the splenocytes of naïve and LmddA-LLO-PSA immunized mice stimulated with PSA peptide for 5 h.

FIGS. 5C and 5D show specific lysis of EL4 cells pulsed with PSA peptide with in vitro stimulated effector T cells from LmddA-LLO-PSA immunized mice and naïve mice at different effector/target ratio using a caspase based assay (shown in FIG. 5C) and a europium based assay (shown in FIG. 5D).

FIG. 5E shows the number of IFNγ spots in naïve and immunized splenocytes obtained after stimulation for 24 h in the presence of PSA peptide or no peptide.

FIGS. 6A-6C show immunization with LmddA-142 induces regression of Tramp-C1-PSA (TPSA) tumors. Mice were left untreated (n=8) (FIG. 6A) or immunized i.p. with LmddA-142 (1×10⁸ CFU/mouse) (n=8) (FIG. 6B) or Lm-LLO-PSA (n=8), (FIG. 6C) on days 7, 14 and 21. Tumor sizes were measured for each individual tumor and the values expressed as the mean diameter in millimeters. Each line represents an individual mouse.

FIG. 7A shows analysis of PSA-tetramer⁺CD8⁺ T cells in the spleens and infiltrating T-PSA-23 tumors of untreated mice and mice immunized with either an Lm control strain or LmddA-LLO-PSA (LmddA-142).

FIG. 7B shows analysis of CD4⁺ regulatory T cells, which were defined as CD25⁺FoxP3⁺, in the spleens and infiltrating T-PSA-23 tumors of untreated mice and mice immunized with either an Lm control strain or LmddA-LLO-PSA.

FIG. 8A shows a schematic representation of the chromosomal region of the Lmdd-143 and LmddA-143 after klk3 integration and actA deletion.

FIG. 8B shows the klk3 gene is integrated into the Lmdd and LmddA chromosome. PCR from chromosomal DNA preparation from each construct using klk3 specific primers amplifies a band of 760 bp corresponding to the klk3 gene.

FIG. 9A shows Lmdd-143 and LmddA-143 secrete the LLO-PSA protein. Proteins from bacterial culture supernatants were precipitated, separated in a SDS-PAGE and LLO and LLO-PSA proteins detected by Western-blot using an anti-LLO and anti-PSA antibodies.

FIG. 9B shows LLO produced by Lmdd-143 and LmddA-143 retains hemolytic activity. Sheep red blood cells were incubated with serial dilutions of bacterial culture supernatants and hemolytic activity measured by absorbance at 590 nm.

FIG. 9C shows Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 10 shows immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIGS. 11A and 11B are related to construction of ADXS31-164. FIG. 11A shows a plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). FIG. 11B shows expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIGS. 12A-12C show immunogenic properties of ADXS31-164. FIG. 12A shows cytotoxic T cell responses elicited by Her2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). FIG. 12B shows IFN-γ secreted by the splenocytes from immunized FVB/N mice into the cell culture medium, measured by ELISA, after 24 hours of in vitro stimulation with mitomycin C treated NT-2 cells. FIG. 12C shows IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the Fig. legend. IFN-γ secretion was detected by an ELISA assay using cell culture supernatants harvested after 72 hours of co-incubation. Each data point was an average of triplicate data +/− standard error. *P value<0.001.

FIG. 13 shows tumor Prevention Studies for Listeria-ChHer2/neu Vaccines Her2/neu transgenic mice were injected six times with each recombinant Listeria-ChHer2 or a control Listeria vaccine Immunizations started at 6 weeks of age and continued every three weeks until week 21. Appearance of tumors was monitored on a weekly basis and expressed as percentage of tumor free mice. *p<0.05, N=9 per group.

FIG. 14 shows the effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. Dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIGS. 15A and 15B show the effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. FIG. 15A shows dot-plots of the Tregs from a representative experiment. FIG. 15B shows the frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIGS. 16A-16C show vaccination with ADXS31-164 can delay the growth of a breast cancer cell line in the brain. Balb/c mice were immunized thrice with ADXS31-164 or a control Listeria vaccine. EMT6-Luc cells (5,000) were injected intracranially in anesthetized mice. FIG. 16A shows ex vivo imaging of the mice was performed on the indicated days using a Xenogen X-100 CCD camera. FIG. 16B shows pixel intensity was graphed as number of photons per second per cm2 of surface area; this is shown as average radiance. FIG. 16C shows expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

FIGS. 17A-C represent a schematic map of a recombinant Listeria protein minigene construct. FIG. 17A represents a construct producing the ovalbumin derived SIINFEKL peptide (SEQ ID NO: 1). FIG. 17B represents a comparable recombinant protein in which a GBM derived peptide has been introduced in place of SIINFEKL by PCR cloning. FIG. 17C represents a construct designed to express 4 separate peptide antigens from a strain of Listeria.

FIG. 18 shows a schematic representation showing the cloning of the different ActA PEST regions in the plasmid backbone pAdv142 (see FIG. 1C) to create plasmids pAdv211, pAdv223 and pAdv224 is shown in. This schematic shows different ActA coding regions were cloned in frame with Listeriolysin 0 signal sequence in the backbone plasmid pAdv142, restricted with XbaI and XhoI.

FIG. 19A shows a tumor regression study using TPSA23 as transplantable tumor model. Three groups of eight mice were implanted with 1×10⁶ tumor cells on day 0 and were treated on day 6, 13 and 20 with 10⁸ CFU of different therapies: LmddA142, LmddA211, LmddA223 and LmddA224. Naïve mice did not receive any treatment. Tumors were monitored weekly and mice were sacrificed if the average tumor diameter was 14-18 mm Each symbol in the graph represents the tumors size of an individual mouse. The experiment was repeated twice and similar results were obtained.

FIG. 19B shows the percentage survival of the naïve mice and immunized mice at different days of the experiment.

FIGS. 20A-B show PSA specific immune responses were examined by tetramer staining (FIG. 20A) and intracellular cytokine staining for IFN-γ (FIG. 20B). Mice were immunized three times at weekly intervals with 10⁸ CFU of different therapies: LmddA142 (ADXS31-142), LmddA211, LmddA223 and LmddA224. For immune assays, spleens were harvested on day 6 after the second boost. Spleens from 2 mice/group were pooled for this experiment. In FIG. 20A, PSA specific T cells in the spleen of naïve, LmddA142, LmddA211, LmddA223 and LmddA224 immunized mice were detected using PSA-epitope specific tetramer staining. Cells were stained with mouse anti-CD8 (FITC), anti-CD3 (Percp-Cy5.5), anti-CD62L (APC) and PSA tetramer-PE and analyzed by FACS Calibur. In FIG. 20B, Intracellular cytokine staining to detect the percentage of IFN-γ secreting CD8+ CD62Llow cells in the naïve and immunized mice after stimulation with 1 μM of PSA specific, H-2Db peptide (HCIRNKSVIL; SEQ ID NO: 59) for 5 h.

FIGS. 21A-C show TPSA23, tumor model was used to study immune response generation in C57BL6 mice by using ActA/PEST2 (LA229) fused PSA and tLLO fused PSA. Four groups of five mice were implanted with 1×10⁶ tumor cells on day 0 and were treated on day 6 and 14 with 10⁸ CFU of different therapies: LmddA274, LmddA142 (ADXS31-142) and LmddA211. Naïve mice did not receive any treatment. On Day 6 post last immunization, spleen and tumor was collected from each mouse. FIG. 21A shows a table showing the tumor volume on day 13 post immunization. PSA specific immune responses were examined by pentamer staining in spleen (FIG. 21B) and in tumor (FIG. 21C). For immune assays, spleens from 2 mice/group or 3 mice/group were pooled and tumors from 5 mice/group was pooled. Cells were stained with mouse anti-CD8 (FITC), anti-CD3 (Percp-Cy5.5), anti-CD62L (APC) and PSA Pentamer-PE and analyzed by FACS Calibur.

FIG. 22 shows a flow chart of a process (manual or automated) that generates the DNA sequence of a personalized plasmid vector comprising one or more neo-epitopes for use in a delivery vector, e.g., Listeria monocytogenes using output data containing all neo-antigens and patient HLA types.

FIG. 23A shows the timeline for B16F10 tumor experiments, including treatments with Lm Neo constructs.

FIG. 23B shows tumor regression with LmddA274, Lm-Neo-12, and Lm-Neo-20, with PBS used as a negative control.

FIG. 23C compares survival of mice with B16F10 tumors following treatment with LmddA274, Lm-Neo-12, or Lm-Neo-20, with PBS used as a negative control.

FIG. 24A-C show expression and secretion levels for PSA-Survivin-SIINFEKL (FIG. 24A), PSA-Survivin without SIINFEKL (FIG. 24B), and Neo 20-SIINFEKL (FIG. 24C).

FIG. 25 shows CD8 T-cell response to the Neo 20 antigen (with C-terminal SIINFEKL tag) or a negative control. The graph indicates the percent SIINFEKL-specific CD8 T-cell response for each condition.

FIG. 26A shows tumor regression with LmddA274, Lm-Neo-12, Lm-Neo-20, and Lm-Neo 30, with PBS used as a negative control.

FIG. 26B compares survival of mice with B16F10 tumors following treatment with LmddA274, Lm-Neo-12, Lm-Neo-20, and Lm-Neo 30, with PBS used as a negative control.

FIG. 27 shows an analysis of peptides from frameshift mutations in prostate adenocarcinoma (PRAD), pancreas adenocarcinoma (PAAD), breast invasive carcinoma (BRCA), ovarian serous cystadenocarcinoma (OV), and thyroid carcinoma (THCA).

FIG. 28 shows B16F10-tumor-bearing mice immunized with Lm constructs that secrete frameshift mutations (Frameshift 1 or Frameshift 2) derived from B16F10 tumor cells have decreased tumor growth compared to tumor bearing animals that were only treated with the empty vector negative control (LmddA-274). The Neo 12 construct was used as a positive control.

It will be appreciated that for simplicity and clarity of illustration, elements shown in the Figs. have not necessarily been drawn to scale. For example, the dimensions of some of the elements may be exaggerated relative to other elements for clarity. Further, where considered appropriate, reference numerals may be repeated among the Figs. to indicate corresponding or analogous elements.

### DETAILED DESCRIPTION

In the following detailed description, numerous specific details are set forth in order to provide a thorough understanding of the disclosure. However, it will be understood by those skilled in the art that the present disclosure may be practiced without these specific details. In other instances, well-known methods, procedures, and components have not been described in detail so as not to obscure the present disclosure.

Neo-antigens derive from mutations in tumor cell DNA (or other diseases or conditions) that result in nonsynonymous mutations. Most of these mutations result in single amino acid substitutions that can bind and be presented by MHC class I molecules for recognition by cytotoxic CD8+ T cells. In some cases, however, the insertion or deletion (indel) of one or two nucleotides can result in the production of frameshift mutations that encode polypeptides with entirely unique amino acid sequences that will be recognized as foreign by the host immune system and represent a rich source of potential neo-antigenic sequences. However, the use of these frameshift-derived polypeptide sequences for T cell targeted immunotherapies has limitations. One of these limitations is the limited level of translation associated with mRNA sequences derived from frameshift mutations. This is the result of a phenomenon known as nonsense-mediated decay, where mRNA sequences with early termination codons, which are generally present in frameshift mutations, are degraded after only one or two rounds of translation. Therefore, proteins derived from nucleotide sequences containing frameshift errors are produced in extremely limited quantities, severely limiting their availability for cross-priming of T cell responses to antigenic peptides that may be present in the frameshift-derived proteins. For this reason, only limited effort has been spent investigating frameshift-derived proteins as targets for T cell mediated immunotherapies.

T cell priming to antigens derived from proteins expressed in non-professional antigen presenting cells, such as most tumor cells, requires the transfer of sufficient quantities of protein to professional antigen presenting cells, such as dendritic cells. This process is termed cross-presentation, and T cell priming that results from cross-presentation is termed cross-priming Because nonsense-mediated decay limits translation of frameshift-associated sequences to only one or two rounds, the amount of protein available for cross-presentation and cross-priming is likely to be insufficient. As such, any immunotherapy that relies on endogenous T cell priming (e.g., checkpoint modulators, adoptive T cell therapies, and so forth) is unlikely to be effective for frameshift-derived antigens. However, the levels of protein expression required to present sufficient antigenic peptide on the surface of a cell to target it for destruction once a CD8+ T cell response has been primed is dramatically lower than that required for cross-priming Therefore, if the T cell priming event can be accomplished by introducing the frameshift-associated antigenic sequences using a recombinant expression system such as the Listeria platform disclosed herein, then it is possible to target frameshift-derived antigens expressed by tumor cells (see, e.g., Example 22 disclosed herein).

In one aspect, disclosed herein is an immunotherapy delivery vector comprising a nucleic acid comprising an open reading frame encoding a recombinant polypeptide comprising a PEST-containing peptide fused to one or more heterologous peptides, wherein the one or more heterologous peptides comprise one or more frameshift-mutation-derived peptides comprising one or more immunogenic neo-epitopes. Such immunotherapy delivery vectors can be, for example, a recombinant Listeria strain. The frameshift-mutation-derived peptides can be, for example, disease-specific or condition-specific.

In another aspect, disclosed herein is an immunogenic composition comprising at least one immunotherapy delivery vector disclosed herein. Such immunogenic compositions can further comprise, for example, an adjuvant.

In another aspect, disclosed herein is a method of treating, suppressing, preventing, or inhibiting a disease or a condition in a subject, comprising administering to the subject an immunotherapy delivery vector disclosed herein or an immunogenic composition disclosed herein, wherein the one or more frameshift-mutation-derived peptides are encoded by a source nucleic acid sequence from a disease-bearing or condition-bearing biological sample from the subject. Such methods can, for example, elicit a personalized anti-disease or anti-condition immune response in the subject, wherein the personalized immune response is targeted to the one or more frameshift-mutation-derived peptides.

In another aspect, disclosed herein is a process for creating a personalized immunotherapy for a subject having a disease or condition, comprising: (a) comparing one or more open reading frames (ORFs) in nucleic acid sequences extracted from a disease-bearing or condition-bearing biological sample from the subject with one or more ORFs in nucleic acid sequences extracted from a healthy biological sample, wherein the comparing identifies one or more nucleic acid sequences encoding one or more peptides comprising one or more immunogenic neo-epitopes encoded within the one or more ORFs from the disease-bearing or condition-bearing biological sample, wherein at least one of the one or more nucleic acid sequences comprises one or more frameshift mutations and encodes one or more frameshift-mutation-derived peptides comprising one or more immunogenic neo-epitopes; and (b) generating an immunotherapy delivery vector comprising a nucleic acid comprising an open reading frame encoding a recombinant polypeptide comprising the one or more peptides comprising the one or more immunogenic neo-epitopes identified in step (a). Optionally, such processes can further comprise storing the immunotherapy delivery vector or the DNA immunotherapy or the peptide immunotherapy for administering to the subject within a predetermined period of time. Optionally, such processes can further comprise administering a composition comprising the immunotherapy vector to the subject, wherein the administering results in the generation of a personalized T-cell immune response against the disease or condition.

In one embodiment, disclosed herein is a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of one or more nonsensical peptides or fragments thereof comprises one or more immunogenic neo-epitopes, and wherein the source is obtained from a disease or condition bearing biological sample of a subject. In another embodiment, the frameshift mutation is in comparison to a source nucleic acid sequence obtained from a healthy biological sample.

In another embodiment, said recombinant Listeria further comprises at least one nucleic acid sequence encoding one or more recombinant polypeptides comprising one or more peptides fused to an immunogenic polypeptide, wherein said one or more peptides comprise one or more immunogenic neo-epitopes. In another embodiment, said one or more peptides are sensical peptides.

In another embodiment, the disclosure relates to an immunotherapy delivery vector comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein said source is obtained from a disease or condition bearing biological sample of a subject.

In another embodiment, said immunotherapy delivery vector further comprises at least one nucleic acid sequence encoding one or more recombinant polypeptides comprising one or more peptides fused to an immunogenic polypeptide, wherein said one or more peptides comprise one or more immunogenic neo-epitopes. In another embodiment, said one or more peptides are sensical peptides.

In another embodiment, at least one frameshift mutation disclosed herein comprises multiple frameshift mutations and the multiple frameshift mutations are present within the same gene. In another embodiment, at least one frameshift mutation disclosed herein comprises multiple frameshift mutations and the multiple frameshift mutations are not present within the same gene.

In another embodiment, at least one frameshift mutation disclosed herein is within an exon encoding region of a gene. In another embodiment, the exon is the last exon of the gene. In another embodiment, one or more nonsensical peptide disclosed herein is expressed in the disease or condition bearing biological sample. In another embodiment, one or more nonsensical peptide disclosed herein does not encode a post-translational cleavage site. In another embodiment, the source nucleic acid sequence comprises one or more regions of microsatellite instability.

In another embodiment, one or more neo-epitopes disclosed herein comprises a T-cell epitope.

In another embodiment, one or more neo-epitopes disclosed herein comprises a cancer or tumor-associated neo-epitope. In another embodiment a cancer of tumor-associated neo-epitope comprises a self-antigen associated with the disease or condition, wherein the self-antigen comprises a cancer or tumor-associated neo-epitope, or a cancer-specific or tumor-specific neo-epitope. In another embodiment, one or more nonsensical peptides disclosed herein comprising one or more neo-epitopes, comprise an infectious disease-associated or disease specific neo-epitope.

In another embodiment, a recombinant Listeria disclosed herein expresses and secretes one or more recombinant polypeptides.

In another embodiment, one or more nonsensical peptides or fragments thereof disclosed herein are each fused to an immunogenic polypeptide. In another embodiment, one or more nonsensical peptides or fragments thereof disclosed herein comprise multiple operably linked nonsensical peptides or fragments thereof from N-terminal to C-terminal, wherein the immunogenic polypeptide is fused to one of the multiple nonsensical peptides or fragments thereof.

In another embodiment, one or more peptides or fragments thereof disclosed herein are each fused to an immunogenic polypeptide. In another embodiment, one or more peptides or fragments thereof disclosed herein comprise multiple operably linked peptides or fragments thereof from N-terminal to C-terminal, wherein the immunogenic polypeptide is fused to one of the multiple peptides or fragments thereof.

In one embodiment, a peptide disclosed herein is a sensical peptide. In another embodiment, a peptide is a nonsensical peptide.

In another embodiment, the immunogenic polypeptide is a mutated Listeriolysin O (LLO) protein, a truncated LLO (tLLO) protein, a truncated ActA protein, or a PEST amino acid sequence. The immunogenic polypeptide can comprise, for example, a PEST-containing peptide.

In another embodiment, one or more recombinant polypeptides disclosed herein is operably linked to a tag at the C-terminal, optionally via a linker sequence. In another embodiment, the tag is selected from a group comprising a 6× Histidine tag, SIINFEKL peptide, 6× Histidine tag operably linked to 6× histidine, and any combination thereof.

In another embodiment, the nucleic acid sequence encoding the recombinant polypeptide encodes components including: phly-tLLO-[nonsensical peptide or fragment thereof-glycine linker₍₄ₓ₎-nonsensical peptide or fragment thereof—glycine linker₍₄ₓ₎]ₙ-SIINFEKL-6× His tag-2× stop codon, wherein the nonsensical peptide or fragment thereof is about twenty-one amino acids long, and wherein n=1-20.

In another embodiment, the nucleic acid sequence encoding the recombinant polypeptide encodes components including: phly-tLLO-[peptide or fragment thereof-glycine linker₍₄ₓ₎-peptide or fragment thereof—glycine linker₍₄ₓ₎]ₙ-SIINFEKL-6× His tag-2× stop codon, wherein the peptide or fragment thereof is about twenty-one amino acids long, and wherein n=1-20.

In another embodiment, at least one nucleic acid sequence disclosed herein encoding a recombinant polypeptide disclosed herein is integrated into the Listeria genome. In another embodiment, at least one nucleic acid sequence encoding the recombinant polypeptide is in a plasmid.

In another embodiment, a Listeria strain disclosed herein is an attenuated Listeria strain. In another embodiment, the Listeria is Listeria monocytogenes.

In another embodiment, the attenuated Listeria disclosed herein comprises a mutation in one or more endogenous genes. In another embodiment, the endogenous gene mutation is selected from an actA gene mutation, a prfA mutation, an actA and inlB double mutation, a dal/dal gene double mutation, or a dal/dat/actA gene triple mutation, or a combination thereof.

In another embodiment, at least one nucleic acid sequence encoding the recombinant polypeptide further comprises a second open reading frame encoding a metabolic enzyme, or wherein the Listeria strain comprises a second nucleic acid sequence comprising an open reading frame encoding a metabolic enzyme. In another embodiment, the metabolic enzyme is an alanine racemase enzyme or a D-amino acid transferase enzyme.

In another embodiment, a nonsensical peptide disclosed herein is acquired by comparing one or more open reading frames (ORFs) in nucleic acid sequences extracted from the disease-bearing biological sample with one or more ORFs in nucleic acid sequences extracted from a healthy biological sample, wherein the comparison identifies one or more frameshift mutations within the nucleic acid sequences, wherein the nucleic acid sequence comprising the mutations encodes one or more nonsensical peptides comprising one or more immunogenic neo-epitopes encoded within one or more ORFs from the disease-bearing biological sample.

In another embodiment, a disease-bearing biological sample disclosed herein is obtained from the subject having a disease or condition. In another embodiment, a healthy biological sample is obtained from the subject having the disease or condition.

In another embodiment, the nonsensical peptide is characterized for neo-epitopes by: (i) generating one or more different peptide sequences from the nonsensical peptide; and optionally, (ii) screening each peptides generated in (i) and selecting for binding by MHC Class I complex or MHC Class II complex to which a T-cell receptor binds to.

In one embodiment, disclosed herein is an immunogenic composition comprising at least one of any one of the Listeria strains as described herein.

In another embodiment, the immunogenic composition as disclosed herein, further comprises an additional adjuvant.

In one embodiment, disclosed herein is a method of eliciting a personalized targeted immune response in a subject having a disease or condition, said method comprising administering to the subject an immunogenic composition as described herein, wherein the immune response is targeted to one or more nonsensical peptides or fragments thereof comprising one or more neo-epitopes present within a disease or condition bearing biological sample of a subject.

In one embodiment, disclosed herein is a method of treating, suppressing, preventing or inhibiting a disease or a condition in a subject, comprising administering to the subject an immunogenic composition as disclosed herein.

In one embodiment, disclosed herein is a method of increasing the ratio of T effector cells to regulatory T cells (Tregs) in the spleen and tumor of a subject, the method comprising the step of administering to the subject an immunogenic composition of as described herein, wherein the T effector cells are targeted to one or more nonsensical peptides comprising one or more neo-epitopes present within a disease or condition bearing biological sample of a subject.

In one embodiment, disclosed herein is a method for increasing neo-epitope-specific T-cells in a subject, the method comprising the step of administering to the subject an immunogenic composition as disclosed herein.

In one embodiment, disclosed herein is a method for increasing survival time of a subject having a tumor or suffering from cancer, or suffering from an infectious disease, the method comprising the step of administering to the subject an immunogenic composition as disclosed herein.

In one embodiment, disclosed herein is a method of reducing tumor or metastases size in a subject, the method comprising the step of administering to the subject an immunogenic composition as disclosed herein.

In another embodiment, the methods disclosed herein further comprise administering a booster treatment.

In another embodiment, the methods disclosed herein elicit a personalized enhanced anti-infectious disease immune response in the subject. In another embodiment, the method elicits a personalized anti-cancer or anti-tumor immune response.

### I. Personalized Immunotherapy

Disclosed herein are personalized immunotherapies such as recombinant Listeria strains. For example, such an immunotherapy delivery vector can comprise a nucleic acid comprising an open reading frame encoding a recombinant polypeptide comprising a PEST-containing peptide fused to one or more heterologous peptides, wherein the one or more heterologous peptides comprise one or more frameshift-mutation-derived peptides comprising one or more immunogenic neo-epitopes (e.g., T cell epitopes). One or more or all of the frameshift mutations can be disease-specific or condition-specific (i.e., present in a source nucleic acid sequence from a biological sample with the disease or condition but not in a source nucleic acid sequence from a healthy biological sample). The source nucleic acid sequence from the disease or condition can comprise, for example, one or more regions of microsatellite instability.

The immunotherapy delivery vector can be any suitable immunotherapy delivery vector, such as a DNA immunotherapy, a peptide immunotherapy, or a recombinant Listeria strain or other bacterial strain.

A frameshift mutation can be anywhere within a gene (e.g., a protein-coding gene). For example, a frameshift mutation can be in the penultimate exon or the last exon of a gene. The frameshift-mutation-derived peptide encoded by a frameshift mutation can be any length. For example, such a frameshift-mutation-derived peptide can be about 8-10, 11-20, 21-40, 41-60, 61-80, 81-100, 101-150, 151-200, 201-250, 251-300, 301-350, 351-400, 401-450, 451-500, or 8-500 amino acids in length. Some such frameshift-mutation-derived peptides do not encode a post-translational cleavage site.

The disease or condition can be any disease or condition comprising neo-epitopes. As an example, the disease or condition can be a cancer or tumor, and the one or more frameshift-mutation-derived peptides comprise a cancer-associated or tumor-associated neo-epitope or a cancer-specific or tumor-specific neo-epitope. For example, the one or more immunogenic neo-epitopes can comprise a self-antigen associated with the disease or condition, wherein the self-antigen comprises a cancer-associated or tumor-associated neo-epitope or a cancer-specific or tumor-specific neo-epitope. Examples of specific tumors or cancers are disclosed elsewhere herein. For example, a tumor or cancer can be a melanoma, lung cancer (e.g., lung squamous cell carcinoma, lung adenocarcinoma, small cell lung cancer), bladder cancer, stomach (gastric) cancer, esophageal cancer (e.g., esophageal adenocarcinoma), colorectal cancer, uterine cancer (endometrial cancer or cancer of the uterus), head and neck cancer, diffuse large B-cell lymphoma, glioblastoma multiforme, ovarian cancer, kidney cell cancer (renal cell carcinoma such as papillary renal cell carcinoma, clear cell renal cell carcinoma, and chromophobe renal cell carcinoma), multiple myeloma, pancreatic cancer, breast cancer, low-grade glioma, chronic lymphocytic leukemia, prostate cancer, neuroblastoma, carcinoid tumor, medulloblastoma, acute myeloid leukemia, thyroid cancer, acute lymphoblastic leukemia, Ewing sarcoma, or rhabdoid tumor. Similarly, a tumor or cancer can be a pancreatic cancer (e.g., pancreatic adenocarcinoma), prostate cancer (e.g., prostate adenocarcinoma), breast cancer (e.g., breast invasive carcinoma), ovarian cancer (e.g., ovarian serous cystadenocarcinoma), or a thyroid cancer (e.g., thyroid carcinoma). Other types of tumors or cancers are also possible. In some examples, the tumor is one with fewer than 120, 110, 100, 90, 80, 70, 60, 50, 40, 30, 20, or 10 tumor-associated or tumor-specific (i.e., not present in a healthy biological sample) nonsynonymous missense mutations, or the cancer is a type of cancer in which the mean or median number of tumor-associated or tumor-specific (i.e., not present in a healthy biological sample) nonsynonymous missense mutations across different patients is fewer than 120, 110, 100, 90, 80, 70, 60, 50, 40, 30, 20, or 10 nonsynonymous missense mutations, or the cancer is one such that at least 10%, 20%, 30%, 40%, 50%, 60%, 70%, 80%, 90%, 95%, or 100% of patients with that type of cancer have a tumor with fewer than 120, 110, 100, 90, 80, 70, 60, 50, 40, 30, 20, or 10 tumor-associated or tumor-specific (i.e., not present in a healthy biological sample) nonsynonymous missense mutations. As another example, the disease or condition can be an infectious disease. For example, the one or more frameshift-mutation-derived peptides comprise an infectious-disease-associated or infectious-disease-specific neo-epitope.

The recombinant polypeptide can comprise any number of neo-epitopes. For example, the recombinant polypeptide can comprise about 1-20 neo-epitopes. Other possibilities are disclosed elsewhere herein.

The one or more heterologous peptides can comprise multiple heterologous peptides. For example, they can comprise multiple heterologous peptides operably linked in tandem, wherein the PEST-containing peptide is fused to one of the multiple heterologous peptides. Likewise, the recombinant polypeptide can comprise multiple frameshift-mutation-derived peptides, wherein each frameshift-mutation-derived peptide is the same or different. Two peptides are different if they differ by at least one amino acid. In some case, the multiple heterologous peptides are operably linked to each other with no intervening sequence (e.g., fused directly to each other via peptide bonds). Alternatively, the multiple heterologous peptides can be operably linked to each other via one or more linkers, such as one or more peptide linkers or one or more 4× glycine linkers. Such linkers are disclosed elsewhere herein.

In some such recombinant polypeptides comprising multiple heterologous peptides, the PEST-containing peptide is operably linked to the N-terminal heterologous peptide. It can be linked directly with no intervening sequence (e.g., fused directly to each other via peptide bonds), or it can be linked via one or more linkers, such as one or more peptide linkers or one or more 4× glycine linkers. Such linkers are disclosed elsewhere herein. Examples of PEST-containing peptides include a mutated listeriolysin O (LLO) protein, a truncated LLO (tLLO) protein, a truncated ActA protein, or a PEST amino acid sequence. Other examples are disclosed elsewhere herein.

The recombinant polypeptide can further comprise one or more tags. The tag(s) can be at the N-terminal end, the C-terminal end, or anywhere within the recombinant polypeptide as disclosed elsewhere herein. For example, the C-terminal end of the recombinant polypeptide can be operably linked to a tag. It can be linked directly with no intervening sequence (e.g., fused directly to each other via peptide bonds), or it can be linked via one or more linkers, such as one or more peptide linkers or one or more 4× glycine linkers. Such linkers are disclosed elsewhere herein. Examples of tags include a 6× histidine tag, a 2× FLAG tag, a 3× FLAG tag, a SIINFEKL peptide, a 6× histidine tag operably linked to a SIINFEKL peptide, a 3× FLAG tag operably linked to a SIINFEKL peptide, a 2× FLAG tag operably linked to a SIINFEKL peptide, and any combination thereof.

Optionally, the open reading frame encoding the recombinant polypeptide comprises two stop codons at 3′ end (e.g., following the sequence encoding the tag. One example of such an open reading frame is operably linked to an hly promoter and encodes components comprising from N-terminus to C-terminus: tLLO-[heterologous peptide]ₙ-(peptide tag(s))-(2× stop codon), wherein n=2-20, and wherein at least one heterologous peptide is a frameshift-mutation-derived peptide. Another example of such an open reading frame is operably linked to an hly promoter and encodes components comprising from N-terminus to C-terminus: tLLO-[(heterologous peptide)-(glycine linker₍₄ₓ₎)]ₙ-(peptide tag(s))-(2× stop codon), wherein n=2-20, and wherein at least one heterologous peptide is a frameshift-mutation-derived peptide.

The one or more heterologous peptides can further comprise peptides that are not frameshift-mutation-derived peptides encoded by frameshift mutations. For example, the one or more heterologous peptides can further comprise one or more nonsynonymous-missense-mutation-derived peptides. As an example, the one or more heterologous peptides can further comprise one or more peptides encoded by a source nucleic acid sequence comprising at least one disease-specific or condition-specific nonsynonymous missense mutation. A nonsynonymous-missense-mutation-derived peptide can be of any length sufficient to elicit a positive immune response (e.g., sufficient to elicit a positive immune response using the Lm technology). For example, it can be about 5-50 amino acids in length, about 8-27 amino acids in length, or about 21 amino acids in length.

Some such immunotherapy delivery vectors comprise recombinant Listeria strains. Examples of variations of recombinant Listeria strains are disclosed elsewhere herein.

In one embodiment, disclosed herein is a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein the one or more nonsensical peptides are encoded by a source nucleic acid sequence comprising at least one frameshift mutation, wherein each of one or more nonsensical peptides or fragments thereof comprises one or more immunogenic neo-epitopes, and wherein the source is obtained from a disease or condition bearing biological sample of a subject.

In one embodiment, a nonsensical peptide comprises at least one immunogenic neo-epitope. In another embodiment, an immunogenic neo-epitope comprises an epitope that has not been previously recognized by the immune system. Neo-epitopes may be associated with tumor antigens and may be found in oncogenic cells. Neo-epitopes may be formed when a protein undergoes further modification within a biochemical pathway, such as glycosylation, phosphorylation or proteolysis. That is, by altering the structure of the protein or a portion thereof, a new or “neo” epitopes or neo-epitopes may be produced.

It will be understood by a skilled artisan that a peptide expressing a somatic mutation or mutations or sequence differences may comprise “neo-epitope.”

It will be further appreciated by a skilled artisan that the term “neo-epitope” may in one embodiment encompass an epitope that is not present in a reference sample, such as a normal non-cancerous or germline cell or tissue, wherein the neo-epitope is found in disease-bearing tissues, for example in a cancer cell. For example, a normal non-cancerous or germline cell may comprise an epitope; however, due to one or more mutations in a cancer cell, the sequence of the epitope is altered so as to result in an immunogenic neo-epitope. In another embodiment, a neo-epitope comprises a mutated epitope. In another embodiment, a neo-epitope has non-mutated sequence on either side of the epitope.

In another embodiment, a neo-epitope is immunogenic. In another embodiment at least one of the one or more neo-epitopes is immunogenic.

In another embodiment, one or more neo-epitopes disclosed herein is presented on an MHC I molecule. In another embodiment, one or more neo-epitopes is presented on a MHC II molecule. In yet another embodiment, one or more neo-epitopes is presented on both an MHC I molecule and an MHC II molecule.

In one embodiment, a neo-epitope is a linear epitope. In another embodiment, a neo-epitope is considered solvent-exposed and therefore accessible to T-cell antigen receptors. In another embodiment, a neo-epitope is a conformational epitope.

In another embodiment, a neo-epitope comprises a T-cell epitope. In another embodiment, a neo-epitope comprises an adaptive immune response epitope. In another embodiment, a neo-epitope is capable of leading to an induction of a T-cell immune response against the neo-epitope or an antigen comprising the same. In another embodiment, one or more neo-epitopes disclosed herein do not include immunosuppressive T-regulatory neo-epitopes. In a further embodiment, a source nucleic acid sequence encoding a nonsensical peptide or fragment thereof, which comprise one or more neo-epitopes, does not encode immunosuppressive epitopes.

In another embodiment, one or more immunogenic neo-epitopes disclosed herein show a score of up to 1.6 on a Kyte Doolittle hydropathy plot.

In another embodiment, a neo-epitope is associated with the disease or condition of the subject. In another embodiment, a neo-epitope is causative of the disease or condition of the subject. In another embodiment, a neo-epitope is present within the disease bearing biological sample. In another embodiment, a neo-epitope is present within the disease bearing biological tissue but is not causative or associated with the disease or condition. In another embodiment, a disease or condition comprises a cancer or tumor growth. In yet another embodiment, a disease or condition comprises an infectious disease or an autoimmune disease.

In another embodiment, the one or more nonsensical peptides comprising one or more immunogenic neo-epitopes, comprises a cancer or tumor-associated neo-epitope or a cancer or tumor-specific neo-epitope.

In another embodiment, an immunogenic neo-epitope or fragment thereof comprises at least a portion of an antigen, for example a Human Papilloma Virus (HPV)-16-E6 antigen, an HPV-16-E7 antigen, an HPV-18-E6 antigen, an HPV-18-E7 antigen, a Her/2-neu antigen, a chimeric Her2 antigen, a Prostate Specific Antigen (PSA), a bivalent PSA antigen, an ERG antigen, an Androgen receptor (AR) antigen, a PAK6 antigen, a Prostate Stem Cell Antigen (PSCA), a NY-ESO-1 antigen, a Stratum Corneum Chymotryptic Enzyme (SCCE) antigen, a Wilms tumor antigen 1 (WT-1), an HIV-1 Gag antigen, human telomerase reverse transcriptase (hTERT) antigen, a Proteinase 3 antigen, a Tyrosinase Related Protein 2 (TRP2) antigen, a High Molecular Weight Melanoma Associated Antigen (HMW-MAA), a synovial sarcoma antigen, a X (SSX)-2 antigen, a carcinoembryonic antigen (CEA), a Melanoma-Associated Antigen E (MAGE-A, MAGE 1, MAGE2, MAGE3, MAGE4), an interleukin-13 Receptor alpha (IL13-R alpha) antigen, a Carbonic anhydrase IX (CAIX) antigen, a survivin antigen, a GP100 antigen, an angiogenic antigen, a ras protein antigen, a p53 protein antigen, a p97 melanoma antigen, a KLH antigen, a MART1 antigen, a TRP-2 antigen, a HSP-70 antigen, a beta-HCG antigen, or a Testisin antigen.

In another embodiment, the HPV antigen is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, an HPV E6 antigen is utilized instead of or in addition to an E7 antigen in a composition or method disclosed herein for treating or ameliorating an HPV-mediated disease, disorder, or symptom. In another embodiment, an HPV-16 E6 and E7 is utilized instead of or in combination with an HPV-18 E6 and E7. In such an embodiment, the recombinant Listeria may express the HPV-16 E6 and E7 from the chromosome and the HPV-18 E6 and E7 from a plasmid, or vice versa. In another embodiment, the HPV-16 E6 and E7 antigens and the HPV-18 E6 and E7 antigens are expressed from a plasmid present in a recombinant Listeria disclosed herein. In another embodiment, the HPV-16 E6 and E7 antigens and the HPV-18 E6 and E7 antigens are expressed from the chromosome of a recombinant Listeria disclosed herein. In another embodiment, the HPV-16 E6 and E7 antigens and the HPV-18 E6 and E7 antigens are expressed in any combination of the above embodiments, including where each E6 and E7 antigen from each HPV strain is expressed from either the plasmid or the chromosome.

In another embodiment, one or more neo-epitopes disclosed herein comprise a self-antigen associated with a disease or condition, wherein the self-antigen comprises a cancer or tumor-associated neo-epitope, or a cancer-specific or tumor-specific neo-epitope. It will be appreciated by a skilled artisan that a cancer or tumor that may be treated by the compositions and methods disclosed herein need not be limited to the cancers or tumors disclosed herein but rather encompass any cancer or tumor, liquid or solid known in the art.

In another embodiment, one or more nonsensical peptides comprising one or more immunogenic neo-epitopes, comprises an infectious-disease-associated or a disease-specific neo-epitope. In another embodiment, an infectious disease disclosed herein comprises a viral or bacterial infection. In another embodiment, the infectious disease is caused by one of the following pathogens: leishmania, Entamoeba histolytica (which causes amebiasis), trichuris, BCG/Tuberculosis, Malaria, Plasmodium falciparum, plasmodium malariae, plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.

In one embodiment, the one or more neo-epitopes disclosed herein comprise at least a portion of a heterologous antigen disclosed herein. It will be appreciated by a skilled artisan that the term “heterologous” may encompass an antigen, or portion thereof, which is not naturally or normally expressed from a bacterium. In one embodiment, a heterologous antigen comprises an antigen not naturally or normally expressed from a Listeria strain.

It will be further appreciated by a skilled artisan that the term “heterologous” as disclosed herein, encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal.

It will be appreciated by a skilled artisan that the term “heterologous antigen” may be referred to herein as “antigenic polypeptide,” “heterologous protein,” “heterologous protein antigen,” “protein antigen,” “antigen fragment,” antigen portion,” “polypeptide,” “immunogenic polypeptide,” “nonsensical peptide,” “immunogenic neo-epitope,” “antigen,” and “neo-epitope,” or their grammatical equivalents and the like, and may encompass a polypeptide, a peptide, a nonsensical peptide or a recombinant peptide as described herein that is processed and presented on MHC class I and/or class II molecules present in a subject's cells leading to the mounting of an immune response when administered to said subject, or in another embodiment, detected by the host. In one embodiment, the antigen may be foreign to the host. In another embodiment, the antigen might be present in the host but the host does not elicit an immune response against it because of immunologic tolerance. In another embodiment, the antigen is a neo-antigen comprising one or more neo-epitopes.

In one embodiment, the disease disclosed herein is an infectious disease. In one embodiment, the infectious disease is one caused by, but not limited to, any one of the following pathogens: leishmania, Entamoeba histolytica (which causes amebiasis), trichuris, BCG/Tuberculosis, Malaria, Plasmodium falciparum, plasmodium malariae, plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In one embodiment, pathogenic protozoans and helminths infections include: amebiasis; malaria; leishmaniasis; trypanosomiasis; toxoplasmosis; pneumocystis carinii; babesiosis; giardiasis; trichinosis; filariasis; schistosomiasis; nematodes; trematodes or flukes; and cestode (tapeworm) infections.

In one embodiment an HPV antigen such as an E6 or E7 antigen disclosed herein is selected from an HPV 6 strain, and HPV 11 strain, HPV 16 strain, an HPV-18 strain, an HPV-31 strain, an HPV-35 strain, an HPV-39 strain, an HPV-45 strain, an HPV-51 strain an HPV-52 strain, an HPV-58 strain or an HPV-59 strain. In another embodiment, the HPV antigen is selected from a high-risk HPV strain. In another embodiment, the HPV strain is a mucosal HPV type. In another embodiment, HPV antigens can be selected from all HPV strains, including non-oncogenic HPVs such as type 6, 11, etc. that cause warts and dysplasias.

In another embodiment, the antigen is Her-2/neu. In another embodiment, the antigen is NY-ESO-1. In another embodiment, the antigen is LMP-1. In another embodiment, the antigen is carboxic anhydrase IX (CAIX). In another embodiment, the antigen is PSMA. In another embodiment, the antigen is HMW-MAA. In another embodiment, the antigen is HIV-1 Gag. In another embodiment, the antigen is PSA (prostate-specific antigen). In another embodiment, the antigen is a bivalent PSA. In another embodiment, the antigen is an ERG. In another embodiment, the antigen is an ERG construct type III. In another embodiment, the antigen is an ERG construct type VI. In another embodiment, the antigen is an androgen receptor (AR). In another embodiment, the antigen is a PAK6. In another embodiment, the antigen comprises an epitope rich region of PAK6. In another embodiment, the antigen is selected from NY-ESO-1, SCCE, HMW-MAA, EGFR-III, baculoviral inhibitor of apoptosis repeat-containing 5 (BIRCS), HIV-1 Gag, Muc1, PSA (prostate-specific antigen), or a combination thereof. In another embodiment, an antigen comprises the wild-type form of the antigen. In another embodiment, an antigen comprises a mutant form of the antigen.

In another embodiment, a Her-2 protein is a protein referred to as “HER-2/neu,” “Erbb2,” “v-erb-b2,” “c-erb-b2,” “neu,” or “cNeu.”

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein harbors 3 H2Dq and at least 17 of the mapped human MHC-class I epitopes of the Her2/neu antigen (fragments EC1, EC2, and IC1). In another embodiment, the chimeric protein harbors at least 13 of the mapped human MHC-class I epitopes (fragments EC2 and IC1). In another embodiment, the chimeric protein harbors at least 14 of the mapped human MHC-class I epitopes (fragments EC1 and IC1). In another embodiment, the chimeric protein harbors at least 9 of the mapped human MHC-class I epitopes (fragments EC1 and IC2).

In one embodiment, the antigen from which the nonsensical peptide disclosed herein is derived is from a fungal pathogen, helminth, or viruses. In other embodiments, the antigen from which the nonsensical peptide disclosed herein is derived is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens tyrosinase, MART-1,), human papilloma virus antigens E1 and E2 from type HPV-16, -18, -31, -33, -35 or -45 human papilloma viruses, mesothelin, or EGFRVIII.

In other embodiments, the nonsensical peptide is derived from an antigen that is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and listeriosis. In another embodiment, the condition disclosed herein is a dysplasia. In another embodiment, the disease is a neoplasia. In another embodiment, the disease is anal intraepithelial neoplasia (AIN). In another embodiment, the disease is vaginal intraepithelial neoplasia (VIN). In another embodiment, the disease is a cervical intraepithelial neoplasia (CIN).

In another embodiment, a condition disclosed herein is a pre-malignant condition or a condition that proceeds to develop into a disease, chronic or acute, if left untreated.

In another embodiment, the antigen from which the peptide disclosed herein is derived is a tumor-associated antigen, which in one embodiment, is one of the following tumor antigens: a ras peptide or p53 peptide associated with advanced cancers. Other tumor-associated antigens known in the art are also contemplated in the present disclosure.

In one embodiment, the nonsensical peptide is derived from a chimeric Her2 antigen described in U.S. Pat. No. 9,084,747, which is hereby incorporated by reference herein in its entirety.

It would be appreciated by a skilled artisan that an “immunogenic neo-epitope” is one that elicits an immune response when administered to a subject alone or in a composition or as part of a vaccine, as disclosed herein. Such a neo-epitope comprises the necessary epitopes in order to elicit either a humoral immune response, and/or an adaptive immune response. In one embodiment, the one or more immunogenic neo-epitopes comprised within one or more nonsensical peptides elicit a humoral immune response upon administration to a subject. In another embodiment, the one or more immunogenic neo-epitopes comprised within one or more nonsensical peptides elicit an adaptive immune response upon administration to a subject. In yet another embodiment, the one or more immunogenic neo-epitopes comprised within one or more nonsensical peptides elicit both a humoral immune response and an adaptive immune response upon administration to a subject.

In another embodiment, the neo-epitope sequences disclosed herein are tumor-specific, metastasis-specific, bacterial-infection-specific, viral-infection-specific, or any combination thereof. Additionally or alternatively, the neo-epitope sequences are inflammation-specific, immune-regulation-molecule-epitope-specific, T-cell-specific, an autoimmune-disease-specific, graft-versus-host disease (GvHD)-specific, or any combination thereof. In a further embodiment, the neo-epitope sequences are associated with a tumor, a cancer, a metastasis, a bacterial infection, a viral infection, an inflammation, an immune regulatory molecule, a T-cell, an autoimmune disease, or any combination thereof. Each possibility represents a separate embodiment of the present disclosure.

In another embodiment, candidate genes comprising neo-epitopes in a disease or condition bearing biological sample may include: Asteroid Homolog 1 (ASTE1), HNF1 Homeobox A (HNF1A), Family With Sequence Similarity 111, Member B (FAM111B), INO80E, chaperonin containing TCP1, subunit 8 (theta)-like 1 (CCT8L1), Globin Transcription Factor 1 (GAFA1), absent in melanoma 2 (AIM2), Synaptonemal Complex Protein 1 (SYCP1), Cysteine/Histidine-Rich 1(CYHR1), Guanylate Binding Protein 3 (GBP3), LOC100127950, LOC100131089, Tripartite Motif Containing 59 (TRIM59), 0-Linked N-Acetylglucosamine (GlcNAc) Transferase (OGT), D070, Fms-Related Tyrosine Kinase 3 Ligand (FLT3L), HPDMPK, Sec63, MAC30X TTK Protein Kinase TTK, Coiled-Coil Domain Containing 43 (CCDC43), Potassium Channel Tetramerization Domain Containing 16 (KCTD16), Mediator Complex Subunit 8 (MEDS), Emopamil Binding Protein-Like (EBPL), Signaling Lymphocytic Activation Molecule Family Member 1 (SLAMF1), SFRS112IP1, Fms-Related Tyrosine Kinase 3 Ligand (FLT3LG), Absent, Small, Or Homeotic)-Like 1 (ASH1L), Regulator Of G-Protein Signaling 22 (RGS22), GINS1, F-Box And Leucine-Rich Repeat Protein 3 (FBXL3), KIAA2018, Ankyrin Repeat Domain 49 (ANKRD49), BEN Domain Containing 5 (BENDS), Corepressor Interacting With RBPJ 1 (CIR1), Homeobox A11 (HOXA11), LOC643677, LOC100128175, Relaxin/Insulin-Like Family Peptide Receptor 2 (RXFP2), Excision Repair Cross-Complementation Group 1 (ERCCS), DNA (cytosine-5-)-methyltransferase 1 (DMT1), Protein tyrosine phosphatases (PTPs), Alstrom Syndrome Protein 1 (ALMS1), chromosome 6 open reading frame 89 (C6ORF89), fibronectin type III domain containing 3B (FNDC3B), beta receptor II (TGFβR2), transforming growth factor, beta receptor I (TGFβR1), Myristoylated alanine-rich C-kinase substrate-1 (MARCKS-1), Myristoylated alanine-rich C-kinase substrate-2 (MARCKS-2), Caudal Type Homeobox 2 (CDX2), TATA box-binding protein-associated factor 1B (TAF1B), Pecanex-Like 2 (PCNXL2/FLJ11383), Baxα+1, activin type 2 receptor (ACVR2), C14orf106/FLJ11186, caspase 5, Transcription Factor 7-Like 2 (TCF7L2/TCF-4), p21/ras, insulin-like growth factor II receptor (IGFIIR), human mismatch binding factor MutS Homolog 3 (hMSH3), or MutS Homolog 6 (hMSH6). Each possibility represents a separate embodiment of the present disclosure.

In another embodiment, the neo-epitope or a portion thereof may be encoded by at least a portion of a gene. In another embodiment, the neo-epitope or a portion thereof may be encoded by one or more of the genes candidates associated with a mutation in a tumor or cancer mentioned herein. Thus, the neo-epitope may be fully encoded by the gene or may be partially encoded by the gene.

In another embodiment, one or more neo-epitopes or a portion thereof may be encoded by at least a portion of a DNA mismatch repair gene. In another embodiment, one or more neo-epitopes may be encoded by at least a portion of a cell cycle regulation related gene. In another embodiment, one or more neo-epitopes may be encoded by at least a portion of an apoptosis regulation related gene. In another embodiment, one or more neo-epitopes may be encoded by at least a portion of an angiogenesis related gene. In another embodiment, one or more neo-epitopes may be encoded by at least a portion of a growth factor or growth factor receptor related gene. In another embodiment, one or more neo-epitopes may be encoded by genes comprising coding mononucleotide repeats (cMNR).

It will be appreciated by a skilled artisan that the term “genome” may encompass the total amount of genetic information in the chromosomes of an organism. It will also be appreciated by a skilled artisan that the term “exome” may encompass the coding regions of a genome, and the term “transcriptome” may encompass the set of all RNA molecules.

In another embodiment, neo-epitopes are determined using exome sequencing or transcriptome sequencing of a disease-bearing tissue or cell. In another embodiment, comparing the entire exome with a wild-type exome or an exome present in a non-disease-bearing tissue or cell in order identifies neo-epitopes. In another embodiment, a selected set of genes is compared to identify neo-epitopes. In another embodiment, the set of genes is tumor/cancer-type-specific, organ-specific, infectious-disease-specific, immune-condition-specific, or cellular-function-specific. In another embodiment, the set of genes comprises one or more genes selected from: apoptosis related genes, growth factor related genes, DNA mismatch repair related genes, cell cycle regulation related gene, and cMNR contacting genes. In certain embodiments, comparison is with genes presented as wild-type or from healthy tissues or cells.

In another embodiment, the set of genes compared between a disease bearing sample and a healthy sample for identifying neo-epitopes comprises any one or more of the genes mentioned herein. In still another embodiment, the set of genes compared between a disease bearing sample and a healthy sample for identifying nonsensical peptides comprising one or more neo-epitopes comprises any one or more of the genes mentioned herein.

In one embodiment, one or more neo-epitopes comprised in a nonsensical peptide are encoded by nucleic acid sequences comprising one or more nucleic acid sequence mutations in comparison to nucleic acid sequences present within a healthy sample. In another embodiment, one or more neo-epitopes are encoded by a nucleic acid sequence comprising an open reading frame (a gene exon). In another embodiment, the mutation is encoded within a gene exon. In another embodiment, the neo-epitope does not comprise a post-translational cleavage site.

In another embodiment, a mutation disclosed herein comprises an insertion of one or more nucleotides, a deletion of one or more nucleotides, a repeat expansion mutation, a duplication of one or more nucleotides, a substitution of one or more nucleotides, a frameshift mutation, and any combination thereof. In another embodiment, a neo-epitope disclosed herein is encoded by a sequence comprises at least one frameshift mutation.

A skilled artisan will appreciate that a nucleic acid disclosed herein may encompass deoxyribonucleic acid (DNA) or ribonucleic acid (RNA), more preferably RNA, most preferably in vitro transcribed RNA (Fv RNA) or synthetic RNA. Nucleic acids as disclosed herein, comprise genomic DNA, cDNA, mRNA, recombinantly produced and chemically synthesized molecules. In another embodiment, a nucleic acid may be present as a single-stranded or double-stranded and linear or covalently circularly closed molecule.

In another embodiment, a nucleic acid is isolated. A skilled artisan will appreciate that the term “isolated nucleic acid” may encompass a nucleic acid (i) that was amplified in vitro, for example via polymerase chain reaction (PCR), (ii) that was produced recombinantly by cloning, (iii) that was purified, for example, by cleavage and separation by gel electrophoresis, or (iv) that was synthesized, for example, by chemical synthesis. A nucleic may be employed for introduction into, i.e. transfection of, cells, in particular, in the form of RNA which can be prepared by in vitro transcription from a DNA template. The RNA may be modified before application by stabilizing sequences, capping, and polyadenylation.

It would be understood by a skilled artisan that the term “mutation” may encompass a change of or difference in the nucleic acid sequence (nucleotide substitution, addition or deletion, early termination or stop) compared to a reference sequence. For example a change or difference present in the biological sample obtained from a subject having a disease or condition, which is not found in healthy non-diseased biological sample.

A “somatic mutation” can occur in any of the cells of the body except the germ cells (sperm and egg) and therefore are not passed on to children. These alterations can (but do not always) cause cancer or other diseases or conditions. In one embodiment, a mutation is a nonsynonymous mutation. The term “nonsynonymous mutation” encompasses a mutation, preferably a nucleotide substitution, which results in an amino acid change such as an amino acid substitution in the translation product.

In the case of an abnormal or disease sample being a tumor or cancer tissue, in one embodiment, a mutation may comprise a “cancer mutation signature.” The term “cancer mutation signature” refers to a set of mutations which are present in cancer cells when compared to non-cancerous reference cells. Included are pre-cancerous or dysplastic tissue, and somatic mutations of same.

In one embodiment, frameshift mutations arise when the normal sequence of codons is disrupted by the insertion or deletion of one or more nucleotides, provided that the number of nucleotides added or removed is not a multiple of three. For instance, if just one nucleotide is deleted from the sequence, then all of the codons including and after the mutation will have a disrupted reading frame. This can result in the incorporation of many incorrect amino acids into the protein. In contrast, if three nucleotides are inserted or deleted, there will be no shift in the codon reading frame; however, there will be either one extra or one missing amino acid in the final protein. Therefore, frameshift mutations result in abnormal protein products with an incorrect amino acid sequence that can be either longer or shorter than the normal protein. Hence, it will be appreciated by a skilled artisan that a frameshift mutation disclosed herein may encompass a genetic mutation caused by a deletion or insertion in a nucleic acid sequence (e.g., DNA/RNA) that shifts the way that the sequence is read or the frame of the sequence that is read and such a mutation changes the amino acid sequence from the site of the mutation. In one embodiment, a nucleic acid comprising a frameshift mutation encodes a nonsensical amino acid sequence from the site of the mutation.

In an embodiment, the number of nucleic acid sequence mutations found in a disease or condition bearing sample in reference to a healthy sample may be in the range of about 1-20, 1-50, 1-80, 1-10², 1-10³, 1-10⁴ or 1-10⁵. Such mutations can be frameshift mutations, missense mutations, nonsynonymous missense mutations, or other types of mutations. For example, the number of frameshift mutations, the number of missense mutations, the number of nonsynonymous missense mutations, or the number of total mutations found in a disease or condition bearing sample in reference to a healthy sample may be in the range of about 1-20, 1-50, 1-80, 1-10², 1-10³, 1-10⁴ or 1-10⁵. In another embodiment, the number of nucleic acid mutations found in a disease or condition bearing sample in reference to a healthy sample may be in the range of about 1-10, 10-20, 20-40, 40-60, 60-80, 80-100, 100-150, 150-200, 200-300, 300-400, 400-500, 500-600, 600-700, 700-800, 800-1000, 1000-1500, 1500-5000, 5000-10000, or 10000-100000. Each possibility represents a separate embodiment of the present disclosure.

In another embodiment, the number of nucleic acid mutations found in a disease or condition bearing sample in reference to a healthy sample is about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 200, 300, 400, 500, 1000, 5000, 10000, 50000 or 100000. Such mutations can be frameshift mutations, missense mutations, nonsynonymous missense mutations, or other types of mutations. For example, the number of frameshift mutations, the number of missense mutations, the number of nonsynonymous missense mutations, or the number of total mutations found in a disease or condition bearing sample in reference to a healthy sample may be about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 200, 300, 400, 500, 1000, 5000, 10000, 50000 or 100000. Each possibility represents a separate embodiment of the present disclosure.

In another embodiment, the number of nucleic acid mutations found is correlated to tumor type. In another embodiment, the number of mutations discovered in a disease or condition bearing sample in comparison to a healthy sample serves as a checkpoint value rating the probability that amount of nucleic acid sequence mutations found is true.

It will be appreciated by a skilled artisan that an insertion or insertion mutation may encompass a change in the number of DNA bases in a nucleic acid sequence caused by an addition/insertion of at least one nucleic acid to the sequence. In another embodiment, an insertion or insertion mutation comprises a frameshift mutation. In another embodiment, the amino acid sequence encoded by the nucleic acid sequence does not function properly. In another embodiment, the amino acid sequence is comprised in a peptide or polypeptide. In another embodiment, the peptide or polypeptide comprises a nonsensical peptide.

It will be appreciated by a skilled artisan that a deletion or a deletion mutation may encompass a change in the number of DNA bases/nucleic acids caused by removal of at least one nucleic acid within a sequence. In another embodiment, deletions remove one or a few base pairs within a gene. In another embodiment, deletions remove an entire gene or several neighboring genes. In another embodiment, a deletion or a deletion mutation comprises a frameshift mutation. In another embodiment, the nucleic acid sequence including the deletion alters the function of the encoded amino acid sequence(s). In another embodiment, the amino acid sequence is comprised in a peptide or polypeptide. In another embodiment, the peptide or polypeptide comprises a nonsensical peptide.

It will be appreciated by a skilled artisan that a duplication or a duplication mutation may encompass duplication of at least one nucleic acid that is abnormally copied one or more times within a nucleic acid sequence. In another embodiment, a duplication or duplication mutation comprises a frameshift mutation. In another embodiment, the duplication mutation alters the function of the encoded amino acid sequence. In another embodiment, the amino acid sequence is comprised in a peptide or polypeptide. In another embodiment, the peptide or polypeptide comprises a nonsensical peptide.

It will be appreciated by a skilled artisan that a repeat expansion may encompass a mutation that increases the number of times that a short sequence is repeated. In another embodiment, a repeat expansion mutation comprises a frameshift mutation. In one embodiment, this type of mutation causes the encoded amino acid sequence to function improperly. In another embodiment, the amino acid sequence is comprised in a peptide or polypeptide. In another embodiment, the peptide or polypeptide comprises a nonsensical peptide.

It will be appreciated by a skilled artisan that a frameshift mutation encompasses a mutation that occurs when the addition or loss of DNA bases (nucleic acids) changes an encoding nucleic acid sequence reading frame, for example, an open reading frame (ORF). A reading frame consists of groups of three bases (a codon), wherein each codon codes for one amino acid. In one embodiment, a frameshift mutation shifts the grouping of these bases and changes the codon(s) encoding an amino acid sequence. In another embodiment, the resulting amino acid sequence is nonfunctional. In an alternative embodiment, the resulting amino acid sequence has partial functionality. In yet another embodiment, the resulting amino acid sequence is fully functional. In another embodiment, the amino acid sequence comprises a peptide or polypeptide. In another embodiment, a peptide or polypeptide that is nonfunctional or has partial functionality comprises a nonsensical peptide.

In another embodiment, frameshift mutations comprise nucleic acid sequences that are a consequence of a mutation or interruption of a splice site, a cancellation of a stop sequence providing read through of a nucleic acid sequence or providing gene fusions, insertion of at least one nucleic acid to the sequence, duplication or deletion or at least one nucleic acid, or a mutation leading to an alternative translation start site. Each possibility represents another embodiment of the present disclosure.

In one embodiment, a frameshift mutation is encoded within the nucleic acid sequence of at least one exon. In another embodiment, the frameshift mutation is encoded within the nucleic acid sequence of the last exon of a gene.

In another embodiment, a frameshift mutation encodes a nonsensical protein. In another embodiment, a frameshift mutation encodes a premature protein termination site. In another embodiment, the frameshift mutation changes the encoded amino acid sequence from the site of the frameshift mutation onward in the 3 prime direction (the C-terminal direction in the encoded amino acid sequence).

In another embodiment, an at least one frameshift mutation comprises multiple frameshift mutations. In another embodiment, the multiple frameshift mutations are present within the same gene. In another embodiment, the multiple frameshift mutations are not present within the same gene.

In another embodiment the frameshift mutation can be a result of microsatellite instability. In another embodiment, the frameshift is within microsatellite instability encoding regions.

A skilled artisan will appreciate that microsatellite instability (MSI) may encompass a change that occurs in the nucleic acid sequences of certain cells (such as tumor cells) in which the number of repeats of microsatellites (short, repeated sequences of nucleic acids) is different than the number of repeats that was in the nucleic acid sequence when it was inherited. In one embodiment, microsatellite instability comprises a defect in the ability to repair mistakes made when DNA is copied in the cell. In another embodiment, microsatellite instability comprises an instability affecting at least two, among the five, consensus mononucleotide repeats (BAT25, BAT26, NR21, NR22, and NR24) within tumor DNA, compared with normal colon DNA. In another embodiment, a nucleic acid sequence disclosed herein encompasses a nucleic acid sequence found in any tumor or cancer having microsatellite instability.

In another embodiment, the frameshift mutation is located within the last exon of a gene. In another embodiment, the frameshift mutation is encoded within the penultimate exon of a gene. It will be appreciated by a skilled artisan that some abnormal mRNAs with a premature termination codon resulting from frameshift mutation(s) are not subject to degradation by the non-sense-mediated mRNA decay (NMD) system. Other abnormal mRNAs with a premature termination codon resulting from frameshift mutation(s) are subject to degradation by the non-sense-mediated mRNA decay (NMD) system. In one embodiment, selecting of neo-epitopes further comprises selecting neo-epitopes and/or nonsensical peptides positioned in the last exon, or the penultimate exon. In one embodiment, the process further comprises eliminating neo-epitopes and/or nonsensical peptides derived from frameshift mutations encoded within the first exon, or any predefined upper limit of exons of a specific gene.

In another embodiment, the frameshift mutation is in comparison to a source nucleic acid sequence of a healthy biological sample.

In another embodiment, at least one frameshift mutation is within an exon encoding region of a gene. In another embodiment, the exon is the last exon of the gene.

In another embodiment, the number of frameshift mutations found in a sample is in the range of about 1-5, 5-10, 1-10, 10-20, 20-30, 20-40, 1-20, 1-40, 1-60, 40-60, 60-80, 80-100, 100-150, 150-200, 200-400, or 400-1000. In another embodiment, the number of frameshift mutations found in a sample is in the range of about 10³-10⁴. In another embodiment, the number of frameshift mutations found in a sample is up to about 5, 10, 20, 30, 40, 50, 60, 70, 80, 90, 100, 200, 300, 400, 500, 10³, 10⁴, or 10⁵. In another embodiment, the number of frameshift mutations in a sample less than about 5, 10, 20, 30, 40, 50, 60, 70, 80, 90, 100, 110, 120, 130, 140, 150, 200, 300, 400, 500, 600, 700, 800, 900, or 1000, or is more than about 5, 10, 20, 30, 40, 50, 60, 70, 80, 90, 100, 110, 120, 130, 140, 150, 200, 300, 400, 500, 600, 700, 800, 900, or 1000. Each possibility represents another embodiment of the present disclosure.

In another embodiment, the neo-epitope is generated from a nonsensical peptide sequence expressed consequent to a frameshift in the nucleic acid sequence.

A skilled artisan will appreciate that the term “nonsensical peptide” encompasses a peptide translated from a sequence harboring a frameshift mutation. At least a portion of or all of such a peptide is encoded by the sequence following a frameshift mutation. Equivalent terms include “frameshift-mutation-derived peptide” or “frameshift peptide.” In another embodiment, the nonsensical peptide comprises a novel amino acid sequence in comparison to healthy sample peptides. In another embodiment, the nonsensical peptide is at least partially functional. In another embodiment, the nonsensical peptide comprises a protein with at least one altered property. In another embodiment, the nonsensical peptide is a functional peptide. A “sensical peptide” is one that is not a nonsensical peptide (i.e., is not a frameshift-mutation-derived peptide and is not encoded by any sequence following a frameshift mutation).

In another embodiment, a vector comprising a nucleic acid sequence encodes the full-length nonsensical peptide. In another embodiment, the nucleic acid sequence encodes at least a fragment of the nonsensical peptide.

In another embodiment, the nonsensical peptide comprises a range of about 1-10 amino acids, 5-10 amino acids, 10-20 amino acids, 20-40 amino acids, 40-60 amino acids, 20-50 amino acids, 60-80 amino acids, 80-100 amino acids, 80-110 amino acids, 100-200 amino acids, 200-300 amino acids, 1-200 amino acids, 200-500 amino acids, 500-1000 amino acids, 1000-5000 amino acids, 5000-10000 amino acids, 1-10⁴ amino acids, or 1-10⁵ amino acids. Each possibility represents another embodiment of the present disclosure.

In another embodiment, each of one or more nonsensical peptides is about 60-100 amino acids in length. In another embodiment, each of the one or more nonsensical peptides can range from very short (e.g. about 10 amino acid sequences) to very long (e.g. over 100 amino acid sequences). In another embodiment, each of the one or more nonsensical peptides is about 8-10, 11-20, 21-40, 41-60, 61-80, 81-100, 101-150, 151-200, 201-250, 251-300, 301-350, 351-400, 401-450, 451-500, or 8-500 or more amino acids in length. For example, each nonsensical peptide can be about 10-450, 10-425, 10-400, 10-375, 10-350, 10-325, 10-300, 10-275, 10-250, 10-225, 10-200, 10-175, 10-150, 10-125, 10-100, 10-75, 10-50, 10-45, 10-40, 10-35, 10-30, 10-25, 10-20, 15-450, 15-425, 15-400, 15-375, 15-350, 15-325, 15-300, 15-275, 15-250, 15-225, 15-200, 15-175, 15-150, 15-125, 15-100, 15-75, 15-50, 15-45, 15-40, 15-35, 15-30, 15-25, or 15-20 amino acids in length. In some embodiments, each nonsensical peptide is at least about 10, 15, 20, 25, 30, 35, 40, 45, or 50 amino acids in length.

In another embodiment, the nonsensical peptide comprises up to about 5 amino acids, 6 amino acids, 8 amino acids, 10 amino acids, 20 amino acids, 30 amino acids, 40 amino acids, 50 amino acids, 60 amino acids, 70 amino acids, 80 amino acids, 100 amino acids, 150 amino acids, 10³, 10⁴, or 10⁵ amino acids. Each possibility represents a separate embodiment of the present disclosure.

In another embodiment, each neo-epitope amino acid sequence is about 21 amino acids in length or is a “21 mer” neo-epitope sequence. In another embodiment, one or more or each neo-epitope amino acid sequence is about 1-100, 5-100, 5-75, 5-50, 5-40, 5-30, 5-20, 5-15 or 5-10 amino acids in length. In yet another embodiment, one or more or each neo-epitope amino acid sequence is 1-100, 1-75, 1-50, 1-40, 1-30, 1-20, 1-15 or 1-10 amino acids in length. In yet another embodiment, one or more or reach neo-epitope amino acid sequence is about 8-11 or 11-16 amino acids in length.

In one embodiment, a neo-epitope is encoded by a nucleotide sequence comprising one mutation. In another embodiment, a neo-epitope is encoded by a nucleotide sequence comprising at least one mutation. In another embodiment, a neo-epitope is encoded by a nucleotide sequence comprising a plurality of mutations. In another embodiment, the mutation comprises an insertion mutation. In another embodiment, the mutation comprises a deletion mutation. In a further embodiment, the mutation comprises a duplication mutation. In another embodiment, the mutation comprises a repeat expansion mutation. In yet another embodiment, the mutation comprises a frameshift mutation.

In one embodiment, one or more neo-epitopes comprise between about 8 to about 27 amino acids each, 5 to 50 amino acids, 8 to 10 amino acids, or 8 to 12 amino acids. In another embodiment, one or more neo-epitope comprises about 21 amino acids each, 8 amino acids, or 27 amino acids. In another embodiment, one or more neo-epitope comprises about 5 amino acids each, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or 120 amino acids each. Each possibility represents a separate embodiment of the present disclosure.

In one embodiment a neo-epitope comprises about 5-30 amino acids flanking the nonsensical amino acid sequence, either N-terminally, C-terminally or both. In another embodiment, a neo-epitope comprises about 11 amino acids flanking each side of a nonsensical amino acid sequence. In another embodiment, a neo-epitope comprises about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids flanking on each side of the nonsensical amino acid sequence. Each possibility represents a separate embodiment of the present disclosure. In another embodiment, a neo-epitope comprises a mutation wherein about 1-50 amino acids are flanking on each side of the nonsensical amino acid sequence.

In one embodiment, a neo-epitope comprises about 5-30 or 1-50 amino acids of a frameshift-mutation-derived peptide or about 5-30 or 1-50 amino acids encoded by the sequence of a gene following a frameshift mutation. In another embodiment, a neo-epitope comprises about 11 amino acids of a frameshift-mutation-derived peptide or about 11 amino acids encoded by the sequence of a gene following a frameshift mutation. In another embodiment, a neo-epitope comprises about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids of a frameshift-mutation-derived peptide or encoded by the sequence of a gene following a frameshift mutation. Each possibility represents a separate embodiment of the present disclosure.

In some embodiments, a frameshift-mutation-derived peptide includes only sequence encoded by the gene sequence downstream of the frameshift mutation. In other embodiments, a frameshift-mutation-derived peptide further includes some amino acids encoded by the gene sequence upstream of the frameshift mutation (e.g., 1, 2, 3, 4, 5, 6, 7, 8, 9, 10, 11, 12, 13, 14, 15, 16, 17, 18, 19, 20, 1-50, 1-40, 1-30, 1-25, 1-20, 1-15, 1-10, or 1-5 amino acids encoded by the gene sequence upstream of the frameshift mutation).

For nonsynonymous-missense-mutation-derived peptides, a neo-epitope can comprise, for example, about 5-30 amino acids flanking the mutated amino acid encoded by the missense mutation, either N-terminally, C-terminally or both. In another embodiment, a neo-epitope comprises about 11 amino acids flanking each side of the mutated amino acid encoded by the missense mutation. In another embodiment, a neo-epitope comprises about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids flanking on each side of the mutated amino acid encoded by the missense mutation. Each possibility represents a separate embodiment of the present disclosure. In another embodiment, a neo-epitope comprises a nonsynonymous-missense-mutation-derived peptide wherein about 1-50 amino acids are flanking on each side of the mutated amino acid encoded by the missense mutation.

In another embodiment, the flanking sequences are symmetrical in amino acid length. For example, a nonsynonymous-missense-mutation-derived peptide can comprise a peptide encoded by the gene having the missense mutation, wherein the peptide comprises the mutated amino acid and flanking sequences encoded by the gene, wherein the flanking sequences are of equal length on each side. In another embodiment, the flanking sequences on each side are non-symmetrical in amino acid length. Additionally or alternatively, varying sizes of neo-epitope inserts are in the range of about 8-27 amino acid sequence long. Additionally or alternatively, varying sizes of neo-epitopes are inserted in the range of about 5-50 amino acid sequence long. Additionally or alternatively, varying sizes of neo-epitope inserts (i.e., a peptide encoding a neo-epitope) are inserted in the range of 10-30, 10-40, 15-30, 15-40, or 15-25 amino acids in length. In another embodiment each neo-epitope insert is 1-10, 10-20, 20-30, or 30-40 amino acids long. In another embodiment, the neo-epitope insert is 1-100, 5-100, 5-75, 5-50, 5-40, 5-30, 5-20, 5-15 or 5-10 amino acids long. In yet another embodiment, the neo-epitope amino acid sequence is 1-100, 1-75, 1-50, 1-40, 1-30, 1-20, 1-15 or 1-10. In another embodiment, each neo-epitope insert is 21 amino acids in length or is a “21-mer” neo-epitope sequence. In yet another embodiment, the neo-epitope amino acid insert is about 8-11 or 11-16 amino acids long.

In another embodiment, a neo-epitope comprises a completely novel sequence in comparison to a healthy biological sample or to the wild-type amino acid sequence. In another embodiment, the neo-epitope comprises an amino acid sequence at least partially different from the parallel sequence in a healthy sample. In another embodiment the neo-epitope comprises an amino acid sequence completely different from the parallel sequence in the healthy sample. In another embodiment, the identity between the neo-epitope and the parallel amino acid sequence from a healthy sample is in the range of about 0-99.999%. In another embodiment, the identity between the neo-epitope and the parallel amino acid sequence from a healthy sample is up to about 99%, 98%, 97%, 96%, 95%, up to 90%, 80%, 70%, 60%, 50%, 40%, 30%, 20%, 10%, 5%, or 1%. Each possibility represents a separate embodiment of the present disclosure.

In another embodiment, the nucleic acid sequence encoding a neo-epitope, encodes an amino acid sequence not affected by post translational proteolytic cleavage. In another embodiment, the nucleic acid sequence encoding a neo-epitope encodes an amino acid sequence affected by post translational ubiquitination and would be directed to the proteome for degradation. In another embodiment, the degraded protein portions may be displayed on the cells of a disease or condition bearing tissue.

A skilled artisan would recognize that the term “about” encompasses a deviance of between 0.0001-5% from the indicated number or range of numbers. In one embodiment, the term “about” comprises a deviance of between 1-10% from the indicated number or range of numbers. In one embodiment, the term “about” comprises a deviance of up to 25% from the indicated number or range of numbers.

In one embodiment, nonsensical peptides are selected and characterized for immunogenicity in order to identify immunogenic neo-epitopes. In another embodiment, nonsensical peptides selected comprise more than 5, 8, 10, 12, 15, 20, 30, 40, 50, 60, 70, 80, 90, or 100 amino acids. Each possibility represents a separate embodiment of the present disclosure.

In another embodiment, characterizing comprises generating all possible neo-epitopes amino acid sequences from the nonsensical peptide.

In one embodiment, the nonsensical peptide comprises at least one immunogenic neo-epitope. In another embodiment, the nonsensical peptide comprises neo-epitopes in the range of about 1 neo-epitope, 2, 3, 4, 5, 1-5, 5-10, 10-20, 20-30, 30-40, 40-50, 50-60, 60-70, 70-80, 80-90, 90-100, 100-200, 200-300, 300-500, 500-10³, or 10³-10⁴ neo-epitopes. Each possibility represents a separate embodiment disclosed herein.

In another embodiment, the nonsensical peptide comprises up to 5 neo-epitopes, 20 neo-epitopes, 50 neo-epitopes, 100 neo-epitopes, 150 neo-epitopes, 200 neo-epitopes, or 500 neo-epitopes. Each possibility represents a separate embodiment disclosed herein.

In another embodiment, the nonsensical peptide or fragment thereof is encoded by at least a fragment of a gene comprising one or more of the genes candidates for mutation in a tumor or cancer disclosed herein.

In another embodiment, the nonsensical peptide or fragment thereof is encoded by at least a fragment of a DNA mismatch repair gene. In another embodiment, the nonsensical peptide is encoded at least a fragment of a cell cycle regulation related gene. In another embodiment, the nonsensical peptide is encoded at least a fragment of an apoptosis regulation related gene. In another embodiment, the nonsensical peptide is encoded at least a fragment of an angiogenesis related gene. In another embodiment, the nonsensical peptide is encoded at least a fragment of a growth factor or growth factor receptor related gene. In another embodiment, the nonsensical peptide is encoded genes comprising coding mononucleotide repeats (cMNR). In another embodiment, the preset disclosure compares the entire exome to identify nonsensical peptide. In another embodiment, the present disclosure compares a selected set of genes to identify nonsensical peptide. In another embodiment, the set of genes is tumor/cancer type specific, organ specific, infectious disease specific, and immune condition specific or cellular function specific. In another embodiment, the set of genes comprises one or more genes selected from: apoptosis related genes, growth factor related genes, DNA mismatch repair related genes, cell cycle regulation related gene, and cMNR contacting genes. Each possibility represents a separate embodiment disclosed herein.

In another embodiment, the nucleic acid sequence encoding one or more neo-epitopes is expressed in the disease or condition-bearing biological sample. In another embodiment, the nucleic acid sequence encoding one or more nonsensical peptide is expressed in the disease- or condition-bearing biological sample. It would be appreciated by a skilled artisan that the term “expressed” encompasses a nucleic acid sequence transcribed and translated.

In one embodiment, the nonsensical peptide and/or neo-epitope is highly expressed in the disease or condition bearing sample cells. It will be appreciated by a skilled artisan that the term “highly expressed” encompasses expression levels higher than the median expression levels of the entire exome. In another embodiment, “highly expressed” comprises expression levels above the expression level of 10%, 20%, 30%, 40%, 50%, 60%, 70%, 80%, 90%, 95% of the genes expressed in a biological sample cell. Each possibility represents a separate embodiment as disclosed herein. In another embodiment, high expression levels comprise expression levels higher than the expression levels of one or more selected gene markers.

In one embodiment, the nonsensical peptide or fragment thereof, produced by a frameshift is transcribed and translated.

In another embodiment, the nonsensical peptide is identified from the comparison of one or more open reading frames (ORFs) in nucleic acid sequences extracted from the disease-bearing biological sample with one or more ORFs in nucleic acid sequences extracted from a healthy biological sample, wherein the comparison identifies one or more frameshift mutations within the nucleic acid sequences, wherein the nucleic acid sequence comprising the mutations encodes one or more nonsensical peptides comprising one or more immunogenic neo-epitopes encoded within one or more ORFs from the disease-bearing biological sample.

In another embodiment, the comparison comprises comparing open reading frame exome of a predefined gene-set selected from a group including: nucleic acid sequences encoding known and predicted cancer or tumor antigens, nucleic acid sequences encoding tumor or cancer-associated antigens, nucleic acid sequences encoding known or predicted tumor or cancer protein markers, nucleic acid sequences encoding known and predicted infectious disease or condition associated genes, nucleic acid sequences encoding genes expressed in the disease-bearing biological sample, nucleic acid sequences comprising regions of microsatellite instability, and any combination thereof.

In one embodiment, a recombinant Listeria strain disclosed herein comprises at least one nucleic acid sequence, wherein the nucleic acid sequence encodes one or more recombinant polypeptides comprising one or more nonsensical peptides or fragments thereof fused to an immunogenic polypeptide. An immunogenic polypeptide can be, for example, a PEST-containing peptide. In another embodiment, the Listeria strain expresses and secretes at least one or recombinant polypeptides comprising one or more nonsensical peptides or fragments thereof fused to an immunogenic polypeptide.

In another embodiment, the Listeria strain expresses and secretes one or more recombinant polypeptides comprising one or more nonsensical peptides or fragments thereof fused to an immunogenic polypeptide, during infection of the subject.

In another embodiment, each Listeria strain comprises a plurality of the nucleic acid sequences, each nucleic acid sequence encoding one or more recombinant polypeptides comprising one or more nonsensical peptides or fragments thereof fused to an immunogenic polypeptide. In another embodiment each Listeria strain comprises a nucleic acid sequence encoding one or more recombinant polypeptides, the recombinant polypeptide comprising one or more nonsensical peptides or fragments thereof fused to an immunogenic polypeptide.

In another embodiment, the nonsensical peptides are determined using exome sequencing or transcriptome sequencing of the disease-bearing tissue or cell. In another embodiment, the nonsensical peptide comprises a nucleic acid sequence encoding a neo-epitope comprising a selected amino acid sequence obtained partially or entirely from the nonsensical peptide. In another embodiment, one or more nonsensical peptides comprising the immunogenic epitopes, have a score of up to 1.6 on the Kyte Doolittle hydropathy plot.

In one embodiment, one or more neo-epitopes are encoded by a source nucleic acid sequence, wherein the source is obtained from a disease or condition bearing biological sample of a subject.

In another embodiment, a peptide, a polypeptide or a recombinant polypeptide as disclosed herein comprise one or more immunogenic neo-epitopes as disclosed herein.

In one embodiment, a recombinant polypeptide comprises a polypeptide encoded by a nucleic acid construct encoding one or more open reading frames encoding one or more polypeptides comprising at least one neo-epitope. In another embodiment, a recombinant polypeptide comprises a fusion polypeptide comprising at least one neo-epitope and at least one immunogenic polypeptide. The immunogenic polypeptide can be, for example, a PEST-containing peptide. In another embodiment, a recombinant polypeptide comprises a polypeptide encoded by a nucleic acid construct encoding one or more open reading frames encoding one or more nonsensical peptides or fragments thereof comprising at least one neo-epitope. In another embodiment, a recombinant polypeptide comprises a fusion polypeptide comprising one or more nonsensical peptides or fragments thereof fused to at least one immunogenic polypeptide.

In one embodiment, the source is obtained from a disease or condition bearing biological sample. In another embodiment, the nucleic acid sequence encoding the recombinant polypeptides disclosed herein is a plasmid insert. In an embodiment, the nucleic acid sequence is at least partially integrated into the genome. In another embodiment, the insert comprises a first open reading frame encoding the recombinant polypeptide. In another embodiment, the open reading frame comprises an immunogenic polypeptide or fragment thereof fused to one or more recombinant polypeptides comprising one or more neo-epitopes as disclosed herein.

In another embodiment, the nucleic acid sequence is in a plasmid within the recombinant Listeria strain. In another embodiment, the plasmid is an integrative plasmid. In another embodiment, the plasmid is an extrachromosomal multicopy plasmid. In another embodiment, the plasmid is stably maintained in the Listeria strain in the absence of antibiotic selection. In another embodiment, the plasmid does not confer antibiotic resistance upon the recombinant Listeria.

In another embodiment, the Listeria strain comprises the nucleic acid molecule comprising one or more neo-epitopes in a single location in the recombinant Listeria genome. In another embodiment, the Listeria strain comprises the nucleic acid molecule comprising one or more neo-epitopes in multiple locations in the Listeria genome.

In another embodiment, the Listeria strain comprises at least one nucleic acid molecule comprising one or more neo-epitopes in one plasmid. In another embodiment, the Listeria strain comprises neo-epitopes in at least two different plasmids, harbored in parallel in the recombinant Listeria strain. In another embodiment, the Listeria strain comprises neo-epitopes in a plurality of different plasmids, harbored in parallel in the recombinant Listeria strain. In another embodiment, the Listeria strain comprises neo-epitopes in one or more locations in the Listeria genome and in one or more different plasmids. The neo-epitopes in each can be the same or different.

In another embodiment, each of the Listeria expresses one or more recombinant polypeptides, each of the recombinant polypeptides comprising about 1-20 the neo-epitopes.

In another embodiment, determination of a number of constructs vs. mutational burden in each nucleic acid sequence is performed to determine efficiency of expression and secretion of neo-epitopes. In another embodiment, determining the amount of neo-epitopes per recombinant polypeptide is preformed to determine best three dimensional folding of the molecule in order to provide presentation of neo-epitopes as to T-cell receptors. In another embodiment, ranges of linear neo-epitopes are tested, starting with about 2, 5, 10, 20, 50, 100 epitopes per recombinant polypeptide or nucleic acid sequence. In another embodiment, ranges of linear neo-epitopes are tested, starting with about 1-5, 5-10, 10-20, 20-50, 50-70, 70-90, 90-110, 110-150, 150-200, 200-250, 300-350, or 400-500 epitopes per recombinant polypeptide or nucleic acid sequence. Each possibility represents a separate embodiment.

In another embodiment, the number of neo-epitopes per recombinant polypeptide, or the number of nucleic acid sequences encoding the recombinant polypeptides to be used, is determined considering the efficiency of translation and/or secretion of multiple epitopes from a single molecule, and or in reference to the number of neo-epitopes.

In another embodiment, the recombinant polypeptide comprises one neo-epitope. In another embodiment, the recombinant polypeptide comprises at least one neo-epitope, two neo-epitopes, 3 neo-epitopes, 4 neo-epitopes, 5 neo-epitopes, 6 neo-epitopes, 7 neo-epitopes, 8 neo-epitopes, 9 neo-epitopes, 10 or more neo-epitopes. In another embodiment, the recombinant polypeptide comprises about 11, 12, 13, 14, 15, 16, 17, 18, 20, 21, 22, 23, 24, 25, 26, 27, 28, 29, 30, 40, 50, 60, 70, 80, 90, or 100 neo-epitopes. In another embodiment, the recombinant polypeptide disclosed herein comprises about 40 neo-epitopes, 50 neo-epitopes, 1-10 neo-epitopes, 1-20, 1-30, 1-40, 1-50, 1-60, 1-70, 1-80, or 1-100 neo-epitopes.

In one embodiment, the recombinant polypeptide comprises at least one nonsensical peptide or fragment thereof. In one embodiment, the nucleic acid sequence encodes at least one nonsensical peptide or fragment thereof. In another embodiment, the recombinant polypeptide comprises at least two different neo-epitopes amino acid sequences. In another embodiment, the recombinant polypeptide comprises one or more neo-epitopes repeats of the same amino acid sequence.

In one embodiment the recombinant polypeptide comprises a plurality of the nonsensical peptides or fragments thereof. In one embodiment the nucleic acid sequence encodes a plurality of the nonsensical peptides or fragments thereof.

In one embodiment, the recombinant polypeptide comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nonsensical peptides or fragments thereof. In one embodiment the recombinant polypeptide comprises one or more nonsensical peptides or fragments thereof in the range of about 1-5, 1-10, 1-20, 1-50, 5-10, 10-20, 20-30, 30-40, 40-50, 50-60, 60-70, 70-80, 80-90, 90-100, 100-150, 150-200, or 200-500. In one embodiment the recombinant polypeptide comprises up to about 5, 10, 20, 30, 40, 50, 60, 70, 80, 90, 100, 150, 200, 250, 300, 350, 400, 450 or 500 nonsensical peptides or fragments thereof. Each possibility presents a separate embodiment.

In one embodiment the nucleic acid sequence encodes about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nonsensical peptides or fragments thereof.

In another embodiment the nucleic acid sequence encodes one or more nonsensical peptides or fragments thereof in range of about 1-5, 1-10, 1-20, 1-50, 5-10, 10-20, 20-30, 30-40, 40-50, 50-60, 60-70, 70-80, 80-90, 90-100, 100-150, 150-200, or 200-500. In another embodiment, the nucleic acid sequence encodes up to about 5, 10, 20, 30, 40, 50, 60, 70, 80, 90, 100, 150, 200, 250, 300, 350, 400, 450 or 500 nonsensical peptides or fragments thereof. In another embodiment, the nucleic acid sequence encodes more than about 5, 10, 20, 30, 40, 50, 60, 70, 80, 90, 100, 150, 200, 250, 300, 350, 400, 450 or 500 of the nonsensical peptides or fragments thereof. Each possibility presents a separate embodiment.

In another embodiment, the recombinant polypeptide comprises an immunogenic polypeptide. The immunogenic polypeptide can be, for example, a PEST-containing peptide. In another embodiment, the recombinant polypeptide comprises at least one immunogenic polypeptide. In another embodiment, the recombinant polypeptide comprises a plurality of immunogenic polypeptides. In another embodiment, the recombinant polypeptide comprises 1, 2, 3, 4, 5, 6, 7, 8, 9, or 10 immunogenic polypeptides.

In another embodiment, the recombinant polypeptide comprising one or more nonsensical peptides are each fused to an immunogenic polypeptide. For example, each of the one or more peptides can be fused to different immunogenic polypeptides or fragments thereof, or the combination of the one or more peptides can be fused to an immunogenic polypeptide or fragment thereof (e.g., an immunogenic polypeptide linked to a first neo-epitope, which is linked to a second neo-epitope, which is linked to a third neo-epitope, and so forth). In another embodiment, a plurality of nonsensical peptides is fused to at least one immunogenic polypeptide. For example, the one or more nonsensical peptides can be linked or fused to each other in tandem, with the N-terminal or C-terminal nonsensical peptide being linked or fused to the immunogenic polypeptide. In another embodiment, one nonsensical peptide is fused to an immunogenic polypeptide. In another embodiment at least one or more nonsensical peptide are fused to at least one immunogenic polypeptide. In another embodiment, the recombinant polypeptide comprises one or more peptides comprising one or more immunogenic nonsensical peptides operatively fused to an immunogenic polypeptide or fragment thereof. In another embodiment, the recombinant polypeptide comprises one or more nonsensical peptides operably linked from N-terminal to C-terminal, wherein the immunogenic polypeptide is fused to one of the one or more nonsensical peptides. In another embodiment, the immunogenic polypeptide is operably linked to the N-terminal nonsensical peptide. In another embodiment, the link is a peptide bond. In another embodiment, the recombinant polypeptide comprises one or more neo-epitopes or fragments thereof that are each fused to an immunogenic polypeptide.

In another embodiment, the recombinant polypeptide comprising one or more nonsensical peptides or fragments thereof comprises multiple operably linked nonsensical peptides or fragments thereof from N-terminal to C-terminal, wherein the immunogenic polypeptide is fused to one of the multiple nonsensical peptides or fragments thereof. In another embodiment, the immunogenic polypeptide is operably linked to the N-terminal nonsensical peptide. In another embodiment, the link is a peptide bond.

In another embodiment, the recombinant polypeptide comprises one or more nonsensical peptides, each nonsensical peptide is connected with a linker sequence to the following nonsensical peptide encoded on the same vector. In another embodiment, the linker is 4× glycine DNA sequence. In another embodiment the linker is a poly-glycine. It will be appreciated by a skilled artisan that other linker sequences known in the art may be used in the methods and compositions disclosed herein (see, e.g., Reddy Chichili, V. P., Kumar, V. and Sivaraman, J. (2013), Linkers in the structural biology of protein—protein interactions. Protein Science, 22: 153-167, which is incorporated by reference herein in its entirety). In yet another embodiment, the linker is selected from a group comprising SEQ ID NOS: 46-56 or any combination thereof.

In another embodiment different linker sequences are distributed between the nonsensical peptides for minimizing repeats. In another embodiment, distributing different linker sequences between the nonsensical peptides reduce secondary structures thereby allowing efficient transcription, translation, secretion, maintenance, or stabilization of the plasmid comprising the insert within the Lm recombinant vector strain population.

In another embodiment, the nucleic acid sequence encoding one or more recombinant polypeptide comprising one or more nonsensical peptides comprises one or more linker sequences incorporated between at least one first nonsensical peptide or fragment thereof and at least one second nonsensical peptides or fragment thereof. In another embodiment, the nucleic acid sequence comprises at least two different linker sequences incorporated between at least one first nonsensical peptide or fragment thereof and at least one second nonsensical peptides or fragment thereof to at least one third nonsensical peptides or fragment thereof.

In another embodiment, one or more linker(s) are selected from a group comprising nucleotide sequences as set forth in SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56. Each possibility represents a separate embodiment of the present disclosure.

In another embodiment, the immunogenic polypeptide is a mutated Listeriolysin O (LLO) protein, a truncated LLO (tLLO) protein, a truncated ActA protein, an ActA-PEST2 fusion, or a PEST amino acid sequence. The immunogenic polypeptide can comprise, for example, a PEST-containing peptide.

In another embodiment, the ActA-PEST2 fusion protein is set forth in SEQ ID NO: 17. In another embodiment, the tLLO protein is set forth in SEQ ID NO: 4. In another embodiment, the ActA is set forth in any one of SEQ ID NOS: 12-18 and 20-21. In another embodiment, the PEST amino acid sequence is selected from the sequences set forth in SEQ ID NOS: 6-11.

In another embodiment, the mutated LLO comprises a mutation in a cholesterol-binding domain (CBD). In another embodiment, the mutation comprises a substitution of residue C484, W491, or W492 of SEQ ID NO: 3, or any combination thereof.

In another embodiment, the final neo-epitope or the final nonsensical peptide encoded by a nucleic acid sequence is fused to a tag sequence followed by a stop codon. It will be appreciated by a skilled artisan that a tag may allow easy detection of the fusion polypeptide or chimeric protein during for example secretion from the Lm vector or when testing construct for affinity to specific T-cells, or presentation by antigen presenting cells.

In another embodiment, one or more recombinant polypeptide is operably linked to a tag at the C-terminal end, optionally via a linker sequence. In another embodiment, the linker sequence encodes a 4× glycine linker. In another embodiment the linker is as described herein.

In another embodiment, the tag sequence is an amino acid or nucleic acid sequence that allows for easy detection of the neo-epitope or the nonsensical peptide. In another embodiment, the tag sequence is an amino acid or nucleic acid sequence that is used for confirmation of secretion of a neo-epitope or nonsensical peptide disclosed herein. It will be appreciated by a skilled artisan that the sequences for the tags may be incorporated into the fusion peptide sequences on the plasmid or phage vector. These tags may be expressed and the antigenic epitopes presented allowing a clinician to follow the immunogenicity of the secreted recombinant polypeptides or nonsensical peptide by following immune responses to these “tag” sequence peptides. Such immune response can be monitored using a number of reagents including but not limited to, monoclonal antibodies and DNA or RNA probes specific for these tags.

In another embodiment, the tag is selected from a group including a 6× histidine tag, SIINFEKL peptide, 6× histidine tag operably linked to 6× histidine, a poly-histidine tag, and any combination thereof. In another embodiment the tag may be a C-terminal SIINFEKL-S-6× HIS tag. In another embodiment, the recombinant polypeptide disclosed herein, comprise any other tag know in the art, including, but not limited to chitin binding protein (CBP), maltose binding protein (MBP), and glutathione-S-transferase (GST), thioredoxin (TRX) and poly(NANP). In one embodiment the tag is selected from the group consisting of: a 6× histidine tag, a 2× FLAG tag, a 3× FLAG tag, a SIINFEKL peptide, a 6× histidine tag operably linked to a SIINFEKL peptide, a 3× FLAG tag operably linked to a SIINFEKL peptide, a 2× FLAG tag operably linked to a SIINFEKL peptide, and any combination thereof. Two or more tags can be used together, such as a 2× FLAG tag and a SIINFEKL tag, a 3× FLAG tag and a SIINFEKL tag, or a 6× His tag and a SIINFEKL tag. If two or more tags are used, they can be located anywhere within the recombinant polypeptide and in any order. For example, the two tags can be at the C-terminus of the recombinant polypeptide, the two tags can be at the N-terminus of the recombinant polypeptide, the two tags can be located internally within the recombinant polypeptide, one tag can be at the C-terminus and one tag at the N-terminus of the recombinant polypeptide, one tag can be at the C-terminus and one internally within the recombinant polypeptide, or one tag can be at the N-terminus and one internally within the recombinant polypeptide.

In another embodiment, the nucleic acid sequence disclosed herein, encodes any other tag know in the art, including, but not limited to chitin binding protein (CBP), maltose binding protein (MBP), a poly-histidine tag, SIINFEKL-S-6× HIS tag, 6× histidine tag, SIINFEKL peptide, and glutathione-S-transferase (GST), thioredoxin (TRX) and poly(NANP).

In another embodiment, the nucleic acid sequence comprises at least one sequence encoding a tag fused to the encoded nonsensical peptide. In another embodiment, the tag comprises the amino acid sequence as set forth in SEQ ID NO: 57.

In another embodiment, the nucleic acid sequence encoding one or more recombinant polypeptides comprises 2 stop codons following the sequence encoding the tag.

In another embodiment, the nucleic acid sequence encoding one or more recombinant polypeptide encodes components including: phly-tLLO-[nonsensical peptide or fragment thereof-glycine linker₍₄ₓ₎-nonsensical peptide or fragment thereof—glycine linker₍₄ₓ₎]ₙ-SIINFEKL-6× His tag-2× stop codon, wherein the nonsensical peptide or fragment thereof is about twenty-one amino acids long, and wherein n=1-20. In another embodiment, the nonsensical peptide or fragment thereof may be the same or different sequence represented in any of the n.

In another embodiment, the nucleic acid sequence encoding one or more recombinant polypeptide encodes components including: phly-tLLO-[neo-epitope-glycine linker₍₄ₓ₎-neo-epitope—glycine linker₍₄ₓ₎]ₙ-SIINFEKL-6× His tag-2× stop codon, wherein the neo-epitope is about twenty-one amino acids long, and wherein n=1-20. In another embodiment, the neo-epitope may be the same or different sequence represented in any of the n.

In another embodiment, the nucleic acid sequence encoding the recombinant polypeptide encodes components including: phly-tLLO-[neo-epitope/nonsensical peptide-glycine linker₍₄ₓ₎-neo-epitope/nonsensical peptide—glycine linker₍₄ₓ₎]ₙ-SIINFEKL-6× His tag-2× stop codon, wherein the neo-epitope/nonsensical peptide is about twenty-one amino acids long, and wherein n=1-20. In another embodiment, the neo-epitope/nonsensical peptide may be the same or different sequence represented in any of the n.

In another embodiment, n represents any integer. In another embodiment n may represent about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In another embodiment, 1≤n≤5, 1≤n≤10, 1≤n≤20, 1≤n≤30, 1≤n≤40, 1≤n≤50, 1≤n≤60, 1≤n≤70, 1≤n≤80, 1≤n≤90, 1≤n≤100, 1≤n≤200, 1≤n≤300, 1≤n≤400, 1≤n≤500, n≤5, n≤10, n≤20, n≤30, n≤40, n≤50, n≤60, n≤70, n≤80, n≤90, n≤100, n≤200, n≤300, n≤400, n≤500, n>5, n>10, n>20, n>30, n>40, n>50, n>60, n>70, n>80, n>90, n>100, n>200, n>300, n>400, or n>500. Each possibility represents a separate embodiment.

In one embodiment, disclosed herein is a nucleic acid construct encoding a recombinant polypeptide comprising the following elements: a PEST-containing peptide fused to a first neo-epitope amino acid sequence (e.g., frameshift-mutation-derived peptide), wherein the first neo-epitope sequence is operably linked to a second neo-epitope amino acid sequence (e.g., fused directly or via a linker sequence), wherein the second neo-epitope sequence is operably linked to at least one additional neo-epitope amino acid sequence (e.g., fused directly or via a linker sequence). Optionally, the PEST-containing peptide is an N-terminal truncated LLO (tLLO). Optionally, the last neo-epitope is operably linked to a tag (e.g., a 3× FLAG tag, a 2× FLAG tag, a 3× FLAG tag in combination with a SIINFEKL peptide, or a 2× FLAG tag in combination with a SIINFEKL peptide) at the C-terminus (e.g., fused directly or via a linker sequence). Optionally, the nucleic acid construct comprises at least 1 stop codon (e.g., 2 stop codons) following the sequence encoding the C-terminus (e.g., following the sequence encoding the tag). In another embodiment, at least one nucleic acid sequence construct encoding a recombinant polypeptide comprising the following elements: an N-terminal truncated LLO (tLLO) fused to a first nonsensical peptide amino acid sequence, wherein said first nonsensical peptide amino acid sequence is operably linked to a second nonsensical peptide amino acid sequence via a linker sequence, wherein said second nonsensical peptide amino acid sequence is operably linked to at least one additional nonsensical peptide amino acid sequence via a linker sequence, and wherein a last nonsensical peptide is operably linked to a histidine tag at the C-terminus via a linker sequence. In another embodiment, said elements are arranged or are operably linked from N-terminus to C-terminus. In another embodiment, each nucleic acid construct comprises at least 1 stop codon following the sequence encoding said 6× histidine (HIS) tag. In another embodiment, each nucleic acid construct comprises 2 stop codons following the sequence encoding said 6× histidine (HIS) tag. In another embodiment, said 6× histidine tag is operably linked at the N-terminus to a SIINFEKL peptide. In another embodiment, said linker is a 4× glycine linker. It would be appreciated by a skilled artisan that a construct disclosed herein may comprise a nonsensical peptide or fragment thereof, which comprises a neo-epitope. In another embodiment, a construct disclosed herein comprises a nonsensical peptide or fragment thereof, which consists of a neo-epitope.

In another embodiment, at least one nucleic acid sequence construct encodes a recombinant polypeptide, comprising an N-terminal truncated LLO fused to a 21 amino acid sequence of a nonsensical peptide flanked by a linker sequence and followed by at least one second neo epitope flanked by another linker and terminated by a SIINFEKL-6× His tag- and 2 stop codons closing the open reading frame: pHly-tLLO-21mer #1-4× glycine linker G1-21mer #2-4× glycine linker G2- . . . -SIINFEKL-6× His tag-2× stop codon. In another embodiment, expression of the above construct is driven by an hly promoter.

It would be appreciated by a skilled artisan that the term “abnormal,” “diseased,” or “unhealthy biological sample” encompasses and may be used interchangeably with “disease-bearing biological sample,” “disease-bearing sample,” or “disease or condition bearing biological sample.” In one embodiment, a biological sample is a tissue, cell(s), blood, sera, any sample obtained from a subject that comprises lymphocytes, any sample obtained from a subject that comprises disease-bearing cells, or any sample obtained from a subject that is healthy but is also comparable to a disease-bearing sample that is obtained from the same subject or similar individual. In another embodiment, the biological sample comprises a tissue, a cell, a blood sample, or a serum sample.

In one embodiment, an abnormal or unhealthy biological sample comprises a tumor tissue or a cancer tissue or a portion thereof. In another embodiment, a tumor or cancer may be a solid tumor. In another embodiment, a tumor or cancer is not a solid tumor or cancer, for example a blood cancer or a breast cancer wherein a tumor does not form. In another embodiment, the tumor or cancer is a liquid tumor or cancer.

In another embodiment, a tumor sample relates to any sample such as a bodily sample derived from a patient containing or being expected of containing tumor or cancer cells. The bodily sample may be any tissue sample such as blood, a tissue sample obtained from the primary tumor or from tumor metastases or any other sample containing tumor or cancer cells. In yet another embodiment, a bodily sample is blood, cells from saliva, or cells from cerebrospinal fluid. In another embodiment, a tumor sample relates to one or more isolated tumor or cancer cells such as circulating tumor cells (CTCs) or a sample containing one or more isolated tumor or cancer cells such as circulating tumor cells (CTCs).

In another embodiment, a tumor or cancer treated by administering a composition, vaccine, immunotherapy, or process disclosed herein comprises a breast cancer or tumor. In another embodiment, a tumor or a cancer comprises is a cervical cancer or tumor. In another embodiment, a tumor or a cancer comprises a Her2 containing tumor or cancer. In another embodiment, a tumor or a cancer comprises melanoma tumor or cancer. In another embodiment, a tumor or a cancer comprises a pancreatic tumor or cancer. In another embodiment, a tumor or a cancer comprises an ovarian tumor or cancer. In another embodiment, a tumor or a cancer comprises a gastric tumor or cancer. In another embodiment, a tumor or a cancer comprises a carcinomatous lesion of the pancreas. In another embodiment, a tumor or a cancer comprises a pulmonary adenocarcinoma tumor or cancer. In another embodiment, a tumor or a cancer comprises a glioblastoma multiforme tumor or cancer. In another embodiment, a tumor or a cancer comprises a colorectal adenocarcinoma tumor or cancer. In another embodiment, a tumor or a cancer comprises a pulmonary squamous adenocarcinoma tumor or cancer. In another embodiment, a tumor or a cancer comprises a gastric adenocarcinoma tumor or cancer. In another embodiment, a tumor or a cancer comprises an ovarian surface epithelial neoplasm (e.g. a benign, proliferative or malignant variety thereof) tumor or cancer. In another embodiment, a tumor or a cancer comprises an oral squamous cell carcinoma tumor or cancer. In another embodiment, a tumor or a cancer comprises a non-small-cell lung carcinoma tumor or cancer. In another embodiment, a tumor or a cancer comprises an endometrial carcinoma tumor or cancer. In another embodiment, a tumor or a cancer comprises a bladder tumor or cancer. In another embodiment, a tumor or a cancer comprises a head and neck tumor or cancer. In another embodiment, a tumor or a cancer comprises a prostate carcinoma tumor or cancer. In another embodiment, a tumor or a cancer comprises a gastric adenocarcinoma tumor or cancer. In another embodiment, a tumor or a cancer comprises an oropharyngeal tumor or cancer. In another embodiment, a tumor or a cancer comprises a lung tumor or cancer. In another embodiment, a tumor or a cancer comprises an anal tumor or cancer. In another embodiment, a tumor or a cancer comprises a colorectal tumor or cancer. In another embodiment, a tumor or a cancer comprises an esophageal tumor or cancer. In another embodiment, a tumor or a cancer comprises a mesothelioma tumor or cancer. Other suitable types of tumors or cancers include a melanoma, lung cancer (e.g., lung squamous cell carcinoma, lung adenocarcinoma, small cell lung cancer), bladder cancer, stomach (gastric) cancer, esophageal cancer (e.g., esophageal adenocarcinoma), colorectal cancer, uterine cancer (endometrial cancer or cancer of the uterus), head and neck cancer, diffuse large B-cell lymphoma, glioblastoma multiforme, ovarian cancer, kidney cell cancer (renal cell carcinoma such as papillary renal cell carcinoma, clear cell renal cell carcinoma, and chromophobe renal cell carcinoma), multiple myeloma, pancreatic cancer, breast cancer, low-grade glioma, chronic lymphocytic leukemia, prostate cancer, neuroblastoma, carcinoid tumor, medulloblastoma, acute myeloid leukemia, thyroid cancer, acute lymphoblastic leukemia, Ewing sarcoma, or rhabdoid tumor. Similarly, a tumor or cancer can be a pancreatic cancer (e.g., pancreatic adenocarcinoma), prostate cancer (e.g., prostate adenocarcinoma), breast cancer (e.g., breast invasive carcinoma), ovarian cancer (e.g., ovarian serous cystadenocarcinoma), or a thyroid cancer (e.g., thyroid carcinoma). Other types of tumors or cancers are also possible. In some examples, the tumor is one with fewer than 120, 110, 100, 90, 80, 70, 60, 50, 40, 30, 20, or 10 tumor-associated or tumor-specific (i.e., not present in a healthy biological sample) nonsynonymous missense mutations, or the cancer is a type of cancer in which the mean or median number of tumor-associated or tumor-specific (i.e., not present in a healthy biological sample) nonsynonymous missense mutations across different patients is fewer than 120, 110, 100, 90, 80, 70, 60, 50, 40, 30, 20, or 10 nonsynonymous missense mutations, or the cancer is one such that at least 10%, 20%, 30%, 40%, 50%, 60%, 70%, 80%, 90%, 95%, or 100% of patients with that type of cancer have a tumor with fewer than 120, 110, 100, 90, 80, 70, 60, 50, 40, 30, 20, or 10 tumor-associated or tumor-specific (i.e., not present in a healthy biological sample) nonsynonymous missense mutations.

In another embodiment, the disease-bearing biological sample is obtained from one location manifesting the disease or condition. In another embodiment, the disease-bearing biological sample is obtained from two different locations manifesting the disease or condition. In another embodiment, the disease-bearing biological sample is obtained from a range of about 2-5 different locations manifesting the disease or condition or about 2-10 different locations manifesting the disease or condition bearing tissue. In another embodiment, one disease-bearing biological sample is obtained from at least one primary tumor and at least a second sample is obtained from a metastasis. In another embodiment, a disease-bearing biological sample is obtained from a primary tumor. In another embodiment, a disease-bearing biological sample is obtained from a metastasis. In another embodiment, one disease-bearing biological sample is obtained from at least one metastasis and at least one second sample is obtained from a different metastasis. In another embodiment, at least one disease-bearing biological sample is obtained from at least one disease or condition bearing tissue and at least one second is obtained from blood or sera.

In another embodiment, an abnormal or unhealthy biological sample comprises non-tumor or cancerous tissue. In another embodiment, an abnormal or unhealthy biological sample comprises cells isolated from a blood sample, cells from saliva, or cells from cerebral spinal fluid. In another embodiment, an abnormal or unhealthy biological sample comprises a sample of any tissue or portion thereof that is considered abnormal or unhealthy.

In one embodiment, other non-tumor or non-cancerous diseases, comprising infectious diseases from which a disease-bearing biological sample can be obtained for analysis according to the process disclosed herein, are encompassed by the present disclosure. In another embodiment, an infectious disease comprises a viral infection. In another embodiment, an infectious disease comprises a chronic viral infection. In another embodiment, an infectious disease comprises a chronic viral illness such as HIV. In another embodiment, an infectious disease comprises a bacterial infection. In another embodiment, the infectious disease is a parasitic infection.

In one embodiment, pathogenic protozoans and helminths infections include: amebiasis; malaria; leishmaniasis; trypanosomiasis; toxoplasmosis; pneumocystis carinii; babesiosis; giardiasis; trichinosis; filariasis; schistosomiasis; nematodes; trematodes or flukes; and cestode (tapeworm) infections.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, rhodococcus equi, Tularemia, Plague (Yersinia pestis), trichomonas. Each possibility represents a separate embodiment of the present disclosure.

In one embodiment, other non-tumor or non-cancerous diseases comprise autoimmune diseases from which a disease-bearing biological sample can be obtained for analysis. It will be appreciated by the skilled artisan that the term “autoimmune disease” encompasses a disease or condition arising from immune reactions directed against an individual's own tissues, organs or manifestation thereof or resulting condition therefrom. It will be appreciated by the skilled artisan that the term “autoimmune disease” encompasses cancers and other disease states where the antibodies that are directed towards self-tissues are not necessarily involved in the disease condition but are still important in diagnostics. Further, in one embodiment, an autoimmune disease comprises a condition that results from, or is aggravated by, the production of autoantibodies by B cells of antibodies that are reactive with normal body tissues and antigens. In other embodiments, the autoimmune disease comprises a disease involving secretion of an autoantibody that is specific for an epitope from a self-antigen (e.g., a nuclear antigen).

Biological samples may be obtained using routine biopsy procedures well known in the art. Biopsies may comprise the removal of cells or tissues from a subject by skilled medical personnel, for example a pathologist. There are many different types of biopsy procedures. The most common types include: (1) incisional biopsy, in which only a sample of tissue is removed; (2) excisional biopsy, in which an entire lump or suspicious area is removed; and (3) needle biopsy, in which a sample of tissue or fluid is removed with a needle. When a wide needle is used, the procedure is called a core biopsy. When a thin needle is used, the procedure is called a fine-needle aspiration biopsy.

In one embodiment, a biological sample disclosed herein is obtained by incisional biopsy. In another embodiment, a biological sample is obtained by an excisional biopsy. In another embodiment, a biological sample is obtained using a needle biopsy. In another embodiment, a needle biopsy is a core biopsy. In another embodiment, a biopsy is a fine-needle aspiration biopsy. In another embodiment, a biological sample is obtained from as part of a blood sample. In another embodiment, a biological sample is obtained as part of a cheek swab. In another embodiment, a biological sample is obtained as part of a saliva sampling. In another embodiment, a biological sample comprises all or part of a tissue biopsy. In another embodiment, a tissue biopsy is taken and cells from that tissue sample are collected, wherein the cells comprise a biological sample of this disclosure. In another embodiment, a biological sample of this disclosure is obtained as part of a cell biopsy. In another embodiment, multiple biopsies may be taken from the same subject. In another embodiment, biopsies from the same subject may be collected from the same tissue or cells. In another embodiment, biopsies from the same subject may be collected from a different tissue of cell source within the subject.

In one embodiment, a biopsy comprises a bone marrow tissue. In another embodiment, a biopsy comprises a blood sample. In another embodiment, a biopsy comprises a biopsy of gastrointestinal tissue, for example esophagus, stomach, duodenum, rectum, colon and terminal ileum. In another embodiment, a biopsy comprises lung tissue. In another embodiment, a biopsy comprises prostate tissue. In another embodiment, a biopsy comprises liver tissue. In another embodiment, a biopsy comprises nervous system tissue, for example a brain biopsy, a nerve biopsy, or a meningeal biopsy. In another embodiment, a biopsy comprises urogenital tissue, for example a renal biopsy, an endometrial biopsy or a cervical conization. In another embodiment, a biopsy comprises a breast biopsy. In another embodiment, a biopsy comprises a lymph node biopsy. In another embodiment, a biopsy comprises a muscle biopsy. In yet another embodiment, a biopsy comprises a skin biopsy. In another embodiment, a biopsy comprises a bone biopsy. In another embodiment, a disease-bearing sample pathology of each sample is examined to confirm a diagnosis of the diseased tissue. In another embodiment, a healthy sample is examined to confirm a diagnosis of the health tissue.

In one embodiment, normal or a healthy biological sample is obtained from the subject. In another embodiment, the normal or healthy biological sample is a non-tumorigenous sample which relates to any sample such as a bodily sample derived from a subject. The sample may be any tissue sample such as healthy cells obtained from a biological sample disclosed herein. In another embodiment, the normal or healthy biological sample is obtained from another individual who in one embodiment is a related individual. In another embodiment, another individual is of the same species as the subject. In another embodiment, another individual is a healthy individual not containing or not being expected of containing a disease-bearing biological sample. In another embodiment, another individual is a healthy individual not containing or not being expected of containing tumor or cancer cells. It will be appreciated by a skilled artisan that the healthy individual may be screened using methods known in the art for the presence of a disease in order to determine that he or she is healthy. A disease-bearing biological sample and a healthy biological sample can both be obtained from the same tissue (e.g., a tissue section containing both tumor tissue and surrounding normal tissue). Preferably, healthy biological samples consist essentially or entirely of normal, healthy cells and can be used in comparison to a disease-bearing biological sample (e.g., a sample thought to comprise cancer cells or a particular type of cancer cells). Preferably, the samples are of the same type (e.g., both blood or both sera). For example, if the disease-bearing biological sample comprises cells, preferably the cells in the healthy biological sample have the same tissue origin as the disease-bearing cells in the disease-bearing biological sample (e.g., lung or brain) and arise from the same cell type (e.g., neuronal, epithelial, mesenchymal, hematopoietic).

In another embodiment, the normal or healthy biological sample is obtained at the same time as the disease-bearing biological sample. A skilled artisan would appreciate that the term “normal or healthy biological sample” encompasses the terms “reference sample” or “reference tissue” and may be used interchangeably throughout, having all the same meanings and qualities. In another embodiment, a reference sample is used to correlate and compare the results obtained in from a tumor specimen. In another embodiment, a reference sample is determined empirically by testing a sufficiently large number of normal specimens from the same species. In another embodiment, the normal or healthy biological sample is obtained at a different time, wherein the time may be such that the normal of healthy sample is obtained prior to obtaining the abnormal or unhealthy sample or afterwards. Methods of obtaining comprise those used routinely in the art for biopsy or blood collection. In another embodiment, a sample is a frozen sample. In another embodiment, a sample is comprised as a tissue paraffin embedded (FFPE) tissue block.

In another embodiment, the disease-bearing biological sample is obtained from the subject having the disease or condition. In another embodiment, the healthy biological sample is obtained from the subject having the disease or condition.

In one embodiment, following obtaining the normal or healthy biological sample, the sample is processed for extracting nucleic acids using techniques and methodologies well known in the art. In another embodiment, nucleic acids extracted comprise DNA. In another embodiment, nucleic acids extracted comprise RNA. In another embodiment, RNA is mRNA. In another embodiment, a next generation sequencing (NGS) library is prepared. Next-generation sequencing libraries may be constructed and may undergo exome or targeted gene capture. In another embodiment, a cDNA expression library is made using techniques known in the art, for example see US20140141992, which is hereby incorporated in full.

### II. Recombinant Listeria Strains

Provided herein are recombinant Listeria strains (e.g., Listeria monocytogenes) for use as personalized immunotherapy delivery vectors. For example, such recombinant Listeria strains can comprise a nucleic acid comprising an open reading frame encoding a recombinant polypeptide comprising a PEST-containing peptide fused to one or more heterologous peptides, wherein the one or more heterologous peptides comprise one or more frameshift-mutation-derived peptides comprising one or more immunogenic neo-epitopes. Such a recombinant Listeria strain can express and secrete the recombinant polypeptide. Different possibilities for each of these elements are as described for immunotherapy delivery vectors in general elsewhere herein.

In some such recombinant Listeria strains, the open reading frame encoding the recombinant polypeptide is integrated into the Listeria genome. Alternatively, the open reading frame encoding the recombinant polypeptide is in a plasmid. The plasmid can be, for example, stably maintained in the recombinant Listeria strain in the absence of antibiotic selection. It is also possible to have a recombinant Listeria strain comprising two such open reading frames—one genomically integrated into the Listeria genome, and one in a plasmid. The two open reading frames can be the same (i.e., encoding for the same recombinant polypeptide) or different (i.e., encoding for two different recombinant polypeptides).

The recombinant Listeria strain can be an attenuated Listeria strain. For example, it can comprise a mutation in one or more endogenous genes. Such a mutation can be selected from, for example, an actA gene mutation, a prfA mutation, an actA and inlB double mutation, a dal/dat gene double mutation, a dal/dat/actA gene triple mutation, or a combination thereof. The mutation can comprise, for example, an inactivation, truncation, deletion, replacement, or disruption of the gene or genes.

In some such recombinant Listeria strains, the nucleic acid comprising the open reading frame encoding the recombinant polypeptide further comprises a second open reading frame encoding a metabolic enzyme. Likewise, the recombinant Listeria strain can further comprise a second nucleic acid comprising an open reading frame encoding a metabolic enzyme. As an example, the metabolic enzyme can be an alanine racemase enzyme or a D-amino acid transferase enzyme.

As a specific example, the recombinant Listeria strain can be a recombinant Listeria monocytogenes strain comprising a deletion of or inactivating mutation in actA, dal, and dat, wherein the nucleic acid comprising the open reading frame encoding the recombinant polypeptide is in an episomal plasmid and comprises a second open reading frame encoding an alanine racemase enzyme or a D-amino acid aminotransferase enzyme, and wherein the PEST-containing peptide is an N-terminal fragment of LLO.

In one embodiment, disclosed herein is a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject.

In another embodiment, a recombinant Listeria strain disclosed herein comprises at least one nucleic acid sequence, the nucleic acid sequence comprising a first open reading frame encoding a fusion polypeptide, wherein the fusion polypeptide comprises a truncated listeriolysin O (tLLO) protein, a truncated ActA protein, or a PEST amino acid sequence fused to one or more nonsensical peptides comprising one or more neo-epitopes. It will be understood by a skilled artisan that one or more nonsensical peptides disclosed herein which comprise one or more neo-epitopes may be immunogenic to start with and their immunogenicity may be enhanced by fusing with or mixing with an immunogenic polypeptide such as a tLLO, a truncated ActA protein or a PEST amino acid sequence. Such an immunogenic polypeptide can be, for example, a PEST-containing peptide.

In one embodiment, a truncated listeriolysin O (LLO) protein comprises a putative PEST sequence. In one embodiment, a truncated ActA protein comprises a PEST-containing amino acid sequence. In another embodiment, a truncated ActA protein comprises a putative PEST-containing amino acid sequence.

In one embodiment, a PEST amino acid (AA) sequence comprises a truncated LLO sequence. In another embodiment, the PEST amino acid sequence comprises KENSISSMAPPASPPASPKTPIEKKHADEIDK (SEQ ID NO: 2). In another embodiment, fusion of an antigen to other LM PEST AA sequences from Listeria will also enhance immunogenicity of the nonsensical peptides. In another embodiment, fusion of a neo-epitope to other LM PEST AA sequences from Listeria will also enhance immunogenicity of the neo-peptides.

The N-terminal LLO protein fragment of methods and compositions disclosed herein comprises, in another embodiment, SEQ ID NO: 4. In another embodiment, the fragment comprises an LLO signal peptide. In another embodiment, the fragment comprises SEQ ID NO: 4. In another embodiment, the fragment consists approximately of SEQ ID NO: 4. In another embodiment, the fragment consists essentially of SEQ ID NO: 4. In another embodiment, the fragment corresponds to SEQ ID NO: 4. In another embodiment, the fragment is homologous to SEQ ID NO: 4. In another embodiment, the fragment is homologous to a fragment of SEQ ID NO: 4. In one embodiment, a truncated LLO used excludes of the signal sequence. In another embodiment, the truncated LLO comprises a signal sequence. It will be clear to those skilled in the art that any truncated LLO without the activation domain, and in particular without cysteine 484, are suitable for methods and compositions disclosed herein. In another embodiment, fusion of a heterologous antigen to any truncated LLO, including the PEST AA sequence, SEQ ID NO: 2, enhances cell mediated and anti-tumor immunity of the antigen. In another embodiment, fusion of a nonsensical peptide to any truncated LLO, including the PEST AA sequence, SEQ ID NO: 2, enhances cell mediated and anti-tumor immunity of the nonsensical peptide.

The LLO protein utilized to construct recombinant polypeptides disclosed herein has, in another embodiment, the sequence set forth in SEQ ID NO: 3 (GenBank Accession No. P13128; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a recombinant polypeptide or vaccine as disclosed herein.

In another embodiment, the N-terminal fragment of an LLO protein utilized in compositions and methods disclosed herein has the sequence set forth in SEQ ID NO: 4.

In another embodiment, the LLO fragment corresponds to about AA 20-442 of an LLO protein utilized herein.

In another embodiment, the LLO fragment has the sequence set forth in SEQ ID NO: 5.

It would be appreciated by a skilled artisan that the terms “N-terminal truncated LLO protein,” “N-terminal LLO fragment,” “truncated LLO protein,” “ALLO,” or their grammatical equivalents may be used interchangeably herein and encompass a fragment of LLO that is non-hemolytic. In another embodiment, the terms encompass an LLO fragment that comprises a putative PEST sequence.

In another embodiment, the LLO fragment is rendered non-hemolytic by deletion or mutation of the activation domain. In another embodiment, the LLO fragment is rendered non-hemolytic by deletion or mutation of region comprising cysteine 484. In another embodiment, the LLO is rendered non-hemolytic by a deletion or mutation of the cholesterol binding domain (CBD) as detailed in U.S. Pat. No. 8,771,702, which is incorporated by reference herein.

In one embodiment, a recombinant protein or polypeptide disclosed herein comprises a listeriolysin O (LLO) protein, wherein the LLO protein comprises a mutation of residues C484, W491, W492, or a combination thereof of the cholesterol-binding domain (CBD) of the LLO protein. In one embodiment, the C484, W491, and W492 residues are residues C484, W491, and W492 of SEQ ID NO: 3, while in another embodiment, they are corresponding residues as can be deduced using sequence alignments, as is known to one of skill in the art. In one embodiment, residues C484, W491, and W492 are mutated. In one embodiment, a mutation is a substitution, in another embodiment, a deletion. In one embodiment, the entire CBD is mutated, while in another embodiment, portions of the CBD are mutated, while in another embodiment, only specific residues within the CBD are mutated.

In another embodiment, the length of the LLO fragment of methods and compositions disclosed herein comprises at least 484 AA. In another embodiment, the length is over 484 AA. In another embodiment, the length is at least 489 AA. In another embodiment, the length is over 489. In another embodiment, the length is at least 493 AA. In another embodiment, the length is over 493. In another embodiment, the length is at least 500 AA. In another embodiment, the length is over 500. In another embodiment, the length is at least 505 AA. In another embodiment, the length is over 505. In another embodiment, the length is at least 510 AA. In another embodiment, the length is over 510. In another embodiment, the length is at least 515 AA. In another embodiment, the length is over 515. In another embodiment, the length is at least 520 AA. In another embodiment, the length is over 520. In another embodiment, the length is at least 525 AA. In another embodiment, the length is over 520. When referring to the length of an LLO fragment herein, the signal sequence is included. Thus, the numbering of the first cysteine in the CBD is 484, and the total number of AA residues is 529.

It would be appreciated by one skilled in the art that the terms “fusion peptide,” “fusion polypeptide,” “recombinant polypeptide,” “chimeric protein,” or “recombinant protein” encompass a peptide or polypeptide comprising two or more amino acid sequences, or two or more proteins, linked together by peptide bonds or other chemical bonds. In another embodiment, the proteins are linked together directly by a peptide or other chemical bond. In another embodiment, the proteins are linked together with one or more AA (e.g. a “spacer”) between the two or more proteins.

In another embodiment, a truncated LLO fragment comprises the first 441 AA of the LLO protein. In another embodiment, the LLO fragment comprises the first 420 AA of LLO. In another embodiment, the LLO fragment is a non-hemolytic form of the wild-type LLO protein.

In another embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly. In another embodiment, the LLO fragment is any other LLO fragment known in the art.

Methods for identifying corresponding residues of a homologous protein are well known in the art, and include, for example, sequence alignment. In one embodiment, a homologous LLO encompassed an LLO sequence disclosed herein of greater than 70%. In another embodiment, a homologous LLO encompasses an LLO sequence disclosed herein of greater than 72%. In another embodiment, a homologous LLO encompasses an LLO sequence disclosed herein of greater than 75%. In another embodiment, a homologous LLO encompasses an LLO sequence disclosed herein of greater than 78%. In another embodiment, a homologous LLO encompasses an LLO sequence disclosed herein of greater than 80%. In another embodiment, a homologous LLO encompasses an LLO sequence disclosed herein of greater than 82%. In another embodiment, a homologous LLO encompasses an LLO sequence disclosed herein of greater than 83%. In another embodiment, a homologous LLO encompasses an LLO sequence disclosed herein of greater than 85%. In another embodiment, a homologous LLO encompasses an LLO sequence disclosed herein of greater than 87%. In another embodiment, a homologous LLO encompasses an LLO sequence disclosed herein of greater than 88%. In another embodiment, a homologous LLO encompasses an LLO sequence disclosed herein of greater than 90%. In another embodiment, a homologous LLO encompasses an LLO sequence disclosed herein of greater than 92%. In another embodiment, a homologous LLO encompasses an LLO sequence disclosed herein of greater than 93%. In another embodiment, a homologous LLO encompasses an LLO sequence disclosed herein of greater than 95%. In another embodiment, a homologous LLO encompasses an LLO sequence disclosed herein of greater than 96%. In another embodiment, a homologous LLO encompasses an LLO sequence disclosed herein of greater than 97%. In another embodiment, a homologous LLO encompasses an LLO sequence disclosed herein of greater than 98%. In another embodiment, a homologous LLO encompasses an LLO sequence disclosed herein of greater than 99%. In another embodiment, a homologous LLO encompasses an LLO sequence disclosed herein of 100%.

A skilled artisan would appreciate that the terms “PEST amino acid sequence,” “PEST sequence,” “PEST sequence peptide,” “PEST peptide,” or “PEST sequence-containing protein or peptide” may be used interchangeably and may encompass a truncated LLO protein, which in one embodiment is an N-terminal LLO, or in another embodiment, a truncated ActA protein. PEST sequence peptides are known in the art and are described in U.S. Pat. No. 7,635,479, and in US Patent Publication No. 2014/0186387, both of which are hereby incorporated in their entirety herein.

In another embodiment, a PEST sequence of prokaryotic organisms can be identified routinely in accordance with methods such as described by Rechsteiner and Roberts (TBS 21:267-271, 1996) for L. monocytogenes. Alternatively, PEST amino acid sequences from other prokaryotic organisms can also be identified based by this method. Other prokaryotic organisms wherein PEST amino acid sequences would be expected to include, but are not limited to, other Listeria species. For example, the L. monocytogenes protein ActA contains four such sequences. These are KTEEQPSEVNTGPR (SEQ ID NO: 6), KASVTDTSEGDLDSSMQSADESTPQPLK (SEQ ID NO: 7), KNEEVNASDFPPPPTDEELR (SEQ ID NO: 8), and RGGIPTSEEFSSLNSGDFTDDENSETTEEEIDR (SEQ ID NO: 9). Also Streptolysin O from Streptococcus sp. contain a PEST sequence. For example, Streptococcus pyogenes Streptolysin 0 comprises the PEST sequence KQNTASTETTTTNEQPK (SEQ ID NO: 10) at amino acids 35-51 and Streptococcus equisimilis Streptolysin 0 comprises the PEST-like sequence KQNTANTETTTTNEQPK (SEQ ID NO: 11) at amino acids 38-54. Further, it is believed that the PEST sequence can be embedded within the antigenic protein. A skilled artisan would appreciate that as disclosed herein the term “fusion” when in relation to PEST sequence fusions, encompasses an antigenic protein comprising both the antigen, for example a nonsensical peptide, and the PEST amino acid sequence either linked at one end of the antigen or embedded within the antigen. In other embodiments, a PEST sequence or PEST containing polypeptide is not part of a fusion protein, nor does the polypeptide include a heterologous antigen.

A skilled artisan would appreciate that the terms “nucleic acid sequence,” “nucleic acid molecule,” “polynucleotide,” or “nucleic acid construct” may be used interchangeably herein and may encompass a DNA or RNA molecule, which may encompass, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also encompasses sequences that include any of the known base analogs of DNA and RNA. The terms may also encompass a string of at least two base-sugar-phosphate combinations. The term may also encompass the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The terms may also encompass artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid comprises any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

In another embodiment, a nucleic acid molecule disclosed herein is expressed from an episomal or plasmid vector. In another embodiment, the plasmid is stably maintained in the recombinant Listeria strain in the absence of antibiotic selection. In another embodiment, the plasmid does not confer antibiotic resistance upon the recombinant Listeria.

In one embodiment, an immunogenic polypeptide or fragment thereof disclosed herein is an ActA protein or fragment thereof. In one embodiment, an ActA protein comprises the sequence set forth in SEQ ID NO: 12.

The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 12 above. In another embodiment, an ActA polypeptide or peptide does not include the signal sequence, AA 1-29 of SEQ ID NO: 12 above.

In one embodiment, a truncated ActA protein comprises an N-terminal fragment of an ActA protein. In another embodiment, a truncated ActA protein is an N-terminal fragment of an ActA protein. In one embodiment, a truncated ActA protein comprises the sequence set forth in SEQ ID NO: 13.

In another embodiment, the ActA fragment comprises the sequence set forth in SEQ ID NO: 13.

In another embodiment, a truncated ActA protein comprises the sequence set forth in SEQ ID NO: 14.

In another embodiment, the ActA fragment is any other ActA fragment known in the art. In another embodiment, the ActA fragment is an immunogenic fragment.

In another embodiment, an ActA protein comprises the sequence set forth in SEQ ID NO: 15. The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 15. In another embodiment, an ActA polypeptide or peptide does not include the signal sequence, AA 1-29 of SEQ ID NO: 15.

In another embodiment, a truncated ActA protein comprises the sequence set forth in SEQ ID NO: 16. In another embodiment, a truncated ActA as set forth in SEQ ID NO: 16 is referred to as ActA/PEST1. In another embodiment, a truncated ActA comprises from the first 30 to amino acid 122 of the full length ActA sequence. In another embodiment, SEQ ID NO: 16 comprises from the first 30 to amino acid 122 of the full length ActA sequence. In another embodiment, a truncated ActA comprises from the first 30 to amino acid 122 of SEQ ID NO: 15. In another embodiment, SEQ ID NO: 16 comprises from the first 30 to amino acid 122 of SEQ ID NO: 15.

In another embodiment, a truncated ActA protein comprises the sequence set forth in SEQ ID NO: 17. In another embodiment, a truncated ActA as set forth in SEQ ID NO: 17 is referred to as ActA/PEST2. In another embodiment, a truncated ActA as set forth in SEQ ID NO: 17 is referred to as LA229. In another embodiment, a truncated ActA comprises from amino acid 30 to amino acid 229 of the full length ActA sequence. In another embodiment, SEQ ID NO: 17 comprises from about amino acid 30 to about amino acid 229 of the full length ActA sequence. In another embodiment, a truncated ActA comprises from about amino acid 30 to amino acid 229 of SEQ ID NO: 15. In another embodiment, SEQ ID NO: 17 comprises from amino acid 30 to amino acid 229 of SEQ ID NO: 15.

In another embodiment, a truncated ActA sequence disclosed herein is further fused to an hly signal peptide at the N-terminus. In another embodiment, the truncated ActA fused to hly signal peptide comprises SEQ ID NO: 18.

In another embodiment, a truncated ActA fused to hly signal peptide is encoded by a sequence comprising SEQ ID NO: 19. In another embodiment, SEQ ID NO: 19 comprises a sequence encoding a linker region (nucleotides 73-78 of SEQ ID NO: 19) that is used to create a unique restriction enzyme site for XbaI so that different polypeptides, heterologous antigens, etc. can be cloned after the signal sequence. Hence, it will be appreciated by a skilled artisan that signal peptidases act on the sequences before the linker region to cleave signal peptide.

In another embodiment, a truncated ActA protein comprises the sequence set forth in SEQ ID NO: 20. In another embodiment, a truncated ActA as set forth in SEQ ID NO: 20 is referred to as ActA/PEST3. In another embodiment, this truncated ActA comprises from the first 30 to amino acid 332 of the full length ActA sequence. In another embodiment, SEQ ID NO: 20 comprises from the first 30 to amino acid 332 of the full length ActA sequence. In another embodiment, a truncated ActA comprises from about the first 30 to amino acid 332 of SEQ ID NO: 15. In another embodiment, SEQ ID NO: 20 comprises from the first 30 to amino acid 332 of SEQ ID NO: 15.

In another embodiment, a truncated ActA protein comprises the sequence set forth in SEQ ID NO: 21. In another embodiment, a truncated ActA as set forth in SEQ ID NO: 21 is referred to as ActA/PEST4. In another embodiment, this truncated ActA comprises from the first 30 to amino acid 399 of the full length ActA sequence. In another embodiment, SEQ ID NO: 21 comprises from the first 30 to amino acid 399 of the full length ActA sequence. In another embodiment, a truncated ActA comprises from the first 30 to amino acid 399 of SEQ ID NO: 15. In another embodiment, SEQ ID NO: 18 comprises from the first 30 to amino acid 399 of SEQ ID NO: 15.

In another embodiment, “truncated ActA” or “ΔActA” encompass a fragment of ActA that comprises a PEST domain. In another embodiment, the terms encompass an ActA fragment that comprises a PEST sequence.

In another embodiment, the recombinant nucleotide encoding a truncated ActA protein comprises the sequence set forth in SEQ ID NO: 22.

In another embodiment, the recombinant nucleotide has the sequence set forth in SEQ ID NO: 22. In another embodiment, the recombinant nucleotide comprises any other sequence that encodes a fragment of an ActA protein.

In another embodiment, the ActA fragment consists of about the first 100 AA of the ActA protein.

In another embodiment, the ActA fragment consists of about residues 1-25. In another embodiment, the ActA fragment consists of about residues 1-50. In another embodiment, the ActA fragment consists of about residues 1-75. In another embodiment, the ActA fragment consists of about residues 1-100. In another embodiment, the ActA fragment consists of about residues 1-125. In another embodiment, the ActA fragment consists of about residues 1-150. In another embodiment, the ActA fragment consists of about residues 1-175. In another embodiment, the ActA fragment consists of about residues 1-200. In another embodiment, the ActA fragment consists of about residues 1-225. In another embodiment, the ActA fragment consists of about residues 1-250. In another embodiment, the ActA fragment consists of about residues 1-275. In another embodiment, the ActA fragment consists of about residues 1-300. In another embodiment, the ActA fragment consists of about residues 1-325. In another embodiment, the ActA fragment consists of about residues 1-338. In another embodiment, the ActA fragment consists of about residues 1-350. In another embodiment, the ActA fragment consists of about residues 1-375. In another embodiment, the ActA fragment consists of about residues 1-400. In another embodiment, the ActA fragment consists of about residues 1-450. In another embodiment, the ActA fragment consists of about residues 1-500. In another embodiment, the ActA fragment consists of about residues 1-550. In another embodiment, the ActA fragment consists of about residues 1-600. In another embodiment, the ActA fragment consists of about residues 1-639. In another embodiment, the ActA fragment consists of about residues 30-100. In another embodiment, the ActA fragment consists of about residues 30-125. In another embodiment, the ActA fragment consists of about residues 30-150. In another embodiment, the ActA fragment consists of about residues 30-175. In another embodiment, the ActA fragment consists of about residues 30-200. In another embodiment, the ActA fragment consists of about residues 30-225. In another embodiment, the ActA fragment consists of about residues 30-250. In another embodiment, the ActA fragment consists of about residues 30-275. In another embodiment, the ActA fragment consists of about residues 30-300. In another embodiment, the ActA fragment consists of about residues 30-325. In another embodiment, the ActA fragment consists of about residues 30-338. In another embodiment, the ActA fragment consists of about residues 30-350. In another embodiment, the ActA fragment consists of about residues 30-375. In another embodiment, the ActA fragment consists of about residues 30-400. In another embodiment, the ActA fragment consists of about residues 30-450. In another embodiment, the ActA fragment consists of about residues 30-500. In another embodiment, the ActA fragment consists of about residues 30-550. In another embodiment, the ActA fragment consists of about residues 1-600. In another embodiment, the ActA fragment consists of about residues 30-604.

In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly. In another embodiment, the ActA fragment is any other ActA fragment known in the art.

It will be appreciated by the skilled artisan that the term “homology,” when in reference to any nucleic acid sequence disclosed herein may encompass a percentage of nucleotides in a candidate sequence that is identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from the sequences disclosed herein of greater than 68%. In another embodiment, “homology” refers to identity to a sequence selected from the sequences disclosed herein of greater than 70%. In another embodiment, “homology” refers to identity to a sequence selected from the sequences disclosed herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7. 6), 5×Denhardt's solution, 10% dextran sulfate, and 20 μg/ml denatured, sheared salmon sperm DNA.

In one embodiment, the recombinant Listeria strain disclosed herein lacks antibiotic resistance genes.

In one embodiment, the recombinant Listeria disclosed herein is capable of escaping the phagolysosome. In one embodiment, the recombinant Listeria disclosed herein is capable of escaping the phagosome.

In another embodiment, the endogenous gene mutation comprised in a Listeria strain disclosed herein, is selected from an actA gene mutation, a prfA mutation, an actA and inlB double mutation, a dal/dal gene double mutation, or a dal/dat/actA gene triple mutation, or a combination thereof.

In one embodiment, the Listeria genome comprises a deletion of the endogenous actA gene, which in one embodiment is a virulence factor. In one embodiment, the heterologous antigen or antigenic polypeptide is integrated in frame with LLO in the Listeria chromosome. In another embodiment, the integrated nucleic acid molecule is integrated in frame with ActA into the actA locus. In another embodiment, the chromosomal nucleic acid encoding ActA is replaced by a nucleic acid molecule encoding an antigen.

In one embodiment, a recombinant Listeria disclosed herein comprises a nucleic acid molecule comprising a first open reading frame encoding recombinant polypeptide comprising one or more nonsensical peptides, wherein the one or more nonsensical peptides comprise one or more neo-epitopes. In another embodiment, the recombinant polypeptide further comprises a truncated LLO protein, a truncated ActA protein or PEST sequence fused to a nonsensical peptide or a fragment thereof as disclosed herein.

In another embodiment, a bacterial signal sequence disclosed herein is a Listerial signal sequence, which in another embodiment, is an hly or an actA signal sequence. In another embodiment, the bacterial signal sequence is any other signal sequence known in the art.

In one embodiment, nucleic acids encoding recombinant polypeptides disclosed herein also comprise a signal peptide or signal sequence. In one embodiment, the bacterial secretion signal sequence encoded by a nucleic acid constructs or nucleic acid molecule disclosed herein is a Listeria secretion signal sequence. In another embodiment, a fusion protein of methods and compositions of the present disclosure comprises an LLO signal sequence from Listeriolysin O (LLO). It will be appreciated by a skilled artisan that an antigen or a peptide comprising one or more neo-epitopes disclosed herein may be expressed through the use of a signal sequence, such as a Listerial signal sequence, for example, the hemolysin (hly) signal sequence or the actA signal sequence. Alternatively, for example, foreign genes can be expressed downstream from a L. monocytogenes promoter without creating a fusion protein. In another embodiment, the signal peptide is bacterial (Listerial or non-Listerial). In one embodiment, the signal peptide is native to the bacterium. In another embodiment, the signal peptide is foreign to the bacterium. In another embodiment, the signal peptide is a signal peptide from Listeria monocytogenes, such as a secA1 signal peptide. In another embodiment, the signal peptide is an Usp45 signal peptide from Lactococcus lactis, or a Protective Antigen signal peptide from Bacillus anthracia. In another embodiment, the signal peptide is a secA2 signal peptide, such the p60 signal peptide from Listeria monocytogenes. In addition, the recombinant nucleic acid molecule optionally comprises a third polynucleotide sequence encoding p60, or a fragment thereof. In another embodiment, the signal peptide is a Tat signal peptide, such as a B. subtilis Tat signal peptide (e.g., PhoD). In one embodiment, the signal peptide is in the same translational reading frame encoding the recombinant polypeptide.

In another embodiment, the secretion signal sequence is from a Listeria protein. In another embodiment, the secretion signal is an ActA₃₀₀ secretion signal. In another embodiment, the secretion signal is an ActA₁₀₀ secretion signal.

In one embodiment, a nucleic acid molecule disclosed herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme complements an endogenous gene that is mutated in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains disclosed herein comprise a mutation in the endogenous dal/dat genes. In another embodiment, the Listeria lacks the dal/dat genes.

In another embodiment, a nucleic acid molecule of the methods and compositions disclosed herein operably linked to a promoter/regulatory sequence. In another embodiment, the first open reading frame of methods and compositions disclosed herein is operably linked to a promoter/regulatory sequence. In another embodiment, the second open reading frame of methods and compositions disclosed herein is operably linked to a promoter/regulatory sequence. In another embodiment, each of the open reading frames are operably linked to a promoter/regulatory sequence.

A skilled artisan would appreciate that the term “metabolic enzyme” may encompass an enzyme involved in synthesis of a nutrient required by the host bacteria. In one embodiment, the term encompasses an enzyme required for synthesis of a nutrient required by the host bacteria. In another embodiment, the term encompasses an enzyme involved in synthesis of a nutrient utilized by the host bacteria. In another embodiment, the term encompasses an enzyme involved in synthesis of a nutrient required for sustained growth of the host bacteria. In another embodiment, the enzyme is required for synthesis of the nutrient.

In another embodiment, the recombinant Listeria is an attenuated auxotrophic strain.

In one embodiment the attenuated strain is Lm dal(−)dat(−) (Lmdd). In another embodiment, the attenuated strains is Lm dal(−)dat(−)ΔactA (LmddA). LmddA is based on a Listeria vaccine vector which is attenuated due to the deletion of virulence gene actA and retains the plasmid for a desired heterologous antigen or truncated LLO expression in vivo and in vitro by complementation of dal gene.

In another embodiment, the attenuated strain is LmddA. In another embodiment, the attenuated strain is LmΔactA. In another embodiment, the attenuated strain is LmΔPrfA. In another embodiment, the attenuated strain is LmΔPrfA*. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of Listeria-based vaccines. In another embodiment, this strain is constructed from the EGD Listeria backbone. In another embodiment, the strain disclosed herein is a Listeria strain that expresses a non-hemolytic LLO.

In another embodiment, the Listeria strain is deficient in a gene encoding a vitamin synthesis gene. In another embodiment, the Listeria strain is deficient in a gene encoding pantothenic acid synthase.

In one embodiment, the generation of strains of Listeria disclosed herein deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as disclosed herein, may be used as targets for mutagenesis of Listeria.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, the metabolic enzyme is an amino acid metabolism enzyme. In another embodiment, the metabolic enzyme catalyzes a formation of an amino acid used for a cell wall synthesis in the recombinant Listeria strain. In another embodiment, the metabolic enzyme is an alanine racemase enzyme. In another embodiment, the metabolic enzyme is a D-amino acid transferase enzyme.

In one embodiment, the auxotrophic Listeria strain comprises an episomal expression vector comprising a metabolic enzyme that complements the auxotrophy of the auxotrophic Listeria strain. In another embodiment, the construct is contained in the Listeria strain in an episomal fashion. In another embodiment, the foreign antigen is expressed from a plasmid vector harbored by the recombinant Listeria strain. In another embodiment, the episomal expression plasmid vector lacks an antibiotic resistance marker. In one embodiment, an antigen of the methods and compositions as disclosed herein is fused to a polypeptide comprising a PEST sequence.

In another embodiment, the Listeria strain is deficient in an amino acid (AA) metabolism enzyme. In another embodiment, the Listeria strain is deficient in a D-glutamic acid synthase gene. In another embodiment, the Listeria strain is deficient in the dat gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described herein.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc.).

In another embodiment, the gene is phoP. In another embodiment, the gene is aroA. In another embodiment, the gene is aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the proteins disclosed herein.

In one embodiment, disclosed herein is a nucleic acid molecule that is used to transform the Listeria in order to arrive at a recombinant Listeria. In another embodiment, the nucleic acid disclosed herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, or a prfA gene. It is to be understood by a skilled artisan, that the virulence gene can be any gene known in the art to be associated with virulence in the recombinant Listeria.

In yet another embodiment, the Listeria strain is an inlA mutant, an inlB mutant, an inlC mutant, an inlJ mutant, prfA mutant, actA mutant, a dal/dat mutant, a prfA mutant, a plcB deletion mutant, or a double mutant lacking both plcA and plcB or actA and inlB. In another embodiment, the Listeria comprise a deletion or mutation of these genes individually or in combination. In another embodiment, the Listeria disclosed herein lack each one of genes. In another embodiment, the Listeria disclosed herein lack at least one and up to ten of any gene disclosed herein, including the actA, prfA, and dal/dat genes. In another embodiment, the prfA Listeria mutant may be completed by a plasmid encoding comprising a nucleic acid sequence a encoding a PrfA mutant protein comprising a D133V mutation.

In one embodiment, the metabolic gene, the virulence gene, etc. is lacking, deleted or mutated in a chromosome of the Listeria strain. In another embodiment, the metabolic gene, virulence gene, etc. is lacking, deleted or mutated in the chromosome and in any episomal genetic element of the Listeria strain. In another embodiment, the metabolic gene, virulence gene, etc. is lacking, deleted or mutated in the genome of the virulence strain.

In one embodiment, the recombinant Listeria strain disclosed herein is attenuated. In another embodiment, the recombinant Listeria strain disclosed herein comprises an inactivating mutation of the endogenous actA and inlC genes. In another embodiment, the recombinant Listeria strain disclosed herein comprises an inactivating mutation of the endogenous actA, inlB, and inlC genes disclosed herein. In another embodiment, the recombinant Listeria strain disclosed herein comprises an inactivating mutation in any single gene or combination of the following genes: actA, dal, dat, inlB, inlC, prfA, plcA, plcB.

It will be appreciated by the skilled artisan that the term “mutation” and grammatical equivalents thereof, encompass any type of mutation or modification to the sequence (nucleic acid or amino acid sequence), and encompass a deletion mutation, a truncation, an inactivation, a disruption, insertion, duplication, frameshift or a translocation. These types of mutations are readily known in the art.

In one embodiment, in order to select for an auxotrophic bacteria comprising a plasmid encoding a metabolic enzyme or a complementing gene disclosed herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present disclosure if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present disclosure.

In another embodiment, once the auxotrophic bacteria comprising the plasmids disclosed herein have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use disclosed herein.

In one embodiment, the N-terminal LLO protein fragment and nonsensical peptide are fused directly to one another. In another embodiment, the genes encoding the N-terminal LLO protein fragment and nonsensical peptide are fused directly to one another. In another embodiment, the N-terminal LLO protein fragment and nonsensical peptide are operably attached via a linker peptide. In another embodiment, the N-terminal LLO protein fragment and nonsensical peptide are attached via a heterologous peptide. In another embodiment, the N-terminal LLO protein fragment is N-terminal to the nonsensical peptide.

In another embodiment, the N-terminal LLO protein fragment is expressed and used alone, i.e., in unfused form. In another embodiment, an N-terminal LLO protein fragment is the N-terminal-most portion of the fusion protein. In another embodiment, a truncated LLO is truncated at the C-terminal to arrive at an N-terminal LLO. In another embodiment, a truncated LLO is a non-hemolytic LLO.

In one embodiment, the N-terminal ActA protein fragment and nonsensical peptide are fused directly to one another. In another embodiment, the genes encoding the N-terminal ActA protein fragment and nonsensical peptide are fused directly to one another. In another embodiment, the N-terminal ActA protein fragment and nonsensical peptide are operably attached via a linker peptide. In another embodiment, the N-terminal ActA protein fragment and nonsensical peptide are attached via a heterologous peptide. In another embodiment, the N-terminal ActA protein fragment is N-terminal to the nonsensical peptide. In another embodiment, the N-terminal ActA protein fragment is expressed and used alone, i.e., in unfused form. In another embodiment, the N-terminal ActA protein fragment is the N-terminal-most portion of the fusion protein. In another embodiment, a truncated ActA is truncated at the C-terminal to arrive at an N-terminal ActA.

In one embodiment, the recombinant Listeria strain disclosed herein expresses the recombinant polypeptide. In another embodiment, the recombinant Listeria strain comprises a plasmid that encodes the recombinant polypeptide. In another embodiment, a recombinant nucleic acid disclosed herein is in a plasmid in the recombinant Listeria strain disclosed herein. In another embodiment, the plasmid is an episomal plasmid that does not integrate into the recombinant Listeria strain's chromosome. In another embodiment, the plasmid is an integrative plasmid that integrates into the Listeria strain's chromosome. In another embodiment, the plasmid is a multicopy plasmid.

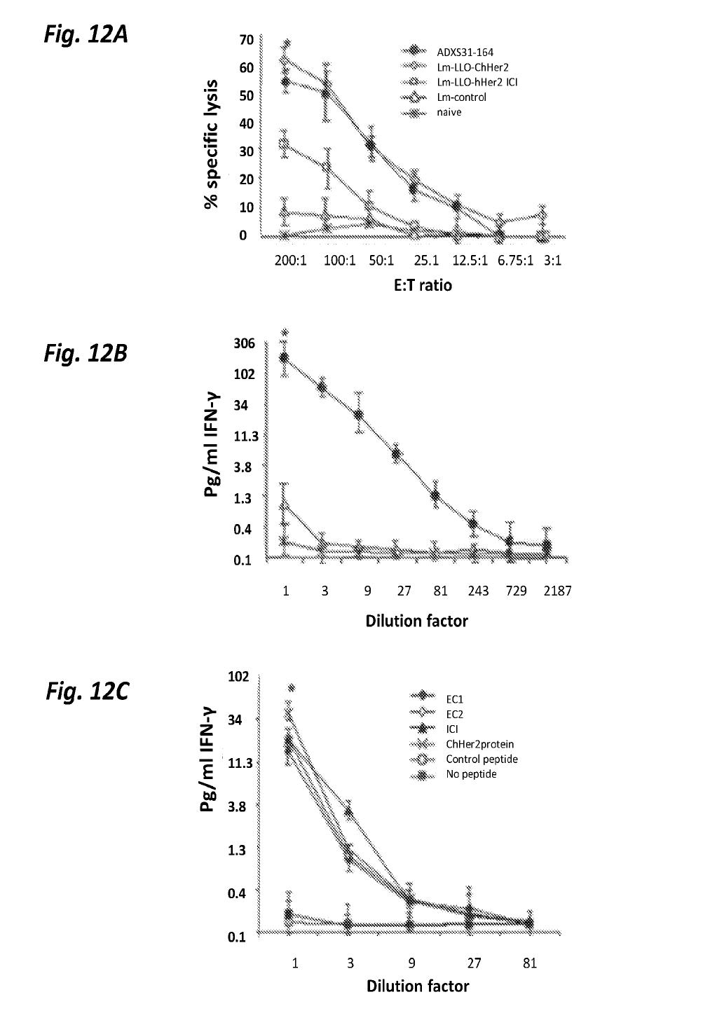

In another embodiment, no CTL activity is detected in naïve animals or mice injected with an irrelevant Listeria vaccine (FIG. 12A). While in another embodiment, the attenuated auxotrophic strain disclosed herein is able to stimulate the secretion of IFN-γ by the splenocytes from wild type FVB/N mice (FIGS. 12B and 12C).

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967.

### III. Delivery Vectors

In one embodiment, a vector disclosed herein is a vector known in the art, including a plasmid or a phage vector. In another embodiment, the construct or nucleic acid molecule is integrated into the Listerial chromosome using a phage vector comprising phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the present disclosure further comprises a phage based chromosomal integration system for clinical applications, where a host strain that is auxotrophic for essential enzymes, including, but not limited to, d-alanine racemase can be used, for example Lmdal(−)dat(−). In another embodiment, in order to avoid a “phage curing step,” a phage integration system based on PSA is used. This requires, in another embodiment, continuous selection by antibiotics to maintain the integrated gene. Thus, in another embodiment, the current disclosure enables the establishment of a phage based chromosomal integration system that does not require selection with antibiotics. Instead, an auxotrophic host strain can be complemented.

In one embodiment, a vector used for delivery of nucleic acids encoding one or more peptides or fragments thereof, or one or more nonsensical peptides or fragments thereof, comprising one or more neo-epitopes is not limited to a recombinant Listeria strain but encompasses any delivery vector known in the art to be useful for delivery nucleic acids or peptides in a mammalian subject. In another embodiment, a vector disclosed herein is a delivery vector known in the art including a bacterial delivery vector, a DNA vaccine delivery vector, an RNA vaccine deliver vector, a virus delivery vector, a virus-like particle, a liposomal delivery vector, or a nucleic acid-loaded nanoparticle. It will be appreciated by one skilled in the art that the term “delivery vectors” refers to a construct which is capable of delivering, and, within certain embodiments expressing, one or more neo-epitopes or peptides comprising one or more neo-epitopes in a host cell. Representative examples of such vectors include viral vectors, nucleic acid expression vectors, naked DNA, and certain eukaryotic cells (e.g., producer cells). In one embodiment, a delivery vector differs from a plasmid or phage vector. In another embodiment, a delivery vector and a plasmid or phage vector of this disclosure are the same. In another embodiment, a bacterial delivery vector used in the methods and compositions disclosed herein is a Listeria monocytogenes strain. In another embodiment, a delivery vector is a bacterial vector, a viral vector, a peptide immunotherapy or vaccine vector, or a DNA immunotherapy or vaccine vector.

In one embodiment, a virus delivery vector may be selected from the following: a retrovirus, an adenovirus, an adeno-associated virus, a herpes virus, a pox virus, a human foamy virus (HFV), a lentivirus or any other virus delivery vector known in the art.

In one embodiment, the immunotherapy delivery vector is a nanoparticle. In another embodiment, the nanoparticle is coated with a cationic polymer or cationic lipid. In another embodiment, the coated nanoparticle further comprises targeting ligands that target the nanoparticle comprising a recombinant nucleic acid sequence disclosed herein to a desired tissue or tumor cell.

In one embodiment, a liposomal delivery vector disclosed herein is a cationic liposome.

In another embodiment, the immunotherapy delivery vector disclosed herein evades the reticuloendothelial system (RES) as it circulates after systemic administration and crosses several barriers before it arrives in the cytoplasm or nucleus of a target cell such as a disease-bearing tissue or a tumor cell.

In one embodiment of the methods and compositions as disclosed herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector,” “phage vector,” or “phagemid” refers to any phage-based recombinant expression system for the purpose of expressing a nucleic acid sequence of the methods and compositions as disclosed herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In another embodiment, the delivery vector further comprises a nucleic acid construct comprising one or more open reading frames encoding one or more one or more immunomodulatory molecule(s). In another embodiment, the Listeria strain further comprises a nucleic acid construct comprising one or more open reading frames encoding one or more one or more immunomodulatory molecule(s). Examples of such molecules include interferon gamma, a cytokine, a chemokine, a T-cell stimulant, and any combination thereof.

In another embodiment, the immunomodulatory molecule is expressed and secreted from said Listeria strain, wherein said molecule is selected from a group comprising interferon gamma, a cytokine, a chemokine, a T-cell stimulant, and any combination thereof.

In one embodiment, the present disclosure provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display plasmid vector to create a fusion protein comprising an amino acid sequence encoded by an open reading frame and the display protein. As appreciated by one of skill in the art, the linker can comprise additional amino acids, such as glycine and other small neutral amino acids.

It will be appreciated by a skilled artisan that the term “endogenous” may encompass an item that has developed or originated within the reference organism or arisen from causes within the reference organism. In another embodiment, endogenous refers to native.

“Stably maintained” refers, in one embodiment, to maintenance of a nucleic acid molecule or plasmid in the absence of selection (e.g., antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In another embodiment, disclosed herein is a recombinant Listeria strain, comprising a nucleic acid molecule operably integrated into the Listeria genome as an open reading frame with an endogenous ActA sequence. In another embodiment, a recombinant Listeria strain of the methods and compositions as disclosed herein comprise an episomal expression plasmid vector comprising a nucleic acid molecule encoding fusion protein comprising an antigen fused to an ActA or a truncated ActA. In one embodiment, the expression and secretion of the antigen is under the control of an actA promoter and an actA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the non-conservative QDNKR (SEQ ID NO: 60) substitution as described in US Patent Publication No. 2014/0186387.

In one embodiment, a fragment disclosed herein is a functional fragment. In another embodiment, a “functional fragment” is an immunogenic fragment that is capable of eliciting an immune response when administered to a subject alone or in a vaccine composition disclosed herein. In another embodiment, a functional fragment has biological activity as will be understood by a skilled artisan and as further disclosed herein.

In one embodiment, the Listeria strain disclosed herein is an attenuated strain. In another embodiment, the Listeria strain disclosed herein is a recombinant strain. In another embodiment, the Listeria strain disclosed herein is a live attenuated recombinant Listeria strain.

The recombinant Listeria strain of methods and compositions of the present disclosure is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, a recombinant Listeria strain of the present disclosure has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by any other method known in the art. In another embodiment, the Listeria has not been passaged.

In another embodiment, a recombinant nucleic acid of the present disclosure is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter.

In another embodiment, inducible and tissue specific expression of the nucleic acid encoding a peptide of the present disclosure is accomplished by placing the nucleic acid encoding the peptide under the control of an inducible or tissue specific promoter/regulatory sequence. Examples of tissue specific or inducible promoter/regulatory sequences which are useful for this purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the disclosure includes the use of any promoter/regulatory sequence, which is either known or unknown, and which is capable of driving expression of the desired protein operably linked thereto. It will be appreciated by the skilled artisan that the term “episomal expression vector” encompasses a nucleic acid plasmid vector which may be linear or circular, and which is usually double-stranded in form and is extrachromosomal in that it is present in the cytoplasm of a host bacteria or cell as opposed to being integrated into the bacteria's or cell's genome. In one embodiment, an episomal expression vector comprises a gene of interest. In another embodiment, episomal vectors persist in multiple copies in the bacterial cytoplasm, resulting in amplification of the gene of interest, and, in another embodiment, viral trans-acting factors are supplied when necessary. In another embodiment, the episomal expression vector may be referred to as a plasmid herein. In another embodiment, an “integrative plasmid” comprises sequences that target its insertion or the insertion of the gene of interest carried within into a host genome. In another embodiment, an inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, in stable transfection procedures, the use of episomal vectors often results in higher transfection efficiency than the use of chromosome-integrating plasmids (Belt, P.B.G.M., et al (1991) Efficient cDNA cloning by direct phenotypic correction of a mutant human cell line (HPRT2) using an Epstein-Barr virus-derived cDNA expression plasmid vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions as disclosed herein may be delivered to cells in vivo, ex vivo, or in vitro by any of a variety of the methods employed to deliver DNA molecules to cells. The plasmid vectors may also be delivered alone or in the form of a pharmaceutical composition that enhances delivery to cells of a subject.

In one embodiment, the term “fused” refers to operable linkage by covalent bonding. In one embodiment, the term includes recombinant fusion (of nucleic acid sequences or open reading frames thereof). In another embodiment, the term includes chemical conjugation. In one embodiment, the term “fused” refers to nucleic acid sequences connected such that a single reading frame is formed. In one embodiment, the term “fused” refers to nucleic acid sequences connected such that a plurality of reading frames is formed. In one embodiment, the term “fused” refers to nucleic acid sequences connected such that a promoter sequence is functionally connected to an open reading frame. In one embodiment, the term “fused” refers to a nucleic acid sequence connected to the N-terminus of a second nucleic acid sequence. In another embodiment, the term “fused” refers to a nucleic acid sequences connected to the C-terminus of a second nucleic acid sequence.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the methods and compositions as disclosed herein. In one embodiment, transforming is accomplished using a plasmid or phage vector.

In another embodiment, conjugation is used to introduce genetic material and/or plasmids into bacteria. Methods for conjugation are well known in the art, and are described, for example, in Nikodinovic J. et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9). Each method represents a separate embodiment of the methods and compositions as disclosed herein.

In one embodiment, the term “attenuation,” refers to a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present disclosure are therefore environmentally safe in that they are incapable of uncontrolled replication.

In another embodiment, the Listeria strain comprises neo-epitopes in the range of about 1-100 neo-epitopes per Listeria. In another embodiment, the Listeria strain comprises neo-epitopes in the range of 100-200 per Listeria. In another embodiment, the Listeria strain comprises up to about 10 neo-epitopes per Listeria. In another embodiment, the Listeria strain comprises up to about 20 neo-epitopes per Listeria. In another embodiment, the Listeria strain comprises up to about 50 neo-epitopes per Listeria. In another embodiment, the Listeria strain comprises up to about 200 neo-epitopes per Listeria. In another embodiment, the Listeria strain comprises up to about 300 neo-epitopes per Listeria. In another embodiment, the Listeria strain comprises up to about 400 neo-epitopes per Listeria. In another embodiment, the Listeria strain comprises up to about 500 neo-epitopes per Listeria. Alternatively, the Listeria strain comprises the neo-epitopes in the range of about 1-5, 5-10, 10-15, 15-20, 10-20, 20-30, 30-40, 40-50, 50-60, 60-70, 70-80, 80-90, 90-100, 5-15, 5-20, 5-25, 15-20, 15-25, 15-30, 15-35, 20-25, 20-35, 20-45, 30-45, 30-55, 40-55, 40-65, 50-65, 50-75, 60-75, 60-85, 70-85, 70-95, 80-95, 80-105 or 95-105. Alternatively, the Listeria strain comprises the neo-epitopes in the range of about 1-100, 5-100, 5-75, 5-50, 5-40, 5-30, 5-20, 5-15 or 5-10. Alternatively, the Listeria strain comprises the neo-epitopes in the range of about 1-100, 1-75, 1-50, 1-40, 1-30, 1-20, 1-15 or 1-10. Alternatively, the Listeria strain comprises the neo-epitopes in the range of about 50-100 per Listeria. Alternatively, the Listeria strain comprises up to about 100 neo-epitopes per Listeria. Alternatively, the Listeria strain comprises up to about 10, up to about 20, up to about 30, up to about 40, or up to about 50 neo-epitopes. Each possibility represents a separate embodiment of the present disclosure.

In another embodiment, the Listeria strain comprises more than about 100 of the neo-epitopes per Listeria. In another embodiment, the Listeria strain comprises more than about 500 neo-epitopes per Listeria. In another embodiment, the Listeria strain comprises one neo-epitope. Alternatively, the Listeria strain comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 neo-epitopes per Listeria.

In another embodiment, a Listeria comprises or expresses one or more non-sensical peptides in the context of a fusion protein with a truncated LLO, truncated ActA or PEST sequence, wherein said one or more non-sensical peptides comprise any number of neo-epitopes disclosed in the above embodiments.

### IV. Process of Personalizing Immunotherapy

Also disclosed herein are processes for personalizing immunotherapy. In one embodiment, a process of this disclosure creates a personalized immunotherapy. In another embodiment, a process of creating a personalized immunotherapy for a subject having a disease or condition comprises identifying and selecting neo-epitopes within mutated and variant antigens (neo-antigens) that are specific to the patient's disease. In another embodiment, a process of this disclosure comprises identifying nucleic acid molecules having at least one frameshift mutation leading to translation of a nonsensical peptide or a portion of a polypeptide that is nonsensical. In another embodiment, a process for creating a personalized immunotherapy for a subject is in order to provide a treatment for the subject. In another embodiment, personalized immunotherapy may be used to treat such diseases as cancer, autoimmune disease, organ transplantation rejection, bacterial infection, viral infection, and chronic viral illnesses such as HIV.

In another embodiment, the process of this disclosure for creating a personalized immunotherapy may comprise use of the extracted nucleic acid from the abnormal or unhealthy sample and the extracted nucleic acid from the normal or healthy reference sample in order to identify somatic mutations or nucleic acid sequence differences present in the abnormal or unhealthy sample as compared with the normal or healthy sample, wherein these sequences having somatic mutations or differences encode an expressed amino acid sequence. In another embodiment, a peptide expressing the somatic mutations or sequence differences, may in certain embodiments, be referred to throughout as “neo-epitopes.” A peptide expressed from a nucleotide sequence comprising at least one frameshift mutation, may in certain embodiments, be referred to as “nonsensical peptides,” wherein these nonsensical peptides comprise one or more neo-epitopes.

An example of such a process for creating a personalized immunotherapy for a subject having a disease or condition comprises: (a) comparing one or more open reading frames (ORFs) in nucleic acid sequences extracted from a disease-bearing or condition-bearing biological sample from the subject with one or more ORFs in nucleic acid sequences extracted from a healthy biological sample, wherein the comparing identifies one or more nucleic acid sequences encoding one or more peptides comprising one or more immunogenic neo-epitopes (e.g., T-cell epitopes) encoded within the one or more ORFs from the disease-bearing or condition-bearing biological sample, wherein at least one of the one or more nucleic acid sequences comprises one or more frameshift mutations and encodes one or more frameshift-mutation-derived peptides comprising one or more immunogenic neo-epitopes; and (b) generating an immunotherapy delivery vector comprising a nucleic acid comprising an open reading frame encoding a recombinant polypeptide comprising the one or more peptides comprising the one or more immunogenic neo-epitopes identified in step (a). The immunotherapy delivery vector can be any type of immunotherapy delivery vector. For example, such a process can be used to create a DNA immunotherapy, a peptide immunotherapy, or a recombinant Listeria strain or other bacterial strain used for immunotherapy.

In one embodiment, the one or more neo-epitopes comprise a plurality of neo-epitopes. Optionally, step (b) can further comprise one or more iterations of randomizing the order of the one or more peptides comprising the plurality of neo-epitopes within the nucleic acid sequence of step (b). Such randomizing can include, for example, randomizing the order of the entire set of one or more peptides comprising the plurality of neo-epitopes, or can comprise randomizing the order of a subset of the one or more peptides comprising a subset of the plurality of neo-epitopes. For example, if the nucleic acid sequence comprises 20 peptides (ordered 1-20) comprising 20 neo-epitopes, the randomizing can comprise randomizing the order of all 20 peptides or can comprise randomizing the order of only a subset of the peptides (e.g., peptides 1-5 or 6-10). Such randomization of the order can facilitate secretion and presentation of the neo-epitopes and of each individual region.

Such methods can further comprise storing the immunotherapy delivery vector for administering to the subject within a predetermined period of time. Likewise, such methods can further comprise administering a composition comprising the immunotherapy vector, the DNA immunotherapy, or the peptide immunotherapy to the subject, wherein the administering results in the generation of a personalized T-cell immune response against the disease or condition.

The disease-bearing or condition-bearing biological sample can be obtained from the subject having the disease or condition. Likewise, the healthy biological sample can be obtained from the subject having the disease or condition. A healthy biological sample can also be obtained from someone other than the subject. Examples of suitable biological samples include a tissue, a cell, a blood sample, or a serum sample.

The comparing in step (a) can be by any suitable means. For example, it can comprise use of a screening assay or screening tool and associated digital software for comparing the one or more ORFs in the nucleic acid sequences extracted from the disease-bearing or condition-bearing biological sample with the one or more ORFs in the nucleic acid sequences extracted from the healthy biological sample. Such associated digital software can comprise access to a sequence database that allows screening of mutations within the ORFs in the nucleic acid sequences extracted from the disease-bearing or condition-bearing biological sample for identification of immunogenic potential of the neo-epitopes.

The nucleic acid sequences extracted from the disease-bearing or condition-bearing biological sample and the nucleic acid sequences extracted from the healthy biological sample can be determined by any means. For example, the nucleic acid sequences extracted from the disease-bearing or condition-bearing biological sample and the nucleic acid sequences extracted from the healthy biological sample can be determined using exome sequencing or transcriptome sequencing.

Such processes can further comprise characterizing the one or more frameshift-mutation-derived peptides for neo-epitopes by generating one or more different peptide sequences from the one or more frameshift-mutation-derived peptides. The one or more different peptide sequences can be of any length sufficient to elicit a positive immune response (e.g., sufficient to elicit a positive immune response using the Lm technology) and can be from any portion of the frameshift-mutation-derived peptide. The one or more different peptide sequences can be further characterized. For example, the one or more different peptide sequences and excluding a peptide sequence if it does not score below a hydropathy threshold predictive of secretability in Listeria monocytogenes as disclosed elsewhere herein. In one example, the scoring is by a Kyte and Doolittle hydropathy index 21 amino acid window, and any peptide sequence scoring above a cutoff of about 1.6 is excluded or is modified to score below the cutoff. The one or more different peptide sequences can also be screened and selected for binding by MHC Class I or MHC Class II to which a T-cell receptor binds.

The frameshift mutations can be anywhere within a protein-coding gene. For example, the frameshift mutation can be in the penultimate exon or the last exon of a gene. A nonsensical peptide encoded by a frameshift mutation can be of any length sufficient to elicit a positive immune response (e.g., sufficient to elicit a positive immune response using the Lm technology). For example, one or more or each of the nonsensical peptides can be about 8-10, 11-20, 21-40, 41-60, 61-80, 81-100, 101-150, 151-200, 201-250, 251-300, 301-350, 351-400, 401-450, 451-500, or 8-500 amino acids in length. Some such nonsensical peptides do not encode a post-translational cleavage site.

The disease or condition can be any disease or condition in which neo-epitopes are present. For example, the disease or condition can be a cancer or tumor. As an example, the one or more immunogenic neo-epitopes can comprise a self-antigen associated with the disease or condition, wherein the self-antigen comprises a cancer-associated or tumor-associated neo-epitope or a cancer-specific or tumor-specific neo-epitope. Examples of tumors or cancers are provided elsewhere herein. For example, the disease or condition can be a tumor with fewer than 120, 110, 100, 90, 80, 70, 60, 50, 40, 30, 20, or 10 nonsynonymous missense mutations that are not present in the healthy biological sample.

The disease or condition can also be an infectious disease. For example, the one or more nonsensical peptides can comprise an infectious-disease-associated or infectious-disease-specific neo-epitope.

The immunotherapy delivery vectors (e.g., recombinant Listeria strains) that can be produced by such processes are described in further detail elsewhere herein. The process can be repeated to create a plurality of immunotherapy delivery vectors, each comprising a different set of one or more immunogenic neo-epitopes. For example, the plurality of immunotherapy delivery vectors can comprise about 2-5, 5-10, 10-15, 15-20, 10-20, 20-30, 30-40, or 40-50 immunotherapy delivery vectors. As another example, the combination of the plurality of immunotherapy delivery vectors can comprise about 5-10, 10-15, 15-20, 10-20, 20-30, 30-40, 40-50, 50-60, 60-70, 70-80, 80-90, 90-100, or 100-200 immunogenic neo-epitopes.

In one embodiment, disclosed herein is a process for creating a personalized immunotherapy for a subject having a disease or condition, the process comprising the steps of: (a) comparing one or more open reading frames (ORFs) in nucleic acid sequences extracted from a disease-bearing biological sample with one or more ORFs in nucleic acid sequences extracted from a healthy biological sample, wherein the comparing identifies one or more nucleic acid sequences comprising at least a frameshift mutation and encoding one or more peptides comprising one or more neo-epitopes encoded within said one or more ORFs from the disease-bearing sample; (b) transforming an attenuated Listeria strain with a vector comprising a nucleic acid sequence encoding one or more peptides comprising the one or more neo-epitopes identified in a.; and, alternatively storing the attenuated recombinant Listeria for administering to the subject at a pre-determined period or administering a composition comprising the attenuated recombinant Listeria strain to the subject, and wherein the administering results in the generation of a personalized T-cell immune response against said disease or said condition; optionally, (c) obtaining a second biological sample from the subject comprising a T-cell clone or T-infiltrating cell from the T-cell immune response and characterizing specific peptides comprising one or more neo-epitopes bound by T-cell receptors on said T cells, wherein said one or more neo-epitopes are immunogenic; (d) screening for and selecting a nucleic acid construct encoding one or more peptides comprising one or more immunogenic neo-epitope identified in (c); and, (e) transforming a second attenuated recombinant Listeria strain with a vector comprising a nucleic acid sequence encoding one or more peptides comprising the one or more immunogenic neo-epitopes; and, alternatively storing said second attenuated recombinant Listeria for administering to the subject at a pre-determined period or administering a second composition comprising the second attenuated recombinant Listeria strain to said subject, wherein the process creates a personalized immunotherapy for the subject. In another embodiment, step (a) comprises comparing one or more open reading frames (ORFs) in nucleic acid sequences extracted from a disease-bearing biological sample with one or more ORFs in nucleic acid sequences extracted from a healthy biological sample, wherein the comparing identifies one or more nucleic acid sequences comprising at least one frameshift mutation, wherein the amino acid sequence encoded by the nucleic acid sequence comprising the frameshift mutation(s) may be screen for one or more nonsensical peptides comprising one or more neo-epitopes encoded within said one or more ORFs from the disease-bearing sample.

In one embodiment, the number of vectors to be used (e.g., a Listeria vector) is determined by taking into consideration predefining groups of: known tumor-associated mutations found in circulating tumor cells; known cancer “driver” mutations; and/or known chemotherapy resistance mutations and giving these priority in the 21 amino acid sequence peptide selection (see Example 19). In another embodiment, this can be accomplished by screening identified mutated genes against the COSMIC (Catalogue of somatic mutations in cancer, cancer.Sanger.ac.uk) or Cancer Genome Analysis or other similar cancer-associated gene database. Further, and in another embodiment, screening for immunosuppressive epitopes (T-reg epitopes, IL-10 inducing T helper epitopes, etc.) is utilized to de-select or to avoid immunosuppressive influences on the vector.

In another embodiment, the step of comparing one or more open reading frames (ORFs) in nucleic acid sequences extracted from a disease-bearing biological sample with one or more ORFs in nucleic acid sequences extracted from a healthy biological sample, further comprises using of a screening assay or screening tool and associated digital software for comparing one or more ORFs in nucleic acid sequences extracted from the disease-bearing biological sample with one or more ORFs in nucleic acid sequences extracted from the healthy biological sample, wherein the associated digital software comprises access to a sequence database that allows screening of mutations within the ORFs in the nucleic acid sequences extracted from the disease-bearing biological sample for identification of immunogenic potential of the neo-epitopes.

In one embodiment, the nucleic acid sequences from disease-bearing and healthy samples are compared in order to identify frameshift mutations. In one embodiment, frameshift sequence variants may create novel or at least partially novel nonsensical peptide sequences that include neo-epitopes as described herein.

In another embodiment, nonsensical peptide or frameshift-mutation-derived peptide sequences can be selected. The selected peptides can then be arranged into one or more candidate orders for a potential recombinant polypeptide. If there are more usable peptides than can fit into a single plasmid, different peptides can be assigned priority ranks as needed/desired and/or split up into different recombinant polypeptides (e.g., for inclusion in different recombinant Listeria strains). Priority rank can be determined by factors such as relative size, priority of transcription, and/or overall hydrophobicity of the translated polypeptide. The peptides can be arranged so that they are joined directly together without linkers, or any combination of linkers between any number of pairs of peptides, as disclosed in more detail elsewhere herein. The number of linear peptides to be included can be determined based on consideration of the number of constructs needed versus the mutational burden, the efficiency of translation and secretion of multiple epitopes from a single plasmid, or the MOI needed for each bacteria or Lm comprising a plasmid. For example, ranges of linear antigenic peptides can be starting, for example, with about 50, 40, 30, 20, or 10 antigenic peptides per plasmid.

In another embodiment, of the disclosure the method as disclosed in any of the herein, additionally comprises the step of screening one or more neo-epitopes, nonsensical peptides comprising one or more neo-epitopes, or recombinant polypeptide comprising one or more neo-epitopes, for hydrophobicity and hydrophilicity.

In another embodiment, a process as described herein, additionally comprises the step of selecting one or more neo-epitopes, nonsensical peptides or recombinant polypeptide comprising one or more neo-epitopes that are hydrophilic.

In another embodiment, a process as described herein, comprising the step of selecting one or more neo-epitopes, peptide comprising one or more neo-epitopes, nonsensical peptides, or recombinant polypeptide comprising one or more neo-epitopes, that have a score of up to 1.6 in the Kyte Doolittle hydropathy plot.

In one embodiment, the hydrophobicity is scaled using the Kyte-Doolittle (Kyte J, Doolittle RF (May 1982). “A simple method for displaying the hydropathic character of a protein.” J. Mol. Biol. 157 (1): 105-32) or other suitable hydropathy plot or other appropriate scale including, but not limited those disclosed by Rose et. al (Rose, G. D. and Wolfenden, R. (1993) Annu. Rev. Biomol. Struct., 22, 381-415.); Kallol M. Biswas, Daniel R. DeVido, John G. Dorsey(2003) Journal of Chromatography A, 1000, 637-655, Eisenberg D (July 1984). Ann. Rev. Biochem. 53: 595-623.); Abraham D. J., Leo A. J. Proteins: Structure, Function and Genetics 2:130-152(1987); Sweet R. M., Eisenberg D. J. Mol. Biol. 171:479-488(1983); Bull H. B., Breese K. Arch. Biochem. Biophys. 161:665-670(1974); Guy H. R. Biophys J. 47:61-70(1985); Miyazawa S., et al., Macromolecules 18:534-552(1985); Roseman M. A. J. Mol. Biol. 200:513-522(1988); Wolfenden R. V., et al. Biochemistry 20:849-855(1981); Wilson K. J; Biochem. J. 199:31-41(1981); Cowan R., Whittaker R. G. Peptide Research 3:75-80(1990); Aboderin A. A. Int. J. Biochem. 2:537-544(1971); Eisenberg D. et al., J. Mol. Biol. 179:125-142(1984); Hopp T. P., Woods K. R. Proc. Natl. Acad. Sci. U.S.A. 78:3824-3828(1981); Manavalan P., Ponnuswamy P. K. Nature 275:673-674(1978).; Black S. D., Mould D. R. Anal. Biochem. 193:72-82(1991); Fauchere J.-L., Pliska V. E. Eur. J. Med. Chem. 18:369-375(1983); Janin J. Nature 277:491-492(1979); Rao M. J. K., Argos P. Biochim Biophys. Acta 869:197-214(1986); Tanford C. J. Am. Chem. Soc. 84:4240-4274(1962); Welling G. W., et al., FEBS Lett. 188:215-218(1985); Parker J. M. R. et al., Biochemistry 25:5425-5431(1986); Cowan R., Whittaker R. G. Peptide Research 3:75-80(1990), all of which are incorporated by reference herein in their entirety. In another embodiment, all epitopes scoring on the scale-appropriate measure to have an unsatisfactorily high level of hydrophobicity to be efficiently secreted are moved from the listing or are de-selected. In another embodiment, all epitopes scoring on the Kyte-Doolittle plot to have an unsatisfactorily high level of hydrophobicity to be efficiently secreted, such as 1.6 or above, are moved from the listing or are de-selected. In another embodiment, each neo-epitope's ability to bind to subject HLA is rated using the Immune Epitope Database (IEDB) analysis resource which comprises: netMHCpan, ANN, SMMPMBEC. SMM, CombLib_Sidney2008, PickPocket, netMHCcons. Other sources include TEpredict (tepredict.sourceforge.net/help.html) or alternative MHC binding measurement scales available in the art.

In one embodiment, once a neo-epitope or a nonsensical peptide is identified, the neo-epitope or a nonsensical peptide, is scored by the Kyte and Doolittle hydropathy index 21 amino acid window, wherein in another embodiment, neo-epitopes scoring above a specific cutoff (around 1.6) are excluded as they are unlikely to be secretable by Listeria monocytogenes. In one embodiment, the portion of a recombinant polypeptide comprising one or more heterologous peptides, the portion of a recombinant polypeptide comprising one or more nonsensical or frameshift-mutation-derived peptides, or the recombinant polypeptide is scored by the Kyte and Doolittle hydropathy index 21 amino acid window. If any region scores above a cutoff (e.g., around 1.6), the peptides can be reordered or shuffled within the recombinant polypeptide using selected parameters or using randomization until an acceptable order of antigenic peptides is found (i.e., one in which no region scores above the cutoff). Alternatively, any problematic peptides can be removed or redesigned to be of a different size, or to shift the sequence of the protein included in the peptide. Alternatively or additionally, one or more linkers between peptides as disclosed elsewhere herein can be added or modified to change the hydrophobicity. In another embodiment, the cut off is selected from the following ranges 1.2-1.4, 1.4-1.6, 1.6-1.8, 1.8-2.0, 2.0-2.2 2.2-2.5, 2.5-3.0, 3.0-3.5, 3.5-4.0, or 4.0-4.5. In one embodiment, the cutoff score used to determine what epitopes are moved from the list or are de-selected is 1.6. In another embodiment, the cutoff is 1.4, 1.5, 1.7, 1.8, 1.9, 2.0, 2.1, 2.2, 2.3, 2.3, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. Each possibility represents a separate embodiment of the present disclosure. In another embodiment, the cut off varies depending on the genus of the delivery vector being used. In another embodiment, the cut off varies depending on the species of the delivery vector being used.

In one embodiment, the neo-epitope or nonsensical peptide or frameshift-mutation-derived peptide is scored by the Kyte and Doolittle hydropathy index 21 amino acid sliding window. In one embodiment, the neo-epitope, the nonsensical peptide, the frameshift-mutation-derived peptide, the portion of a recombinant polypeptide comprising the one or more heterologous peptides, the portion of a recombinant polypeptide comprising the one or more nonsensical or frameshift-mutation-derived peptides, or the recombinant polypeptide is scored by the Kyte and Doolittle hydropathy index 21 amino acid sliding window. In another embodiment, the sliding window size is selected from the group comprising 9, 11, 13, 15, 17, 19, and 21 amino acids. In another embodiment, the sliding window size is 9-11 amino acids, 11-13 amino acids, 13-15 amino acids, 15-17 amino acids, 17-19 amino acids or 19-21 amino acids. Each possibility represents a separate embodiment of the present disclosure.

In another embodiment, each neo-epitope's or a nonsensical peptide's ability to bind to subject HLA is rated using the Immune Epitope Database (IED) or any other substitute database and associated digital software as known in the art. In another embodiment, other binding prediction services and related databases that are used include NetMHCpan server (http://www.cbs.dtu.dk/services/NetMHCpan/), The IMGT/HLA Database (https://www.ebi.ac.uk/ipd/imgt/hla/), Bimas—HLA Peptide Binding Predictions (http://www-bimas.cit.nih.gov/molbio/hla_bind/), Rankpep: prediction of binding peptides to Class I and Class II MHC molecules (http://imed.med.ucm.es/Tools/rankpep.html), SYFPEITHI database for MHC ligands and peptide motifs (http://www.syfpeithi.de/), and artificial neural network (ANN) (http://ann.thwien.de/index.php?title=Main_Page). Each possibility represents a separate embodiment of the present disclosure.

In another embodiment, Major Histocompatibility Complex (MHC) I and/or II binding affinity is predicted across all possible 9- and 10-mer peptides. In another embodiment, the affinity was predicted across all possible neo-epitopes that can be generated from a sequence comprising a mutation or encoding a nonsensical peptide formed by a frameshift. In another embodiment, the prediction is performed for sequences about 21 amino acids in size (21 mer). In another embodiment, the prediction is performed for sequences include at least about 5, 6, 7, 8, 9, 10, 11, 12, 13, 14, 15, 16, 17, 18, 19, 20, 21, 22, 23, 24, 25, 26, 27, 28, 29, or 30 amino acids. Each possibility represents a separate embodiment of the present disclosure. In another embodiment, the prediction is performed for sequences that are about 5, 6, 7, 8, 9, 10, 11, 12, 13, 14, 15, 16, 17, 18, 19, 20, 21, 22, 23, 24, 25, 26, 27, 28, 29, or 30 amino acids long. Each possibility represents a separate embodiment of the present disclosure. In another embodiment, the prediction is performed for sequences in the range of about 8-12 amino acids, 5-10, 5-12, 5-15, 5-25, 5-35, 8-15 or in the range of about 5-50 amino acids. Each possibility represents a separate embodiment of the present disclosure. In another embodiment, the prediction is performed for sequences of variable or similar amino acids length.

In another embodiment, the estimated abundance of neo-epitopes across a plurality of tumors (and a broad array of HLA alleles) is about 1.5 HLA-binding peptides with IC₅₀<500 nM per point mutation and about 4 binding peptides per frameshift mutation.

In another embodiment, it will be appreciated by the skilled artisan that relative binding ability of different nonsensical peptides to a specific MHC molecule can be directly assessed by competition experiments. The value IC₅₀ refers in one embodiment to the peptide concentration that leads to 50% inhibition of a standard peptide, and the relative binding energy can be described as the ratio between the IC₅₀ of the standard peptide and that of a test peptide. In another embodiment, these values may be correlated to the predicted HLA peptide bindings.

In another embodiment, the skilled artisan will appreciate that binding prediction criteria in the field of HLA peptide binding prediction may be defined as: peptides with IC₅₀<150 nM as strong binders, IC₅₀ of 150 to 500 nM as intermediate to weak binders, and IC₅₀>500 nM as nonbinders. In another embodiment, the cutoff for HLA binding peptides is about IC₅₀<50 nM, IC₅₀<100 nM, IC₅₀<150 nM, IC₅₀<200 nM, IC₅₀<250 nM, IC₅₀<300 nM, IC₅₀<350 nM, IC₅₀<400 nM, IC₅₀<450 nM, IC₅₀<500 nM, IC₅₀<550 nM, IC₅₀<600 nM, IC₅₀<650 nM, IC₅₀<700 nM, or IC₅₀<750 nM. Each possibility represents a separate embodiment of the present disclosure.

In another embodiment, the cutoff for HLA binding peptides is in the range of about 0<IC₅₀<150 nM, 0<IC₅₀<200 nM, 0<IC₅₀<250 nM, 0<IC₅₀<350 nM, 0<IC₅₀<400 nM, 0<IC₅₀<450 nM, 0<IC₅₀<550 nM, 0<IC₅₀<600 nM, 0<IC₅₀<650 nM, 0<IC₅₀<700 nM, 0<IC₅₀<750 nM, 0<IC₅₀<800 nM. Each possibility represents a separate embodiment of the present disclosure.

In one embodiment, neo-epitopes or nonsensical peptides identified from a disease-bearing sample may be presented on major histocompatibility complex class I molecules (MHCI). In one embodiment, a peptides containing a neo-epitope mutation is immunogenic and is recognized as a ‘non-self’ neo-antigen by the adaptive immune system. In another embodiment, use of a one or more neo-epitope sequence comprised in a peptide, a recombinant polypeptide, or a fusion polypeptide provides a targeting immunotherapy, which may, in certain embodiments therapeutically activate a T-cell immune responses to the disease or condition. In another embodiment, use of a one or more neo-epitope sequence comprised in a nonsensical peptide, a polypeptide, or a fusion polypeptide provides a targeting immunotherapy, which may, in certain embodiments therapeutically activate an adaptive immune responses to a disease or condition.

In another embodiment, the process comprises the step of screening one or more neo-epitope(s) or a nonsensical peptide(s) for immunosuppressive epitopes, and removing them from the neo-epitopes identified. In one embodiment, these immunosuppressive epitopes are as presented in the sequence or are artificially created as a result of the splicing together of epitope sequences and linkers.

In another embodiment, the process comprises the step of screening one or more neo-epitope(s) or a nonsensical peptide(s) for T-regulatory activating epitopes, and removing them from the neo-epitopes identified.

In another embodiment, the process comprises the step of screening one or more neo-epitope(s) or a nonsensical peptide(s) for epitopes expressed by the disease or condition bearing biological sample, and removing not expressed epitopes from the neo-epitopes identified.

In another embodiment, the process comprises the step of screening one or more neo-epitope(s) or a nonsensical peptide(s) for epitopes not comprising a post-translational cleavage site.

In another embodiment, the process comprises the step of screening for one or more nucleic acid sequences comprising a frameshift mutation. In another embodiment the process comprises the step of identifying frameshift mutations encoded in a last exon of a gene.

In another embodiment, a process disclosed herein, additionally comprising the step of screening for one or more expressed nonsensical peptides.

In another embodiment, a process disclosed herein, additionally comprising the step of screening one or more neo-epitope(s) or a nonsensical peptide(s) for expressed epitopes, and removing not expressed epitopes from the neo-epitopes identified.

In another embodiment, selecting nonsensical peptides and/or neo-epitopes further comprises the step of screening for highly expressed nonsensical peptides and/or neo-epitopes.

In one embodiment, the nonsensical peptide or fragment thereof and/or a neo-epitope may accumulate in the disease or condition bearing sample. In another embodiment, the present disclosure further comprises eliminating nonsensical peptide or fragment thereof not accumulated up to a certain threshold in the disease or condition bearing biological sample. In one embodiment, the neo-epitope containing peptide may accumulate in the disease or condition bearing sample. In another embodiment, the present disclosure further comprises eliminating neo-epitope containing peptides not accumulated up to a certain threshold in the disease or condition bearing biological sample. In one embodiment the accumulation is detectable by protein detecting means as known in the art, such as ELISA, protein chip, Western blot, florescent tagging, and others.

In another embodiment, a process disclosed herein, comprising the step of acquiring said nonsensical peptide by comparing of one or more open reading frames (ORFs) in nucleic acid sequences extracted from the disease-bearing biological sample with one or more ORFs in nucleic acid sequences extracted from a healthy biological sample, wherein the comparison identifies one or more frameshift mutations within said nucleic acid sequences, wherein the nucleic acid sequence comprising the mutations encodes one or more nonsensical peptides comprising one or more immunogenic neo-epitopes encoded within one or more ORF from the disease-bearing biological sample.

All samples are analyzed for novel genetic sequencing within ORFs. Methods for comparing one or more open reading frames (ORFs) in nucleic acid sequences extracted from the disease-bearing biological sample and healthy biological sample comprise the use of screening assays or screening tools and associated digital software. Methods for performing bioinformatics analyses are known in the art, for example, see US 2013/0210645, US 2014/0045881, and WO 2014/052707, which are each incorporated in full in this application.

According to another embodiment, of the present disclosure, comparing sequences comprises comparing entire exome open reading frames. Additionally or alternatively comparing sequences comprises comparing entire proteome.

Human tumors typically harbor a remarkable number of somatic mutations. Yet, identical mutations in any particular gene are rarely found across tumors (and are even at low frequency for the most common driver mutations). Thus, in one embodiment, a process of this disclosure comprehensively identifying patient-specific tumor frameshift mutations provides a target for a personalized immunotherapy.

In another embodiment, a process disclosed herein, comprising the step of comparing open reading frame exome of a predefined gene-set selected from a group comprising: nucleic acid sequences encoding known and predicted cancer or tumor antigens, nucleic acid sequences encoding tumor or cancer-associated antigens, nucleic acid sequences encoding known or predicted tumor or cancer protein markers, nucleic acid sequences encoding known and predicted infectious disease or condition associated genes, nucleic acid sequences encoding genes expressed in the disease-bearing biological sample, nucleic acid sequences comprising regions of microsatellite instability, and any combination thereof.

In another embodiment, a step in a process of creating a personalized immunotherapy is to obtain an abnormal or unhealthy biological sample, from a subject having a disease or condition. In another embodiment, a disease is an infectious disease, or a tumor or cancer. In another embodiment, the disease, tumor, cancer, condition is disclosed throughout the present disclosure. In another embodiment, the disease is a localized disease. In another embodiment, the disease is a tumor or cancer, an autoimmune disease, an infectious disease, a viral infectious disease, or a bacterial infectious disease.

In another embodiment, a method disclosed herein is disclosed, comprising the steps of: (a) identifying, isolating and expanding T cell clones or T-infiltrating cells that respond against the disease; and, (b) screening for and identifying one or more nonsensical peptides comprising one or more immunogenic neo-epitopes loaded on specific MHC Class I or MHC Class II molecules to which a T-cell receptor on the T cells binds.

In another embodiment, the step of screening for and identifying comprises T-cell receptor sequencing, multiplex based flow cytometry, or high-performance liquid chromatography. In another embodiment, the sequencing comprises using associated digital software and database.

In one embodiment, triplicates of each sample obtained according to the disclosure herein are sequenced by DNA exome sequencing. Nonsensical peptides created by frameshift mutations will display the entire sequence of the mutated peptide that is encoded until a stop codon. Additionally or alternatively, frameshift mutations encode a nonsensical peptide comprising at least a portion of a neo-epitope. In another embodiment, the frameshift mutation encodes a nonsensical peptide comprising at least one neo-epitope. In another embodiment, the nonsensical peptide comprises a plurality of different amino acid sequences, as potential neo-epitopes. In an embodiment the potential neo-epitopes are screened, characterized, rated, selected, and any combination thereof, by the means and methods of the present disclosure.

In another embodiment, a neo-epitope comprises a unique tumor or cancer neo-epitope, a cancer-specific or tumor-specific epitope. In another embodiment, a neo-epitope is immunogenic. In another embodiment, a neo-epitope is recognized by T-cells. In another embodiment, a peptide comprising one or more neo-epitopes activates a T-cell response against a tumor or cancer, wherein the response is personalized to the subject. In another embodiment, a neo-epitope comprises a unique epitope related to an infectious disease. In one embodiment, the infectious disease epitope directly correlates with the disease. In an alternate embodiment, the infectious disease epitope is associated with the infectious disease.

In another embodiment, a step of including a linker sequence between the neo-epitopes sequences. The linker is any linker sequence known in the art. In another embodiment, the linker comprises 4× glycine. In another embodiment, the linker comprises poly-glycine. In yet another embodiment, the linker is selected from a group comprising SEQ ID NOS: 46-56 accordingly, and any combination thereof.

In another embodiment, a step of connecting a tag, as described herein, to the neo-epitopes or nonsensical peptides. In another embodiment, the tag is any tag known in the art. In another embodiment, the tag is selected from SIINFEKL-S-6× HIS tag, 6× HIS tag, SIINFEKL tag, any poly-histidine tag. In one embodiment, connecting the tag is to the C-terminal or to the N-terminal of the recombinant polypeptide or the nucleic acid sequence. In one embodiment connecting the tag to the nucleic acid sequence comprises generating an open reading frame encoding the tag and comprising the neo-epitope(s) or nonsensical peptide(s), and, optionally the linker(s), and optionally an immunogenic polypeptide. In one embodiment the tag is selected from the group consisting of: a 6× histidine tag, a 2× FLAG tag, a 3× FLAG tag, a SIINFEKL peptide, a 6× histidine tag operably linked to a SIINFEKL peptide, a 3× FLAG tag operably linked to a SIINFEKL peptide, a 2× FLAG tag operably linked to a SIINFEKL peptide, and any combination thereof. Two or more tags can be used together, such as a 2× FLAG tag and a SIINFEKL tag, a 3× FLAG tag and a SIINFEKL tag, or a 6× His tag and a SIINFEKL tag. If two or more tags are used, they can be located anywhere within the recombinant polypeptide and in any order. For example, the two tags can be at the C-terminus of the recombinant polypeptide, the two tags can be at the N-terminus of the recombinant polypeptide, the two tags can be located internally within the recombinant polypeptide, one tag can be at the C-terminus and one tag at the N-terminus of the recombinant polypeptide, one tag can be at the C-terminus and one internally within the recombinant polypeptide, or one tag can be at the N-terminus and one internally within the recombinant polypeptide.

In another embodiment, a step of connecting a linker sequence connected to a tag to the neo-epitopes or to the nonsensical peptides.

In another embodiment, at step of detecting the secretion of the neo-epitope, peptide or recombinant polypeptides (fusion/chimeric) is detected using a protein, molecule or antibody (or fragment thereof) that specifically binds to a polyhistidine (His) tag or SIINFEKL-S-6× HIS tag. In another embodiment, at step of detecting the secretion of the neo-epitope, peptide or recombinant polypeptides (fusion/chimeric) is detected using a protein, molecule or antibody (or fragment thereof) that specifically binds to a 2× FLAG tag or a 3× FLAG tag or any other tag disclosed herein.

In another embodiment, a peptide vaccine comprises one or more nonsensical peptides comprising one or more immunogenic neo-epitopes, wherein each nonsensical peptide is fused to or mixed with an immunogenic polypeptide or fragment thereof. In another embodiment, the immunogenic polypeptide is a mutated Listeriolysin O (LLO) protein, a truncated LLO (tLLO) protein, a truncated ActA protein, or a PEST amino acid sequence. In another embodiment, the immunogenic polypeptide is as described throughout the present disclosure. For example, the immunogenic polypeptide can comprise a PEST-containing peptide.

In one embodiment, the system or process further comprises culturing and characterizing the Listeria strain to confirm expression and secretion of the T-cell neo-epitope. In one embodiment, the system or process further comprises culturing and characterizing the Listeria strain to confirm expression and secretion of the adaptive immune response neo-epitope. In one embodiment, the system or process further comprises culturing and characterizing the Listeria strain to confirm expression and secretion of the one or more nonsensical peptides.

In another embodiment, the process disclosed herein allows the generation of a personalized enhanced anti-disease, or anti-infection, or anti-infectious disease, or anti-tumor immune response in the subject having a disease. In another embodiment, the process allows personalized treatment or prevention of the disease, or the infection or infectious disease, or the tumor or cancer in a subject. In another embodiment, the process increases survival time in the subject having the disease, or the infection or infectious disease, or the tumor or cancer.

In one embodiment, the present disclosure comprises the step of generating an immunogenic composition comprising the recombinant Listeria strain disclosed herein, the recombinant polypeptide disclosed herein, or the nucleic acid sequence disclosed herein, and a pharmaceutical acceptable carrier. In one embodiment, the present disclosure comprises the step of generating an immunogenic composition comprising the combination of any one or more of the recombinant Listeria strain disclosed herein, the recombinant polypeptide disclosed herein, and the nucleic acid sequence disclosed herein, with a pharmaceutical acceptable carrier.

### V. Compositions and Methods of Use Thereof

In one embodiment, compositions disclosed herein are immunogenic compositions. Such immunogenic compositions can comprise at least one immunotherapy delivery vector as disclosed herein or at least one recombinant Listeria strain disclosed herein. Such immunogenic compositions can also further comprise an adjuvant.

Some such immunogenic compositions comprise multiple immunotherapy delivery vectors or multiple recombinant Listeria strains as disclosed herein. Each immunotherapy delivery vector or recombinant Listeria strain can comprise or encode a different recombinant polypeptide as disclosed herein or can comprise a different set of one or more immunogenic neo-epitopes. For example, the plurality of immunotherapy delivery vectors or recombinant Listeria strains can comprise, for example, 2-5, 5-10, 10-15, 15-20, 10-20, 20-30, 30-40, or 40-50 immunotherapy delivery vectors or recombinant Listeria strains. Likewise, the plurality of immunotherapy delivery vectors or recombinant Listeria strains can comprise, for example, about 5-10, 10-15, 15-20, 10-20, 20-30, 30-40, 40-50, 50-60, 60-70, 70-80, 80-90, 90-100, or 100-200 immunogenic neo-epitopes.

The immunogenic compositions, immunotherapy delivery vectors, or recombinant Listeria strains can be used in methods of treating, suppressing, preventing, or inhibiting a disease or a condition in a subject, comprising administering to the subject the immunogenic composition(s), immunotherapy delivery vector(s), or recombinant Listeria strain(s), wherein the one or more frameshift-mutation-derived peptides are encoded by a source nucleic acid sequence from a disease-bearing or condition-bearing biological sample from the subject. Such methods can elicit a personalized anti-disease or anti-condition immune response in the subject, wherein the personalized immune response is targeted to the one or more frameshift-mutation-derived peptides. For example, the disease or condition can be a condition or tumor. As disclosed elsewhere herein, such methods can further comprise administering a booster treatment.

In one embodiment, a Listeria disclosed herein induces a strong innate stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J. Immunol. 2001 Feb. 15; 166(4):2276-82, incorporated herein by reference in its entirety). In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, administration of compositions disclosed herein induces the production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fins-like tyrosine kinase (sFlt)-1; or soluble endoglin (sEng). In one embodiment, a Listeria of the present disclosure is involved in the release of anti-angiogenic factors, and, therefore, in one embodiment, has a therapeutic role in addition to its role as a plasmid vector for introducing an antigen to a subject. Each Listeria strain and type thereof represents a separate embodiment of the present disclosure.

The immune response induced by methods and compositions as disclosed herein is, in another embodiment, a T cell response. In another embodiment, the immune response comprises a T cell response. In another embodiment, the response is a CD8+ T cell response. In another embodiment, the response comprises a CD8⁺ T cell response. Each possibility represents a separate embodiment as disclosed herein. In another embodiment, the administering results in the generation of a personalized T-cell immune response against a disease or condition.

In another embodiment, administration of compositions disclosed herein increases the number of antigen-specific T cells. In another embodiment, administration of compositions activates co-stimulatory receptors on T cells. In another embodiment, administration of compositions induces proliferation of memory and/or effector T cells. In another embodiment, administration of compositions increases proliferation of T cells. In another embodiment, administration of compositions elicits an enhanced anti-tumor T cell response in a subject. In another embodiment, administration of compositions to inhibit tumor-mediated immunosuppression in a subject. In another embodiment, administration of compositions increases the ratio or T effector cells to regulatory T cells (Tregs) in the spleen and tumor of a subject.

In another embodiment, administering the composition to the subject generates a personalized enhanced anti-disease, or anti-condition immune response in the subject. In another embodiment, the immune response comprises an anti-cancer or anti-tumor response. In another embodiment, the immune response comprises an anti-infectious disease response.

As used throughout, the terms “composition” and “immunogenic composition” are interchangeable having all the same meanings and qualities.

In another embodiment, an immunogenic composition disclosed herein comprising a recombinant Listeria strain and further comprising an antibody or a functional fragment thereof for concomitant or sequential administration of each component is also referred to as a “combination therapy”. It is to be understood by a skilled artisan that a combination therapy may also comprise additional components, antibodies, therapies, etc.

A skilled artisan will appreciate that the term “pharmaceutical composition” may encompass a composition suitable for pharmaceutical use, for example, to administer to a subject in need.

In one embodiment, disclosed herein is a pharmaceutical composition comprising the recombinant Listeria strain disclosed herein and a pharmaceutically acceptable carrier.

In another embodiment, disclosed herein is a pharmaceutical composition comprising a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject, and a pharmaceutically acceptable carrier.

In another embodiment, a “Listeria vaccine” or “vaccine” when used in reference to a Listeria is used interchangeably with “Listeria immunotherapy” or “immunotherapy” herein. In another embodiment an immunotherapy disclosed herein comprises at least one recombinant Listeria strain disclosed herein, and a pharmaceutically acceptable carrier.

In another embodiment, a pharmaceutical composition comprising a recombinant Listeria strain comprising at least one nucleic acid sequence, each nucleic acid sequence encoding one or more recombinant polypeptides comprising one or one or more immunogenic neo-epitopes fused to an immunogenic polypeptide, wherein one or more of the neo-epitopes are encoded by a source nucleic acid sequence comprising at least one mutation, and wherein the source is obtained from a disease or condition bearing biological sample of a subject, and a pharmaceutically acceptable carrier. For example, the at least one mutation can be a nonsynonymous missense mutation or a somatic nonsynonymous missense mutation.

In another embodiment, disclosed herein is a pharmaceutical composition comprising a recombinant Listeria strain comprising at least one nucleic acid sequence, each nucleic acid sequence encoding one or more recombinant polypeptides comprising one or one or more immunogenic neo-epitopes, wherein one or more of the neo-epitopes are encoded by a source nucleic acid sequence, and wherein the source is obtained from a disease or condition bearing biological sample of a subject, and a pharmaceutically acceptable carrier.

In another embodiment, disclosed herein is a pharmaceutical composition comprising the nucleic acid sequence molecule disclosed herein, and a pharmaceutically acceptable carrier. In another embodiment, the present disclosure provides a DNA vaccine comprising a nucleic acid sequence molecule disclosed herein, and a pharmaceutically acceptable carrier.

In another embodiment, disclosed herein is a pharmaceutical composition comprising a vaccinia virus strain or virus-like particle disclosed herein and a pharmaceutically acceptable carrier.

In another embodiment, disclosed herein is a pharmaceutical composition comprising the recombinant polypeptide comprising one or more neo-epitopes disclosed herein and a pharmaceutically acceptable carrier. In another embodiment, a peptide vaccine comprises one or more recombinant polypeptides comprising one or more neo-epitopes disclosed herein, and a pharmaceutically acceptable carrier.

In another embodiment, disclosed herein is a pharmaceutical composition comprising the nonsensical peptide or fragment thereof comprising one or more neo-epitopes disclosed herein and a pharmaceutically acceptable carrier. In another embodiment, a peptide vaccine, DNA vaccine, vaccinia virus or virus-like particle, or recombinant Listeria disclosed herein comprises or express (where applicable) one or more nonsensical peptides or fragments thereof comprising one or more neo-epitopes disclosed herein and a pharmaceutically acceptable carrier.

A skilled artisan would appreciate that the term “pharmaceutical composition” encompasses a therapeutically effective amount of the active ingredient or ingredients including at least one of: one or more recombinant Listeria strains, one or more recombinant polypeptide comprising one or more nonsensical peptides comprising at least one neo-epitope, at least one nucleic acid sequence encoding one or more neo-epitopes, one or more nonsensical peptide or fragment thereof, all as disclosed herein, and any combination thereof, together with a pharmaceutically acceptable carrier or diluent. It is to be understood that the term a “therapeutically effective amount” refers to that amount which provides a therapeutic effect for a given condition and administration regimen.

In another embodiment, disclosed herein is a recombinant vaccine vector comprising a nucleotide acid sequence molecule also disclosed herein. In another embodiment, the vector is an expression vector. In another embodiment, the expression vector is a plasmid. In another embodiment, the present disclosure provides a method for the introduction of a nucleotide molecule of the present disclosure into a cell. Methods for constructing and utilizing recombinant vectors are well known in the art and are described, for example, in Sambrook et al. (2001, Molecular Cloning: A Laboratory Manual, Cold Spring Harbor Laboratory, New York), and in Brent et al. (2003, Current Protocols in Molecular Biology, John Wiley & Sons, New York). In another embodiment, the vector is a bacterial vector. In other embodiments, the vector is selected from Salmonella sp., Shigella sp., BCG, L. monocytogenes and S. gordonii. In another embodiment, one or more peptides are delivered by recombinant bacterial vectors modified to escape phagolysosomal fusion and live in the cytoplasm of the cell. In another embodiment, the vector is a viral vector. In other embodiments, the vector is selected from Vaccinia, Avipox, Adenovirus, AAV, Vaccinia virus NYVAC, Modified vaccinia strain Ankara (MA), Semliki Forest virus, Venezuelan equine encephalitis virus, herpes viruses, and retroviruses. In another embodiment, the vector is a naked DNA vector. In another embodiment, the vector is any other vector known in the art. Each possibility represents a separate embodiment of the present disclosure.

In another embodiment, a composition comprising a Listeria strain disclosed herein further comprises an adjuvant. In another embodiment, a composition comprising at least one of: one or more recombinant Listeria strain, one or more recombinant polypeptides comprising one or more neo-epitopes, at least one nucleic acid sequence encoding one or more neo-epitopes, one or more nonsensical peptide or fragment thereof, of the present disclosure, further comprises an adjuvant. In one embodiment, a composition of the present disclosure further comprises an adjuvant.

In another embodiment an immunogenic composition comprises the vector comprising the nucleic acid sequence comprising the recombinant polypeptide comprising one or more nonsensical peptides or fragment thereof fused to an immunogenic polypeptide or fragment thereof and an adjuvant. In another embodiment an immunogenic composition comprises the recombinant polypeptide comprising one or more nonsensical peptides or fragment thereof fused to an immunogenic polypeptide or fragment thereof and an adjuvant.

In one embodiment the composition comprises an adjuvant as known in the art or as disclosed herein. The adjuvant utilized in methods and compositions of the present disclosure is, in another embodiment, a granulocyte/macrophage colony-stimulating factor (GM-CSF) protein, a GM-CSF protein, a nucleotide molecule encoding GM-CSF, saponin QS21, monophosphoryl lipid A, SBAS2, an unmethylated CpG-containing oligonucleotide, an immune-stimulating cytokine, a nucleotide molecule encoding an immune-stimulating cytokine, a quill glycoside, a bacterial mitogen, or a bacterial toxin. Yet another example of a suitable adjuvant is detoxified listeriolysin O (dtLLO) protein. One example of a dtLLO suitable for use as an adjuvant is encoded by SEQ ID NO: 67. A dtLLO encoded by a sequence at least 90%, 95%, 96%, 97%, 98%, or 99% identical to SEQ ID NO: 67 is also suitable for use as an adjuvant. Each possibility represents a separate embodiment of the disclosure. In another embodiment, the adjuvant is or comprises any other adjuvant known in the art.

In one embodiment, an immunogenic composition disclosed herein comprises a recombinant Listeria strain disclosed herein.

In one embodiment, a composition comprises a recombinant Listeria monocytogenes (Lm) strain. In one embodiment, an immunogenic composition comprises a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject. In another embodiment, a nonsensical peptide or fragment thereof is fused to a truncated LLO, a truncated ActA or PEST sequence.

In one embodiment, an immunogenic composition comprises at least one recombinant Listeria strain comprising at least one nucleic acid sequence, each nucleic acid sequence encoding one or more recombinant polypeptides comprising one or more immunogenic neo-epitopes, wherein one or more of the neo-epitopes are encoded by a source nucleic acid sequence comprising at least one mutation, and wherein the source is obtained from a disease or condition bearing biological sample of a subject.

In one embodiment, an immunogenic composition of comprises at least one recombinant Listeria strain comprising at least one nucleic acid sequence, each nucleic acid sequence encoding one or more recombinant polypeptides comprising one or more immunogenic neo-epitopes fused to an immunogenic polypeptide, wherein one or more of the neo-epitopes are encoded by a source nucleic acid sequence comprising at least one mutation, and wherein the source is obtained from a disease or condition bearing biological sample of a subject.

In another embodiment an immunogenic composition comprises the vector comprising the nucleic acid sequence comprising the recombinant polypeptide comprising one or more nonsensical peptides or fragment thereof fused to an immunogenic polypeptide or fragment thereof. In another embodiment, an immunogenic composition of this disclosure comprises at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject.

In one embodiment, an immunogenic composition disclosed herein comprises one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of the one or more nonsensical peptides or fragments thereof comprises one or more immunogenic neo-epitopes, wherein one or more of the neo-epitopes, wherein one or more of the neo-epitopes are encoded by a source nucleic acid sequence, and wherein the source is obtained from a disease or condition bearing biological sample of a subject.

In one embodiment, an immunogenic composition comprises at least one nucleic acid sequence encoding one or more immunogenic neo-epitopes, and wherein the source is obtained from a disease or condition bearing biological sample of a subject.

In one embodiment, an immunogenic composition disclosed herein comprises a recombinant Listeria, a delivery vector or expression vector disclosed herein. In one embodiment, the source nucleic acid is obtained from a disease or condition bearing biological sample. In yet another embodiment, a disease or condition disclosed herein is an infectious disease, autoimmune disease, organ transplantation rejection, a tumor or a cancer. In another embodiment, the infectious disease comprises a viral or bacterial infection.

In another embodiment, each component of the immunogenic compositions disclosed herein is administered prior to, concurrently with, or after another component of the immunogenic compositions disclosed herein.

The compositions disclosed herein, in another embodiment, are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment, the compositions are administered orally, and are thus formulated in a form suitable for oral administration, i.e., as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment, of the present disclosure, the active ingredient is formulated in a capsule. In accordance with this embodiment, the compositions of the present disclosure comprise, in addition to the active compound and the inert carrier or diluent, a hard gelating capsule.

In another embodiment, compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In one embodiment, a subject is administered a dose of the any of the compositions of the present disclosure every 1-2 weeks, every 2-3 weeks, every 3-4 weeks, every 4-5 weeks, every 6-7 weeks, every 7-8 weeks, or every 9-10 weeks in order to achieve the intended elicitation of an immune response targeted at the subject's disease or condition. In one embodiment, a subject is administered a dose of the any of the compositions of the present disclosure every 1-2 months, every 2-3 months, every 3-4 months, every 4-5 months, every 6-7 months, every 7-8 months, or every 9-10 months in order to achieve the intended elicitation of an immune response targeted at the subject's disease or condition.

In one embodiment, repeat administrations (booster doses) of compositions or vaccines of this disclosure may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. Assessment may be determined by any of the techniques known in the art, including diagnostic methods such as imaging techniques, analysis of serum tumor markers, biopsy, or the presence, absence or amelioration of tumor associated symptoms.

In one embodiment, a subject is administered a booster dose every 1-2 weeks, every 2-3 weeks, every 3-4 weeks, every 4-5 weeks, every 6-7 weeks, every 7-8 weeks, or every 9-10 weeks in order to achieve the intended anti-tumor response. In one embodiment, a subject is administered a booster dose every 1-2 months, every 2-3 months, every 3-4 months, every 4-5 months, every 6-7 months, every 7-8 months, or every 9-10 months in order to achieve the intended elicitation of an immune response targeted at the subject's disease or condition.

It is also to be understood that administration of such compositions enhance an immune response, or increase a T effector cell to regulatory T cell ratio or elicit an anti-tumor immune response, as further disclosed herein. In one embodiment, disclosed herein is methods of use which comprise administering a composition comprising the described Listeria strains, and further comprising an antibody or functional fragment thereof. In another embodiment, methods of use comprise administering more than one antibody disclosed herein, which may be present in the same or a different composition, and which may be present in the same composition as the Listeria or in a separate composition. Each possibility represents a different embodiments of the methods disclosed herein.

It will be understood by the skilled artisan that the term “administering” encompasses bringing a subject in contact with a composition of the present disclosure. In one embodiment, administration can be accomplished in vitro, i.e. in a test tube, or in vivo, i.e. in cells or tissues of living organisms, for example humans. In one embodiment, methods disclosed herein encompass administering the Listeria strains and compositions thereof to a subject.

In one embodiment, a vaccine comprises a composition as disclosed herein. In another embodiment the vaccine further comprises an adjuvant, and/or a pharmaceutical carrier.

In another embodiment, methods disclosed herein comprises at least a single administration of an composition, vaccine, and/or Listeria strain, as disclosed herein, wherein in another embodiment, methods comprise multiple administrations of a composition, vaccine, and/or Listeria strain. Each possibility represents a separate embodiment of methods disclosed herein.

In one embodiment, methods disclosed herein comprise a single administration of recombinant Listeria. In another embodiment, Listeria is administered twice. In another embodiment, Listeria is administered three times. In another embodiment, Listeria is administered four times. In another embodiment, Listeria is administered more than four times. In another embodiment, Listeria is administered multiple times. In another embodiment, Listeria is administered at regular intervals, which in one embodiment, may be daily, weekly, every two weeks, every three weeks, or every month. Each possibility represents a separate embodiment of a method disclosed herein.

In one embodiment, methods comprise administering a composition disclosed herein a single time. In another embodiment, a composition is administered twice. In another embodiment, a composition is administered three times. In another embodiment, a composition is administered four times. In another embodiment, a composition is administered more than four times. In another embodiment, a composition is administered multiple times. In another embodiment, a composition is administered at regular intervals, which in one embodiment, may be daily, weekly, every two weeks, every three weeks, or every month. Each possibility represents a separate embodiment of the methods disclosed herein.

In one embodiment, methods comprise administering a vaccine a single time. In another embodiment, a vaccine is administered twice. In another embodiment, a vaccine is administered three times. In another embodiment, a vaccine is administered four times. In another embodiment, a vaccine is administered more than four times. In another embodiment, a vaccine is administered multiple times. In another embodiment, a vaccine is administered at regular intervals, which in one embodiment, may be daily, weekly, every two weeks, every three weeks, or every month. Each possibility represents a separate embodiment of methods disclosed herein.

It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

In one embodiment, a delivery vector refers to the recombinant Listeria as disclosed herein, the nucleic acid sequence encoding one or more nonsensical peptides or neo-epitopes as disclosed herein, the recombinant polypeptide comprising one or more nonsensical peptides or neo-epitopes as disclosed herein, the nucleic acid sequence encoding one or more nonsensical peptides as disclosed herein, or the recombinant polypeptide comprising one or more nonsensical peptides as disclosed herein.

In another embodiment, a composition disclosed herein comprises at least one delivery vector and any combination thereof of different or same delivery vectors.

In one embodiment the DNA molecule or nucleic sequence molecule refer to one or more, but not limited to, a plasmid or artificial chromosome, comprising the nucleic acid sequence encoding the recombinant polypeptide comprising one or more neo-epitopes.

In one embodiment the DNA molecule or nucleic sequence molecule refer to one or more, but not limited to, a plasmid or artificial chromosome, comprising the nucleic acid sequence encoding the recombinant polypeptide comprising one or more neo-epitopes fused to an immunogenic polypeptide.

In one embodiment the DNA molecule or nucleic sequence molecule refer to one or more, but not limited to, a plasmid or artificial chromosome, comprising the nucleic acid sequence encoding the recombinant polypeptide comprising one or more nonsensical peptides or fragments thereof fused to an immunogenic polypeptide, wherein the nonsensical peptide comprising one or more neo-epitopes.

In one embodiment, a personalized immunotherapy composition, as disclosed herein, comprises one or more delivery vectors as disclosed herein. In one embodiment, a personalized immunotherapy composition disclosed herein comprises one or more Listeria strain(s) as disclosed in any of the above. In another embodiment, a personalized immunotherapy composition comprises a mixture of 1-2, 1-5, 1-10, 1-20 or 1-40 recombinant delivery vectors, each vector expressing one or more neo-epitopes. In another embodiment, the mixture comprises a plurality of delivery vectors (e.g., recombinant Listeria strains,) each delivery vector comprising a different set of one or more neo-epitopes. A first set of neo-epitopes can be different from a second set if it includes one neo-epitope that the second set does not. Likewise, a first set of neo-epitopes can be different from a second set if it does not include a neo-epitopes that the second set does include. For example, a first set and a second set of neo-epitopes can include one or more of the same neo-epitopes and can still be different sets, or a first set can be different from a second set of neo-epitopes by virtue of not including any of the same neo-epitopes. In another embodiment, a personalized immunotherapy composition comprises a mixture of 1-2, 1-5, 1-10, 1-20 or 1-40 recombinant delivery vectors, each vector expressing one or more nonsensical peptides or fragments thereof. Each possibility represents a separate embodiment.

In another embodiment, a personalized immunotherapy composition comprises a mixture of 1-2, 1-5, 1-10, 1-20 or 1-40 recombinant delivery vectors, each vector expressing one or more neo-epitopes in the context of a fusion protein with a truncated LLO protein, a truncated ActA protein or a PEST amino acid sequence. In one embodiment, the individual delivery vectors present in the mixture of delivery vectors are administered concomitantly to a subject as part of a therapy. In another embodiment, the individual delivery vectors present in the mixture of delivery vectors are administered sequentially to a subject as part of a therapy. Each possibility represents a separate embodiment.

In one embodiment, disclosed herein, an immunogenic composition comprising one or more recombinant delivery vectors produced by the process disclosed herein. In one embodiment, disclosed herein, an immunogenic mixture of compositions comprising one or more recombinant delivery vectors produced by the process disclosed herein. In another embodiment, each of said delivery vector in said mixture comprises a nucleic acid sequence encoding a recombinant polypeptide or chimeric protein comprising one or more neo-epitopes.

It would be appreciated by one skilled in the art that the term “recombinant delivery vectors” encompasses a recombinant Listeria strain delivery vector, a polypeptide delivery vector, or a DNA molecule delivery vector as described herein.

In another embodiment, each mixture of compositions comprises 1-5, 5-10, 10-15, 15-20, 10-20, 20-30, 30-40, 40-50, 50-60, 60-70, 70-80, or 80-100 delivery vectors. Each possibility represents a separate embodiment.

In one embodiment, disclosed herein, an immunogenic mixture of compositions comprising one or more recombinant Listeria strains produced by the process disclosed herein. In another embodiment, each of said Listeria in the mixture comprises at least one nucleic acid sequence encoding a recombinant polypeptide or chimeric protein comprising one or more neo-epitopes. In another embodiment, each mixture comprises 1-5, 5-10, 10-15, 15-20, 10-20, 20-30, 30-40, or 40-50, or 50-100 recombinant Listeria strains. Each possibility represents a separate embodiment.

In another embodiment, the composition comprises a plurality or combination of Listeria strains, wherein each strain comprises the nucleic acid construct comprising one or more open reading frames encoding one or more peptides comprising at least one neo-epitope. In another embodiment, the composition comprises a plurality or combination of Listeria strains, wherein each strain comprises the nucleic acid construct comprising one or more open reading frames encoding one or more nonsensical peptides or fragments thereof comprising one or more neo-epitope.

In another embodiment a composition may include a plurality of neo-epitopes. In another embodiment, the composition comprises at least two different neo-epitopes amino acid sequences. In another embodiment, the composition expresses at least two different neo-epitopes amino acid sequences.

In another embodiment, the composition comprises neo-epitopes in the range of about 1-5, 5-10, 10-15, 15-20, 10-20, 20-30, 30-40, 40-50, 50-60, 60-70, 70-80, 80-90, 90-100, 100-200, 200-300, 300-400, 400-500, or 500-1000 neo-epitopes. Each possibility represents a separate embodiment. In another embodiment, the composition comprises the neo-epitopes in the range of about 50-100 neo-epitopes. In another embodiment, the composition comprises the neo-epitopes in the range of about 1-100 neo-epitopes. In another embodiment, the composition comprises above about 100 neo-epitopes. In another embodiment, the composition comprises up to about 10 neo-epitopes. In another embodiment, the composition comprises up to about 20 neo-epitopes. In another embodiment, the composition comprises up to about 50 neo-epitopes. In another embodiment, the composition comprises up to about 100 neo-epitopes. In another embodiment, the composition comprises up to about 500 neo-epitopes. In another embodiment, the composition comprises up to about 1000 neo-epitopes. In another embodiment, the composition comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 neo-epitopes. Each possibility represents a separate embodiment. In another embodiment, the composition comprises about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 neo-epitopes. Each possibility represents a separate embodiment.

In one embodiment, any composition comprising a Listeria strain described herein may be used in the methods disclosed herein.

In another embodiment, the composition further comprises at least one immunomodulatory molecule, wherein the molecule is selected from a group comprising Interferon gamma, a cytokine, a chemokine, a T-cell stimulant, and any combination thereof.

In one embodiment, administrating the Listeria strain to a subject having said disease or condition generates an immune response targeted to the subject's disease or condition. In another embodiment, the Listeria strain is a personalized immunotherapy vector for said subject, targeted to said subject's disease or condition. In another embodiment, the personalized immunotherapy increases survival time in the subject having the disease or condition. In another embodiment, the personalized immunotherapy reduces tumor size or metastases size in the subject having the disease or condition. In another embodiment, the personalized immunotherapy protects against metastases in the subject having the disease or condition.

In one embodiment, a method for increasing survival time of a subject having a tumor or suffering from cancer, or suffering from an infectious disease, comprises the step of administering to the subject the immunogenic composition as described throughout the present disclosure.

In another embodiment, a method for increasing survival time of a subject having a tumor or suffering from cancer, or suffering from an infectious disease, comprises the step of administering to the subject the personalized immunotherapy composition or vaccine disclosed herein.

In another embodiment, disclosed herein is a method of increasing survival of a subject suffering from cancer or having a tumor, the method comprising the step of administering to the subject an immunogenic composition comprising a recombinant Listeria vaccine strain, as described herein, comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to one or more neo-epitopes or nonsensical peptides or fragments thereof comprising one or more neo-epitopes.

In another embodiment, a method for increasing survival time of a subject having a tumor or suffering from cancer, or suffering from an infectious disease, comprises the step of administering to the subject an immunogenic composition comprising a recombinant Listeria strain disclosed herein.

In another embodiment, disclosed herein is a method for increasing survival time of a subject having a tumor or suffering from cancer, or suffering from an infectious disease, the method comprising the step of administering to the subject the immunogenic composition comprising at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein the source is obtained from a disease or condition bearing biological sample of a subject.

In one embodiment, a method of eliciting a personalized targeted immune response in a subject having a disease or condition, wherein the immune response is targeted to one or more nonsensical peptides or fragments thereof comprising one or more neo-epitopes present within a disease or condition bearing biological sample of a subject, comprises administering to the subject the immunogenic composition as described herein.

In another embodiment, disclosed herein is a method of eliciting a personalized targeted immune response in a subject having a disease or condition, wherein the immune response is targeted to a nonsensical peptide or fragment thereof comprising one or more neo-epitopes present within a disease or condition bearing tissue of a subject, comprising administering to the subject the immunogenic composition comprising at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein the source is obtained from a disease or condition bearing biological sample of a subject.

In one embodiment, a method of eliciting an immune response targeted to at least one neo-epitope present in a disease or condition bearing tissue or cell in a subject having the disease or condition, comprises the step of administering the personalized immunotherapy composition or vaccine as disclosed herein to the subject.

In one embodiment, a method of eliciting a targeted immune response in a subject having a disease or condition, comprises administering to the subject the immunogenic composition or vaccine as disclosed herein, wherein administrating the Listeria strain generates a personalized immunotherapy targeted to the subject's disease or condition.

In one embodiment, a method of treating, suppressing, preventing or inhibiting a disease or a condition in a subject, comprises administering to the subject the immunogenic composition as described herein.

In one embodiment, a method of treating, suppressing or inhibiting disease or condition in a subject, comprises the step of administrating a personalized immunotherapy composition or vaccine as described herein, for targeting the disease or condition.

In another embodiment, disclosed herein is a method of treating, suppressing, preventing or inhibiting disease or condition in a subject, comprising administering to the subject the immunogenic composition comprising at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of said one or more nonsensical peptides or fragments thereof comprises one or more immunogenic neo-epitopes, and wherein the source is obtained from a disease or condition bearing biological sample of a subject.

In another embodiment, a method comprises treating a tumor or a cancer or an infection or an infectious disease in a subject, comprises the step of administering to the subject an immunogenic composition comprising the recombinant Listeria strain disclosed herein.

In one embodiment, a method of increasing the ratio of T effector cells to regulatory T cells (Tregs) in the spleen and tumor of a subject, wherein the T effector cells are targeted to one or more nonsensical peptides comprising one or more neo-epitopes present within a disease or condition bearing biological sample of a subject, comprises the step of administering to the subject the immunogenic composition of as described herein.

In another embodiment, disclosed herein is a method of increasing a ratio of T effector cells to regulatory T cells (Tregs) in the spleen and tumor microenvironments of a subject, comprising administering the immunogenic composition disclosed herein. In another embodiment, increasing a ratio of T effector cells to regulatory T cells (Tregs) in the spleen and tumor microenvironments in a subject allows for a more profound anti-tumor response in the subject.

In another embodiment, a method of increasing the ratio of T effector cells to regulatory T cells (Tregs) in the spleen and tumor of a subject, wherein the T effector cells are targeted to a neo-epitope present within a disease or condition bearing tissue of a subject, comprises the step of administering to the subject personalized immunotherapy composition or vaccine as disclosed herein.

In another embodiment, disclosed herein is a method of increasing the ratio of T effector cells to regulatory T cells (Tregs) in the spleen and tumor of a subject, wherein the T effector cells are targeted to one or more nonsensical peptides comprising one or more neo-epitopes present within a disease or condition bearing tissue of a subject, the method comprising the step of administering to the subject the immunogenic composition comprising at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein the source is obtained from a disease or condition bearing biological sample of a subject.

In another embodiment, the T effector cells comprise CD4+FoxP3− T cells. In another embodiment, the T effector cells are CD4+FoxP3− T cells. In another embodiment, the T effector cells comprise CD4+FoxP3− T cells and CD8+ T cells. In another embodiment, the T effector cells are CD4+FoxP3− T cells and CD8+ T cells. In another embodiment, the regulatory T cells is a CD4+FoxP3+ T cell.

In another embodiment, the immune response is a T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response.

Following the administration of the immunogenic compositions disclosed herein, the methods disclosed herein induce the expansion of T effector cells in peripheral lymphoid organs leading to an enhanced presence of T effector cells at the tumor site. In another embodiment, the methods disclosed herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs. It will be appreciated by the skilled artisan that peripheral lymphoid organs include, but are not limited to, the spleen, peyer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decrease the frequency of Tregs, but not the total number of Tregs at these sites.

In one embodiment, a method for increasing neo-epitope-specific T-cells in a subject, comprises the step of administering to the subject the immunogenic composition as described herein.

In another embodiment, disclosed herein is a method for increasing neo-epitope-specific T-cells in a subject, the method comprising the step of administering to the subject the immunogenic composition comprising at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein the source is obtained from a disease or condition bearing biological sample of a subject.

In another embodiment, disclosed herein is a method of increasing antigen-specific T cells in a subject suffering from cancer or having a tumor, comprises the step of administering to the subject an immunogenic composition, as described herein, wherein the fusion polypeptide comprises a truncated listeriolysin O (LLO) protein, a truncated ActA protein, or a PEST amino acid sequence fused to one or more neo-epitopes or nonsensical peptides or fragments thereof comprising one or more neo-epitopes.

In another embodiment, a method for increasing antigen-specific T-cells in a subject, comprises the step of administering to the subject a personalized immunotherapy composition or vaccine, wherein the recombinant polypeptide comprises one or more neo-epitopes or nonsensical peptides or fragments thereof.

In another embodiment, a method for increasing antigen-specific T-cells in a subject, comprises the step of administering to the subject an immunogenic composition comprising a recombinant Listeria strain disclosed herein.

In one embodiment, a method of reducing tumor or metastases size in a subject, comprises the step of administering to the subject the immunogenic composition as described herein.

In another embodiment, disclosed herein is a method of reducing tumor or metastases size in a subject, the method comprising the step of administering to the subject the immunogenic composition comprising at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein the source is obtained from a disease or condition bearing biological sample of a subject.

In another embodiment, a method of protecting a subject from an infectious disease, comprises the step of administering to the subject a personalized immunotherapy composition or vaccine as disclosed herein.

In another embodiment, a method of protecting a subject against a tumor or cancer, comprises the step of administering to the subject the immunogenic composition disclosed herein.

In another embodiment, a method of inhibiting or delaying the onset of cancer in a subject, comprises the step of administering to the subject a personalized immunotherapy composition or vaccine as disclosed herein.

In one embodiment, the method elicits a personalized anti-cancer or anti-tumor immune response.

In one embodiment, disclosed herein is a method of eliciting an enhanced anti-tumor T cell response in a subject, the method comprising the step of administering to the subject an effective amount of an immunogenic composition comprising a recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein one or more nonsensical peptides are encoded by a source nucleic acid sequence comprising at least one frameshift mutation, wherein each of the one or more nonsensical peptides or fragments thereof comprises one or more immunogenic neo-epitopes, wherein the source is obtained from a disease or condition bearing biological sample of a subject, and wherein the method further comprises a step of administering an effective amount of a composition comprising an immune check-point inhibitor antagonist.

In one embodiment, disclosed herein is a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to one or more neo-epitopes, or nonsensical peptides or fragments thereof comprising one or more neo-epitopes, wherein the method further comprises a step of administering an effective amount of a composition comprising an immune checkpoint inhibitor antagonist.

In one embodiment, the composition comprises one or more checkpoint inhibitors. In another embodiment, checkpoint inhibitors may include one or more antibody. In another embodiment, one or more antibodies may include an anti-PD-L1/PD-L2 antibody or fragment thereof; an anti-PD-1 antibody or fragment thereof; anti-CTLA-4 antibody or fragment thereof; anti-B7-H4 antibody or fragment thereof; and any combination thereof. Other immune checkpoint inhibitor antagonists include a PD-1 signaling pathway inhibitor, a CD-80/86 and CTLA-4 signaling pathway inhibitor, a T cell membrane protein 3 (TIM3) signaling pathway inhibitor, an adenosine A2a receptor (A2aR) signaling pathway inhibitor, a lymphocyte activation gene 3 (LAGS) signaling pathway inhibitor, a killer immunoglobulin receptor (KIR) signaling pathway inhibitor, a CD40 signaling pathway inhibitor, or any other antigen-presenting cell/T cell signaling pathway inhibitor.

In one embodiment, the composition comprises one or more of a T cell stimulator, such as an antibody or functional fragment thereof binding to a T-cell receptor co-stimulatory molecule, an antigen presenting cell receptor binding co-stimulatory molecule, or a member of the TNF receptor superfamily. The T-cell receptor co-stimulatory molecule can comprise, for example, CD28 or ICOS. The antigen presenting cell receptor binding co-stimulatory molecule can comprise, for example, a CD80 receptor, a CD86 receptor, or a CD46 receptor. The TNF receptor superfamily member can comprise, for example, glucocorticoid-induced TNF receptor (GITR), OX40 (CD134 receptor), 4-1BB (CD137 receptor), or TNFR25. See, e.g., WO2016100929, WO2016011362, and WO2016011357, each of which is incorporated by reference in its entirety for all purposes.

In one embodiment, repeat administrations (doses) of compositions disclosed herein may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. Assessment may be determined by any of the techniques known in the art, including diagnostic methods such as imaging techniques, analysis of serum tumor markers, biopsy, or the presence, absence or amelioration of tumor associated symptoms.

In one embodiment, disclosed herein are methods and compositions for preventing, treating and vaccinating against a heterologous antigen-expressing tumor and inducing an immune response against sub-dominant epitopes of the heterologous antigen, while preventing an escape mutation of the tumor.

In one embodiment, the methods and compositions for preventing, treating and vaccinating against a heterologous antigen-expressing tumor comprise the use of a truncated Listeriolysin (tLLO) protein. In another embodiment, the methods and compositions disclosed herein comprise a recombinant Listeria overexpressing tLLO. In another embodiment, the tLLO is expressed from a plasmid within the Listeria.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. In another embodiment, “treating” refers inter alia to delaying progression, expediting remission, inducing remission, augmenting remission, speeding recovery, increasing efficacy of or decreasing resistance to alternative therapeutics, or a combination thereof. The terms “reducing”, “suppressing” and “inhibiting” refer in another embodiment, to lessening or decreasing. In another embodiment, the terms “inhibiting” and “suppressing” refer to prophylactic or preventative measures, wherein the object is to prevent or lessen the targeted pathologic condition or disease, as described hereinabove. In another embodiment, treating may include directly affecting or curing the disease, disorder or condition and/or related symptoms, while suppressing or inhibiting may include preventing, reducing the severity of, delaying the onset of, reducing symptoms associated with the disease, disorder or condition, or a combination thereof. In one embodiment, “prophylaxis,” “prophylactic,” “preventing” or “inhibiting” refers, inter alia, to delaying the onset of symptoms, preventing relapse to a disease, decreasing the number or frequency of relapse episodes, increasing latency between symptomatic episodes, or a combination thereof. In one embodiment, “suppressing” refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. Each possibility may represent a separate embodiment.

In one embodiment the vaccine, composition, or recombinant Listeria strain is administered in a therapeutically effective amount. A skilled artisan would appreciate that the term “therapeutically effective amount”, in reference to the treatment of tumor, encompasses an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a vaccine disclosed herein for purposes of treatment of tumor may be determined empirically and in a routine manner.

In another embodiment, a method of inducing regression of a tumor in a subject, comprises the step of administering to the subject the immunogenic composition disclosed herein. In another embodiment, a method of reducing the incidence or relapse of a tumor or cancer, comprises the step of administering to the subject the immunogenic composition disclosed herein. In another embodiment, a method of suppressing the formation of a tumor in a subject, comprises the step of administering to the subject the immunogenic composition disclosed herein. In another embodiment, a method of inducing a remission of a cancer in a subject, comprises the step of administering to the subject the immunogenic composition disclosed herein.

In one embodiment, the method comprises the step of co-administering the recombinant Listeria with an additional therapy. In another embodiment, the additional therapy is surgery, chemotherapy, an immunotherapy, a radiation therapy, antibody-based immunotherapy, or a combination thereof. In another embodiment, the additional therapy precedes administration of the recombinant Listeria. In another embodiment, the additional therapy follows administration of the recombinant Listeria. In another embodiment, the additional therapy is an antibody therapy. In another embodiment, the recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ, TNF-α, and other cytokines known in the art to enhance cellular immune response, some of which can be found in U.S. Pat. No. 6,991,785, incorporated by reference herein.

In one embodiment, the methods disclosed herein further comprise the step of co-administering an immunogenic composition disclosed herein with a indoleamine 2,3-dioxygenase (IDO) pathway inhibitor. IDO pathway inhibitors for use in the present disclosure include any IDO pathway inhibitor known in the art, including but not limited to, 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an ana-IDO antibody or a small molecule IDO inhibitor. In another embodiment, the compositions and methods disclosed herein are also used in conjunction with, prior to, or following a chemotherapeutic or radiotherapeutic regiment. In another embodiment, IDO inhibition enhances the efficiency of chemotherapeutic agents.

In one embodiment, disclosed herein is a method of eliciting a personalized anti-tumor response in a subject, the method comprising the step of concomitantly or sequentially administering to the subject an immunogenic mixture composition disclosed herein. In another embodiment, disclosed herein is a method of preventing or treating a tumor in a subject, the method comprising the step of concomitantly or sequentially administering to the subject the immunogenic mixture of compositions disclosed herein. In one embodiment, a composition comprising at least one recombinant Listeria strain selected from the mixture of compositions may be administered simultaneously (i.e., in the same medicament), concurrently (i.e., in separate medicaments administered one right after the other in any order) or sequentially in any order with at least another recombinant Listeria strain selected from said mixture of compositions. Sequential administration is particularly useful when a drug substance comprising a recombinant Listeria strain disclosed herein is in different dosage forms (one agent is a tablet or capsule and another agent is a sterile liquid) and/or are administered on different dosing schedules, e.g., one composition from the mixture of compositions comprising one Listeria strain is administered at least daily and another that is administered less frequently, such as once weekly, once every two weeks, or once every three weeks.

In another embodiment, the personalized immunotherapy composition elicits an immune response targeted against one or more neo-epitopes. In another embodiment, the personalized immunotherapy composition elicits an immune response targeted against one or more nonsensical peptides or fragments thereof.

In an effort to treat a subject having an autoimmune disease, disclosed herein are immunogenic compositions and process to identify auto-reactive neo-epitopes, wherein the method or process comprises methods to immunize a subject having an autoimmune disease against these auto-reactive neo-epitopes, in order to induce tolerance mediated by antibodies or immunosuppressor cells, for examples Tregs or MDSCs.

In one embodiment, an autoimmune disease comprises a systemic autoimmune disease. The term “systemic autoimmune disease” refers to a disease, disorder or a combination of symptoms caused by autoimmune reactions affecting more than one organ. In another embodiment, a systemic autoimmune disease includes, but is not limited to, Anti-GBM nephritis (Goodpasture's disease), Granulomatosis with polyangiitis (GPA), microscopic polyangiitis (MPA), systemic lupus erythematosus (SLE), polymyositis (PM) or Celiac disease.

In one embodiment, an autoimmune disease comprises a connective tissue disease. A skilled artisan would appreciate that the term “connective tissue disease” encompasses a disease, condition or a combination of symptoms caused by autoimmune reactions affecting the connective tissue of the body. In another embodiment, a connective tissue disease includes, but is not limited to, systemic lupus erythematosus (SLE), polymyositis (PM), systemic sclerosis or mixed connective tissue disease (MCTD).

In one embodiment, other non-tumor or non-cancerous diseases, including organ transplantation rejection from which a disease-bearing biological sample can be obtained for analysis according to the process disclosed herein. In another embodiment, the rejected organ is a solid organ, including but not limited to a heart, a lung, a kidney, a liver, pancreas, intestine, stomach, testis, cornea, skin, heart valve, a blood vessel, or bone. In another embodiment, the rejected organs include but are not limited to a blood tissue, bone marrow, or islets of Langerhans cells.

In an effort to treat a transplant subject having a rejection of the transplanted organ or is experiencing graft v. host disease (GVhD), in one embodiment, methods to identify auto-reactive neo-epitopes are disclosed herein, wherein the process comprises methods to immunize a subject having an autoimmune disease against these auto-reactive neo-epitopes, in order to induce tolerance mediated by antibodies or immunosuppressor cells, for examples Tregs or MDSCs.

In one embodiment, the method as described herein, further comprising administering a booster treatment. In another embodiment, administering elicits a personalized enhanced anti-infectious disease immune response in the subject. In another embodiment, administering elicits an enhanced anti-infectious disease, or anti-condition personalized immune response in the subject. In another embodiment, the method elicits an anti-cancer or anti-tumor personalized immune response. In another embodiment, a method further comprises boosting the subject with an immunogenic composition comprising an attenuated Listeria strain disclosed herein. In another embodiment, a method comprises the step of administering a booster dose of the immunogenic composition comprising the recombinant Listeria strain disclosed herein. In another embodiment the booster includes one or more DNA molecule/nucleic acid sequence/nucleic acid construct/nucleic acid vector as described herein. In another embodiment the booster includes one or more recombinant polypeptide/chimeric protein/peptide/fusion peptide as described herein.

In another embodiment the booster comprises at least one recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject.

In another embodiment the booster comprises at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject.

In another embodiment the booster comprises one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of the one or more nonsensical peptides or fragments thereof comprises one or more immunogenic neo-epitopes, and wherein the source is obtained from a disease or condition bearing biological sample of a subject.

In another embodiment the booster comprises one or more recombinant polypeptides comprising one or more immunogenic neo-epitopes, wherein one or more of the neo-epitopes, wherein one or more of the neo-epitopes are encoded by a source nucleic acid sequence comprising at least one mutation, and wherein the source is obtained from a disease or condition bearing biological sample of a subject.

In another embodiment, a method disclosed herein further comprises the step of boosting the subject with a recombinant Listeria strain or an antibody or functional fragment thereof, as disclosed herein. In another embodiment, the recombinant Listeria strain used in the booster inoculation is the same as the strain used in the initial “priming” inoculation. In another embodiment, the booster strain is different from the priming strain. In another embodiment, the antibody used in the booster inoculation binds the same antigen as the antibody used in the initial “priming” inoculation. In another embodiment, the booster antibody is different from the priming antibody.

In another embodiment, the same doses are used in the priming and boosting inoculations. In another embodiment, a larger dose is used in the booster. In another embodiment, a smaller dose is used in the booster.

In another embodiment, the methods disclosed herein further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations.

In another embodiment, the booster dose is an alternate form of the immunogenic composition. In another embodiment, the methods further comprise the step of administering to the subject a booster immunogenic composition. In one embodiment, the booster dose follows a single priming dose of the immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria disclosed herein is experimentally determined by the skilled artisan. In another embodiment, the dose is experimentally determined by a skilled artisan. In another embodiment, the period between a prime and a boost dose is 1 week, in another embodiment, it is 2 weeks, in another embodiment, it is 3 weeks, in another embodiment, it is 4 weeks, in another embodiment, it is 5 weeks, in another embodiment, it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). US Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. All of the above references are herein incorporated by reference in their entireties.

In one embodiment, a treatment protocol encompassed by the disclosure is therapeutic. In another embodiment, the protocol is prophylactic. In another embodiment, the compositions disclosed herein are used to protect people at risk for cancer such as breast cancer or other types of tumors because of familial genetics or other circumstances that predispose them to these types of ailments as will be understood by a skilled artisan. In another embodiment, an immunotherapy or a vaccine disclosed herein is used as a cancer immunotherapy after debulking of tumor growth by surgery, conventional chemotherapy or radiation treatment. Following such treatments, the immunotherapy or vaccine is administered so that the CTL response to the tumor antigen destroys remaining metastases and prolongs remission from the cancer. In another embodiment, immunotherapies or vaccines are used to effect the growth of previously established tumors and to kill existing tumor cells.

In another embodiment, one or more neo-epitope sequence comprised in a peptide, a recombinant polypeptide, or a fusion polypeptide is used to provide a therapeutic anti-tumor or anti-cancer T-cell immune response. In another embodiment, use of one or more neo-epitope sequence comprised in a peptide, a recombinant polypeptide, or a fusion polypeptide provides a targeting immunotherapy, which may, in certain embodiments therapeutically activate an anti-tumor or anti-cancer adaptive immune response. In another embodiment, a one or more neo-epitope sequence comprised in a peptide, a recombinant polypeptide, or a fusion polypeptide is used to provide a therapeutic anti-autoimmune disease T-cell immune response. In another embodiment, use of a one or more neo-epitope sequence comprised in a peptide, a recombinant polypeptide, or a fusion polypeptide provides a targeting immunotherapy, which may, in certain embodiments therapeutically activate an anti-autoimmune disease adaptive immune response. In another embodiment, a one or more neo-epitope sequence comprised in a peptide, a recombinant polypeptide, or a fusion polypeptide is used to provide a therapeutic anti-infectious disease T-cell immune response. In another embodiment, use of a one or more neo-epitope sequence comprised in a peptide, a recombinant polypeptide, or a fusion polypeptide provides a targeting immunotherapy, which may, in certain embodiments therapeutically activate an anti-infectious disease adaptive immune response. In another embodiment, a one or more neo-epitope sequence comprised in a peptide, a recombinant polypeptide, or a fusion polypeptide is used to provide a therapeutic anti-organ transplantation rejection T-cell immune response. In another embodiment, use of a one or more neo-epitope sequence comprised in a peptide, a recombinant polypeptide, or a fusion polypeptide provides a targeting immunotherapy, which may, in certain embodiments therapeutically activate an anti-organ transplantation rejection adaptive immune response.

In another embodiment, wherein the presence of an immunogenic response correlates with a presence of one or more immunogenic neo-epitopes. In another embodiment, a recombinant Listeria comprises nucleic acid encoding neo-epitopes comprising T-cell epitopes, or adaptive immune response epitopes, or any combination thereof.

In another embodiment, a one or more nonsensical peptide sequence comprised in a peptide, a recombinant polypeptide, or a fusion polypeptide is used to provide a therapeutic anti-tumor or anti-cancer T-cell immune response. In another embodiment, use of a one or more nonsensical peptide sequence comprised in a peptide, a recombinant polypeptide, or a fusion polypeptide provides a targeting immunotherapy, which may, in certain embodiments therapeutically activate an anti-tumor or anti-cancer adaptive immune response. In another embodiment, a one or more nonsensical peptide sequence is used to provide a therapeutic anti-autoimmune disease T-cell immune response. In another embodiment, one or more nonsensical peptide sequence is used to activate an anti-autoimmune disease adaptive immune response. In another embodiment, a one or more nonsensical peptide sequence is used to provide a therapeutic anti-infectious disease T-cell immune response. In another embodiment, one or more nonsensical peptide sequences used in activating an anti-infectious disease adaptive immune response. In another embodiment, a one or more nonsensical peptide sequence is used to provide a therapeutic anti-organ transplantation rejection T-cell immune response. In another embodiment, one or more nonsensical peptide sequence comprised in a peptide, a recombinant polypeptide, or a fusion polypeptide provides a targeting immunotherapy, which may, in certain embodiments therapeutically is used to activate an anti-organ transplantation rejection adaptive immune response.

In another embodiment, the presence of an immunogenic response correlates with a presence of one or more immunogenic nonsensical peptides. In another embodiment, a recombinant Listeria comprises nucleic acid encoding one or more nonsensical peptides or fragments thereof comprising T-cell epitopes, or adaptive immune response epitopes, or any combination thereof.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

A skilled artisan would appreciate that the term “method” encompasses manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It will be appreciated by a skilled artisan that the term “plurality” may encompass an integer above 1. In one embodiment, the term refers to a range of 1-10, 10-20, 20-30, 30-40, 40-50, 60-70, 70-80, 80-90, or 90-100. Each possibility represents a separate embodiment.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Listing of Embodiments

The subject matter disclosed herein includes, but is not limited to, the following embodiments.

1. A recombinant Listeria strain comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein said source is obtained from a disease or condition bearing biological sample of a subject.

2. The recombinant Listeria strain of embodiment 1, wherein said frameshift mutation is in comparison to a source nucleic acid sequence of a healthy biological sample.

3. The recombinant Listeria strain of any one of embodiments 1-2, wherein said at least one frameshift mutation comprises multiple frameshift mutations and said multiple frameshift mutations are present within the same gene in said recombinant Listeria strain.

4. The recombinant Listeria strain of any one of embodiments 1-2, wherein said at least one frameshift mutation comprises multiple frameshift mutations and said multiple frameshift mutations are not present within the same gene in said recombinant Listeria strain.

5. The recombinant Listeria strain of any one of embodiments 1-6, wherein said at least one frameshift mutation is within an exon encoding region of a gene.

6. The recombinant Listeria strain of embodiment 7, wherein said exon is the last exon of said gene.

7. The recombinant Listeria strain of any one of embodiments 1-8, wherein each of said one or more nonsensical peptides is about 60-100 amino acids in length.

8. The recombinant Listeria strain of any one of embodiments 1-9, wherein said one or more nonsensical peptide is expressed in said disease or condition bearing biological sample.

9. The recombinant Listeria strain of any one of embodiments 1-10, wherein said one or more nonsensical peptide does not encode a post-translational cleavage site.

10. The recombinant Listeria strain of any one of embodiments 1-11, wherein said source nucleic acid sequence comprises one or more regions of microsatellite instability.

11. The recombinant Listeria strain of any one of embodiments 1-12, wherein said one or more neo-epitopes comprises a T-cell epitope.

12. The recombinant Listeria strain of any one of embodiments 1-13, wherein said one or more neo-epitopes comprises a self-antigen associated with said disease or condition, wherein said self-antigen comprises a cancer or tumor-associated neo-epitope, or a cancer-specific or tumor-specific neo-epitope.

13. The recombinant Listeria strain of embodiment 14, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor, or a metastasis of any one of said cancers or tumors.

14. The recombinant Listeria strain of any one of embodiments 1-15, wherein said one or more nonsensical peptides comprising one or more neo-epitopes comprising an infectious disease-associated or disease specific neo-epitope.

15. The recombinant Listeria strain of any one of embodiments 1-16, wherein said recombinant Listeria expresses and secretes said one or more recombinant polypeptides.

16. The recombinant Listeria strain of any one of embodiments 1-17, each of said recombinant polypeptides comprising about 1-20 said neo-epitopes.

17. The recombinant Listeria strain of any one of embodiments 1-18, wherein said one or more nonsensical peptides or fragments thereof are each fused to an immunogenic polypeptide.

18. The recombinant Listeria strain of any one of embodiments 1-17, wherein said one or more nonsensical peptides or fragments thereof comprise multiple operatively linked nonsensical peptides or fragments thereof from N-terminal to C-terminal, and wherein said immunogenic polypeptide is fused to one of said multiple nonsensical peptides or fragments thereof.

19. The recombinant Listeria of embodiment 18, wherein said immunogenic polypeptide is operatively linked to the N-terminal nonsensical peptide.

20. The recombinant Listeria of embodiment 22, wherein said link is a peptide bond.

21. The recombinant Listeria of any one of embodiments 1-20, wherein said immunogenic polypeptide is a mutated Listeriolysin O (LLO) protein, a truncated LLO (tLLO) protein, a truncated ActA protein, or a PEST amino acid sequence.

22. The recombinant Listeria of any one of embodiments 1-21, wherein said one or more recombinant polypeptides is operatively linked to a tag at the C-terminal, optionally via a linker sequence.

23. The recombinant Listeria of embodiment 22, wherein said linker sequence encodes a 4× glycine linker.

24. The recombinant Listeria of any one of embodiments 22-23, wherein said tag is selected from a group comprising a 6× Histidine tag, SIINFEKL peptide, 6× Histidine tag operatively linked to 6× histidine, and any combination thereof.

25. The recombinant Listeria of any one of embodiments 22-24, wherein said nucleic acid sequence encoding said recombinant polypeptide comprises 2 stop codons following the sequence encoding said tag.

26. The recombinant Listeria of any one of embodiments 1-25, wherein said nucleic acid sequence encoding said recombinant polypeptide encodes components comprising: pHly-tLLO-[nonsensical peptide or fragment thereof-glycine linker₍₄ₓ₎-nonsensical peptide or fragment thereof—glycine linker₍₄ₓ₎]ₙ-SIINFEKL-6× His tag-2× stop codon, wherein said nonsensical peptide or fragment thereof is twenty-one amino acids long, and wherein n=1-20.

27. The recombinant Listeria of embodiment 26, wherein said nonsensical peptide or fragment thereof may be the same or different.

28. The recombinant Listeria strain of any one of embodiments 1-27, wherein said at least one nucleic acid sequence encoding said recombinant polypeptide is integrated into the Listeria genome.

29. The recombinant Listeria strain of any one of embodiments 1-27, wherein said at least one nucleic acid sequence encoding said recombinant polypeptide is in a plasmid.

30. The recombinant Listeria strain of embodiment 29, wherein said plasmid is stably maintained in said Listeria strain in the absence of antibiotic selection.

31. The recombinant Listeria strain of any one of embodiments 1-30, wherein said Listeria strain is an attenuated Listeria strain.

32. The recombinant Listeria strain of embodiment 31, wherein said attenuated Listeria comprises a mutation in one or more endogenous genes.

33. The recombinant Listeria strain of embodiment 32, wherein said endogenous gene mutation is selected from an actA gene mutation, a prfA mutation, an actA and inlB double mutation, a dal/dal gene double mutation, or a dal/dat/actA gene triple mutation, or a combination thereof.

34. The recombinant Listeria strain of any one of embodiments 32-33, wherein said mutation comprises an inactivation, truncation, deletion, replacement or disruption of the gene or genes.

35. The recombinant Listeria strain of any one of embodiments 1-34, wherein said at least one nucleic acid sequence encoding said recombinant polypeptide further comprises a second open reading frame encoding a metabolic enzyme, or wherein said Listeria strain comprises a second nucleic acid sequence comprising an open reading frame encoding a metabolic enzyme.

36. The recombinant Listeria strain of embodiment 35, wherein said metabolic enzyme is an alanine racemase enzyme or a D-amino acid transferase enzyme.

37. The recombinant Listeria strain of any one of embodiments 1-36, wherein said Listeria is Listeria monocytogenes.

38. The recombinant Listeria strain of any one of embodiments 1-37, wherein said nonsensical peptide is acquired from the comparison of one or more open reading frames (ORF) in nucleic acid sequences extracted from said disease-bearing biological sample with one or more ORF in nucleic acid sequences extracted from a healthy biological sample, wherein said comparison identifies one or more frameshift mutations within said nucleic acid sequences, wherein said nucleic acid sequence comprising said mutations encodes one or more nonsensical peptides comprising one or more immunogenic neo-epitopes encoded within said one or more ORF from said disease-bearing biological sample.

39. The recombinant Listeria strain of any one of embodiment 1-38, wherein said disease-bearing biological sample is obtained from said subject having said disease or condition.

40. The recombinant Listeria strain of any one of embodiments 2 and 38, wherein said healthy biological sample is obtained from said subject having said disease or condition.

41. The recombinant Listeria strain of any one of embodiments 1-40, wherein said biological sample comprises a tissue, a cell, a blood sample, or a serum sample.

42. The recombinant Listeria strain of any one of embodiments 1-41, wherein said nonsensical peptide is characterized for neo-epitopes by:

(i) generating one or more different peptide sequences from said nonsensical peptide; and optionally,

(ii) screening each said peptides generated in (i) and selecting for binding by MHC Class I or MHC Class II to which a T-cell receptor binds to.

43. The recombinant Listeria strain of any one of embodiments 1-42, wherein said recombinant Listeria further comprises at least one nucleic acid sequence encoding one or more recombinant polypeptides comprising one or more peptides fused to an immunogenic polypeptide, wherein said one or more peptides comprise one or more immunogenic neoepitopes.

44. The recombinant Listeria strain of embodiment 43, wherein said one or more peptides or fragments thereof comprise multiple operatively linked peptides or fragments thereof from N-terminal to C-terminal, and wherein said immunogenic polypeptide is fused to one of said multiple peptides or fragments thereof.

45. The recombinant Listeria of any one of embodiments 43-44, wherein said immunogenic polypeptide is a mutated Listeriolysin O (LLO) protein, a truncated LLO (tLLO) protein, a truncated ActA protein, or a PEST amino acid sequence.

46. The recombinant Listeria of any one of embodiments 43-45, wherein said one or more recombinant polypeptides is operatively linked to a tag at the C-terminal, optionally via a linker sequence.

47. The recombinant Listeria of embodiment 46, wherein said linker sequence encodes a 4× glycine linker.

48. The recombinant Listeria of any one of embodiments 46-47, wherein said tag is selected from a group comprising a 6× Histidine tag, SIINFEKL peptide, 6× Histidine tag operatively linked to 6× histidine, and any combination thereof.

49. The recombinant Listeria of any one of embodiments 46-48, wherein said nucleic acid sequence encoding said recombinant polypeptide comprises 2 stop codons following the sequence encoding said tag.

50. The recombinant Listeria of any one of embodiments 43-49, wherein said nucleic acid sequence encoding said recombinant polypeptide encodes components comprising: pHly-tLLO-[peptide or fragment thereof-glycine linker₍₄ₓ₎-peptide or fragment thereof—glycine linker₍₄ₓ₎]ₙ-SIINFEKL-6× His tag-2× stop codon, wherein said peptide or fragment thereof is about twenty-one amino acids long, and wherein n=1-20.

51. The recombinant Listeria of embodiment 50, wherein said peptide or fragment comprises a different amino acid sequence.

52. An immunogenic composition comprising at least one of any one of the Listeria strains of any one of embodiments 1-51.

53. The immunogenic composition of embodiment 52, further comprising an additional adjuvant.

54. The immunogenic composition of embodiment 53, wherein said additional adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

55. A method of eliciting a personalized targeted immune response in a subject having a disease or condition, said method comprising administering to said subject the immunogenic composition of any one of embodiments 52-54, wherein said personalized immune response is targeted to one or more nonsensical peptides or fragments thereof comprising one or more neo-epitopes present within a disease or condition bearing biological sample of said subject.

56. A method of treating, suppressing, preventing or inhibiting a disease or a condition in a subject, said method comprising administering to said subject the immunogenic composition of any one of embodiments 52-54.

57. A method of increasing the ratio of T effector cells to regulatory T cells (Tregs) in the spleen and tumor of a subject, said method comprising the step of administering to the subject the immunogenic composition of any one of embodiments 52-54, wherein said T effector cells are targeted to one or more nonsensical peptides comprising one or more neo-epitopes present within a disease or condition bearing biological sample of a subject.

58. A method for increasing neo-epitope-specific T-cells in a subject, said method comprising the step of administering to said subject the immunogenic composition of any one of embodiments 52-54.

59. A method for increasing survival time of a subject having a tumor or suffering from cancer, or suffering from an infectious disease, said method comprising the step of administering to said subject the immunogenic composition of any one of embodiments 52-54.

60. A method of reducing tumor or metastases size in a subject, said method comprising the step of administering to said subject the immunogenic composition of any one of embodiments 52-54.

61. The method of any one of embodiments 52-54, further comprising administering a booster treatment.

62. The method of any one of embodiments 52-54, wherein said administering elicits a personalized enhanced anti-infectious disease immune response in said subject.

63. The method of any one of embodiments 52-54, wherein said method elicits a personalized anti-cancer or anti-tumor immune response.

64. An immunotherapy delivery vector comprising at least one nucleic acid sequence, each nucleic acid sequence encoding one or more recombinant polypeptides comprising one or more nonsensical peptides or fragments thereof fused to an immunogenic polypeptide, wherein said one or more nonsensical peptides are encoded by a source nucleic acid sequence comprising at least one frameshift mutation, wherein each of said one or more nonsensical peptides or fragments thereof comprises one or more immunogenic neo-epitopes, and wherein said source is obtained from a disease or condition bearing biological sample of a subject.

65. The immunotherapy delivery vector of embodiment 64, wherein said frameshift mutation is in comparison to a source nucleic acid sequence of a healthy biological sample.

66. The immunotherapy delivery vector of any one of embodiments 64-65, wherein said at least one frameshift mutation comprises multiple frameshift mutations and said multiple frameshift mutations are present within the same gene in said recombinant Listeria.

67. The immunotherapy delivery vector of any one of embodiments 64-65, wherein said at least one frameshift mutation comprises multiple frameshift mutations and said multiple frameshift mutations are not present within the same gene in said recombinant Listeria.

68. The immunotherapy delivery vector of any one of embodiments 64-67, wherein said at least one frameshift mutation is within an exon encoding region of a gene.

69. The immunotherapy delivery vector of embodiment 68, wherein said exon is the last exon of said gene.

70. The immunotherapy delivery vector of any one of embodiments 64-69, wherein each of said one or more nonsensical peptides is about 60-100 amino acids in length.

71. The immunotherapy delivery vector of any one of embodiments 64-70, wherein said one or more nonsensical peptide is expressed in said disease or condition bearing biological sample.

72. The immunotherapy delivery vector of any one of embodiments 64-71, wherein said one or more nonsensical peptide does not encode a post-translational cleavage site.

73. The immunotherapy delivery vector of any one of embodiments 64-72, wherein said source nucleic acid sequence comprises one or more regions of microsatellite instability.

74. The immunotherapy delivery vector of any one of embodiments 64-73, wherein said one or more neo-epitopes comprises a T-cell epitope.

75. The immunotherapy delivery vector of any one of embodiments 64-74, wherein said one or more neo-epitopes comprises a self-antigen associated with said disease or condition, wherein said self-antigen comprises a cancer or tumor-associated neo-epitope, or a cancer-specific or tumor-specific neo-epitope.

76. The immunotherapy delivery vector of embodiment 75, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor, or a metastasis of any one of said cancers or tumors.

77. The immunotherapy delivery vector of any one of embodiments 64-76, wherein said one or more nonsensical peptides comprising one or more neo-epitopes comprising an infectious disease-associated or disease specific neo-epitope.

78. The immunotherapy delivery vector of any one of embodiments 64-77, wherein said recombinant Listeria expresses and secretes said one or more recombinant polypeptides.

79. The immunotherapy delivery vector of any one of embodiments 64-78, wherein said one or more nonsensical peptides or fragments thereof are each fused to an immunogenic polypeptide.

80. The immunotherapy delivery vector of any one of embodiments 64-79, wherein said one or more nonsensical peptides or fragments thereof comprise multiple operatively linked nonsensical peptides or fragments thereof from N-terminal to C-terminal, and wherein said immunogenic polypeptide is fused to one of said multiple nonsensical peptides or fragments thereof.

81. The immunotherapy delivery vector of embodiment 80, wherein said immunogenic polypeptide is operatively linked to the N-terminal nonsensical peptide.

82. The immunotherapy delivery vector of embodiment 81, wherein said link is a peptide bond.

83. The immunotherapy delivery vector of any one of embodiments 64-82, wherein said immunogenic polypeptide is a mutated Listeriolysin O (LLO) protein, a truncated LLO (tLLO) protein, a truncated ActA protein, or a PEST amino acid sequence.

84. The immunotherapy delivery vector of any one of embodiments 64-83, wherein said one or more recombinant polypeptides is operatively linked to a tag at the C-terminal, optionally via a linker sequence.

85. The immunotherapy delivery vector of embodiment 84, wherein said linker sequence encodes a 4× glycine linker.

86. The immunotherapy delivery vector of any one of embodiments 84-85, wherein said tag is selected from a group comprising a 6× Histidine tag, SIINFEKL peptide, 6× Histidine tag operatively linked to 6× histidine, and any combination thereof.

87. The immunotherapy delivery vector of any one of embodiments 84-86, wherein said nucleic acid sequence encoding said recombinant polypeptide comprises 2 stop codons following the sequence encoding said tag.

88. The immunotherapy delivery vector of any one of embodiments 64-87, wherein said nucleic acid sequence encoding said recombinant polypeptide encodes components comprising: pHly-tLLO-[nonsensical peptide or fragment thereof-glycine linker₍₄ₓ₎-nonsensical peptide or fragment thereof—glycine linker₍₄ₓ₎]ₙ-SIINFEKL-6× His tag-2× stop codon, wherein said nonsensical peptide or fragment thereof is twenty-one amino acids long, and wherein n=1-20.

89. The immunotherapy delivery vector of any one of embodiments 64-88, wherein said nonsensical peptide is acquired from the comparison of one or more open reading frames (ORF) in nucleic acid sequences extracted from said disease-bearing biological sample with one or more ORF in nucleic acid sequences extracted from a healthy biological sample, wherein said comparison identifies one or more frameshift mutations within said nucleic acid sequences, wherein said nucleic acid sequence comprising said mutations encodes one or more nonsensical peptides comprising one or more immunogenic neo-epitopes encoded within said one or more ORF from said disease-bearing biological sample.

90. The immunotherapy delivery vector of any one of embodiment 64-89, wherein said disease-bearing biological sample is obtained from said subject having said disease or condition.

91. The immunotherapy delivery vector of any one of embodiments 65 and 89, wherein said healthy biological sample is obtained from said subject having said disease or condition.

92. The immunotherapy delivery vector of any one of embodiments 64-91, wherein said biological sample comprises a tissue, a cell, a blood sample, or a serum sample.

93. The immunotherapy delivery vector of any one of embodiments 64-92, wherein said nonsensical peptide is characterized for neo-epitopes by:

(i) generating one or more different peptide sequences from said nonsensical peptide; and optionally,

(ii) screening each said peptides generated in (i) and selecting for binding by MHC Class I or MHC Class II to which a T-cell receptor binds to.

94. The immunotherapy delivery vector of any one of embodiments 64-93, wherein said immunotherapy delivery vector further comprises at least one nucleic acid sequence encoding one or more recombinant polypeptides comprising one or more peptides fused to an immunogenic polypeptide, wherein said one or more peptides comprise one or more immunogenic neoepitopes.

95. The immunotherapy delivery vector of embodiment 94, wherein said one or more peptides or fragments thereof comprise multiple operatively linked peptides or fragments thereof from N-terminal to C-terminal, and wherein said immunogenic polypeptide is fused to one of said multiple peptides or fragments thereof.

96. The immunotherapy delivery vector of any one of embodiments 94-95, wherein said immunogenic polypeptide is a mutated Listeriolysin O (LLO) protein, a truncated LLO (tLLO) protein, a truncated ActA protein, or a PEST amino acid sequence.

97. The immunotherapy delivery vector of any one of embodiments 94-96, wherein said one or more recombinant polypeptides is operatively linked to a tag at the C-terminal, optionally via a linker sequence.

98 The immunotherapy delivery vector of embodiment 97, wherein said linker sequence encodes a 4× glycine linker.

99. The immunotherapy delivery vector of any one of embodiments 97-98, wherein said tag is selected from a group comprising a 6× Histidine tag, SIINFEKL peptide, 6× Histidine tag operatively linked to 6× histidine, and any combination thereof.

100. The immunotherapy delivery vector of any one of embodiments 97-99, wherein said nucleic acid sequence encoding said recombinant polypeptide comprises 2 stop codons following the sequence encoding said tag.

101. The immunotherapy delivery vector of any one of embodiments 94-100, wherein said nucleic acid sequence encoding said recombinant polypeptide encodes components comprising: pHly-tLLO-[peptide or fragment thereof-glycine linker₍₄ₓ₎-peptide or fragment thereof—glycine linker₍₄ₓ₎]ₙ-SIINFEKL-6× His tag-2× stop codon, wherein said peptide or fragment thereof is about twenty-one amino acids long, and wherein n=1-20.

102. The immunotherapy delivery vector of embodiment 101, wherein said peptide or fragment comprises a different amino acid sequence.

103. An immunogenic composition comprising at least one of any one of the Listeria strains of any one of embodiments 64-102.

104. The immunogenic composition of embodiment 103, further comprising an additional adjuvant.

105. The immunogenic composition of embodiment 104, wherein said additional adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

106. A method of eliciting a personalized targeted immune response in a subject having a disease or condition, said method comprising administering to said subject the immunogenic composition of any one of embodiments 103-105, wherein said personalized immune response is targeted to one or more nonsensical peptides or fragments thereof comprising one or more neo-epitopes present within a disease or condition bearing biological sample of said subject.

107. A method of treating, suppressing, preventing or inhibiting a disease or a condition in a subject, said method comprising administering to said subject the immunogenic composition of any one of embodiments 103-105.

108. A method of increasing the ratio of T effector cells to regulatory T cells (Tregs) in the spleen and tumor of a subject, said method comprising the step of administering to the subject the immunogenic composition of any one of embodiments 103-105, wherein said T effector cells are targeted to one or more nonsensical peptides comprising one or more neo-epitopes present within a disease or condition bearing biological sample of a subject.

109. A method for increasing neo-epitope-specific T-cells in a subject, said method comprising the step of administering to said subject the immunogenic composition of any one of embodiments 103-105.

110. A method for increasing survival time of a subject having a tumor or suffering from cancer, or suffering from an infectious disease, said method comprising the step of administering to said subject the immunogenic composition of any one of embodiments 103-105.

111. A method of reducing tumor or metastases size in a subject, said method comprising the step of administering to said subject the immunogenic composition of any one of embodiments 103-105.

112. The method of any one of embodiments 106-111, further comprising administering a booster treatment.

113. The method of any one of embodiments 106-111, wherein said administering elicits a personalized enhanced anti-infectious disease immune response in said subject.

114. The method of any one of embodiments 106-111, wherein said method elicits a personalized anti-cancer or anti-tumor immune response.

The subject matter disclosed herein also includes, but is not limited to, the following embodiments.

1. An immunotherapy delivery vector comprising a nucleic acid comprising an open reading frame encoding a recombinant polypeptide comprising a PEST-containing peptide fused to one or more heterologous peptides, wherein the one or more heterologous peptides comprise one or more frameshift-mutation-derived peptides comprising one or more immunogenic neo-epitopes.

2. The immunotherapy delivery vector of embodiment 1, wherein the one or more frameshift-mutation-derived peptides are encoded by a source nucleic acid sequence comprising at least one disease-specific or condition-specific frameshift mutation.

3. The immunotherapy delivery vector of embodiment 2, wherein the source nucleic acid sequence comprises one or more regions of microsatellite instability.

4. The immunotherapy delivery vector of any preceding embodiment, wherein the at least one frameshift mutation is within the penultimate exon or the last exon of a gene.

5. The immunotherapy delivery vector of any preceding embodiment, wherein each of the one or more frameshift-mutation-derived peptides is about 8-10, 11-20, 21-40, 41-60, 61-80, 81-100, 101-150, 151-200, 201-250, 251-300, 301-350, 351-400, 401-450, 451-500, or 8-500 amino acids in length.

6. The immunotherapy delivery vector of any preceding embodiment, wherein the one or more frameshift-mutation-derived peptides do not encode a post-translational cleavage site.

7. The immunotherapy delivery vector of any preceding embodiment, wherein the one or more immunogenic neo-epitopes comprise a T-cell epitope.

8. The immunotherapy delivery vector of any preceding embodiment, wherein the one or more frameshift-mutation-derived peptides comprise a cancer-associated or tumor-associated neo-epitope or a cancer-specific or tumor-specific neo-epitope.

9. The immunotherapy delivery vector of embodiment 8, wherein the tumor or cancer comprises a breast cancer or tumor, a cervical cancer or tumor, a Her2-expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor, or a metastasis of any one of the cancers or tumors.

10. The immunotherapy delivery vector of any one of embodiments 1-7, wherein the one or more frameshift-mutation-derived peptides comprise an infectious-disease-associated or infectious-disease-specific neo-epitope.

11. The immunotherapy delivery vector of any preceding embodiment, wherein the recombinant polypeptide comprises about 1-20 neo-epitopes.

12. The immunotherapy delivery vector of any preceding embodiment, wherein the one or more heterologous peptides comprise multiple heterologous peptides operably linked in tandem, wherein the PEST-containing peptide is fused to one of the multiple heterologous peptides.

13. The immunotherapy delivery vector of embodiment 12, wherein the recombinant polypeptide comprises multiple frameshift-mutation-derived peptides, wherein each frameshift-mutation-derived peptide is different.

14. The immunotherapy delivery vector of embodiment 12 or 13, wherein the multiple heterologous peptides are fused directly to each other with no intervening sequence.

15. The immunotherapy delivery vector of embodiment 12 or 13, wherein the multiple heterologous peptides are operably linked to each other via one or more peptide linkers or one or more 4× glycine linkers.

16. The immunotherapy delivery vector of any one of embodiments 12-15, wherein the PEST-containing peptide is operably linked to the N-terminal heterologous peptide.

17. The immunotherapy delivery vector of any preceding embodiment, wherein the PEST-containing peptide is a mutated listeriolysin O (LLO) protein, a truncated LLO (tLLO) protein, a truncated ActA protein, or a PEST amino acid sequence.

18. The immunotherapy delivery vector of any preceding embodiment, wherein the C-terminal end of the recombinant polypeptide is operably linked to a tag.

19. The immunotherapy delivery vector of embodiment 18, wherein the C-terminal end of the recombinant polypeptide is operably linked to a tag by a peptide linker or a 4× glycine linker.

20. The immunotherapy delivery vector of embodiment 18 or 19, wherein the tag is selected from the group consisting of: a 6× histidine tag, a 2× FLAG tag, a 3× FLAG tag, a SIINFEKL peptide, a 6× histidine tag operably linked to a SIINFEKL peptide, a 3× FLAG tag operably linked to a SIINFEKL peptide, a 2× FLAG tag operably linked to a SIINFEKL peptide, and any combination thereof.

21. The immunotherapy delivery vector of any one of embodiments 18-20, wherein the open reading frame encoding the recombinant polypeptide comprises two stop codons following the sequence encoding the tag.

22. The immunotherapy delivery vector of any preceding embodiment, wherein the open reading frame encoding the recombinant polypeptide is operably linked to an hly promoter and encodes components comprising from N-terminus to C-terminus: tLLO-[heterologous peptide]ₙ-(peptide tag(s))-(2× stop codon), wherein n=2-20, and wherein at least one heterologous peptide is a frameshift-mutation-derived peptide,

or wherein the open reading frame encoding the recombinant polypeptide is operably linked to an hly promoter and encodes components comprising from N-terminus to C-terminus: tLLO-[(heterologous peptide)-(glycine linker₍₄ₓ₎)]ₙ-(peptide tag(s))-(2× stop codon), wherein n=2-20, and wherein at least one heterologous peptide is a frameshift-mutation-derived peptide.

23. The immunotherapy delivery vector of any preceding embodiment, wherein the one or more heterologous peptides further comprise one or more nonsynonymous-missense-mutation-derived peptides.

24. The immunotherapy delivery vector of embodiment 23, wherein the one or more nonsynonymous-missense-mutation-derived peptides are encoded by a source nucleic acid sequence comprising at least one disease-specific or condition-specific nonsynonymous missense mutation.

25. The immunotherapy delivery vector of embodiment 23 or 24, wherein each of the one or more nonsynonymous-missense-mutation-derived peptides is about 5-50 amino acids in length or about 8-27 amino acids in length.

26. The immunotherapy delivery vector of any preceding embodiment, wherein the immunotherapy delivery vector is a recombinant Listeria strain.

27. The immunotherapy delivery vector of embodiment 26, wherein the recombinant Listeria strain expresses and secretes the recombinant polypeptide.

28. The immunotherapy delivery vector of embodiment 26 or 27, wherein the open reading frame encoding the recombinant polypeptide is integrated into the Listeria genome.

29. The immunotherapy delivery vector of embodiment 26 or 27, wherein the open reading frame encoding the recombinant polypeptide is in a plasmid.

30. The immunotherapy delivery vector of embodiment 29, wherein the plasmid is stably maintained in the recombinant Listeria strain in the absence of antibiotic selection.

31. The immunotherapy delivery vector of any one of embodiments 26-30, wherein the Listeria strain is an attenuated Listeria strain.

32. The immunotherapy delivery vector of embodiment 31, wherein the attenuated Listeria comprises a mutation in one or more endogenous genes.

33. The immunotherapy delivery vector of embodiment 32, wherein the endogenous gene mutation is selected from an actA gene mutation, a prfA mutation, an actA and inlB double mutation, a dal/dat gene double mutation, a dal/dat/actA gene triple mutation, or a combination thereof, and wherein the mutation comprises an inactivation, truncation, deletion, replacement, or disruption of the gene or genes.

34. The immunotherapy delivery vector of any one of embodiments 26-33, wherein the nucleic acid comprising the open reading frame encoding the recombinant polypeptide further comprises a second open reading frame encoding a metabolic enzyme, or wherein the recombinant Listeria strain further comprises a second nucleic acid comprising an open reading frame encoding a metabolic enzyme.

35. The immunotherapy delivery vector of embodiment 34, wherein the metabolic enzyme is an alanine racemase enzyme or a D-amino acid transferase enzyme.

36. The immunotherapy delivery vector of any one of embodiments 26-35, wherein the Listeria is Listeria monocytogenes.

37. The immunotherapy delivery vector of embodiment 36, wherein the recombinant Listeria strain comprises a deletion of or inactivating mutation in actA, dal, and dat, wherein the nucleic acid comprising the open reading frame encoding the recombinant polypeptide is in an episomal plasmid and comprises a second open reading frame encoding an alanine racemase enzyme or a D-amino acid aminotransferase enzyme, and wherein the PEST-containing peptide is an N-terminal fragment of LLO.

38. An immunogenic composition comprising at least one immunotherapy delivery vector of any one of embodiments 1-37.

39. The immunogenic composition of embodiment 38, further comprising an adjuvant.

40. The immunogenic composition of embodiment 49, wherein the adjuvant comprises a granulocyte/macrophage colony-stimulating factor (GM-CSF) protein, a nucleotide molecule encoding a GM-CSF protein, saponin QS21, monophosphoryl lipid A, an unmethylated CpG-containing oligonucleotide, or a detoxified, nonhemolytic form of LLO (dtLLO).

41. A method of treating, suppressing, preventing, or inhibiting a disease or a condition in a subject, comprising administering to the subject the immunogenic composition of any one of embodiments 38-40, wherein the one or more frameshift-mutation-derived peptides are encoded by a source nucleic acid sequence from a disease-bearing or condition-bearing biological sample from the subject.

42. The method of embodiment 42, wherein the method elicits a personalized anti-disease or anti-condition immune response in the subject, wherein the personalized immune response is targeted to the one or more frameshift-mutation-derived peptides.

43. The method of embodiment 41 or 42, wherein the disease or condition is a cancer or tumor.

44. The method of any one of embodiments 41-43, further comprising administering a booster treatment.

45. A process for creating the immunotherapy delivery vector of any one of embodiments 1-37 that is personalized for a subject having a disease or condition, comprising:

(a) comparing one or more open reading frames (ORFs) in nucleic acid sequences extracted from a disease-bearing or condition-bearing biological sample from the subject with one or more ORFs in nucleic acid sequences extracted from a healthy biological sample, wherein the comparing identifies one or more nucleic acid sequences encoding one or more peptides comprising one or more immunogenic neo-epitopes encoded within the one or more ORFs from the disease-bearing or condition-bearing biological sample, wherein at least one of the one or more nucleic acid sequences comprises one or more frameshift mutations and encodes one or more frameshift-mutation-derived peptides comprising one or more immunogenic neo-epitopes; and

(b) generating an immunotherapy delivery vector comprising a nucleic acid comprising an open reading frame encoding a recombinant polypeptide comprising the one or more peptides comprising the one or more immunogenic neo-epitopes identified in step (a).

46. The process of embodiment 45, further comprising storing the immunotherapy delivery vector for administering to the subject within a predetermined period of time.

47. The process of embodiment 45 or 46, further comprising administering a composition comprising the immunotherapy vector to the subject, wherein the administering results in the generation of a personalized T-cell immune response against the disease or condition.

48. The process of any one of embodiments 45-47, wherein the disease-bearing or condition-bearing biological sample is obtained from the subject having the disease or condition.

49. The process of any one of embodiments 45-48, wherein the healthy biological sample is obtained from the subject having the disease or condition.

50. The process of any one of embodiments 45-49, wherein the disease-bearing or condition-bearing biological sample and the healthy biological sample each comprises a tissue, a cell, a blood sample, or a serum sample.

51. The process of any one of embodiments 45-50, wherein the comparing in step (a) comprises use of a screening assay or screening tool and associated digital software for comparing the one or more ORFs in the nucleic acid sequences extracted from the disease-bearing or condition-bearing biological sample with the one or more ORFs in the nucleic acid sequences extracted from the healthy biological sample,

wherein the associated digital software comprises access to a sequence database that allows screening of mutations within the ORFs in the nucleic acid sequences extracted from the disease-bearing or condition-bearing biological sample for identification of immunogenic potential of the neo-epitopes.

52. The process of any one of embodiments 45-51, wherein the nucleic acid sequences extracted from the disease-bearing or condition-bearing biological sample and the nucleic acid sequences extracted from the healthy biological sample are determined using exome sequencing or transcriptome sequencing.

53. The process of any one of embodiments 45-52, wherein the one or more frameshift-mutation-derived peptides are characterized for neo-epitopes by generating one or more different peptide sequences from the one or more frameshift-mutation-derived peptides.

54. The process of embodiment 53, further comprising scoring each of the one or more different peptide sequences and excluding a peptide sequence if it does not score below a hydropathy threshold predictive of secretability in Listeria monocytogenes.

55. The process of embodiment 54, wherein the scoring is by a Kyte and Doolittle hydropathy index 21 amino acid window, and any peptide sequence scoring above a cutoff of about 1.6 is excluded or is modified to score below the cutoff.

56. The process of any one of embodiments 53-55, further comprising screening each of the one or more different peptide sequences and selecting for binding by MHC Class I or MHC Class II to which a T-cell receptor binds.

57. The process of any one of embodiments 45-56, wherein the process is repeated to create a plurality of immunotherapy delivery vectors, each comprising a different set of one or more immunogenic neo-epitopes.

58. The process of embodiment 57, wherein the plurality of immunotherapy delivery vectors comprises 2-5, 5-10, 10-15, 15-20, 10-20, 20-30, 30-40, or 40-50 immunotherapy delivery vectors.

59. The process of embodiment 57 or 58, wherein the combination of the plurality of immunotherapy delivery vectors comprises about 5-10, 10-15, 15-20, 10-20, 20-30, 30-40, 40-50, 50-60, 60-70, 70-80, 80-90, 90-100, or 100-200 immunogenic neo-epitopes.

60. The process of any one of embodiments 45-59, wherein the disease or condition is a tumor with fewer than 120, 110, 100, 90, 80, 70, 60, 50, 40, 30, 20, or 10 nonsynonymous missense mutations that are not present in the healthy biological sample.

While certain features have been illustrated and described herein, many modifications, substitutions, changes, and equivalents will now occur to those of ordinary skill in the art. It is, therefore, to be understood that the appended claims are intended to cover all such modifications and changes.

In the following examples, numerous specific details are set forth in order to provide a thorough understanding of the disclosure herein. In other instances, well-known methods, procedures, and components have not been described in detail so as not to obscure the present disclosure.

### EXAMPLES

### Example 1: Construction of Attenuated Listeria Strain-LmddΔactA and Insertion of the Human Klk3 Gene in Frame to the Hly Gene in the Lmdd and Lmdda Strains

### Materials and Methods

A recombinant Lm was developed that secretes PSA fused to tLLO (Lm-LLO-PSA), which elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector. We recently developed a new strain for the PSA vaccine based on the pADV142 plasmid, which has no antibiotic resistance markers, and referred as LmddA-142 (Table 1). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains.

| Plasmids | Features |
|---|---|
| pGG55 | pAM401/pGB354 shuttle plasmid with gram(−) |
| | and gram(+) cm resistance, LLO-E7 expression |
| | cassette and a copy of Lm prfA gene |
| pTV3 | Derived from pGG55 by deleting cm genes and |
| | inserting the Lm dal gene |
| pADV119 | Derived from pTV3 by deleting the prfA gene |
| pADV134 | Derived from pADV119 by replacing the Lm dal gene |
| | by the Bacillus dal gene |
| pADV142 | Derived from pADV134 by replacing HPV16 e7 |
| | with klk3 |
| pADV168 | Derived from pADV134 by replacing HPV16 e7 with |
| | hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly |
| | gene in the chromosome |
| LmddA-143 | 10403S dal⁽⁻⁾ dat⁽⁻⁾ actA⁽⁻⁾ with klk3 |
| | fused to the hly gene in the chromosome |
| LmddA-168 | 10403S dal⁽⁻⁾ dat⁽⁻⁾ actA⁽⁻⁾ pADV168 |
| Lmdd-143/134 | Lmdd-143 pADV134 |
| LmddA-143/134 | LmddA-143 pADV134 |
| Lmdd-143/168 | Lmdd-143 pADV168 |
| LmddA-143/168 | LmddA-143 pADV168 |

The sequence of the plasmid pAdv142 (6523 bp) was as set forth in SEQ ID NO: 23. This plasmid was sequenced at Genewiz facility from the E. coli strain on Feb. 20, 2008.

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat ΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in 4actA/pKSV7 (pAdv120).

TABLE 2
Sequence of primers that was used for
the amplification of DNA sequences
upstream and downstream of actA.

| Primer | Sequence | SEQ ID NO: |
|---|---|---|
| Adv271-actAF1 | cg GAATTCGGATCCgcgccaaatcattggttgang | 24 |
| Adv272-actAR1 | gcgaGTCGACgtcggggttaatcgtaatgcaattggc | 25 |
| Adv273-actAF2 | gcgaGTCGACccatacgacgttaancttgcaatg | 26 |
| Adv274-actAR2 | gataCTGCAGGGATCCttcccttctcggtaatcagtcac | 27 |

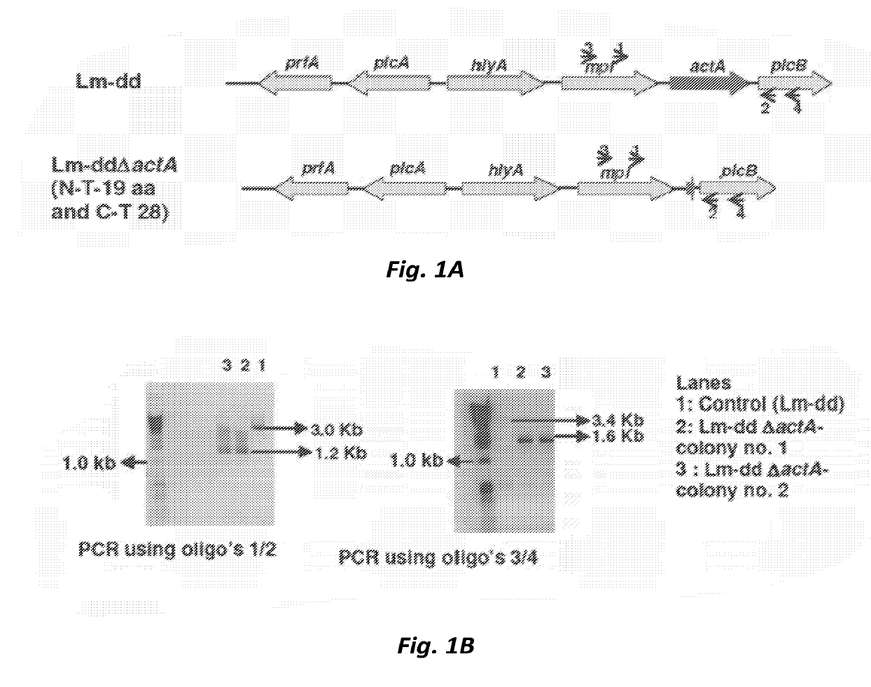

The deletion of the gene from its chromosomal location was verified using primers that bind externally to the actA deletion region, which are shown in FIG. 1A and FIG. 1B as primer 3 (Adv 305-tgggatggccaagaaattc, SEQ ID NO: 28) and primer 4 (Adv304-ctaccatgtcttccgttgcttg; SEQ ID NO: 29). The PCR analysis was performed on the chromosomal DNA isolated from Lmdd and LmddΔactA. The sizes of the DNA fragments after amplification with two different sets of primer pairs ½ and ¾ in Lmdd chromosomal DNA was expected to be 3.0 kb and 3.4 kb. On the other hand, the expected sizes of PCR using the primer pairs ½ and ¾ for the LmddΔactA was 1.2 kb and 1.6 kb. Thus, PCR analysis in FIG. 1A and FIG. 1B confirms that the 1.8 kb region of actA was deleted in the LmddΔactA strain. DNA sequencing was also performed on PCR products to confirm the deletion of actA containing region in the strain, LmddΔactA.

### Example 2: Construction of the Antibiotic-Independent Episomal Expression System for Antigen Delivery by Lm Vectors

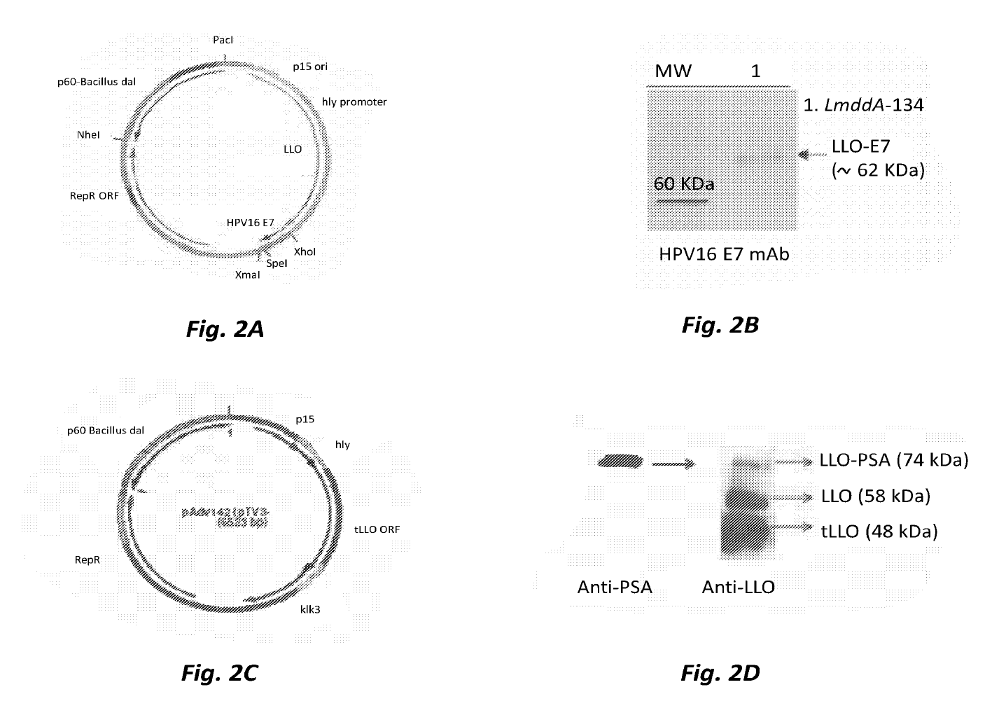

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3: In Vitro and In Vivo Stability of the Strain LmddA-LLO-PSA

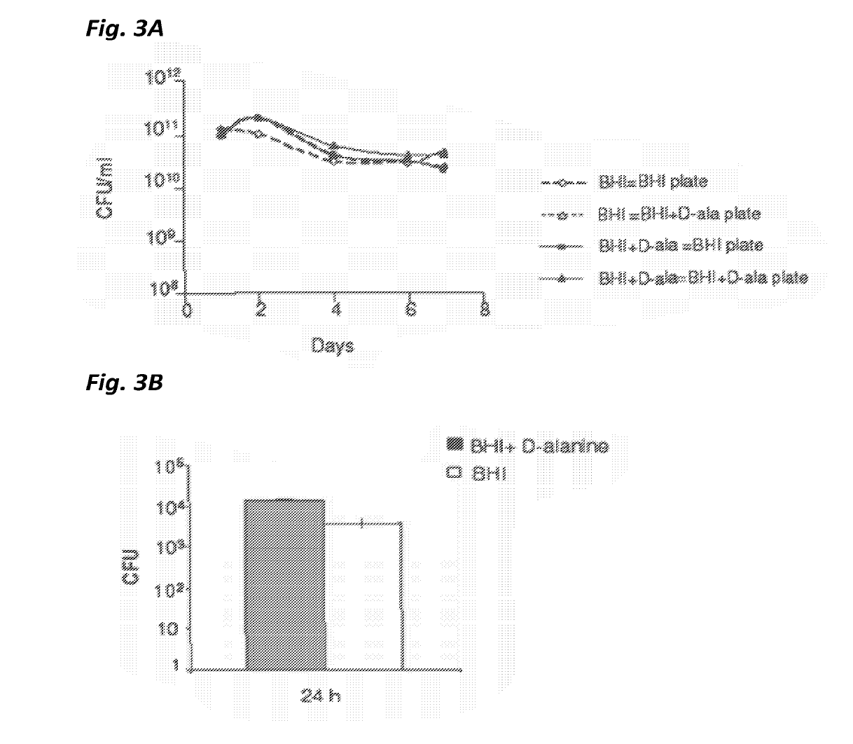

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 3A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 mg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 3B).

### Example 4: In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

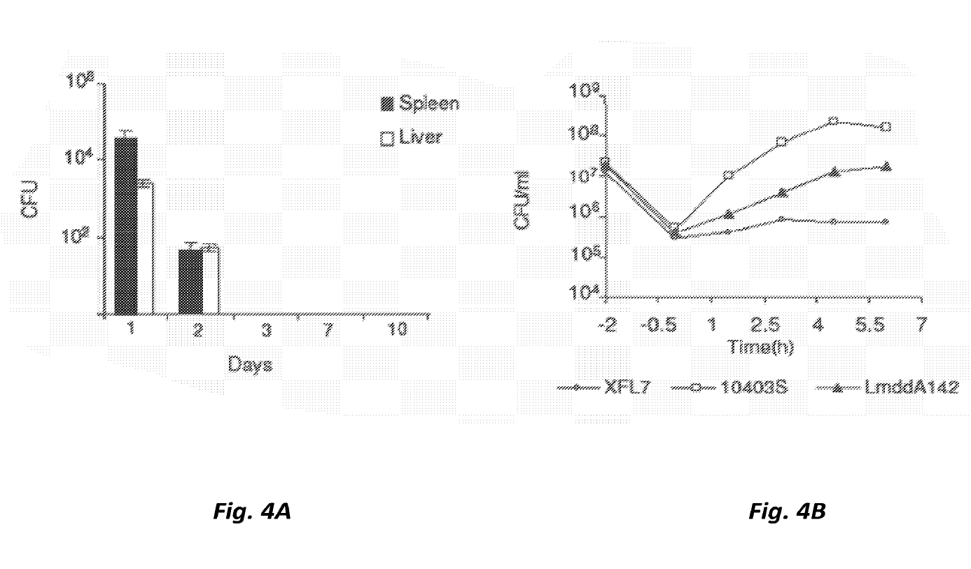

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 4A).

To determine if the attenuation of LmddA-LLO-PSA attenuated the ability of the strain LmddA-LLO-PSA to infect macrophages and grow intracellularly, a cell infection assay was performed. Mouse macrophage-like cell line such as J774A.1, were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 4B). The results indicate that LmddA-LLO-PSA has the ability to infect macrophages and grow intracytoplasmically.

### Example 5: Immunogenicity of the Strain-LmddA-LLO-PSA in C57BL/6 Mice

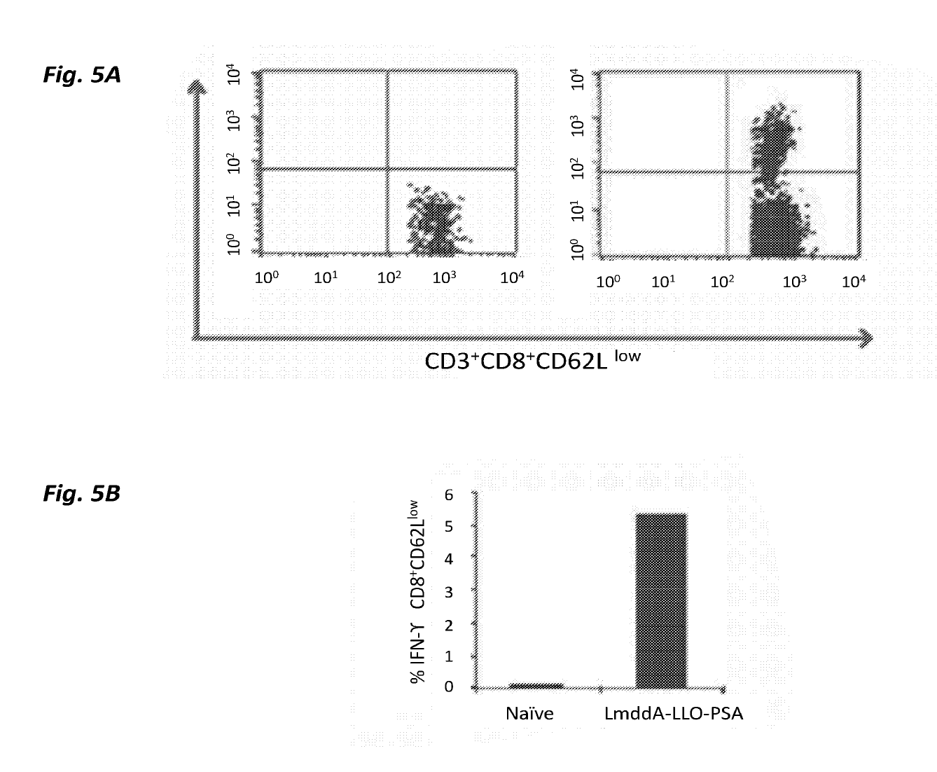

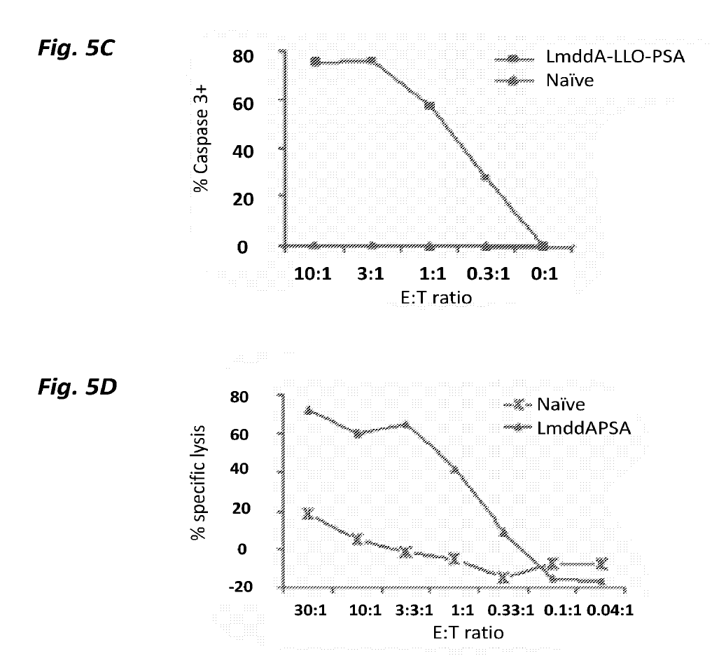

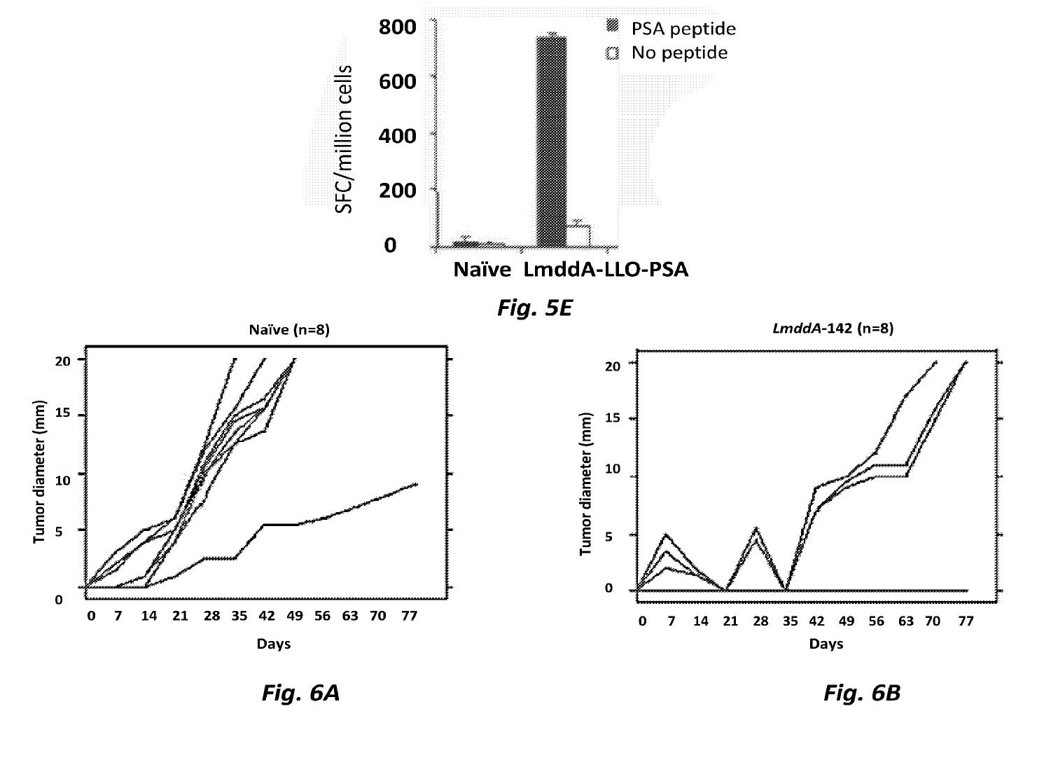

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 5A). The functional ability of the PSA-specific T cells to secrete IFN-γ after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naïve mice (FIG. 5B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 5C) and Europium release (FIG. 5D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, a 20-fold increase in the number of spots for IFN-γ in splenocytes from mice immunized with LmddA-LLO-PSA stimulated with specific peptide when compared to the splenocytes of the naïve mice was observed (FIG. 5E).

### Example 6: Immunization with the LmddA -142 Strains Induces Regression of a Tumor Expressing PSA and Infiltration of the Tumor by PSA-Specific CTLs

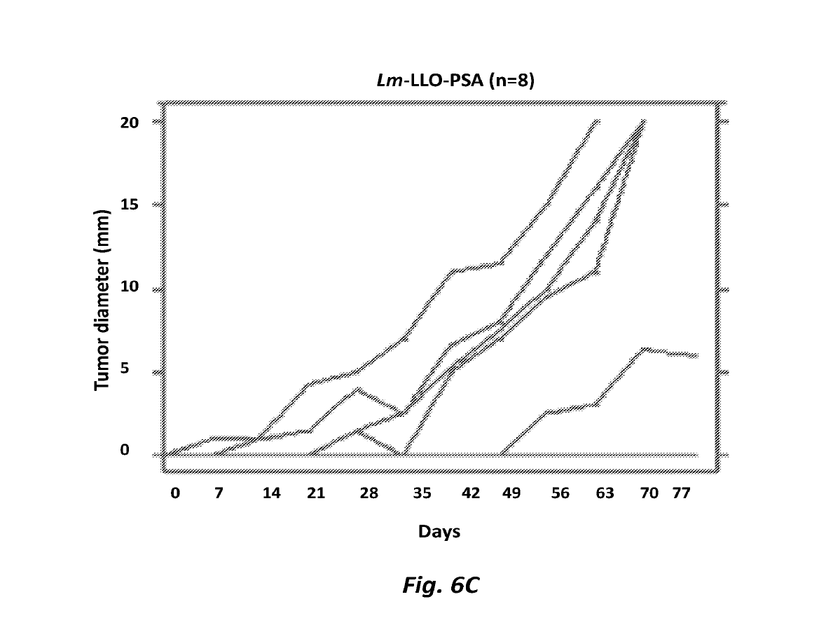

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 6A). The mice immunized with LmddA-142 were all tumor-free until day 35 and gradually 3 out of 8 mice developed tumors, which grew at a much slower rate as compared to the naïve mice (FIG. 6B). Five out of eight mice remained tumor free through day 70. As expected, Lm-LLO-PSA-vaccinated mice had fewer tumors than naïve controls and tumors developed more slowly than in controls (FIG. 6C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C1 tumors that were engineered to express PSA in more than 60% of the experimental animals (FIG. 6B), compared to none in the untreated group (FIG. 6A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naïve or control (Lm-LLO-E7) Listeria, or with LmddA-LLO-PSA. Tumors were excised on day 21 and were analyzed for the population of CD8⁺CD62L^{low} PSA^{tetramer+} and CD4⁺CD25^{±}FoxP3⁺ regulatory T cells infiltrating in the tumors.

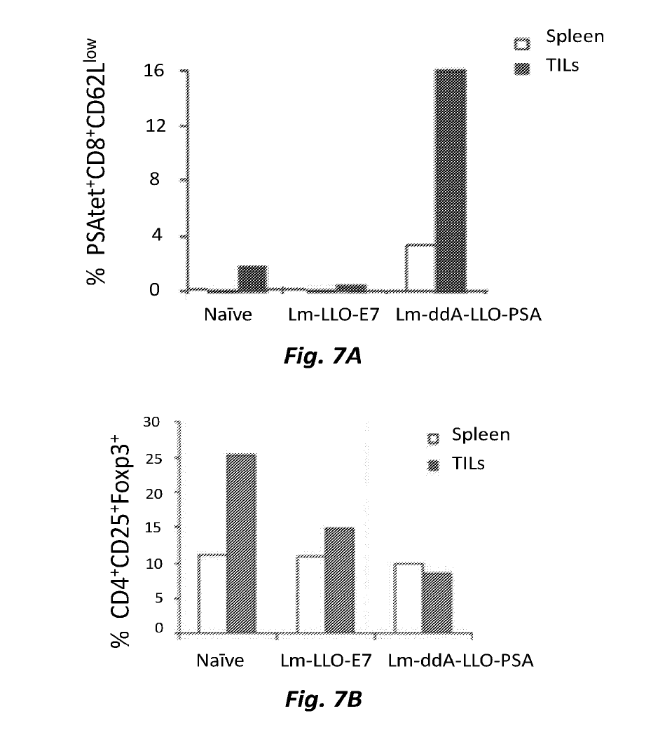

A very low number of CD8⁺CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8⁺CD62L^{low} PSA^{tetramer+} cells in spleen was 7.5 fold less than in tumor (FIG. 7A).

In addition, the presence of CD4⁺/CD25⁺/Foxp3⁺ T regulatory cells (Tregs) in the tumors of untreated mice and Listeria immunized mice was determined. Interestingly, immunization with Listeria resulted in a considerable decrease in the number of CD4⁺CD25^{±}FoxP3⁺T-regs in tumor but not in spleen (FIG. 7B). However, the construct LmddA-LLO-PSA had a stronger impact in decreasing the frequency of CD4⁺CD25⁺FoxP3⁺T-regs in tumors when compared to the naïve and Lm-LLO-E7 immunized group (FIG. 7B).

Thus, the LmddA-142 vaccine can induce PSA-specific CD8⁺ T cells that are able to infiltrate the tumor site (FIG. 7A). Interestingly, immunization with LmddA-142 was associated with a decreased number of regulatory T cells in the tumor (FIG. 7B), probably creating a more favorable environment for an efficient anti-tumor CTL activity.

### Example 7: Lmdd-143 and LmddA -143 Secretes a Functional LLO Despite the PSA Fusion

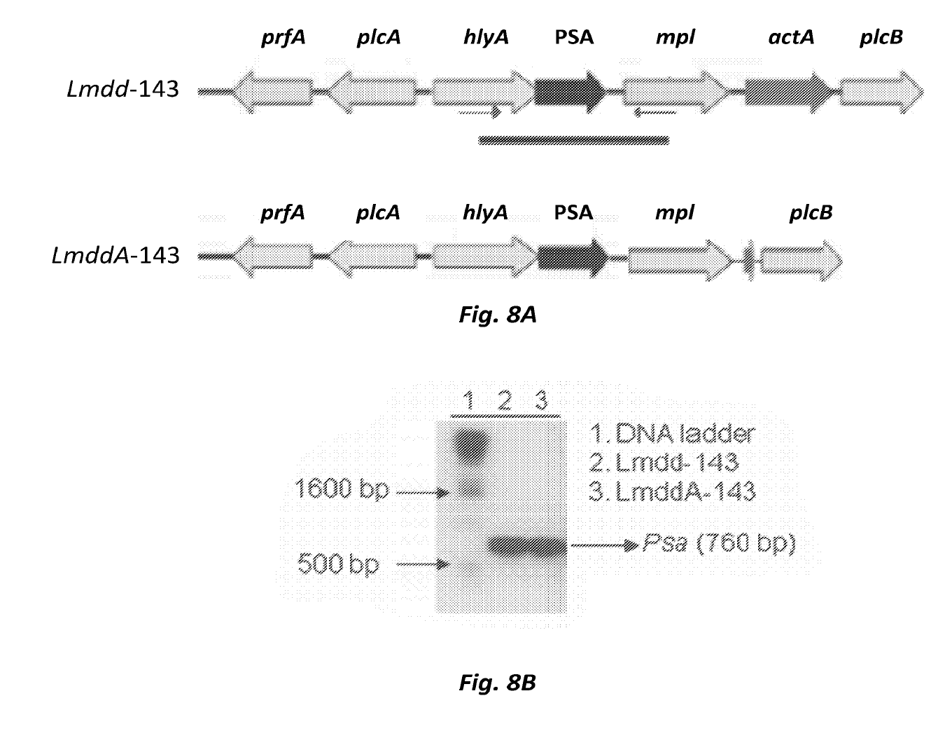

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie 1992; 74 (7-8) p705-711), which has a temperature-sensitive replicon, carrying the hly-k1k3-mpl recombination cassette. Because of the plasmid excision after the second recombination event, the antibiotic resistance marker used for integration selection is lost. Additionally, the actA gene is deleted in the LmddA-143 strain (FIG. 8A). The insertion of klk3 in frame with hly into the chromosome was verified by PCR (FIG. 8B) and sequencing (data not shown) in both constructs.

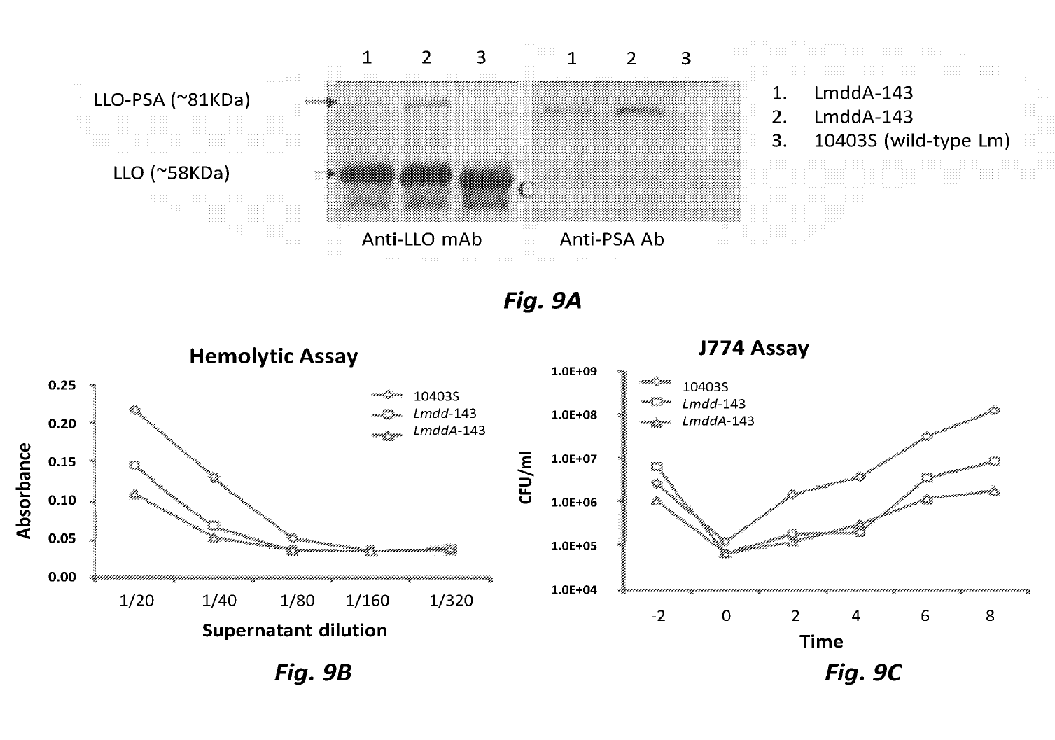

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 9A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 9B). In agreement with these results, both Lmdd-143 and LmddA-143 were able to replicate intracellularly in the macrophage-like J774 cell line (FIG. 9C).

### Example 8: Both Lmdd-143 and LmddA -143 Elicit Cell-Mediated Immune Responses Against the PSA Antigen

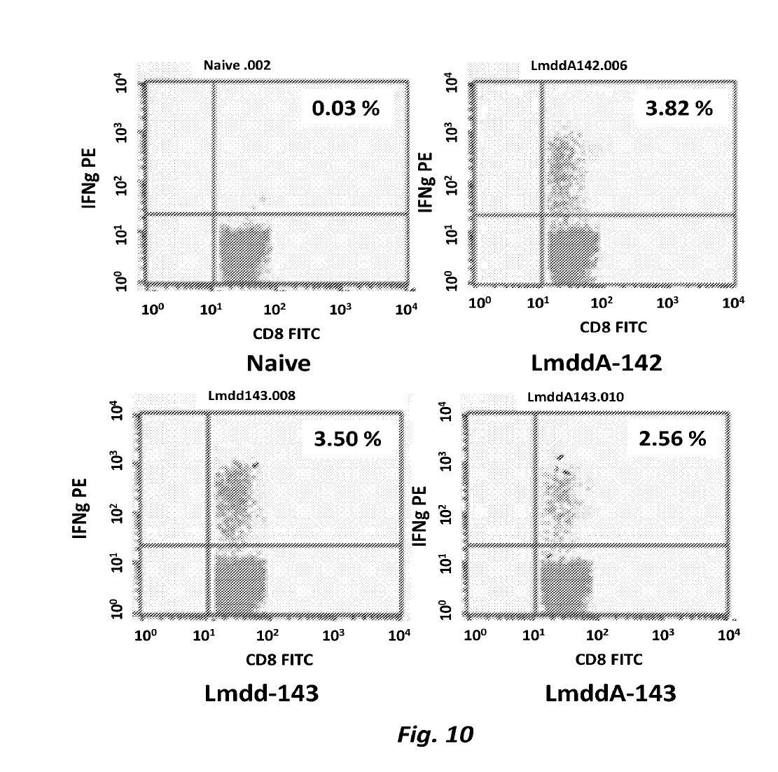

After showing that both Lmdd-143 and LmddA-143 were able to secrete PSA fused to LLO, the question of if these strains could elicit PSA-specific immune responses in vivo was investigated. C57Bl/6 mice were either left untreated or immunized twice with the Lmdd-143, LmddA-143 or LmddA-142. PSA-specific CD8⁺ T cell responses were measured by stimulating splenocytes with the PSA₆₅₋₇₄ peptide and intracellular staining for IFN-γ. As shown in FIG. 10, the immune response induced by the chromosomal and the plasmid-based vectors is similar.

### Materials and Methods (Examples 9-15)

Oligonucleotides were synthesized by Invitrogen (Carlsbad, Calif.) and DNA sequencing was done by Genewiz Inc., South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). Her2/neu HLA-A2 peptides were synthesized by EZbiolabs (Westfield, Ind.). Complete RPMI 1640 (C-RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-Her2/neu antibody was purchased from Sigma.

### Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N Her2/neu transgenic mice, which overexpress the rat Her2/neu onco-protein were housed and bred at the animal core facility at the University of Pennsylvania. The NT-2 tumor cell line expresses high levels of rat Her2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, Minn.) and was grown in complete C-RPMI medium. Bioluminescent work was conducted under guidance by the Small Animal Imaging Facility (SAIF) at the University of Pennsylvania (Philadelphia, Pa.).

### Listeria Constructs and Antigen Expression

Her2/neu-pGEM7Z was kindly provided by Dr. Mark Greene at the University of Pennsylvania and contained the full-length human Her2/neu (hHer2) gene cloned into the pGEM7Z plasmid (Promega, Madison Wis.). This plasmid was used as a template to amplify three segments of hHer-2/neu, namely, EC1, EC2, and IC1, by PCR using pfx DNA polymerase (Invitrogen) and the oligos indicated in Table 3.

TABLE 3
Primers for cloning of human Her-2 chimera.

| | | | Amino Acid |
|---|---|---|---|
| | | Base Pair | Region or |
| | DNA Sequence | Region | Junctions |
| Her-2-Chimera (F) | TGATCTCGAGACCCACCTGGACATGCTC | 120-510 | 40-170 |
| | (SEQ ID NO: 30) | | |
| HerEC1-EC2F | CTACCAGGACACGATTTTGTGGAAGAATATCCA | 510/1077 | 170/359 |
| (Junction) | GGAGTTTGCTGGCTGC | | |
| | (SEQ ID NO: 31) | | |
| HerEC1-EC2R | GCAGCCAGCAAACTCCTGGATATTCTTCCACAA | | |
| (Junction) | AATCGTGTCCTGGTAG | | |
| | (SEQ ID NO: 32) | | |
| HerEC2-ICIF | CTGCCACCAGCTGTGCGCCCGAGGGCAGCAGAA | 1554/2034 | 518/679 |
| (Junction) | GATCCGGAAGTACACGA | | |
| | (SEQ ID NO: 33) | | |
| HerEC2-ICIR | TCGTGTACTTCCGGATCTTCTGCTGCCCTCGGG | | |
| (Junction) | CGCACAGCTGGTGGCAG | | |
| | (SEQ ID NO: 34) | | |
| Her-2-Chimera (R) | GTGGCCCGGGTCTAGATTAGTCTAAGAGGCAGC | 2034-2424 | 679-808 |
| | CATAGG | | |
| | (SEQ ID NO: 35) | | |

The Her-2/neu chimera construct was generated by direct fusion by the SOEing PCR method and each separate hHer-2/neu segment as templates. Primers are shown in Table 4.

TABLE 4
Sequence of primers for amplification of different
segments human Her2 regions.

| | | Base Pair | Amino Acid |
|---|---|---|---|
| | DNA Sequence | Region | Region |
| Her-2-EC1(F) | CCGCCTCGAGGCCGCGAGCACCCAAGTG | 58-979 | 20-326 |
| | (SEQ ID NO: 36) | | |
| Her-2-EC1(R) | CGCGACTAGTTTAATCCTCTGCTGTCACCTC | | |
| | (SEQ ID NO: 37) | | |
| Her-2-EC2(F) | CCGCCTCGAGTACCTTTCTACGGACGTG | 907-1504 | 303-501 |
| | (SEQ ID NO: 38) | | |
| Her-2-EC2(R) | CGCGACTAGTTTACTCTGGCCGGTTGGCAG | | |
| | (SEQ ID NO: 39) | | |
| Her-2-IC1(F) | CCGCCTCGAGCAGCAGAAGATCCGGAAGTAC | 2034-3243 | 679-1081 |
| | (SEQ ID NO: 40) | | |
| Her-2-IC1(R) | CGCGACTAGTTTAAGCCCCTTCGGAGGGTG | | |
| | (SEQ ID NO: 41) | | |

ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (250 μg/ml). In some experiments similar Listeria strains expressing hHer2/neu (Lm-hHer2) fragments were used for comparative purposes. In all studies, an irrelevant Listeria construct (Lm-control) was included to account for the antigen independent effects of Listeria on the immune system. Lm-controls were based on the same Listeria platform as ADXS31-164 (LmddA-ChHer2), but expressed a different antigen such as HPV16-E7 or NY-ESO-1. Expression and secretion of fusion proteins from Listeria were tested. Each construct was passaged twice in vivo.

### Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naïve. NT-2 cells were grown in vitro, detached by trypsin and treated with mitomycin C (250 μg/ml in serum free C-RPMI medium) at 37° C. for 45 minutes. After 5 washes, they were co-incubated with splenocytes harvested from immunized or naïve animals at a ratio of 1:5 (Stimulator: Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm. Percent specific lysis was defined as (lysis in experimental group-spontaneous lysis)/(Maximum lysis-spontaneous lysis).

Interferon-γ Secretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naïve. Splenocytes from FVB/N mice were isolated one week after the last immunization and co-cultured in 24 well plates at 5×10⁶ cells/well in the presence of mitomycin C treated NT-2 cells in C-RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system. Samples from supernatants were obtained 24 or 72 hours later and tested for the presence of interferon-γ (IFN-γ) using mouse IFN-γ Enzyme-linked immunosorbent assay (ELISA) kit according to manufacturer's recommendations.

### Tumor Studies in Her2 Transgenic Animals

Six weeks old FVB/N rat Her2/neu transgenic mice (9-14/group) were immunized 6 times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNAlater at −20° C. In order to determine the effect of mutations in the Her2/neu protein on the escape of these tumors, genomic DNA was extracted using a genomic DNA isolation kit, and sequenced.

### Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C-RPMI medium. Tumors were minced using a sterile razor blade and digested with a buffer containing DNase (12U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FBS. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

### Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.15.0 (2006). For all FVB/N rat Her2/neu transgenic studies we used 8-14 mice per group, for all wild-type FVB/N studies we used at least 8 mice per group unless otherwise stated. All studies were repeated at least once except for the long term tumor study in Her2/neu transgenic mouse model.

### Example 9: Generation of L. Monocytogenes Strains that Secrete LLO Fragments Fused to her-2 Fragments: Construction of ADXS31-164

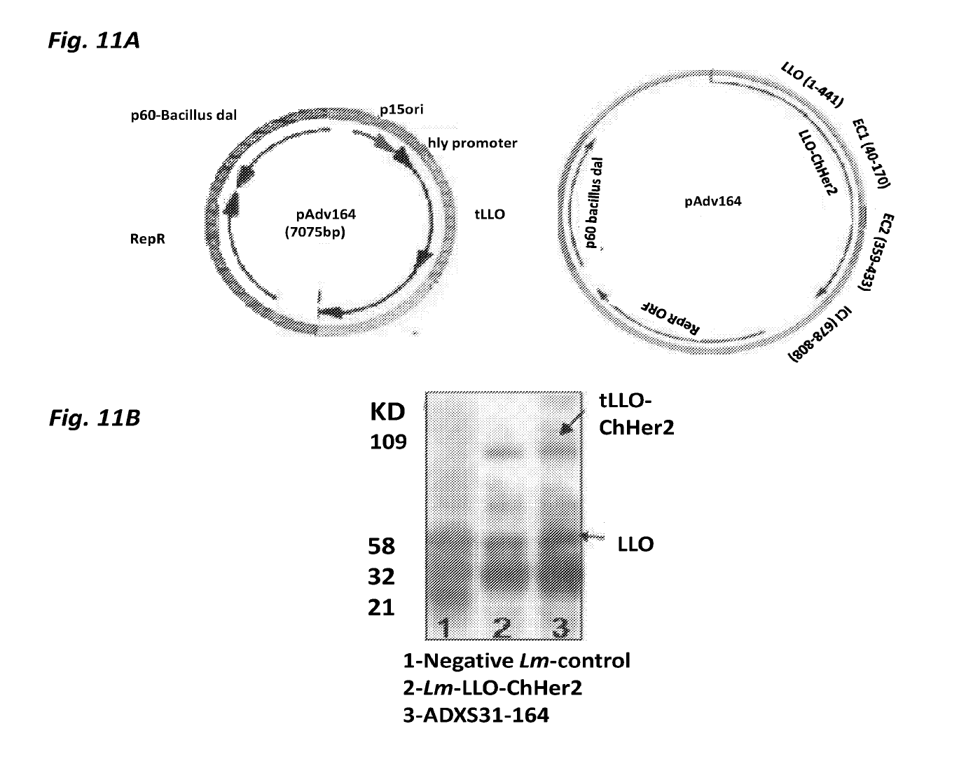

Construction of the chimeric Her2/neu gene (ChHer2) was as follows. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 11A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria vaccine strains. 2) Unlike pAdv138, pAdv164 does not harbor a copy of the prfA gene in the plasmid (see sequence below and FIG. 11A), as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 11B) as a band of ˜104 KD was detected by an anti-LLO antibody using Western Blot analysis. The Listeria backbone strain expressing only tLLO was used as negative control.

The pAdv164 sequence (7075 base pairs) (see FIGS. 11A and 11B) is set forth in SEQ ID NO: 58.

### Example 10: ADXS31-164 is as Immunogenic as Lm-LLO-ChHER2

Immunogenic properties of ADXS31-164 in generating anti-Her2/neu specific cytotoxic T cells were compared to those of the Lm-LLO-ChHer2 vaccine in a standard CTL assay. Both vaccines elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria vaccine (FIG. 12A). ADXS31-164 was also able to stimulate the secretion of IFN-γ by the splenocytes from wild type FVB/N mice (FIG. 12B). This was detected in the culture supernatants of these cells that were co-cultured with mitomycin C treated NT-2 cells, which express high levels of Her2/neu antigen (FIG. 12C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 42 or KIFGSLAFL SEQ ID NO: 43) or intracellular (RLLQETELV SEQ ID NO: 44) domains of the Her2/neu molecule (FIG. 12C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 11: ADXS31-164 was More Efficacious than Lm-LLO-ChHER2 in Preventing the Onset of Spontaneous Mammary Tumors

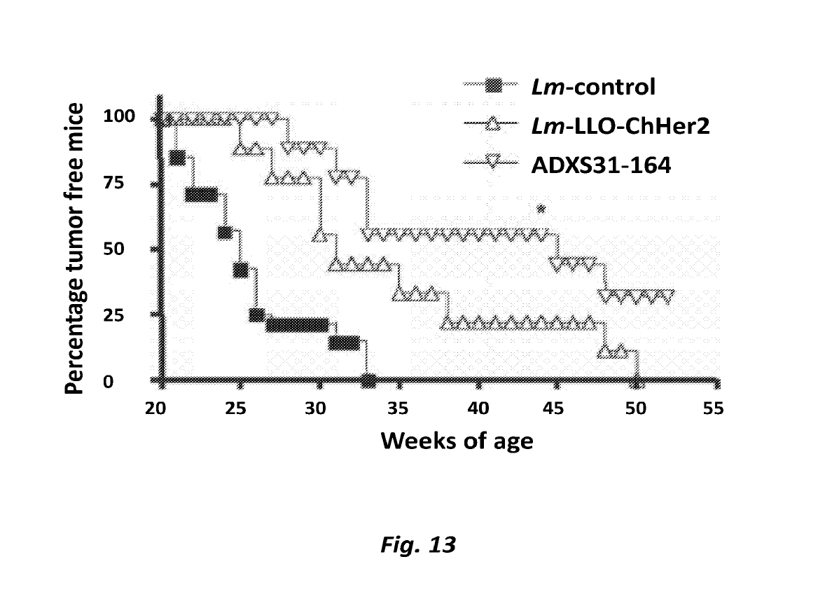

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Listeria-Her2/neu recombinant vaccines caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 13). These results indicate that despite being more attenuated, ADXS31-164 is more efficacious than Lm-LLO-ChHer2 in preventing the onset of spontaneous mammary tumors in Her2/neu transgenic animals.

### Example 12: Mutations in HER2/Neu Gene Upon Immunization with ADXS31-164

Mutations in the MHC class I epitopes of Her2/neu have been considered responsible for tumor escape upon immunization with small fragment vaccines or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control vaccines. Mutations were not observed within the Her-2/neu gene of any vaccinated tumor samples suggesting alternative escape mechanisms (data not shown).

### Example 13: ADXS31-164 Causes a Significant Decrease in Intra-Tumoral T Regulatory Cells

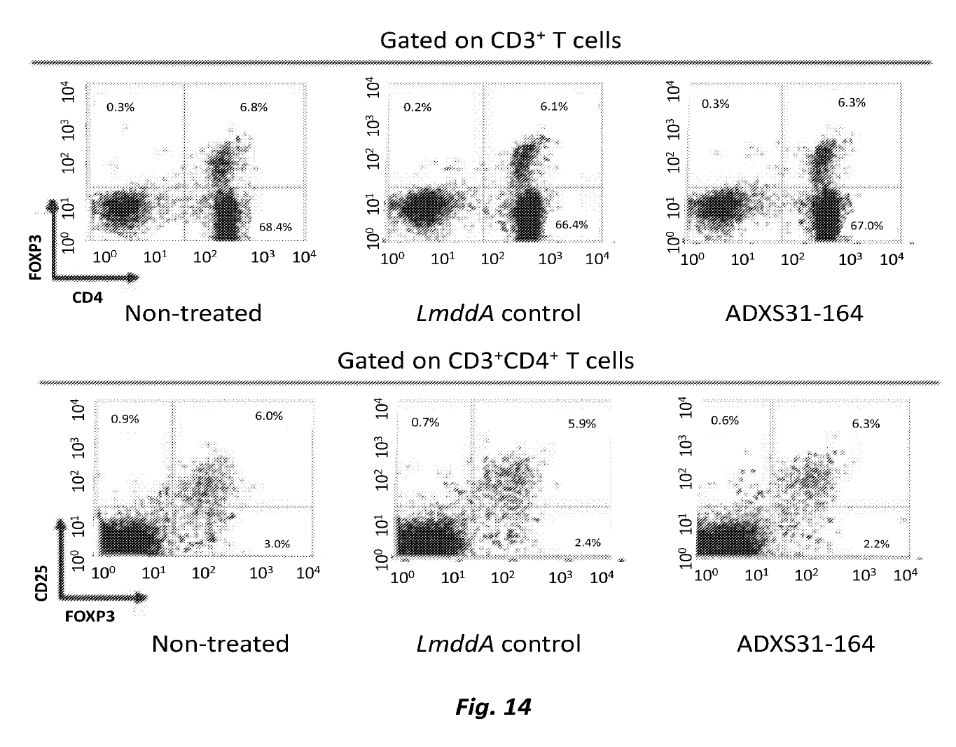

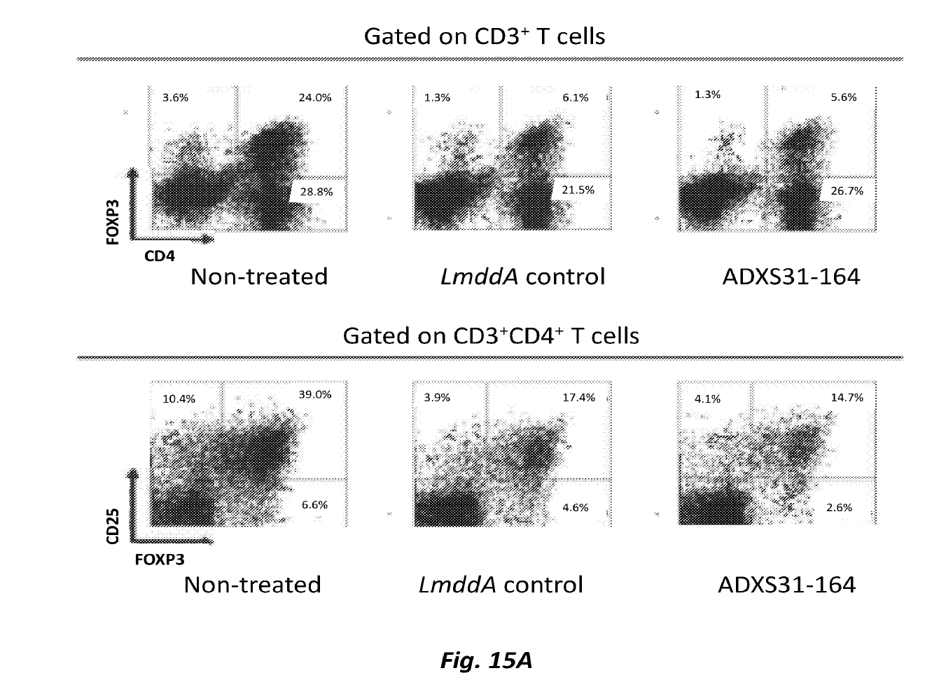

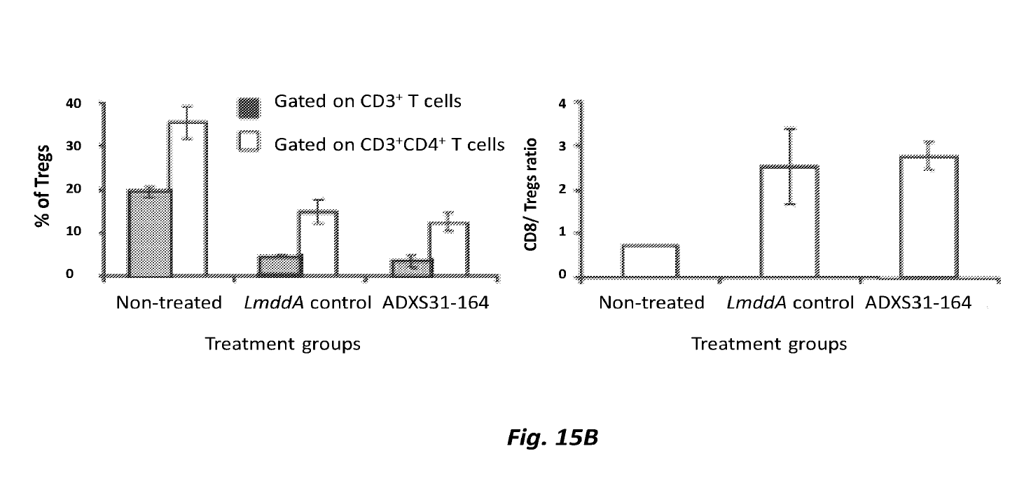

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria vaccine or the naïve animals (FIG. 14). In contrast, immunization with the Listeria vaccines caused a considerable impact on the presence of Tregs in the tumors (FIG. 15A). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 15B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm) ±SD, 6.71±0.43, n=5] than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 14: Peripheral Immunization with ADXS31-164 can Delay the Growth of a Metastatic Breast Cancer Cell Line in the Brain

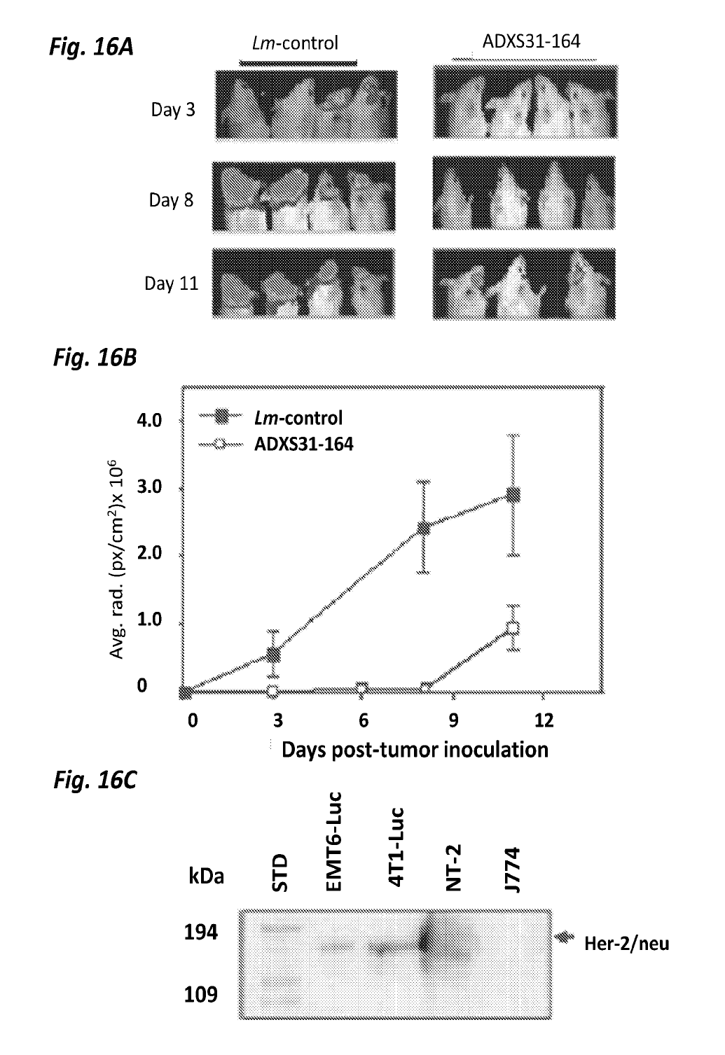

Mice were immunized IP with ADXS31-164 or irrelevant Lm-control vaccines and then implanted intra-cranially with 5,000 EMT6-Luc tumor cells, expressing luciferase and low levels of Her2/neu (FIG. 16A). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 16A and 16B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

### Example 15: Peptide “Minigene” Expression System

### Materials and Methods

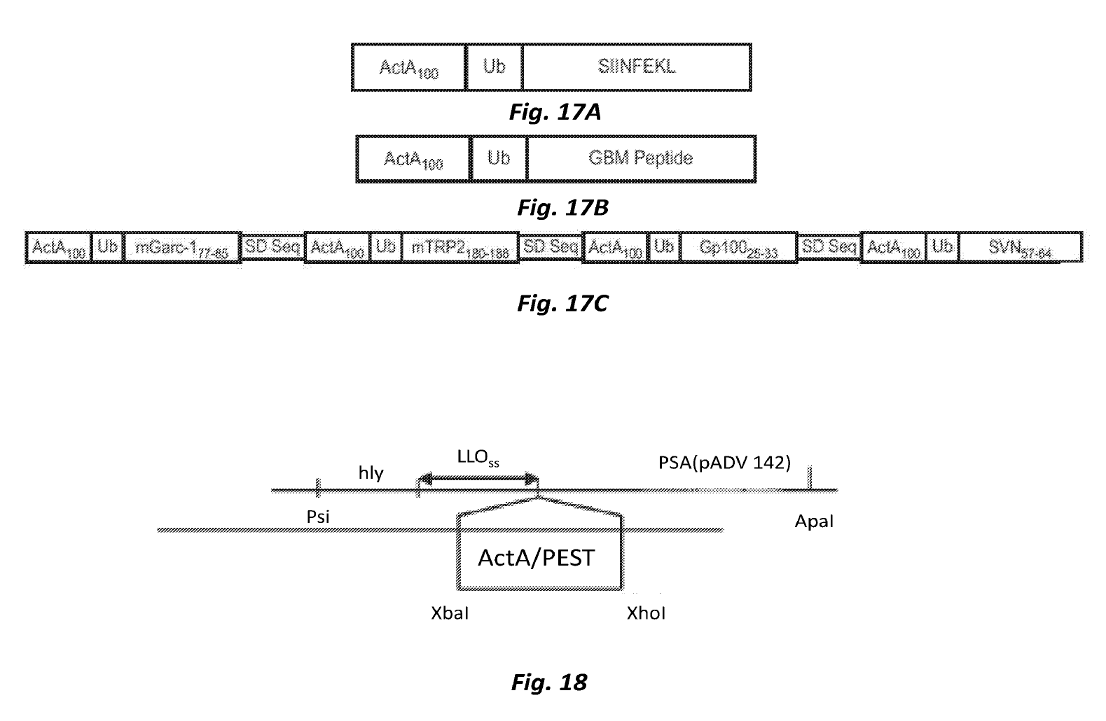

This expression system is designed to facilitate cloning of panels of recombinant proteins containing distinct peptide moieties at the carboxy-terminus. This is accomplished by a simple PCR reaction utilizing a sequence encoding one of the SS-Ub-Peptide constructs as a template. By using a primer that extends into the carboxy-terminal region of the Ub sequence and introducing codons for the desired peptide sequence at the 3′ end of the primer, a new SS-Ub-Peptide sequence can be generated in a single PCR reaction. The 5′ primer encoding the bacterial promoter and first few nucleotides of the ActA signal sequence is the same for all constructs. The constructs generated using this strategy are represented schematically in FIGS. 17A-17C. In this example, two constructs are described. One contains a model peptide antigen presented on mouse MHC class I and the second construct indicates where a therapeutically relevant peptide, such as one derived from a human glioblastoma (GBM) TAA, would be substituted. For clarity, we have designated the constructs diagramed in FIGS. 17A-C as containing an ActA₁₋₁₀₀ secretion signal. However, an LLO based secretion signal could be substituted with equal effect.

One of the advantages of the proposed system is that it will be possible to load cells with multiple peptides using a single Listeria vector construct. Multiple peptides will be introduce into recombinant attenuated Listeria (e.g. prfA mutant Listeria or a dal/dat/actA mutant Listeria) using a modification of the single peptide expression system described above. A chimeric protein encoding multiple distinct peptides from sequential SS-Ub-Peptide sequences encoded in one insert. Shine-Dalgarno ribosome binding sites are introduced before each SS-Ub-Peptide coding sequence to enable separate translation of each of the peptide constructs. FIG. 17C demonstrates a schematic representation of a construct designed to express 4 separate peptide antigens from one strain of recombinant Listeria. Since this is strictly a representation of the general expression strategy, we have included 4 distinct MHC class I binding peptides derived from known mouse or human tumor associated- or infectious disease antigens.

### Materials & Methods (Examples 16-18)

Plasmid pAdv142 and strain LmddA142 have been described above at Example 1. Additional details are provided below.

Construction of Plasmid pAdv142 and Strain LmddA142

This plasmid is next generation of the antibiotic free plasmid, pTV3 that was previously constructed by Verch et al. The unnecessary copy of the virulence gene transcription activator, prfA was deleted from plasmid pTV3 since Lm-ddA contains a copy of prfA gene in the chromosome. Therefore, the presence of prfA gene in the dal containing plasmid was not essential. Additionally, the cassette for p60-Listeria dal at the NheI/PacI restriction site was replaced by p60-Bacillus subtilis dal (claim) resulting in the plasmid pAdv134. Further, pAdv134 was restricted with XhoI/XmaI to clone human PSA, klk3 resulting in the plasmid, pAdv142. The new plasmid pAdv 142 (FIG. 2C) contains dal_{Bs} and its expression was under the control of Lm p60 promoter. The shuttle plasmid pAdv142 could complement the growth of both E. coli ala drx MB2159 as well as Lmdd in the absence of exogenous addition of D-alanine. The antigen expression cassette in the plasmid pAdv 142 consists of hly promoter and tLLO-PSA fusion protein (FIG. 18).

The plasmid pAdv142 was transformed to the Listeria background strain, LmddA resulting in LmddA142 or ADXS31-142. The expression and secretion of LLO-PSA fusion protein by the strain, ADXS31-142 was confirmed by western analysis using anti-LLO and anti-PSA antibody and is shown in FIG. 2D. There was stable expression and secretion of LLO-PSA fusion protein by the strain, ADXS31-142 after two in vivo passages in C57BL/6 mice.

### Construction of LmddA211, LmddA223 and LmddA224 Strains

The different ActA/PEST regions were cloned in the plasmid pAdv142 to create the three different plasmids pAdv211, pAdv223 and pAdv224 containing different truncated fragments of ActA protein.

LLO Signal Sequence (LLOss)-ActAPEST2 (pAdv211)/LmddA211.

First two fragments PsiI-LLOss-XbaI (817 bp in size) and LLOss-XbaI-ActA-PEST2 (602 bp in size) were amplified and then fused together by using SOEing PCR method with an overlap of 25 bases. This PCR product now contains PsiI-LLOss-XbaI-ActAPEST2-XhoI a fragment of 762 bp in size. The new PsiI-LLOss-XbaI-ActAPEST2-XhoI PCR product and pAdv142 (LmddA-PSA) plasmid were digested with PsiI/XhoI restriction enzymes and purified. Ligation was set up and transformed into MB2159 electro competent cells and plated onto LB agar plates. The PsiI-LLOss-XbaI-ActAPEST2/pAdv 142 (PSA) clones were selected and screened by insert-specific PCR reaction PsiI-LLOss-XbaI-ActAPEST2/pAdv 142 (PSA) clones #9, 10 were positive and the plasmid purified by mini preparation. Following screening of the clones by PCR screen, the inserts from positive clones were sequenced. The plasmid PsiI-LLOss-XbaI-ActAPEST2/pAdv 142 (PSA) referred as pAdv211.10 was transformed into Listeria LmddA mutant electro competent cells and plated onto BHI/strep agar plates. The resulting LmddA211 strain was screened by colony PCR. Several Listeria colonies were selected and screened for the expression and secretion of endogenous LLO and ActAPEST2-PSA (LA229-PSA) proteins. There was stable expression of ActAPEST2-PSA fusion proteins after two in vivo passages in mice.

LLOss-ActAPEST3 and PEST4.

ActAPEST3 and ActAPEST4 fragments were created by PCR method. PCR products containing LLOss-XbaI-ActAPEST3-XhoI (839 bp in size) and LLOss-XbaI-ActAPEST4-XhoI a fragments (1146 bp in size) were cloned in pAdv142. The resulting plasmid pAdv223 (PsiI-LLOss-XbaI-ActAPEST3-XhoI/pAdv 142) and pAdv224 (PsiI-LLOss-XbaI-ActAPEST4/pAdv 142) clones were selected and screened by insert-specific PCR reaction. The plasmids pAdv223 and pAdv224 were transformed to the LmddA backbone resulting in LmddA223 and LmddA224, respectively. Several Listeria colonies were selected and screened for the expression and secretion of endogenous LLO, ActAPEST3-PSA (LmddA223) or ActAPEST4-PSA (LmddA224) proteins. There was stable expression and secretion of the fusion protein ActAPEST3-PSA (LmddA223) or ActAPEST4-PSA (LmddA224) after two in vivo passages in mice.

### Experimental Plan 1

The therapeutic efficacy of the ActA-PEST-PSA (PEST3, PEST2 and PEST4 sequences) and tLLO-PSA using TPSA23 (PSA expressing tumor model) were evaluated and compared. Untreated mice were used as control group. In parallel evaluated the immune responses were also using intracellular cytokine staining for interferon—gamma and PSA tetramer staining.

For the Tumor Regression Study.

Ten groups of eight C57BL/6 mice (7 weeks old males) were implanted subcutaneously with 1×10⁶ of TPSA23 cells on day 0. On Day 6 they received immunization which was followed by 2 booster doses which were 1 week apart. Tumor growth was monitored every week until they reached a size of 1.2 cm in average diameter.

Immunogenicity Study.

Two groups of C57BL/6 mice (7 weeks old males) were immunized 3 times with one week interval with the vaccines listed in the table below. Six days after the last boost injection, mice were sacrificed, and the spleens will be harvested and the immune responses were tested for tetramer staining and IFN-γ secretion by intracellular cytokine staining.

### Experimental Plan 2

This experiment was a repeat of Experimental plan 1, however, the Naïve, tLLO, ActA/PEST2-PSA and tLLO-PSA groups were only included. Similar to Experimental plan 1, the therapeutic efficacy was evaluated using TPSA23 (PSA expressing tumor model). Five C57BL/6 mice per group were implanted subcutaneously with 1×10⁶ of TPSA23 cells on day 0. On Day 6 they received immunization (1×10⁸ CFU/mL) which was followed by booster 1 week later. Spleen and tumor was collected on day 6 post last treatment. The immune response was monitored using PSA pentamer staining in both spleen and tumor.

Materials & Methods.

TPSA23 cells are cultured in complete medium. Two days prior to implanting tumor cells in mice, TPSA23 cells were sub-cultured in complete media. On the day of the experiment (Day 0), cells were trypsinized and washed twice with PBS. Cells were counted and re-suspended at a concentration of 1×10⁶ cells/200 ul in PBS/mouse for injection. Tumor cells were injected subcutaneously in the flank of each mouse.

Complete Medium for TPSA23 Cells.

Complete medium for TPSA23 cells was prepared by mixing 430 ml of DMEM with Glucose, 45 ml of fetal calf serum (FCS), 25 ml of Nu-Serum IV, 5 ml 100× L-Glutamine, 5 ml of 100 mM Na-Pyruvate, 5 ml of 10,000U/mL Penicillin/Streptomycin. 0.005 mg/ml of Bovine Insulin and 10 nM of Dehydroisoandrosterone was added to the flask while splitting cells.

Complete Medium for Splenocytes (c-RPMI).

Complete medium was prepared by mixing 450 ml of RPMI 1640, 50 ml of fetal calf serum (FCS), 5 ml of 1M HEPES, 5 ml of 100× Non-essential amino acids (NEAA), 5 ml of 100× L-Glutamine, 5 ml of 100 mM Na-Pyruvate, 5 ml of 10,000U/mL Penicillin/Streptomycin and 129 ul of 14.6M 2-Mercaptoethanol.

### Preparing Isolated Splenocytes

Work was performed in biohazard hood. Spleens were harvested from experimental and control mice groups using sterile forceps and scissors. They were transport in 15 ml tubes containing 10 ml PBS to the lab. Spleen from each mouse was processed separately. Spleen was taken in a sterile Petri dish and mashed using the back of plunger from a 3 mL syringe. Spleen cells were transferred to a 15 ml tube containing 10 ml of RPMI 1640. Cells were pelleted by centrifugation at 1,000 RPM for 5 min at 4° C. The supernatant was discarded in 10% bleach. Cell pellet was gently broken by tapping. RBC was lysed by adding 2 ml of RBC lysis buffer per spleen to the cell pellet. RBC lysis was allowed for 2 min Immediately, 10 ml of c-RPMI medium was added to the cell suspension to deactivate RBC lysis buffer. Cells were pelleted by centrifugation at 1,000 RPM for 5 min at 4° C. The supernatant was discarded and cell pellet was re-suspended in 10 ml of c-RPMI and passed through a cell strainer. Cells were counted using hemocytometer and the viability was checked by mixing 10 ul of cell suspension with 90 ul of Trypan blue stain. About 2×10⁶ cells were used for pentamer staining. (Note: each spleen should yield 1-2×10⁸ cells).

Preparing Single Cell Suspension from Tumors Using Miltenyi Mouse Tumor Dissociation Kit

Enzyme mix was prepared by adding 2.35 mL of RPMI 1640, 100 μL of Enzyme D, 50 μL of Enzyme R, and 12.5 μL of Enzyme A into a gentleMACS C Tube. Tumor (0.04-1 g) was cut into small pieces of 2-4 mm and transferred into the gentleMACS C Tube containing the enzyme mix. The tube was attached upside down onto the sleeve of the gentle MACS Dissociator and the Program m_impTumor_02 was run. After termination of the program, C Tube was detached from the gentle MACS Dissociator. The sample was incubated for 40 minutes at 37° C. with continuous rotation using the MACSmix Tube Rotator. After completion of incubation the C tube was again attached upside down onto the sleeve of the gentle MACS Dissociator and the program m_impTumor_03 was run twice. The cell suspension was filtered through 70 μm filter placed on a 15 mL tube. The filter was also washed with 10 mL of RPMI 1640. The cells were centrifuged at 300×g for 7 minutes. The supernatant was discarded and the cells were re-suspended in 10 ml of RPMI 1640. At this point one can divide the cells for pentamer staining.

### Pentamer Staining of Splenocytes

The PSA-specific T cells were detected using commercially available PSA-H-2D^{b} pentamer from ProImmune using manufacturers recommended protocol. Splenocytes were stained for CD8, CD62L, CD3 and Pentamer. While tumor cells were stained for CD8, CD62L, CD45 and Pentamer. The CD3⁺CD8⁺CD62L^{low} cells were gated to determine the frequency of CD3⁺CD8⁺CD62L^{low} PSA pentamer⁺ cells. The stained cells were acquired and analyzed on FACS Calibur using Cell quest software.

Materials Needed for Pentamer Staining.

Splenocytes (preparation described above), Pro5® Recombinant MHC PSA Pentamer conjugated to PE. (Note: Ensure that the stock Pentamer is stored consistently at 4° C. in the dark, with the lid tightly closed), anti-CD3 antibody conjugated to PerCP Cy5.5, anti-CD8 antibody conjugated to FITC and anti-CD62L antibody conjugated to APC, wash buffer (0.1% BSA in PBS) and fix solution (1% heat inactivated fetal calf serum (HI-FCBS), 2.5% formaldehyde in PBS).

Standard Staining Protocol.

Pro5® PSA Pentamer was centrifuged in a chilled microcentrifuge at 14,000×g for 5-10 minutes to remove any protein aggregates present in the solution. These aggregates may contribute to non-specific staining if included in test volume. 2×10⁶ splenocytes were allocated per staining condition and 1 ml of wash buffer was added per tube. Cells were centrifuged at 500×g for 5 min in a chilled centrifuge at 4° C. The cell pellet was re-suspended in the residual volume (˜50 μl). All tubes were chilled on ice for all subsequent steps, except where otherwise indicated. 10 μl of labeled Pentamer was added to the cells and mixed by pipetting. The cells were incubated at room temperature (22° C.) for 10 minutes, shielded from light. Cells were washed with 2 ml of wash buffer per tube and re-suspend in residual liquid (˜50 μl). An optimal amount of anti-CD3, anti-CD8 and anti-CD62L antibodies were added (1:100 dilution) and mixed by pipetting. Single stain control samples were also made at this point. Samples were incubated on ice for 20 minutes, shielded from light. Cells were washed twice with 2 ml wash buffer per tube. The cell pellet was re-suspended in the residual volume (˜50 μl). 200 μl of fix solution was added to each tube and vortexed. The tubes were stored in dark in the refrigerator until ready for data acquisition. (Note: the morphology of the cell changes after fixing, so it is advisable to leave the samples for 3 hours before proceeding with data acquisition. Samples can be stored for up to 2 days).

Intracellular Cytokine Staining (IFN-γ) Protocol.

2×10⁷ cells/ml splenocytes were taken in FACS tubes and 100 μl of Brefeldin A (BD Golgi Plug) was added to the tube. For stimulation, 2 μM Peptide was added to the tube and the cells were incubated at room temperature for 10-15 minutes. For positive control samples, PMA (10 ng/ml) (2×) and ionomycin (1 μg/ml) (2×) was added to corresponding tubes. 100 μl of medium from each treatment was added to the corresponding wells in a U-bottom 96-well plate. 100 μl of cells were added to the corresponding wells (200 μl final volume−medium+cells). The plate was centrifuged at 600 rpm for 2 minutes and incubated at 37° C. 5% CO₂ for 5 hours. Contents from the plate was transferred to FACS tubes. 1 ml of FACS buffer was added to each tube and centrifuged at 1200 rpm for 5 min. The supernatant was discarded. 200 μl of 2.4G2 supernatant and 10 μl of rabbit serum was added to the cells and incubated for 10 minutes at room temperature. The cells were washed with 1 mL of FACS buffer. The cells were collected by centrifugation at 1200 rpm for 5 minutes. Cells were suspended in 50 μl of FACS buffer containing the fluorochrome-conjugated monoclonal antibodies (CD8 FITC, CD3 PerCP-Cy5.5, CD62L APC) and incubated at 4° C. for 30 minutes in the dark. Cells were washed twice with 1 mL FACS buffer and re-suspended in 200 μl of 4% formalin solution and incubated at 4° C. for 20 min. The cells were washed twice with 1 mL FACS buffer and re-suspended in BD Perm/Wash (0.25 ml/tube) for 15 minutes. Cells were collected by centrifugation and re-suspended in 50 μl of BD Perm/Wash solution containing the fluorochrome-conjugated monoclonal antibody for the cytokine of interest (IFNg-PE). The cells were incubated at 4° C. for 30 minutes in the dark. Cells were washed twice using BD Perm/Wash (1 ml per tube) and re-suspended in 200 μl FACS buffer prior to analysis.

### Results

### Example 16: Vaccination with Recombinant Listeria Constructs Leads to Tumor Regression

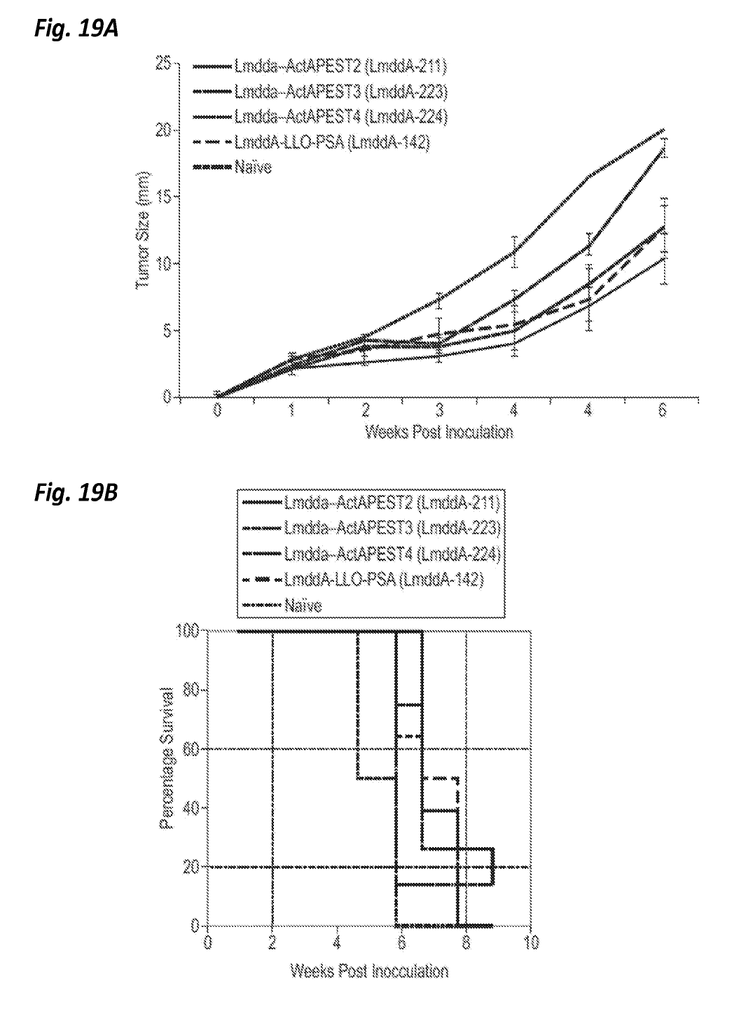

The data showed that by week 1, all groups had developed tumor with the average size of 2-3 mm. On week 3 (Day 20) mice immunized with ActA/PEST2 (also known as “LA229”)-PSA, ActA/PEST3-PSA and ActA/PEST3-PSA and LmddA-142 (ADXS31-142), which expresses a tLLO fused to PSA showed, tumor regression and slow down of the tumor growth. By week 6, all mice in naïve and most in ActAPEST4-PSA treated group had big tumors and had to be euthanized (FIG. 19A). However, LmddA-142, ActA-PEST2 and ActA-PEST3 mice groups showed better tumor regression and survival rate (FIGS. 19A and 19B).

### Example 17: Vaccination with Recombinant Listeria Generates High Levels of Antigen-Specific T Cells

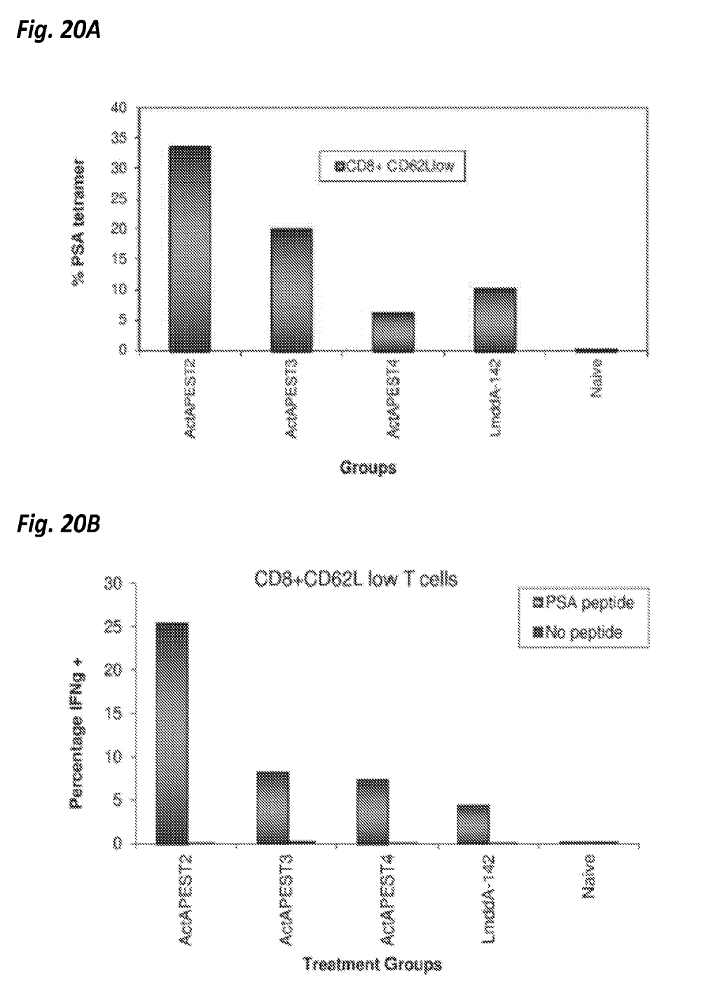

LmddA-ActAPEST2-PSA vaccine generated high levels of PSA-specific T cells response compared to LmddA-ActAPEST (3 or 4)—PSA, or LmddA-142 (FIG. 20A). The magnitude of PSA tetramer specific T cells in PSA-specific vaccines was 30 fold higher than naïve mice. Similarly, higher levels of IFN-γ secretion was observed for LmddA-ActAPEST2-PSA vaccine in response to stimulation with PSA-specific antigen (FIG. 20B).

### Example 18: Vaccination with ActA/PEST2 (LA229) Generates a High Number of Antigen-Specific CD8+ T Cells in Spleen

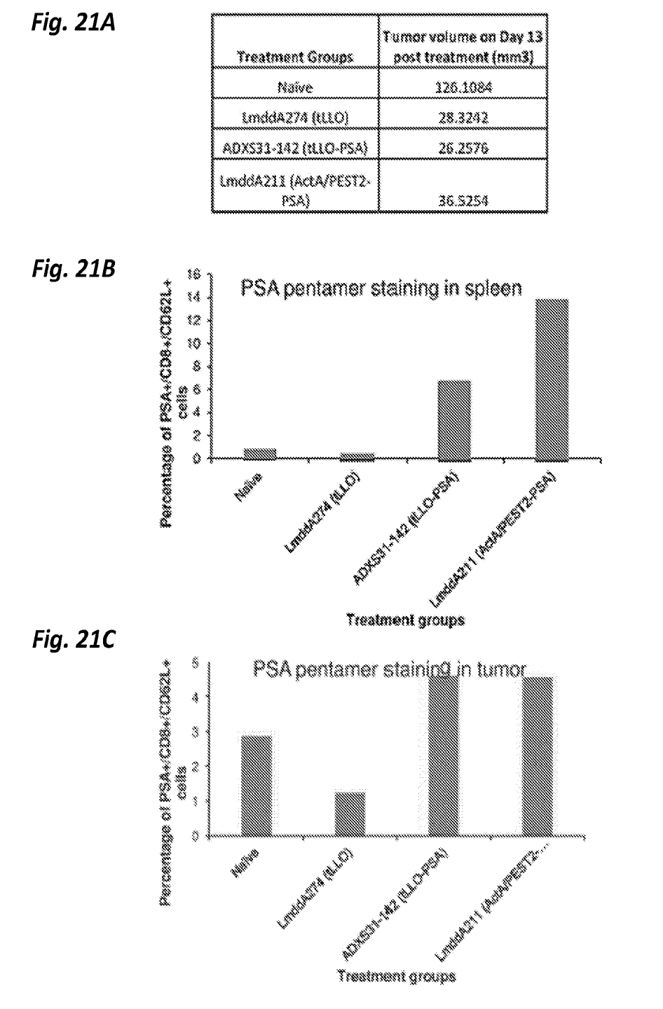

Lm expressing ActA/PEST2 fused PSA was able to generate higher numbers of PSA specific CD8+ T cells in spleen compared to Lm expressing tLLO fused PSA or tLLO treated group. The number of PSA specific CD8+ T cells infiltrating tumors were similar for both Lm-tLLO-PSA and Lm-ActA/PEST2-PSA immunized mice (FIGS. 21B and 21C). Also, tumor regression ability of Lm expressing ActA/PEST2-PSA was similar to that seen for LmddA-142 which expresses tLLO-PSA (FIG. 21A).

### Example 19: Construction of a Neo-Epitope Expression Vector

Constructing the Lm vector comprising one or more neo-epitope is performed using the steps detailed below.

### Whole Genome Sequencing

First, comparative whole genome sequencing including locating nonsynonymous mutations present in approximately >20% of tumor cells is performed and the results are provided in FASTA format. Matched normal/tumor samples from whole exomes are sequenced by an outside vendor, and output data is given in the preferred FASTA format listing all neo-antigens as 21 amino acid sequence peptides, for example a peptide having 10 non-mutant amino acids on either side of a mutant amino acid. Also included are patient HLA types.

DNA and RNA from a biological sample obtained from human tissue (or any non-human animal) are extracted in triplicates. Another source of neo-antigens could be from sequencing metastases or circulating tumor cells. They may contain additional mutations that are not resident in the initial biopsy but could be included in the vector to specifically target cytotoxic T cells (CTC's) or metastases that have mutated differently than the primary biopsy that was sequenced. Triplicates of each sample are sequenced by DNA exome sequencing. In brief, 3 μg purified genomic DNA (gDNA) are fragmented to about 150-200 bp using an ultrasound device. Fragments are end repaired, 5′ phosphorylated, 3′ adenylated, and then Illumina paired end adapters are ligated to the gDNA fragments according to the manufacturer's instructions. Enriched pre capture and flow cell specific sequences are added using Illumina PE PCR primers. About 500 ng of adapter ligated, PCR enriched gDNA fragments are hybridized to biotinylated exome (human exome or any other non-human animal exome e.g. mouse, guinea pig, rat, dog, sheep). RNA library baits for 24 hrs at 65° C. Hybridized gDNA/RNA bait complexes are then removed using streptavidin coated magnetic beads, washed and the RNA baits cleaved off. These eluted gDNA fragments are PCR amplified and then sequenced on an Illumina sequencing apparatus.

### RNA Gene Expression Profiling (RNA-Seq)

Barcoded mRNA-seq cDNA libraries are prepared in triplicates from a total of about 5 μg of total RNA, then, in brief, mRNA are isolated and fragmented. Following, mRNA fragments are converted to cDNA and connected to specific Illumina adaptors, clustered and sequenced according to standard illumine protocol. The output sequence reads are aligned to a referenced sequence (RefSeq). Genome alignments and transcriptome alignments are made. Reads are also aligned to exon-exon junctions. Expression values are determined by intersecting read coordinates with those of RefSeq transcripts, counting overlapping exon and exon junction reads, and normalized to standard normalizing units such as RPKM expression units (Reads which map per Kilobase of transcript per Million mapped reads).

### Detecting Mutations

Fragments of isolated gDNA from a disease or condition bearing tissue sample are aligned to referenced matched gDNA of a healthy tissue, by vendor available software, e.g. Samtools, GATK, and Somatic Sniper.

About 10 flanking amino acids on each side of the detected mutation are incorporated to accommodate class1 MHC-1 presentation, in order to provide at least some of the different HLA TCR reading frames.

Table 5 shows a sample list of 50 neo-epitope peptides wherein each mutation is indicated by a Bolded amino acid letter and is flanked by 10 amino acids on each side providing a 21 amino acid peptide neo-epitope.

TABLE 5

| Name | Sequence¹ | SEQ ID NO: |
|---|---|---|
| MUT1 | FMVAVAHVAAFLLEDRAVCV | 68 |
| MUT2 | AENVEQVLVTSIQGAVDYPDP | 69 |
| MUT3 | SFKKKFEECQHNIIKLQNGHT | 70 |
| MUT4 | SALIESLNQKTQSTGDHPQPT | 71 |
| MUT5 | KAYLPVNESFAFTADLRSNTG | 72 |
| MUT6 | HTLLEITEESGAVLVDKSDSD | 73 |
| MUT7 | SVMCTYSPPLDKLFCQLAKTC | 74 |
| MUT8 | ESGKHKYRQTAMFTATMPPAV | 75 |
| MUT9 | AAPSAASSPADVQSLKKAMSS | 76 |
| MUTT0 | SQLFSLNPRGRSLVTAGRIDR | 77 |
| MUT11 | SLARGPLSEAGLALFDPYSKE | 78 |
| MUT12 | QKKLCHLSSTGLPRETIASLP | 79 |
| MUT13 | LTASNMEGKSWPSEVLVCTTS | 80 |
| MUT14 | YAAQQHETFLTNGDRAGFLIG | 81 |
| MUTTS | QAKVPFSEETQNLILPYISDM | 82 |
| MUT16 | CNRAGEKHCFSSNEAARDFGG | 83 |
| MUT17 | RNPQFLDPVLAYLMKGLCEKP | 84 |
| MUT18 | TFCERGKQEAKLLAERSRFED | 85 |
| MUT19 | APLEWLRYFDKKETFLMLCGM | 86 |
| MUT20 | KAFLHWYTGEAMDEMEFTLAE | 87 |
| MUT21 | DEVALVEGVQSLGFTYLRLKD | 88 |
| MUT22 | DFSQLQRNILPSNPRVTRFHI | 89 |
| MUT23 | ISTNGSFIRLLDAFKGVVMHT | 90 |
| MUT24 | ITPPTTTTKKARVSTPKPATP | 91 |
| MUT25 | NYNTSHLNNDVWQIFENPVDW | 92 |
| MUT26 | QKTLHNLLRKVVPSFSAEIER | 93 |
| MUT27 | VELCPGNKYEMRRHGTTHSLV | 94 |
| MUT28 | GIDKLTQLKKPFLVNNIGNKI | 95 |
| MUT29 | GTTILNCFHDVLSGKLSGGS | 96 |
| MUT30 | PSFQEFVDWENVSPELNSTDQ | 97 |
| MUT31 | PALVEEYLERGNFVANDLDWL | 98 |
| MUT32 | ELKACKPNGKRNPYCEVSMGS | 99 |
| MUT33 | SPFPAAVILRDALHMARGLKY | 100 |
| MUT34 | QQLDTYILKNVVAFSRTDKYR | 101 |
| MUT35 | SFVGQTRVLMINGEEVEEIEL | 102 |
| MUT36 | AFFINFIAIYHHASRAIPFGT | 103 |
| MUT37 | GLALPNNYCDVCLGDSKINKK | 104 |
| MUT38 | EGQISIAKYENCPKDNPMYYC | 105 |
| MUT39 | NFKRKRVAAFQKNLIEMSELE | 106 |
| MUT40 | KMKGELGMMLILQNVIQKTTT | 107 |
| MUT41 | SIECKGIDKEINESKNTHLDI | 108 |
| MUT42 | ELEAAIETVVCTFFTFAGREG | 109 |
| MUT43 | SLSHREREQMKATLNYEDHCF | 110 |
| MUT44 | HIKAFDRTFANNPGPMVVFAT | 111 |
| MUT45 | ITSNFVIPSEYWVEEKEEKQK | 112 |
| MUT46 | GLVTFQAFIDVMSRETTDTDT | 113 |
| MUT47 | HLLGRLAAIVGKQVLLGRKVV | 114 |
| MUT48 | HWNDLAVIPAGVVHNWDFEPR | 115 |
| MUT49 | SMDHKTGTIAMQNTTQLRSRY | 116 |
| MUT50 | QPLRRLVLHVVSAAQAERLAR | 117 |

¹Bolded letter indicates mutated amino acid

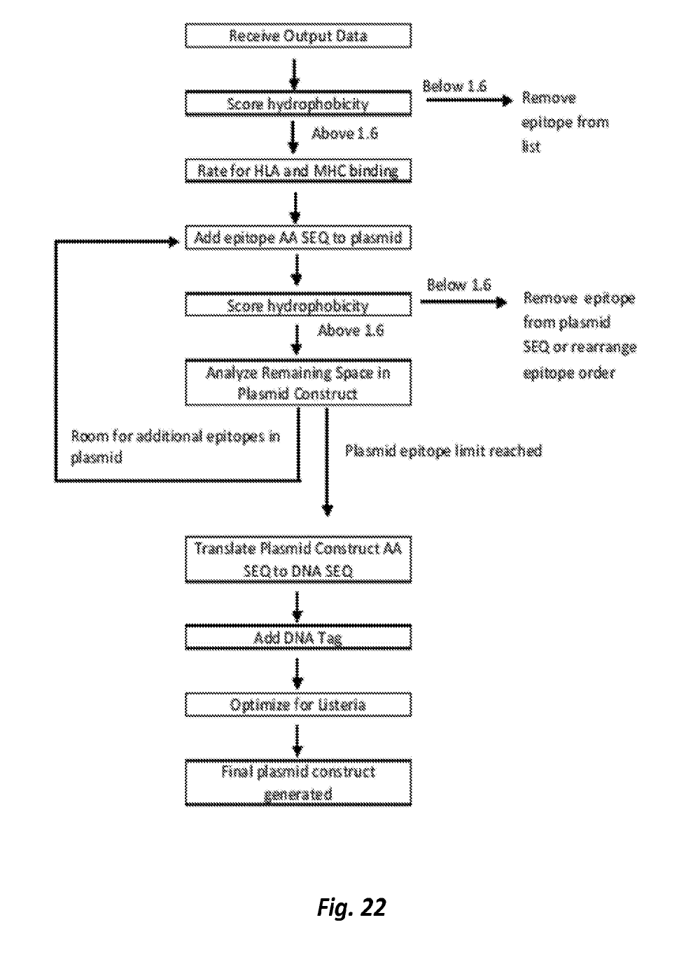

Output FASTA file is used to design patient-specific constructs, either manually or by programmed script according to one or more of criteria detailed below. The programmed script automates the creation of the personalized plasma construct containing one or more neo-epitopes for each subject using a series of protocols (FIG. 22). The output FASTA file is inputted and after running the protocols, the DNA sequence of a LM vector including one or more neo-epitopes is outputted. The software program is useful for creating personalized immunotherapy for each subject.

Prioritization of Neo-Epitopes for Incorporation into Constructs.

Neo-epitopes are scored by Kyte and Doolittle hydropathy index 21 amino acid window, all scoring above cutoff (around 1.6) are excluded as they are unlikely to be secretable by Listeria monocytogenes. The remaining 21 amino acid long peptides are then scored for their ability to bind patient HLA (for example by using IEDB, Immune epitope database and analysis source, http://www.iedb.org/) and ranked by best MHC binding score from each 21 amino acid sequence peptide. Cut-offs may be different for different expression vectors such as Salmonella.

Determination of the number of constructs vs. mutational burden, are performed to determine efficiency of expression and secretion of neo-epitopes. Ranges of linear neo-epitopes are tested, starting with about 50 epitopes per vector. In certain cases constructs will include at least one neo-epitope per vector. The number of vectors to be used is determined considering for example the efficiency of translation and secretion of multiple epitopes from a single vector, and the MOI needed for each Lm vector harboring specific neo-epitopes, or in reference to the number of neo-epitopes. Another consideration can be by predefining groups of known tumor-associated mutations/mutations found in circulating tumor cells/known cancer “driver” mutations/known chemotherapy resistance mutations and giving them priority in the 21 amino acid sequence peptide selection. This can be accomplished by screening identified mutated genes against the COSMIC (Catalogue of somatic mutations in cancer, cancer.Sanger.ac.uk) or Cancer Genome Analysis or other similar cancer-associated gene database. Further, screening for immunosuppressive epitopes (T-reg epitopes, IL-10 inducing T helper epitopes, etc.) is utilized to de-selected or to avoid immunosuppressive influences on the vector. Selected codons are codon optimized to efficient translation and secretion according to specific Listeria strain. Example for codons optimized for L. monocytogenes as known in the art is presented in Table 6.

TABLE 6
Preliminary Listeria monocytogenes preferred (most common) codon
table.

| | | | | |
|---|---|---|---|---|
| A = GCA | G = GGT | L = TTA | Q = CAA | V = GTT |
| C = TGT | H = CAT | M = ATG | R = CGT | W = TGG |
| D = GAT | I = ATT | N = AAC | S = TCT | Y = TAT |
| E = GAA | K = AAA | P = CCA | T = ACA | STOP = TAA |
| F = TTC | | | | |

The remaining 21amino acid peptide neo-epitopes are assembled into a pAdv134-MCS (SEQ ID NO: 45) plasmid, or optionally into pAdv134, exchanging the LLO-E7 cassette as shown in Example 8 above, to create the tLLO-neo-epitope-tag fusion polypeptide. The compatible insert as an amino acid sequence and the whole insert are rechecked by Kyte and Doolittle test to confirm no hydropathy problems across the whole construct. If needed, the insert order is rearranged or the problem 21 amino acid sequence peptides is removed from construct.

The construct amino acid sequence is reverse translated into the corresponding DNA sequence for DNA synthesis/cloning into pAdv134-MCS (SEQ ID NO: 45). Nucleotides 2400-2453 refer to a multi-cloning site by outside vendor. Individual 21 amino acid peptides sequences and the SIINFEKL-6× His tag DNA sequences (for example SEQ ID NO: 57) are optimized for expression and secretion in L. monocytogenes while the 4× glycine linker sequences are one of eleven preset DNA sequences (G1-G11, SEQ ID NO: 46-56). Linker sequence codons are varied to avoid excess repetition to better enable DNA synthesis. Examples of the different sequence codons (G1-G11, SEQ ID NO: 46-56) for 4× glycine linkers are presented in Table 7.

TABLE 7
4x glycine linker DNA sequences
and terminal tag sequence.

| | | SEQ |
|---|---|---|
| Name | Sequence | ID NO: |
| G1 | GGTGGTGGAGGA | 46 |
| G2 | GGTGGAGGTGGA | 47 |
| G3 | GGTGGAGGAGGT | 48 |
| G4 | GGAGGTGGTGGA | 49 |
| G5 | GGAGGAGGTGGT | 50 |
| G6 | GGAGGTGGAGGT | 51 |
| G7 | GGAGGAGGAGGT | 52 |
| G8 | GGAGGAGGTGGA | 53 |
| G9 | GGAGGTGGAGGA | 54 |
| G10 | GGTGGAGGAGGA | 55 |
| G11 | GGAGGAGGAGGA | 56 |
| C-terminal SIINFEKL and | ARSIINFEKLSHHHHHH | 57 |
| 6xHis AA sequence | | |

Each neo-epitope is connected with a linker sequence to the following neo-epitope encoded on the same vector. The final neo-epitope in an insert is fused to a TAG sequence followed by a stop codon. The TAG fused is set forth in SEQ ID NO: 57, a C-terminal SIINFEKL and 6× His amino acid sequence. The TAG allows for easy detection of the tLLO-neo-epitope during for example secretion from the Lm vector or when testing construct for affinity to specific T-cells, or presentation by antigen presenting cells. The linker is 4× glycine DNA sequence, selected from a group comprising G1-G11 (SEQ ID NOS: 46-56) accordingly, or any combination thereof.

If there are more usable 21 amino acid peptides than can fit into a single plasmid (maximum payload currently being tested), the different 21 amino acid peptides are designated into 1^{st}, 2^{nd} etc. construct by priority rank as needed/desired. The priority of assignment to one of multiple vectors composing the entire set of desired neo-epitopes is determined based on factors like relative size, priority of transcription, and overall hydrophobicity of the translated polypeptide.

In one embodiment, the construct structure disclosed herein comprises a nucleic acid sequence encoding a N terminal truncated LLO fused to one or more 21 mer neo-epitope(s) amino acid sequence flanked by a linker sequence and followed by at least one second neo epitope flanked by another linker and terminated by a SIINFEKL-6× His tag- and 2 stop codons closing the open reading frame: pHly-tLLO-21mer #1-4× glycine linker G1-21mer #2-4× glycine linker G2- . . . -SIINFEKL-6× His tag-2× stop codon. In another embodiment, the above construct's expression is driven by an hly gene promoter sequence or other suitable promoter sequence known in the art and further disclosed herein. It will be appreciated by a skilled artisan that each 21 mer neo-epitope sequence may also be fused to an immunogenic polypeptide such as a tLLO, truncated ActA or PEST amino acid sequence disclosed herein.

Different linker sequences are distributed between the neo-epitopes for minimizing repeats. This reduces possible secondary structures thereby allowing efficient transcription, translation, secretion, maintenance, or stabilization of the plasmid including the insert within the Lm recombinant vector strain population.

DNA synthesis is achieved by ordering nucleotide sequence from a vendor comprising the construct including the open reading frame comprising tLLO or tActA or ActA or PEST amino acid sequence fused to at least one neo-epitope. Additionally or alternatively multiple neo-epitopes are separated by one or more linker 4× glycine sequences. Additionally or alternatively inserts are constructed to comprise the desired sequence by molecular biology technics for example: by sewing PCR with specific over lapping primers and specific primers, or ligating different nucleotide sequences by an appropriate enzyme (e.g. Ligase), optionally following dissection by restriction enzymes, and any combination thereof.

In an embodiment different linker sequences are distributed between the neo-epitopes for minimizing repeats. This reduces possible secondary structures thereby allowing efficient transcription, translation, secretion, maintenance, or stabilization of the plasmid including the insert within the Lm recombinant vector strain population.

Selected DNA inserts are synthesized by technics standard in the art (e.g., PCR, DNA replication—bio-replication, oligonucleotide chemical synthesis) and cloned to a plasmid, for example as presented in Example 8. Plasmid is then transfected or conjugated into Lm vector. Additionally or alternatively, the insert is integrated into a phage vector and inserted into Lm vector by phage infection. Confirmation of construct is performed utilizing technics known in the art, for example bacterial colony PCR with insert specific primers, or purifying the plasmid and sequencing at least a portion comprising the insert.

### Example 20: Therapeutic Effects of Lm Neo-Antigen Constructs in B16F10 Murine Melanoma Model

After nonsynonymous mutations are identified in cancer cells that are not present in corresponding healthy cells, major efforts are typically invested to determine the mutational functional impact, such as cancer driver versus passenger status, to form a basis for selecting therapeutic targets. However, little attention has been devoted to either define the immunogenicity of these mutations or characterize the immune responses they elicit. From the immunologic perspective, mutations may be particularly potent vaccination targets, as they can create neo-antigens that are not subject to central immune tolerance. When attention has been devoted to define the immunogenicity of these mutations or characterize the immune responses they elicit, efforts are typically directed to narrowing down the nonsynonymous mutations to a single mutation to be included in a peptide for immunization. For example, in Castle et al., 962 nonsynonymous point mutations were identified in B16F10 murine melanoma cells, with 563 of those mutations in expressed genes. Fifty of these mutations were selected based on selection criteria including low false discovery rate (FDR) confident value, location in an expressed gene, and predicted immunogenicity. Out of these 50, only 16 were found to elicit immune responses in immunized mice, and only 11 of the 16 induced an immune response preferentially recognizing the mutated epitope. Two of the mutations were then found to induce tumor growth inhibition. See, e.g., Castle et al. (2012) Cancer Res. 72(5):1081-1091, herein incorporated by reference in its entirety for all purposes. In the constructs described in the following experiments, however, our data suggest that Neo 20 and Neo 30 are better at controlling tumor growth. In our constructs, Neo-12 contains the 12 most immunogenic epitopes. Neo 12 contains both tumor controlling epitopes (Mut30 and Mut44, as disclosed above in Table 5 in Example 19). Neo 20 contains Mut30-Mut2-Mut3-Mut3-Mut4 . . . Mut19). Neo 30 contains Mut30-Mut2-Mut3 . . . Mut-29). Neo 20 and Neo 30 only contain one of the tumor controlling epitopes identified by Castle (Mut30), and then they contain both immunogenic and non-immunogenic epitopes. Despite not having multiple tumor-controlling epitopes, and containing many non-tumor-controlling and even non-immunogenic epitopes, our data suggest that Neo 20 and Neo 30 are better at controlling tumor growth.

### Experiment 1

To determine therapeutic response generated by Lm neo-antigen constructs, a tumor regression study was designed to examine the therapeutic effects of such constructs on tumor growth in the B16F10 C57Bl/6 murine melanoma model. Specifically, Lm neo-antigen vectors were designed with 12 neo-antigens (Lm-Castle 12, containing Mut30, Mut5, Mut17, Mut20, Mut22, Mut24, Mut25, Mut44, Mut46, Mut48, and Mut50) or 20 neo-antigens (Lm-Castle 20, containing Mut30, Mut2, Mut3, Mut4, Mut5, Mut6, Mut7, Mut8, Mut9, Mut10, Mut11, Mut12, Mut13, Mut14, Mut15, Mut16, Mut17, Mut18, Mut19, and Mut20) identified by Castle et al. and as set forth in Table 5 in Example 19. See, e.g., Castle et al. (2012) Cancer Res. 72(5):1081-1091, herein incorporated by reference in its entirety for all purposes.

Tumor Cell Line Expansion.

The B16F10 melanoma cell line was cultured in c-RPMI containing 10% FBS (50 mL) and 1× Glutamax (5 mL). The c-RPMI media includes the following components:

| | |
|---|---|
| RPMI 1640 | 450 mL |
| FCS | 50 mL |
| HEPES | 5 mL |
| NEAA | 5 ml |
| L-Glutamine | 5 mL |
| Na-Pyruvate | 5 mL |
| Pen/step | 5 mL |
| 2-ME (14.6M) | 129 μL |

Tumor Inoculation.

On Day 0, B16F10 cells were trypsinized and washed twice with media. Cells were counted and re-suspended at a concentration of 1×10⁵ cells/200 uL of PBS for injection. B16F10 cells were then implanted subcutaneously in the right flank of each mouse. Mice were vaccinated on Day 3 of the study. Tumors were measured and recorded twice per week until reaching a size of 12 mm in diameter. Once tumors met sacrifice criteria, mice were euthanized, and tumors were excised and measured.

Immunotherapy Treatment.

On Day 3, immunotherapies and treatments began. Groups were treated with Lm (IP), and boosted twice. Details are listed in Table 8.

TABLE 8
Treatment schedule.

| | B16F10 Tumor | | | |
|---|---|---|---|---|
| | Inoculation | Dose 1: Treatments | | |
| Groups | 1 × 10⁵ | at 1 week intervals | Dose 2: | Dose 3: |
| (10 mice/group) | cells/200uL/mouse | 21JAN16 | 28FEB16 | 10FEB16 |
| 1 - PBS ONLY | 18JAN16 | 200 uL/mouse | 200uL/mouse | NA |
| (neg control) | | | | |
| 2 - Poly (I:C) ONLY | 18JAN16 | (50 ug in 200 uL | (50 ug in 200 uL | NA |
| (50 ug in 200 uL PBS) | | PBS-SQ) | PBS-SQ) | |
| (neg control) | | | | |
| 3 - LmddA-274 ONLY | 18JAN16 | 1 × 10⁸ IP | 1 × 10⁸ IP | NA |
| (neg control) | | | | |
| 4 - Lm-Castle 12 | 18JAN16 | 1 × 10⁸ IP | 1 × 10⁸ IP | 1 × 10⁸ IP |
| (SEQ ID NO: 118) | | | | |
| 5 - Lm Castle 20 | 18JAN16 | 1 × 10⁸ IP | 1 × 10⁸ IP | 1 × 10⁸ IP |
| (SEQ ID NO: 119) | | | | |

Immunotherapy Treatment Preparation.

1. PBS ONLY—200 uL/mouse IP.

2. LmddA-274 (Titer: 1.5×10⁹ CFU/mL)
a. Thaw 1 vial from −80° C. in 37° C. water bath.
b. Spin at 14, 000 rpm for 2 min and discard supernatant.
c. Wash 2 times with 1 mL PBS and discard PBS.
d. Re-suspend in PBS to a final concentration of 5×10⁸ CFU/mL.

3. Lm-Castle 12 (Titer: 1.59×10⁹CFU/mL and Lm-Castle 20 (Titer: 1.6×10⁹ CFU/mL)
a. Thaw 1 vial from −80° C. in 37° C. water bath.
b. Spin at 14, 000 rpm for 2 min and discard supernatant.
c. Wash 2 times with 1 mL PBS and discard PBS.
d. Re-suspend in PBS to a final concentration of 5×10⁸ CFU/mL.

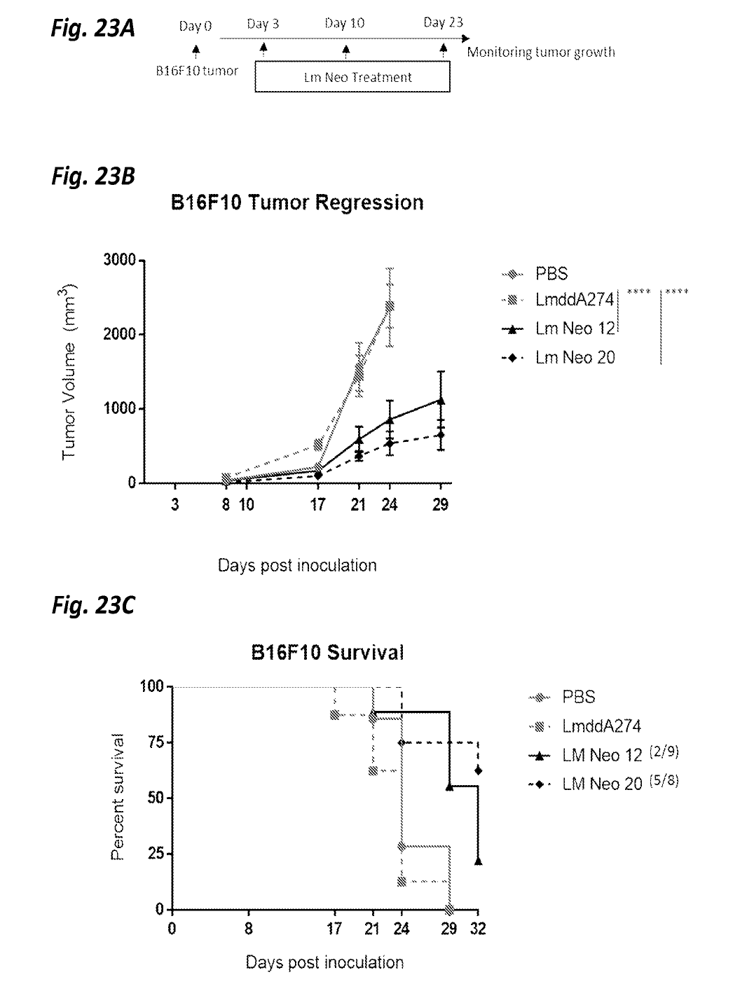

As shown in FIG. 23B, growth of tumors was inhibited by Lm-Neo 12 and Lm-Neo 20 as compared with the control groups (PBS and LmddA274). LmddA274 is the listeria control, and is an empty vector. It includes the truncated LLO (tLLO), however no neo-epitopes are attached. In addition, Lm-Neo 20, which contained 20 neo-antigens, inhibited tumor growth to a greater extent than Lm-Neo 12, which contained 12 neo-antigens. Likewise, Lm-Neo 20 and Lm-Neo 12 each result in increased survival time when compared with the control groups, with Lm-Neo 20 providing the greatest protective effect (FIG. 23C). These data show that vaccination with Lm carrying neo-epitopes is able to confer antitumoral effects, and increasing the number of neo-epitopes increases the antitumoral effects.

### Experiment 2

To further compare therapeutic responses generated by different Lm neo-antigen constructs, a tumor regression study was designed to examine the therapeutic effects of such constructs on tumor growth in the B16F10 C57Bl/6 murine melanoma model. Specifically, Lm neo-antigen vectors were designed with 12 neo-antigens (Lm-Castle 12), 20 neo-antigens (Lm-Castle 20), or 39 neo-antigens (Lm-Castle 39; no linker, no 20-29 (Lm-Castle 30)) identified by Castle et al. See, e.g., Castle et al. (2012) Cancer Res. 72(5):1081-1091, herein incorporated by reference in its entirety for all purposes.

Tumor Cell Line Expansion.

The B16F10 melanoma cell line was cultured in c-RPMI containing 10% FBS (50 mL) and 1× Glutamax (5 mL).

Tumor Inoculation.

On Day 0, B16F10 cells were trypsinized and washed twice with media. Cells were counted and re-suspended at a concentration of 1×10⁵ cells/200 uL of PBS for injection. B16F10 cells were then implanted subcutaneously in the right flank of each mouse. Mice were vaccinated on Day 4 of the study. Tumors were measured and recorded twice per week until reaching a size of 1500 mm³ in volume. Once tumors met sacrifice criteria, mice were euthanized, and tumors were excised and measured.

Immunotherapy Treatment.

On Day 4, immunotherapies and treatments began. Animals were treated once every 7 days until the end of the study. Groups were treated with either PBS, LmddA274, Lm-Castle 12, Lm-Castle 20, Lm-Castle 39 no linker no 20-29, detailed in Table 9.

TABLE 9
Treatment schedule.

| | B16F10 Tumor | | | | | |
|---|---|---|---|---|---|---|
| Groups | Inoculation 1 × 10⁵ | Dose 1: | Dose 2: | Dose 3: | Dose 4: | Dose 5: |
| (10 = N/group) | cells/200 uL/mouse | 1 Mar. 2016 | 8 Mar. 2016 | 15 Mar. 2016 | 22 Mar. 2016 | 29 Mar. 2016 |
| 1-PBS ONLY | 26 Feb. 2016 | 200 uL/ | 200 uL/ | 200 uL/ | 200 uL/ | 200 uL/ |
| (neg control) | | Mouse IP | Mouse IP | Mouse IP | Mouse IP | Mouse IP |
| 2-LmddA-274 | 26 Feb. 2016 | 1 × 10⁸ IP | 1 × 10⁸ IP | 1 × 10⁸ IP | 1 × 10⁸ IP | 1 × 10⁸ IP |
| ONLY | | | | | | |
| (neg control) | | | | | | |
| 3- Lm Castle 12 | 26 Feb. 2016 | 1 × 10⁸ IP | 1 × 10⁸ IP | 1 × 10⁸ IP | 1 × 10⁸ IP | 1 × 10⁸ IP |
| (SEQ ID NO: 118) | | | | | | |
| 4- Lm Castle 20 | 26 Feb. 2016 | 1 × 10⁸ IP | 1 × 10⁸ IP | 1 × 10⁸ IP | 1 × 10⁸ IP | 1 × 10⁸ IP |
| (SEQ ID NO: 119) | | | | | | |
| 5- Lm Castle 39 | 26 Feb. 2016 | 1 × 10⁸ IP | 1 × 10⁸ IP | 1 × 10⁸ IP | 1 × 10⁸ IP | 1 × 10⁸ IP |
| (no link no 20-29) | | | | | | |
| (also called Lm | | | | | | |
| Castle 30) | | | | | | |
| (SEQ ID NO: 120) | | | | | | |

Immunotherapy Treatment Preparation.
1. PBS ONLY ˜200 uL/mouse IP.
2. LmddA-274 (Titer: 1.7×10⁹CFU/mL)
   a. Thaw 1 vial from −80° C. in 37° C. water bath.
   b. Spin at 14,000 rpm for 2 min and discard supernatant.
   c. Wash 2 times with 1 mL PBS and discard PBS.
   d. Re-suspend in PBS to a final concentration of 5×10⁸ CFU/mL.
3. Lm-Castle 12 (Titer: 1.59×10⁹CFU/mL and Lm-Castle 20 (Titer: 1.6×10⁹ CFU/mL) and Lm-Castle 39)Titer: 1×10⁹CFU/mL)
   a. Thaw 1 vial from −80° C. in 37° C. water bath.
   b. Spin at 14,000 rpm for 2 min and discard supernatant.
   c. Wash 2 times with 1 mL PBS and discard PBS.
   d. Re-suspend in PBS to a final concentration of 5×10⁸ CFU/mL.

Harvesting Details.

The spleen from each mouse was collected in an individual tube containing 5 mL of c-RPMI medium. Detailed steps are described below. All tumors were excised and measured at termination of the study.
1. Harvest spleens using sterile forceps and scissors.
2. Mash each spleen in wash medium (RPMI only) using two glass slides or the back of plunger from a 3 mL syringe.
3. Transfer cells in the medium to a 15 mL tube.
4. Pellet cells at 1,000 RPM for 5 min at room temperature.
5. Discard supernatant, re-suspend cells in the remaining wash buffer gently, and add 2 mL RBC lysis buffer per spleen to the cell pellet. Mix cells gently with lysis buffer by tapping the tube and wait for 1 min
6. Immediately add 10 mL of c-RPMI medium to the cell suspension to deactivate the lysis buffer.
7. Spin cells at 1,000 for 5 min at room temperature.
8. Pass the cells through a cell strainer and wash them one more time with 10 mL c-RPMI.
9. Count cells using hemocytometer/moxi flow and check the viability by Trypan blue staining. Each spleen should yield ˜1-2×10⁸ cells.
10. Divide the cells for staining
11. Follow immudex dextramer staining protocol: with the one exception of adding the cell surface antibodies (CD8, CD62L) in 2.4G2 instead of staining buffer (www.immudex.com/media/12135/tf1003.03_general_staining_procedure_mhc_d extramer.pdf).

CD8+ T Cell Response.

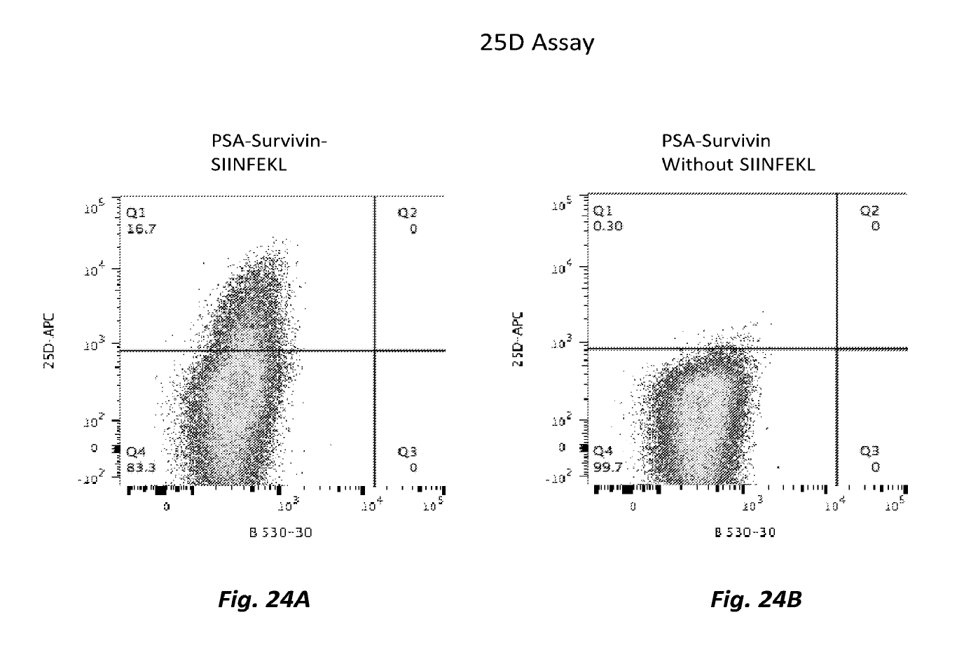

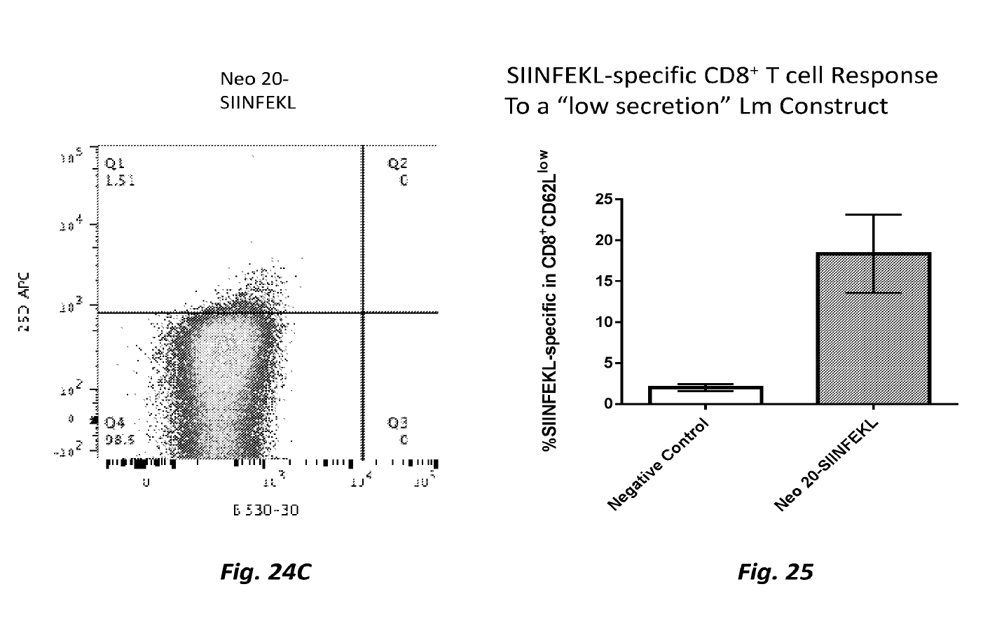

25D assays were done as explained above to measure expression and secretion of the Lm-Neo 20 construct in antigen presenting cells. FIG. 24A is a positive control (PSA-Survivin-SIINFEKL), FIG. 24B is a negative control (PSA-Survivin without SIINFEKL), and FIG. 24C is the Lm-Neo 20 (with SIINFEKL tag at C-terminus). As indicated in FIG. 24, the Lm-Neo 20 expresses and is secreted, but only at low levels compared to the positive control. However, despite these low secretion levels, a specific CD8+ T cell response to SIINFEKL was observed. FIG. 25 shows the SIINFEKL-specific CD8+ T cell response to the “low secretion” Lm-Neo 20 construct. As shown in FIG. 25, approximately 20% of the CD8+ T cells are specific for antigens in the Lm Neo 20 construct.

Antitumor Effects.

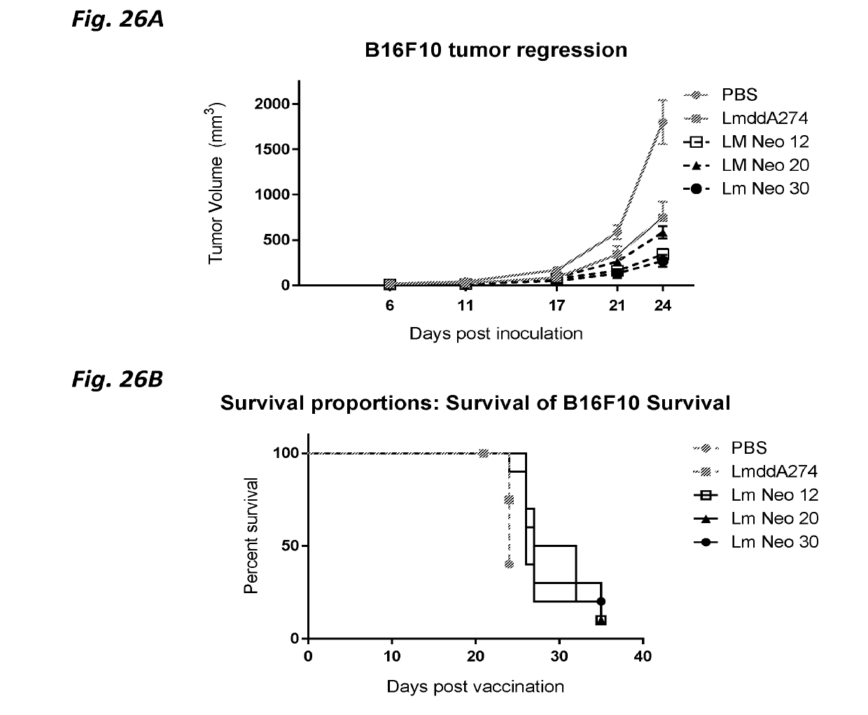

As shown in FIG. 26A, growth of tumors was inhibited by Lm-Neo 12, Lm-Neo 20, and Lm-Neo 30 as compared with the control groups (PBS and LmddA274). In addition, Lm-Neo 30, which contained 30 neo-antigens, inhibited tumor growth to a greater extent than Lm-Neo 20, which contained 20 neo-antigens, which inhibited tumor growth to a greater extent than Lm-Neo 12, which contained 12 neo-antigens. Likewise, Lm-Neo 30, Lm-Neo 20, and Lm-Neo 12 each result in increased survival time when compared with the control groups, with Lm-Neo 30 providing the greatest protective effect and Lm-Neo 20 providing the next greatest protective effect (FIG. 23C). These data show that vaccination with Lm carrying neo-epitopes is able to confer antitumoral effects, and increasing the number of neo-epitopes increases the antitumoral effects.

### Example 21: Identification of Potential Neo-Antigens Resulting from Frameshift Mutations

Levels of neo-epitopes based on nonsynonymous somatic missense mutations vary significantly across and within indications. Examples of variations across and within indications are shown in Table 10.

TABLE 10
Neo-epitope levels based on nonsynonymous somatic mutations
vary significantly across and within indications.

| | | % Tumors with Nonsynonymous Mutations in Range | | | | | |
|---|---|---|---|---|---|---|---|
| Tumor Type | Median | <40 | <120 | >120 | >200 | >400 | >1000 |
| Melanoma | 396 | 2% | 14% | 86% | 74% | 48% | 15% |
| Lung squamous cell | 245 | 2% | 13% | 87% | 61% | 18% | 2% |
| carcinoma | | | | | | | |
| Lung adenocarcinoma | 193 | 12% | 34% | 66% | 49% | 21% | 4% |
| Lung small cell | 175 | 4% | 27% | 73% | 36% | 10% | |
| Bladder | 155 | 9% | 40% | 60% | 37% | 11% | 3% |
| Stomach | 129 | 7% | 48% | 52% | 34% | 26% | 20% |
| Esophageal adenocarcinoma | 117 | 5% | 54% | 46% | 11% | 4% | 1% |
| Colorectal | 96 | 6% | 68% | 32% | 15% | 13% | 7% |
| Uterus | 95 | 7% | 58% | 42% | 39% | 30% | 11% |
| Head and neck | 95 | 20% | 69% | 31% | 16% | 4% | |
| Diffuse large B-cell | 94 | 14% | 59% | 41% | 14% | | |
| lymphoma | | | | | | | |
| Glioblastoma multiforme | 61 | 16% | 96% | 4% | 2% | 1% | 1% |
| Ovarian | 50 | 34% | 94% | 6% | 1% | 1% | |
| Kidney papillary cell | 48 | 18% | 100% | | | | |
| Kidney clear cell | 46 | 36% | 100% | 0% | 0% | 0% | |
| Multiple myeloma | 42 | 38% | 97% | 3% | 2% | | |
| Pancreas | 32 | 77% | 100% | | | | |
| Breast | 28 | 70% | 96% | 4% | 2% | | |
| Low-grade glioma | 26 | 75% | 100% | | | | |
| Chronic lymphocytic | 23 | 92% | 100% | | | | |
| leukemia | | | | | | | |
| Prostate | 22 | 90% | 98% | 2% | 0% | | |
| Neuroblastoma | 17 | 89% | 99% | 1% | 1% | | |
| Carcinoid | 16 | 100% | 100% | | | | |
| Kidney chromophobe | 12 | 97% | 98% | 2% | 2% | 2% | |
| Medulloblastoma | 11 | 100% | 100% | | | | |
| Acute myeloid leukemia | 10 | 92% | 96% | 4% | 2% | 1% | |
| Thyroid | 10 | 100% | 100% | | | | |
| ALL | 9 | 96% | 100% | | | | |
| Ewing sarcoma | 9 | 95% | 100% | | | | |
| Rhabdoid tumor | 5 | 100% | 100% | | | | |

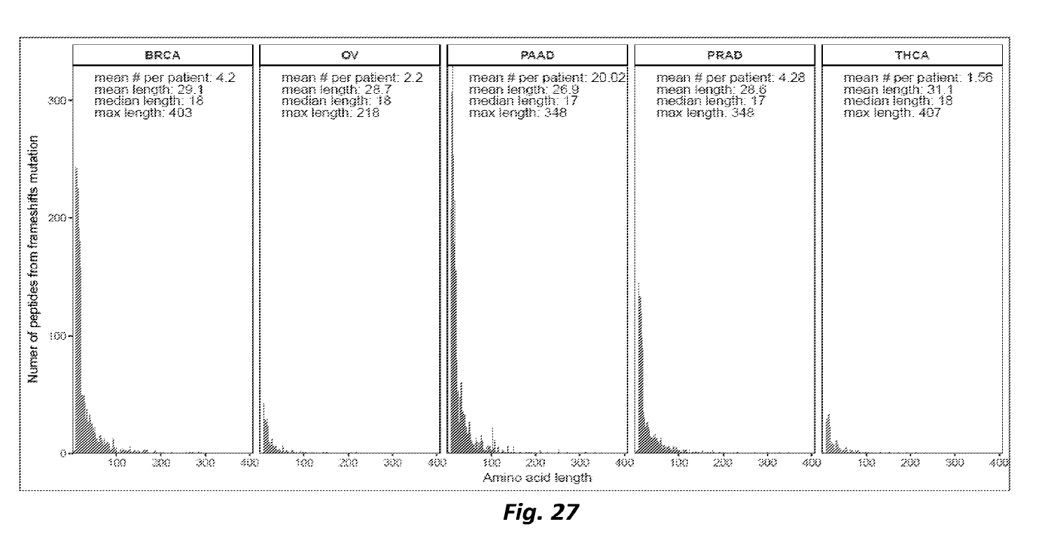

High neo-epitope presence may be an important factor for response, and tumors with fewer neo-epitopes may be less likely to respond. To increase the number of potential neo-epitopes for tumors having a low number of tumor-specific, nonsynonymous, somatic, missense mutations, nonsensical peptides encoded by genes with tumor-specific frameshift mutations can be used. Mutation data was obtained from the Cancer Genome Atlas (TCGA) for prostate adenocarcinoma (PRAD), pancreas adenocarcinoma (PAAD), breast invasive carcinoma (BRCA), ovarian serous cystadenocarcinoma (OV), and thyroid carcinoma. Patients in these disease cohorts are characterized by low mutation rates for single nucleotide variants (SNVs) (low missense mutation rates). Identification of neo-antigens generated from frameshift mutations can expand the targets for the Lm technology. To that end, we identified every frameshift mutation for each patient within a TCGA disease cohort and calculated the resulting neo-antigen peptide (Table 11, FIG. 27). The average number of neo-antigen peptides from frameshift mutations ranged from 1.56 in thyroid carcinoma to 20.02 in pancreatic adenocarcinoma. Mean length of peptide sequence ranged from 26.90 in pancreatic adenocarcinoma to 31.10 in thyroid carcinoma. The maximum peptide length across all cohorts was 403 amino acids long. MHC Class I molecules can bind peptides 8-11 amino acids in length. Non-self peptide sequences generated by frameshift mutations have the potential to present numerous peptide fragments that will elicit a positive immune response with Lm technology.

TABLE 11
Frameshift mutations in PAAD, PRAD, BRCA, OV, and THCA
cohorts.

| | mean # of | | | max |
|---|---|---|---|---|
| | frameshift | mean | median | peptide |
| Cancer type | mutations | length of | length of | length per |
| (TCGA abbreviation) | per patient | peptide | peptide | cohort |
| Pancreatic | 20.02 | 26.90 | 17 | 348 |
| adenocarcinoma (PAAD) | | | | |
| Prostate adenocarcinoma | 4.28 | 28.60 | 17 | 348 |
| (PRAD) | | | | |
| Breast invasive | 4.20 | 29.10 | 18 | 403 |
| carcinoma (BRCA) | | | | |
| Ovarian serous | 2.20 | 28.87 | 18 | 218 |
| cystadenocarcinoma | | | | |
| (OV) | | | | |
| Thyroid carcinoma | 1.56 | 31.10 | 18 | 407 |
| (THCA) | | | | |

### Example 22: Neo-Antigens Derived from Tumor-Specific Frameshift Mutations are Able to Control Tumor Growth

To determine if Lm constructs containing neo-antigens derived from frameshift mutations are able to control tumor growth, a tumor regression study was done to examine the therapeutic effects of Lm neoantigen vectors (Lm Neo 12, Lm Frameshift 1, and Lm Frameshift 2) as compared to the empty vector negative control strain LmddA-274. The Lm B16F10 frameshift 1 chimeric protein is set forth in SEQ ID NO: 61 (encoded by SEQ ID NO: 62). The Lm B16F10 frameshift 2 chimeric protein is set forth in SEQ ID NO: 63 (encoded by SEQ ID NO: 64). A third Lm B16F10 frameshift chimeric protein is set forth in SEQ ID NO: 65 (encoded by SEQ ID NO: 66).

Tumor Cell Line Expansion:

B16F10 melanoma cells were cultured in c-RPMI containing 10% FBS (50 mL), and 1× Glutamax (5 mL).

Tumor Inoculation:

On Day 0, (26 Sep. 16), B16F10 cells were trypsinized and washed twice with media. Cells were counted and re-suspended at a concentration of 1×10⁵ cells/200 uL of PBS for injection. B16F10 cells were implanted subcutaneously in the right flank of each mouse. All animals were placed into randomized groups. Mice were vaccinated on Day 3 of the study (29 Sep. 16).

Vaccine/Lm Treatment:

Vaccines and treatments began on Day 3. Groups were treated with Lm (200 uL/IP/mouse) and boosted indefinitely (details listed in Table 12).

Vaccine/Treatment Preparation:
a. Thaw 1 vial from −80° C. in 37 C water bath.
b. Spin at 14,000 rpm for 2 min and discard supernatant.
c. Wash 2 times with 1 mL PBS and discard PBS.
d. Re-suspend in PBS to a final concentration of 5×10⁸ CFU/mL.

TABLE 12
Treatment schedule.

| | Dose 1: | | | |
|---|---|---|---|---|
| | 29SEP16 | | | |
| | Treatments | | | |
| Groups | at 1 week | Dose 2: | Dose 3: | Dose 4: |
| (10 mice/group) | intervals | 06OCT16 | 13OCT16 | 20OCT16 |
| 1 - LmddA-274 | 1 × 10⁸ IP | 1 × 10⁸ IP | 1 × 10⁸ IP | 1 × 10⁸ IP |
| (neg control) Titer: | | | | |
| 1.7 × 10⁹ CFU/mL | | | | |
| 2 - Lm Neo 12 | 1 × 10⁸ IP | 1 × 10⁸ IP | 1 × 10⁸ IP | 1 × 10⁸ IP |
| (Castle 12) | | | | |
| (positive control) | | | | |
| Titer: 1 × 10⁹ CFU/mL | | | | |
| 3 - Frameshift 1 | 1 × 10⁸ IP | 1 × 10⁸ IP | 1 × 10⁸ IP | 1 × 10⁸ IP |
| (FS1) Titer: | | | | |
| 1.5 × 10⁹ CFU/mL | | | | |
| 4 - Frameshift 2 | 1 × 10⁸ IP | 1 × 10⁸ IP | 1 × 10⁸ IP | 1 × 10⁸ IP |
| (FS2) Titer: | | | | |
| 1.21 × 10⁹ CFU/mL | | | | |

Results:

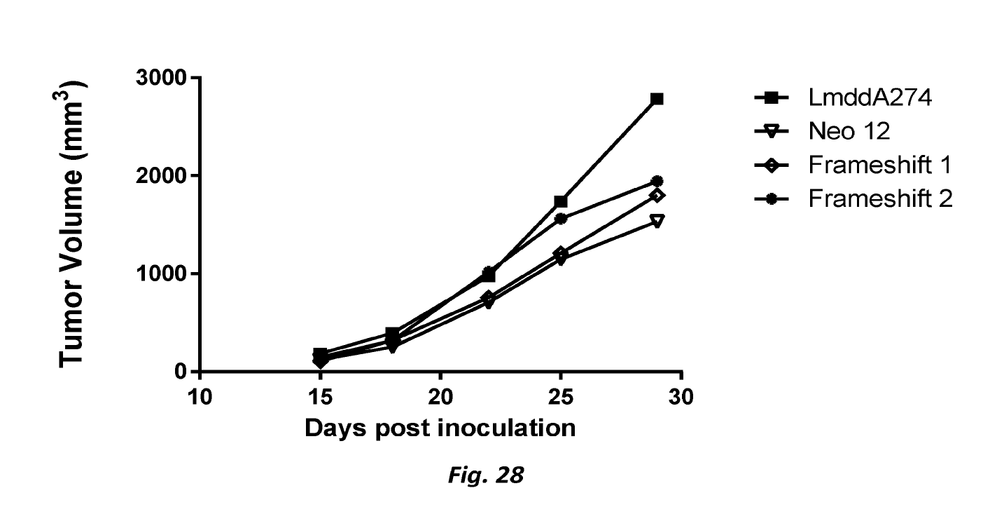

As shown in FIG. 28, B16F10-tumor-bearing mice immunized with Lm constructs that secrete frameshift mutations (Frameshift 1 or Frameshift 2) derived from B16F10 tumor cells have decreased tumor growth compared to tumor-bearing animals that were treated only with the empty vector negative control (LmddA-274). Neo 12 was used as a positive control.

While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

1. An immunotherapy delivery vector comprising a nucleic acid comprising an open reading frame encoding a recombinant polypeptide comprising a PEST-containing peptide fused to one or more heterologous peptides, wherein the one or more heterologous peptides comprise one or more frameshift-mutation-derived peptides comprising one or more immunogenic neo-epitopes.

2. The immunotherapy delivery vector of claim 1, wherein the one or more frameshift-mutation-derived peptides are encoded by a source nucleic acid sequence comprising at least one disease-specific or condition-specific frameshift mutation.

3. The immunotherapy delivery vector of claim 2, wherein the source nucleic acid sequence comprises one or more regions of microsatellite instability.

4. The immunotherapy delivery vector of any preceding claim, wherein the at least one frameshift mutation is within the penultimate exon or the last exon of a gene.

5. The immunotherapy delivery vector of any preceding claim, wherein each of the one or more frameshift-mutation-derived peptides is about 8-10, 11-20, 21-40, 41-60, 61-80, 81-100, 101-150, 151-200, 201-250, 251-300, 301-350, 351-400, 401-450, 451-500, or 8-500 amino acids in length.

6. The immunotherapy delivery vector of any preceding claim, wherein the one or more frameshift-mutation-derived peptides do not encode a post-translational cleavage site.

7. The immunotherapy delivery vector of any preceding claim, wherein the one or more immunogenic neo-epitopes comprise a T-cell epitope.

8. The immunotherapy delivery vector of any preceding claim, wherein the one or more frameshift-mutation-derived peptides comprise a cancer-associated or tumor-associated neo-epitope or a cancer-specific or tumor-specific neo-epitope.

9. The immunotherapy delivery vector of claim 8, wherein the tumor or cancer comprises a breast cancer or tumor, a cervical cancer or tumor, a Her2-expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor, or a metastasis of any one of the cancers or tumors.

10. The immunotherapy delivery vector of any one of claims 1-7, wherein the one or more frameshift-mutation-derived peptides comprise an infectious-disease-associated or infectious-disease-specific neo-epitope.

11. The immunotherapy delivery vector of any preceding claim, wherein the recombinant polypeptide comprises about 1-20 neo-epitopes.

12. The immunotherapy delivery vector of any preceding claim, wherein the one or more heterologous peptides comprise multiple heterologous peptides operably linked in tandem, wherein the PEST-containing peptide is fused to one of the multiple heterologous peptides.

13. The immunotherapy delivery vector of claim 12, wherein the recombinant polypeptide comprises multiple frameshift-mutation-derived peptides, wherein each frameshift-mutation-derived peptide is different.

14. The immunotherapy delivery vector of claim 12 or 13, wherein the multiple heterologous peptides are fused directly to each other with no intervening sequence.

15. The immunotherapy delivery vector of claim 12 or 13, wherein the multiple heterologous peptides are operably linked to each other via one or more peptide linkers or one or more 4× glycine linkers.

16. The immunotherapy delivery vector of any one of claims 12-15, wherein the PEST-containing peptide is operably linked to the N-terminal heterologous peptide.

17. The immunotherapy delivery vector of any preceding claim, wherein the PEST-containing peptide is a mutated listeriolysin O (LLO) protein, a truncated LLO (tLLO) protein, a truncated ActA protein, or a PEST amino acid sequence.

18. The immunotherapy delivery vector of any preceding claim, wherein the C-terminal end of the recombinant polypeptide is operably linked to a tag.

19. The immunotherapy delivery vector of claim 18, wherein the C-terminal end of the recombinant polypeptide is operably linked to a tag by a peptide linker or a 4× glycine linker.

20. The immunotherapy delivery vector of claim 18 or 19, wherein the tag is selected from the group consisting of: a 6× histidine tag, a 2× FLAG tag, a 3× FLAG tag, a SIINFEKL peptide, a 6× histidine tag operably linked to a SIINFEKL peptide, a 3× FLAG tag operably linked to a SIINFEKL peptide, a 2× FLAG tag operably linked to a SIINFEKL peptide, and any combination thereof.

21. The immunotherapy delivery vector of any one of claims 18-20, wherein the open reading frame encoding the recombinant polypeptide comprises two stop codons following the sequence encoding the tag.

22. The immunotherapy delivery vector of any preceding claim, wherein the open reading frame encoding the recombinant polypeptide is operably linked to an hly promoter and encodes components comprising from N-terminus to C-terminus: tLLO-[heterologous peptide]ₙ-(peptide tag(s))-(2× stop codon), wherein n=2-20, and wherein at least one heterologous peptide is a frameshift-mutation-derived peptide,
or wherein the open reading frame encoding the recombinant polypeptide is operably linked to an hly promoter and encodes components comprising from N-terminus to C-terminus: tLLO-[(heterologous peptide)-(glycine linker₍₄ₓ₎)]ₙ-(peptide tag(s))-(2× stop codon), wherein n=2-20, and wherein at least one heterologous peptide is a frameshift-mutation-derived peptide.

23. The immunotherapy delivery vector of any preceding claim, wherein the one or more heterologous peptides further comprise one or more nonsynonymous-missense-mutation-derived peptides.

24. The immunotherapy delivery vector of claim 23, wherein the one or more nonsynonymous-missense-mutation-derived peptides are encoded by a source nucleic acid sequence comprising at least one disease-specific or condition-specific nonsynonymous missense mutation.

25. The immunotherapy delivery vector of claim 23 or 24, wherein each of the one or more nonsynonymous-missense-mutation-derived peptides is about 5-50 amino acids in length or about 8-27 amino acids in length.

26. The immunotherapy delivery vector of any preceding claim, wherein the immunotherapy delivery vector is a recombinant Listeria strain.

27. The immunotherapy delivery vector of claim 26, wherein the recombinant Listeria strain expresses and secretes the recombinant polypeptide.

28. The immunotherapy delivery vector of claim 26 or 27, wherein the open reading frame encoding the recombinant polypeptide is integrated into the Listeria genome.

29. The immunotherapy delivery vector of claim 26 or 27, wherein the open reading frame encoding the recombinant polypeptide is in a plasmid.

30. The immunotherapy delivery vector of claim 29, wherein the plasmid is stably maintained in the recombinant Listeria strain in the absence of antibiotic selection.

31. The immunotherapy delivery vector of any one of claims 26-30, wherein the Listeria strain is an attenuated Listeria strain.

32. The immunotherapy delivery vector of claim 31, wherein the attenuated Listeria comprises a mutation in one or more endogenous genes.

33. The immunotherapy delivery vector of claim 32, wherein the endogenous gene mutation is selected from an actA gene mutation, a prfA mutation, an actA and inlB double mutation, a dal/dat gene double mutation, a dal/dat/actA gene triple mutation, or a combination thereof, and wherein the mutation comprises an inactivation, truncation, deletion, replacement, or disruption of the gene or genes.

34. The immunotherapy delivery vector of any one of claims 26-33, wherein the nucleic acid comprising the open reading frame encoding the recombinant polypeptide further comprises a second open reading frame encoding a metabolic enzyme, or wherein the recombinant Listeria strain further comprises a second nucleic acid comprising an open reading frame encoding a metabolic enzyme.

35. The immunotherapy delivery vector of claim 34, wherein the metabolic enzyme is an alanine racemase enzyme or a D-amino acid transferase enzyme.

36. The immunotherapy delivery vector of any one of claims 26-35, wherein the Listeria is Listeria monocytogenes.

37. The immunotherapy delivery vector of claim 36, wherein the recombinant Listeria strain comprises a deletion of or inactivating mutation in actA, dal, and dat, wherein the nucleic acid comprising the open reading frame encoding the recombinant polypeptide is in an episomal plasmid and comprises a second open reading frame encoding an alanine racemase enzyme or a D-amino acid aminotransferase enzyme, and wherein the PEST-containing peptide is an N-terminal fragment of LLO.

38. An immunogenic composition comprising at least one immunotherapy delivery vector of any one of claims 1-37.

39. The immunogenic composition of claim 38, further comprising an adjuvant.

40. The immunogenic composition of claim 49, wherein the adjuvant comprises a granulocyte/macrophage colony-stimulating factor (GM-CSF) protein, a nucleotide molecule encoding a GM-CSF protein, saponin QS21, monophosphoryl lipid A, an unmethylated CpG-containing oligonucleotide, or a detoxified, nonhemolytic form of LLO (dtLLO).

41. A method of treating, suppressing, preventing, or inhibiting a disease or a condition in a subject, comprising administering to the subject the immunogenic composition of any one of claims 38-40, wherein the one or more frameshift-mutation-derived peptides are encoded by a source nucleic acid sequence from a disease-bearing or condition-bearing biological sample from the subject.

42. The method of claim 42, wherein the method elicits a personalized anti-disease or anti-condition immune response in the subject, wherein the personalized immune response is targeted to the one or more frameshift-mutation-derived peptides.

43. The method of claim 41 or 42, wherein the disease or condition is a cancer or tumor.

44. The method of any one of claims 41-43, further comprising administering a booster treatment.

45. A process for creating the immunotherapy delivery vector of any one of claims 1-37 that is personalized for a subject having a disease or condition, comprising:
(a) comparing one or more open reading frames (ORFs) in nucleic acid sequences extracted from a disease-bearing or condition-bearing biological sample from the subject with one or more ORFs in nucleic acid sequences extracted from a healthy biological sample, wherein the comparing identifies one or more nucleic acid sequences encoding one or more peptides comprising one or more immunogenic neo-epitopes encoded within the one or more ORFs from the disease-bearing or condition-bearing biological sample, wherein at least one of the one or more nucleic acid sequences comprises one or more frameshift mutations and encodes one or more frameshift-mutation-derived peptides comprising one or more immunogenic neo-epitopes; and
(b) generating an immunotherapy delivery vector comprising a nucleic acid comprising an open reading frame encoding a recombinant polypeptide comprising the one or more peptides comprising the one or more immunogenic neo-epitopes identified in step (a).

46. The process of claim 45, further comprising storing the immunotherapy delivery vector for administering to the subject within a predetermined period of time.

47. The process of claim 45 or 46, further comprising administering a composition comprising the immunotherapy vector to the subject, wherein the administering results in the generation of a personalized T-cell immune response against the disease or condition.

48. The process of any one of claims 45-47, wherein the disease-bearing or condition-bearing biological sample is obtained from the subject having the disease or condition.

49. The process of any one of claims 45-48, wherein the healthy biological sample is obtained from the subject having the disease or condition.

50. The process of any one of claims 45-49, wherein the disease-bearing or condition-bearing biological sample and the healthy biological sample each comprises a tissue, a cell, a blood sample, or a serum sample.

51. The process of any one of claims 45-50, wherein the comparing in step (a) comprises use of a screening assay or screening tool and associated digital software for comparing the one or more ORFs in the nucleic acid sequences extracted from the disease-bearing or condition-bearing biological sample with the one or more ORFs in the nucleic acid sequences extracted from the healthy biological sample,
wherein the associated digital software comprises access to a sequence database that allows screening of mutations within the ORFs in the nucleic acid sequences extracted from the disease-bearing or condition-bearing biological sample for identification of immunogenic potential of the neo-epitopes.

52. The process of any one of claims 45-51, wherein the nucleic acid sequences extracted from the disease-bearing or condition-bearing biological sample and the nucleic acid sequences extracted from the healthy biological sample are determined using exome sequencing or transcriptome sequencing.

53. The process of any one of claims 45-52, wherein the one or more frameshift-mutation-derived peptides are characterized for neo-epitopes by generating one or more different peptide sequences from the one or more frameshift-mutation-derived peptides.

54. The process of claim 53, further comprising scoring each of the one or more different peptide sequences and excluding a peptide sequence if it does not score below a hydropathy threshold predictive of secretability in Listeria monocytogenes.

55. The process of claim 54, wherein the scoring is by a Kyte and Doolittle hydropathy index 21 amino acid window, and any peptide sequence scoring above a cutoff of about 1.6 is excluded or is modified to score below the cutoff.

56. The process of any one of claims 53-55, further comprising screening each of the one or more different peptide sequences and selecting for binding by MHC Class I or MHC Class II to which a T-cell receptor binds.

57. The process of any one of claims 45-56, wherein the process is repeated to create a plurality of immunotherapy delivery vectors, each comprising a different set of one or more immunogenic neo-epitopes.

58. The process of claim 57, wherein the plurality of immunotherapy delivery vectors comprises 2-5, 5-10, 10-15, 15-20, 10-20, 20-30, 30-40, or 40-50 immunotherapy delivery vectors.

59. The process of claim 57 or 58, wherein the combination of the plurality of immunotherapy delivery vectors comprises about 5-10, 10-15, 15-20, 10-20, 20-30, 30-40, 40-50, 50-60, 60-70, 70-80, 80-90, 90-100, or 100-200 immunogenic neo-epitopes.

60. The process of any one of claims 45-59, wherein the disease or condition is a tumor with fewer than 120, 110, 100, 90, 80, 70, 60, 50, 40, 30, 20, or 10 nonsynonymous missense mutations that are not present in the healthy biological sample.


SEQUENCE LISTING (120 sequences)

SEQ ID NO: 1 - protein, 8 aa, Gallus gallus
SIINFEKL

SEQ ID NO: 2 - protein, 32 aa, Listeria monocytogenes
KENSISSMAPPASPPASPKTPIEKKHADEIDK

SEQ ID NO: 3 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 4 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 5 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 6 - protein, 14 aa, Listeria monocytogenes
KTEEQPSEVNTGPR

SEQ ID NO: 7 - protein, 28 aa, Listeria monocytogenes
KASVTDTSEGDLDSSMQSADESTPQPLK

SEQ ID NO: 8 - protein, 20 aa, Listeria monocytogenes
KNEEVNASDFPPPPTDEELR

SEQ ID NO: 9 - protein, 33 aa, Listeria monocytogenes
RGGIPTSEEFSSLNSGDFTDDENSETTEEEIDR

SEQ ID NO: 10 - protein, 17 aa, Streptococcus pyogenes
KQNTASTETTTTNEQPK

SEQ ID NO: 11 - protein, 17 aa, Streptococcus equisimilis
KQNTANTETTTTNEQPK

SEQ ID NO: 12 - protein, 633 aa, Listeria monocytogenes
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 13 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 14 - protein, 100 aa, Listeria monocytogenes
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 15 - protein, 639 aa, Listeria monocytogenes
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 16 - protein, 93 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 17 - protein, 200 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 18 - protein, 226 aa, Listeria monocytogenes
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 19 - DNA, 678 bp, Listeria monocytogenes
atgaaaaaaataatgctagtttttattacacttatattagttagtctaccaattgcgcaacaaactgaagcatctagagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaa

SEQ ID NO: 20 - protein, 303 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 21 - protein, 370 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 22 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 23 - DNA, 6523 bp, Artificial Sequence
  Plasmid DNA
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 24 - DNA, 36 bp, Artificial Sequence
  Primer
cggaattcggatccgcgccaaatcattggttgattg

SEQ ID NO: 25 - DNA, 37 bp, Artificial Sequence
  Primer
gcgagtcgacgtcggggttaatcgtaatgcaattggc

SEQ ID NO: 26 - DNA, 35 bp, Artificial Sequence
  Primer
gcgagtcgacccatacgacgttaattcttgcaatg

SEQ ID NO: 27 - DNA, 39 bp, Artificial Sequence
  Primer
gatactgcagggatccttcccttctcggtaatcagtcac

SEQ ID NO: 28 - DNA, 19 bp, Artificial Sequence
  Primer
tgggatggccaagaaattc

SEQ ID NO: 29 - DNA, 22 bp, Artificial Sequence
  Primer
ctaccatgtcttccgttgcttg

SEQ ID NO: 30 - DNA, 28 bp, Artificial Sequence
  Primer
tgatctcgagacccacctggacatgctc

SEQ ID NO: 31 - DNA, 49 bp, Artificial Sequence
  Primer
ctaccaggacacgattttgtggaagaatatccaggagtttgctggctgc

SEQ ID NO: 32 - DNA, 49 bp, Artificial Sequence
  Primer
gcagccagcaaactcctggatattcttccacaaaatcgtgtcctggtag

SEQ ID NO: 33 - DNA, 50 bp, Artificial Sequence
  Primer
ctgccaccagctgtgcgcccgagggcagcagaagatccggaagtacacga

SEQ ID NO: 34 - DNA, 50 bp, Artificial Sequence
  Primer
tcgtgtacttccggatcttctgctgccctcgggcgcacagctggtggcag

SEQ ID NO: 35 - DNA, 39 bp, Artificial Sequence
  Primer
gtggcccgggtctagattagtctaagaggcagccatagg

SEQ ID NO: 36 - DNA, 28 bp, Artificial Sequence
  Primer
ccgcctcgaggccgcgagcacccaagtg

SEQ ID NO: 37 - DNA, 31 bp, Artificial Sequence
  Primer
cgcgactagtttaatcctctgctgtcacctc

SEQ ID NO: 38 - DNA, 28 bp, Artificial Sequence
  Primer
ccgcctcgagtacctttctacggacgtg

SEQ ID NO: 39 - DNA, 30 bp, Artificial Sequence
  Primer
cgcgactagtttactctggccggttggcag

SEQ ID NO: 40 - DNA, 31 bp, Artificial Sequence
  Primer
ccgcctcgagcagcagaagatccggaagtac

SEQ ID NO: 41 - DNA, 30 bp, Artificial Sequence
  Primer
cgcgactagtttaagccccttcggagggtg

SEQ ID NO: 42 - protein, 9 aa, Homo sapiens
HLYQGCQVV

SEQ ID NO: 43 - protein, 9 aa, Homo sapiens
KIFGSLAFL

SEQ ID NO: 44 - protein, 9 aa, Homo sapiens
RLLQETELV

SEQ ID NO: 45 - DNA, 5851 bp, Artificial Sequence
  Plasmid DNA
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgaggagctcctgcagtctagagtcgacactagtggatccagatctcccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 46 - DNA, 12 bp, Artificial Sequence
  Linker
ggtggtggagga

SEQ ID NO: 47 - DNA, 12 bp, Artificial Sequence
  Linker
ggtggaggtgga

SEQ ID NO: 48 - DNA, 12 bp, Artificial Sequence
  Linker
ggtggaggaggt

SEQ ID NO: 49 - DNA, 12 bp, Artificial Sequence
  Linker
ggaggtggtgga

SEQ ID NO: 50 - DNA, 12 bp, Artificial Sequence
  Linker
ggaggaggtggt

SEQ ID NO: 51 - DNA, 12 bp, Artificial Sequence
  Linker
ggaggtggaggt

SEQ ID NO: 52 - DNA, 12 bp, Artificial Sequence
  Linker
ggaggaggaggt

SEQ ID NO: 53 - DNA, 12 bp, Artificial Sequence
  Linker
ggaggaggtgga

SEQ ID NO: 54 - DNA, 12 bp, Artificial Sequence
  Linker
ggaggtggagga

SEQ ID NO: 55 - DNA, 12 bp, Artificial Sequence
  Linker
ggtggaggagga

SEQ ID NO: 56 - DNA, 12 bp, Artificial Sequence
  Linker
ggaggaggagga

SEQ ID NO: 57 - protein, 17 aa, Artificial Sequence
  His tagged peptide
ARSIINFEKLSHHHHHH

SEQ ID NO: 58 - DNA, 7075 bp, Artificial Sequence
  Plasmid DNA
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 59 - protein, 10 aa, Homo sapiens
HCIRNKSVIL

SEQ ID NO: 60 - protein, 5 aa, Artificial Sequence
  PEST motif substitution
QDNKR

SEQ ID NO: 61 - protein, 649 aa, Artificial Sequence
  Chimeric protein
GAEGGEGVSVPPLLSLRLLELLRESARPLCSRHRVWHRSWLGHGPPSSFGHLQWRDHPVPRPPWAVPTWPRWRLYSTLPEAPTGPPGRGKGDPDRRRHCPCFLETPRWPRDSGDTLHHLVCFQEGLDCGRVAGSSPRRGNHHGFAGKPGGRERVHCQDICVQRGGRRALFKLRGAGRPPQGCFRVEPEAQAFGFFQCQSLFRLLPSGPKVNDGHRSRRWHSLDLYPHLCSHLDLPKQSQEVIRLCESISELLYRMGSPALVILKISAQLRITAEVCRLPTAAQGAPYVITRLVESSPQHPLALTGECCPSAWPTARPLLTPSCIPCCDTNTAGAARSSSHDLSGRRRSRGGARCSASGLCRQRTQDGHSLGAPDSGSGGSPPCCILKLLASDWLGCTSGLVSLHSRRGRRLECLQSQPPEPMTSTNPPSFPLRKRESGRIPVRKSSSQMPSVGLPGDFLHPPQAQHTERHTDNFGKYNPSDYLQGTAGPGLALAQCSATTVCLHSKDGHPKLSLLVLPTSQWWYYFLGPASSSTCGQSANIQGVQRSLEGWQTCWGRSRKHWGSTWNGSARLSPGSTLWVMRICLRSLGIARTWLSCRSTSRKCSPAFPASSGRPGRVGPTGEKRRHGGQRTERHCGPPWKNWSHWRKR

SEQ ID NO: 62 - DNA, 2004 bp, Artificial Sequence
  Nucleic acid encoding chimeric protein
ggtgcggaaggtggtgagggcgtaagtgttcctccacttttatctcttcgtttgttagaacttttgcgtgaatctgcacgtccgctttgttctcgtcaccgtgtatggcatcgttcatggttaggacatggtccaccaagttcttttggtcacctacaatggcgtgaccaccctgtaccaagaccaccatgggctgttccaacatggcctcgctggcgcttatacagtacgcttcctgaagcgccgactggcccaccaggtcgtggaaaaggagacccagaccgtcgtcgtcactgtccatgtttccttgagactccacgctggccacgcgattcaggagatactttacaccatttagtatgttttcaagaaggtctagattgtggacgtgttgctggtagcagccctcgtcgtggtaatcatcatggatttgcaggaaaaccaggtggtcgtgaacgcgtgcactgtcaagacatctgcgttcagcgcggtggacgccgtgcacttttcaaactacgcggcgctggccgtccacctcaaggatgtttccgtgtagaacctgaagcacaagcattcggttttttccaatgtcaatcacttttccgtcttcttcctagtggtccaaaagttaatgacggtcatcgttcacgtcgctggcacagccttgatttatatccacatttatgtagtcacttggatcttcctaaacaatctcaagaagtaatccgtctttgtgaatctattagtgaattactttatcgtatgggttctccagctctggtaattttgaaaatctcagctcaacttcgtatcactgcagaagtatgccgtttacctactgcagctcaaggcgcaccatacgttattactcgtttagttgaaagctctcctcaacacccattagcattaactggtgaatgttgtccgtctgcatggccaacagctcgtccactattaacaccttcttgcattccatgctgtgacacaaatacagctggagcagctagatcatcctcacatgatttatctggacgtcgtcgttcacgtggtggcgctcgttgttcagcatctggtttgtgtcgccaacgtacacaagacggccattctttaggagcaccagatagtggctctggaggttcccctccatgttgcattttaaaacttcttgcaagtgattggttaggatgtacgtcaggtttagtatctctacattctcgtcgaggtcgtcgtcttgaatgtttacaatctcaaccaccagaaccaatgacttccacaaatcctccaagttttcctttacgtaagcgtgaatcgggtcgtattccagttcgcaaatcttctagccaaatgccatcagtaggcctaccgggtgattttttacaccctcctcaagctcaacacacagaacgtcacactgacaacttcggcaaatacaacccaagcgattatttgcagggtactgctggtccaggcttagcactagcacaatgttctgctacgacagtttgtcttcatagtaaagatggacacccaaaattatctttattagtccttccaacaagccaatggtggtattactttttaggtccagcttcatcttctacttgtggtcaatctgcaaatattcaaggcgttcaacgcagtttagaaggctggcaaacatgttggggccgttcccgtaagcactggggtagcacttggaatggctccgcaagattgtccccaggatctaccctttgggttatgcgtatttgtttacgtagtttgggcattgcacgtacatggttaagctgtcgttcaacgtctcgtaaatgttctcccgcattcccagcgtcatctggtcgtccaggccgtgttggtccaaccggcgaaaaacgtcgccacggaggtcaacgcaccgaacgtcactgcggtccaccatggaaaaactggagccattggcgtaaacgcgctcgttctatcattaacttcgaaaaattatctcatcaccatcatcaccattaataa

SEQ ID NO: 63 - protein, 242 aa, Artificial Sequence
  Chimeric protein
NIQGVQRSLEGWQTCWGRSRKHWGSTWNGSARLSPGSTLWVMRICLRSLGIARTWLSCRSTSRKCSPAFPASSSTLPEAPTGPPGRGKGDPDRRRHCPCFLETPRWPRDSGDTLHHLVCFQEGLDCGRVAGSSPRRGNHHGFAGKPGGRERVHCQDICVQRGGRRALFKLRGAGRPPQGCFRVEPEAQAFGFFQCQSLFRLLPSGPKVNDGHRSRRWHSLDLYPHLCSHLDLPKQSQEVIRL

SEQ ID NO: 64 - DNA, 783 bp, Artificial Sequence
  Nucleic acid encoding chimeric protein
aacattcaaggtgtacaacgttctctagaaggttggcaaacatgttggggtcgttctcgtaagcactggggaagcacatggaacggctctgctcgtttatctccaggttctacgttatgggtaatgcgtatctgtctacgttccctaggtatcgcgcgtacttggttaagctgccgctccacttctcgcaaatgttctccagcatttccggcatcgtcatctactctaccagaagctccaactggaccaccaggtcgtggtaaaggcgatccagaccgtcgtcgtcactgtccatgtttcttagaaactccacgttggccaagagatagcggagacacattgcatcatctagtatgtttccaagaaggtttagactgtggtcgtgttgcaggttccagcccacgtagaggtaaccaccacggcttcgccggtaaaccaggtggtcgtgaacgtgttcattgccaagatatctgtgtacaacgtggaggtcgtcgtgctttatttaaattacgaggagcgggtcgtcctccacaaggttgttttcgcgttgaaccagaagcacaagcatttggattctttcagtgtcaatctcttttccgtcttttgcctagtggaccaaaagtaaatgacggacatcgctctcggcgttggcactcattagatttgtatcctcatctttgtagtcatctagatctaccaaaacaatctcaagaagtaattcgtcttgctcgttctatcattaacttcgaaaaattatctcatcaccatcatcaccattaataa

SEQ ID NO: 65 - protein, 472 aa, Artificial Sequence
  Chimeric protein
DALVKDSEENCKNWSNSKRRLQKKKAPSRSGMSERNDLFSFTFPRPDLACGATWIRRDDTWGGGNRDLNPVGKQFKEWHCSLCSVCARNAINMEYTIDIFFCPNLVAALETIQNMSIKRLYSIPSTEKKENLWTKFILARGGEEGGIRTEDFFAALDLKACPPSPSAGSWTEDIKLEEKKKNPSRNGSHLIAVTVFLWDFVLYVPQASIIEDDESGTENREGQFTALSDGVDKQMTGNFPQEIRSFKCWTLDTLHPDVKKYISDHMKVHSPSPCLSYKQQSHSNLKKISFSSFSTYLCPYLTMDQSAKSIKEKKNMKYWSCCRRKNFRFRGGLRRRGSMAPLRFSGVAQQPLSNGAIRELSAKACTTETVSFLRKKKVKLRIIYGEENWRNRRKRSQLDVMEQLEPKCPPKCPPKCPPCVFLLNQPVGNHPIPGGNRPSQQTVEIKGKAQKCFSFSMIFSFLPYSMSYSGHF

SEQ ID NO: 66 - DNA, 1473 bp, Artificial Sequence
  Nucleic acid encoding chimeric protein
gacgctcttgtaaaagattcagaagaaaactgtaagaactggtcaaatagtaaacgtcgccttcaaaagaaaaaagcaccatcccgttctggtatgtccgaacgcaatgatttatttagtttcacattcccacgccctgaccttgcgtgcggcgcaacctggatccgtcgtgatgatacttggggcgggggtaatcgcgatttaaatccagttgggaaacaattcaaagaatggcattgctctctttgtagtgtttgtgcacgtaacgctattaacatggaatatacgatcgacatttttttttgtcctaacttagtagctgctttagaaactattcaaaacatgagcattaaacgtttatattctatcccatccactgaaaaaaaagaaaacttatggacaaaattcattttagcgcgtggtggtgaagaagggggaattcgtacagaagatttctttgctgcacttgatttaaaagcttgtccaccaagcccatcagcaggcagttggacggaagacatcaaacttgaagaaaaaaaaaaaaaccctagccgtaacggctctcatctaattgcagtaacagtattcttgtgggattttgtgttatatgtacctcaagcatcgattattgaggacgatgaaagtggtacagaaaacagagaaggtcaattcactgctctttcagacggtgtagataaacaaatgacaggaaattttcctcaagaaatccgttccttcaaatgttggactttagatacactacatccagatgttaaaaaatatatctcagaccatatgaaagttcattctccttcaccatgtcttagctacaaacaacaatctcattctaacttaaaaaaaatctcattctcaagcttctctacatacctatgtccatatttaacgatggaccaatctgcaaaatctataaaagaaaaaaagaatatgaaatactggtcttgttgtcgccgcaaaaactttcgttttcgtggtggtctgcgtcgtcgtggatctatggcgcctctacgtttcagtggagttgctcaacagccactttctaatggtgctatccgcgaacttagtgctaaagcatgcacaacagaaacagtgagtttccttcgtaagaaaaaagttaaattgcgtattatttatggtgaagaaaattggcgcaaccgtcgtaaacgctctcaacttgacgtaatggaacaacttgaaccaaaatgtccacctaaatgtcctccaaaatgtccaccttgtgttttcttattaaaccaacctgttggcaaccacccaatccctggaggtaaccgtccatcccaacaaacagtagaaatcaaagggaaagcacaaaaatgtttctctttttctatgattttctcctttttaccttatagtatgtcttactccggccacttcgctcgttctatcattaacttcgaaaaattatctcatcaccatcatcaccattaataa

SEQ ID NO: 67 - protein, 529 aa, Artificial Sequence
  Detoxified listeriolysin O
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKEATGLAWEAARTVIDDRNLPLVKNRNISIWGTTLYPKYSNKVDNPIE

SEQ ID NO: 68 - protein, 20 aa, Artificial Sequence
  Mutant peptide
FMVAVAHVAAFLLEDRAVCV

SEQ ID NO: 69 - protein, 21 aa, Artificial Sequence
  Mutant peptide
AENVEQVLVTSIQGAVDYPDP

SEQ ID NO: 70 - protein, 21 aa, Artificial Sequence
  Mutant peptide
SFKKKFEECQHNIIKLQNGHT

SEQ ID NO: 71 - protein, 21 aa, Artificial Sequence
  Mutant peptide
SALIESLNQKTQSTGDHPQPT

SEQ ID NO: 72 - protein, 21 aa, Artificial Sequence
  Mutant peptide
KAYLPVNESFAFTADLRSNTG

SEQ ID NO: 73 - protein, 21 aa, Artificial Sequence
  Mutant peptide
HTLLEITEESGAVLVDKSDSD

SEQ ID NO: 74 - protein, 21 aa, Artificial Sequence
  Mutant peptide
SVMCTYSPPLDKLFCQLAKTC

SEQ ID NO: 75 - protein, 21 aa, Artificial Sequence
  Mutant peptide
ESGKHKYRQTAMFTATMPPAV

SEQ ID NO: 76 - protein, 21 aa, Artificial Sequence
  Mutant peptide
AAPSAASSPADVQSLKKAMSS

SEQ ID NO: 77 - protein, 21 aa, Artificial Sequence
  Mutant peptide
SQLFSLNPRGRSLVTAGRIDR

SEQ ID NO: 78 - protein, 21 aa, Artificial Sequence
  Mutant peptide
SLARGPLSEAGLALFDPYSKE

SEQ ID NO: 79 - protein, 21 aa, Artificial Sequence
  Mutant peptide
QKKLCHLSSTGLPRETIASLP

SEQ ID NO: 80 - protein, 21 aa, Artificial Sequence
  Mutant peptide
LTASNMEGKSWPSEVLVCTTS

SEQ ID NO: 81 - protein, 21 aa, Artificial Sequence
  Mutant peptide
YAAQQHETFLTNGDRAGFLIG

SEQ ID NO: 82 - protein, 21 aa, Artificial Sequence
  Mutant peptide
QAKVPFSEETQNLILPYISDM

SEQ ID NO: 83 - protein, 21 aa, Artificial Sequence
  Mutant peptide
CNRAGEKHCFSSNEAARDFGG

SEQ ID NO: 84 - protein, 21 aa, Artificial Sequence
  Mutant peptide
RNPQFLDPVLAYLMKGLCEKP

SEQ ID NO: 85 - protein, 21 aa, Artificial Sequence
  Mutant peptide
LECERGKQEAKLLAERSRFED

SEQ ID NO: 86 - protein, 21 aa, Artificial Sequence
  Mutant peptide
APLEWLRYFDKKELELMLCGM

SEQ ID NO: 87 - protein, 21 aa, Artificial Sequence
  Mutant peptide
KAFLHWYTGEAMDEMEFTEAE

SEQ ID NO: 88 - protein, 21 aa, Artificial Sequence
  Mutant peptide
DEVALVEGVQSLGFTYLRLKD

SEQ ID NO: 89 - protein, 21 aa, Artificial Sequence
  Mutant peptide
DFSQLQRNILPSNPRVTRFHI

SEQ ID NO: 90 - protein, 21 aa, Artificial Sequence
  Mutant peptide
ISTNGSFIRLLDAFKGVVMHT

SEQ ID NO: 91 - protein, 21 aa, Artificial Sequence
  Mutant peptide
ITPPTTTTKKARVSTPKPATP

SEQ ID NO: 92 - protein, 21 aa, Artificial Sequence
  Mutant peptide
NYNTSHLNNDVWQIFENPVDW

SEQ ID NO: 93 - protein, 21 aa, Artificial Sequence
  Mutant peptide
QKTLHNLLRKVVPSFSAEIER

SEQ ID NO: 94 - protein, 21 aa, Artificial Sequence
  Mutant peptide
VELCPGNKYEMRRHGTTHSLV

SEQ ID NO: 95 - protein, 21 aa, Artificial Sequence
  Mutant peptide
GIDKLTQLKKPFLVNNKINKI

SEQ ID NO: 96 - protein, 20 aa, Artificial Sequence
  Mutant peptide
GTTILNCFHDVLSGKLSGGS

SEQ ID NO: 97 - protein, 21 aa, Artificial Sequence
  Mutant peptide
PSFQEFVDWENVSPELNSTDQ

SEQ ID NO: 98 - protein, 21 aa, Artificial Sequence
  Mutant peptide
PALVEEYLERGNFVANDLDWL

SEQ ID NO: 99 - protein, 21 aa, Artificial Sequence
  Mutant peptide
ELKACKPNGKRNPYCEVSMGS

SEQ ID NO: 100 - protein, 21 aa, Artificial Sequence
  Mutant peptide
SPFPAAVILRDALHMARGLKY

SEQ ID NO: 101 - protein, 21 aa, Artificial Sequence
  Mutant peptide
QQLDTYILKNVVAFSRTDKYR

SEQ ID NO: 102 - protein, 21 aa, Artificial Sequence
  Mutant peptide
SFVGQTRVLMINGEEVEETEL

SEQ ID NO: 103 - protein, 21 aa, Artificial Sequence
  Mutant peptide
AFFINFIAIYHHASRAIPFGT

SEQ ID NO: 104 - protein, 21 aa, Artificial Sequence
  Mutant peptide
GLALPNNYCDVCLGDSKINKK

SEQ ID NO: 105 - protein, 21 aa, Artificial Sequence
  Mutant peptide
EGQISIAKYENCPKDNPMYYC

SEQ ID NO: 106 - protein, 21 aa, Artificial Sequence
  Mutant peptide
NFKRKRVAAFQKNLIEMSELE

SEQ ID NO: 107 - protein, 21 aa, Artificial Sequence
  Mutant peptide
KMKGELGMMLILQNVIQKTTT

SEQ ID NO: 108 - protein, 21 aa, Artificial Sequence
  Mutant peptide
SIECKGIDKEINESKNTHLDI

SEQ ID NO: 109 - protein, 21 aa, Artificial Sequence
  Mutant peptide
ELEAAIETVVCTFFTFAGREG

SEQ ID NO: 110 - protein, 21 aa, Artificial Sequence
  Mutant peptide
SLSHREREQMKATLNYEDHCF

SEQ ID NO: 111 - protein, 21 aa, Artificial Sequence
  Mutant peptide
HIKAFDRTFANNPGPMVVFAT

SEQ ID NO: 112 - protein, 21 aa, Artificial Sequence
  Mutant peptide
ITSNFVIPSEYWVEEKEEKQK

SEQ ID NO: 113 - protein, 21 aa, Artificial Sequence
  Mutant peptide
GLVTFQAFIDVMSRETTDTDT

SEQ ID NO: 114 - protein, 21 aa, Artificial Sequence
  Mutant peptide
HLLGRLAAIVGKQVLLGRKVV

SEQ ID NO: 115 - protein, 21 aa, Artificial Sequence
  Mutant peptide
HWNDLAVIPAGVVHNWDFEPR

SEQ ID NO: 116 - protein, 21 aa, Artificial Sequence
  Mutant peptide
SMDHKTGTIAMQNTTQLRSRY

SEQ ID NO: 117 - protein, 21 aa, Artificial Sequence
  Mutant peptide
QPLRRLVLHVVSAAQAERLAR

SEQ ID NO: 118 - DNA, 945 bp, Artificial Sequence
  Plasmid DNA
ccatctttccaagaattcgttgattgggaaaacgtttctccagaattaaactctacagatcaaggtggtggaggaaaagcatatttaccagttaacgaatctttcgcattcacagcagatttacgttctaacacaggtggtggaggtggacgtaacccacaattcttagatccagttttagcatatttaatgaaaggtttatgtgaaaaaccaggtggaggaggtaaagcattcttacattggtatacaggtgaagcaatggatgaaatggaattcacagaagcagaaggaggtggtggagatttctctcaattacaacgtaacattttaccatctaacccacgtgttacacgtttccatattggaggaggtggtattacaccaccaacaacaacaacaaaaaaagcacgtgtttctacaccaaaaccagcaacaccaggaggtggaggtaactataacacatctcatttaaacaacgatgtttggcaaattttcgaaaacccagttgattggggaggaggaggtcatattaaagcattcgatcgtacattcgcaaacaacccaggtccaatggttgttttcgcaacaggaggaggtggaattacatctaacttcgttattccatctgaatattgggttgaagaaaaagaagaaaaacaaaaaggaggtggaggaggtttagttacattccaagcattcattgatgttatgtctcgtgaaacaacagatacagatacaggtggaggaggacattggaacgatttagcagttattccagcaggtgttgttcataactgggatttcgaaccacgtggaggaggaggacaaccattacgtcgtttagttttacatgttgtttctgcagcacaagcagaacgtttagcacgtgcacgttctattattaacttcgaaaaattatctcatcatcatcatcatcattaataa

SEQ ID NO: 119 - DNA, 1485 bp, Artificial Sequence
  Plasmid DNA
ccatctttccaagaattcgttgattgggaaaacgtttctccagaattaaactctacagatcaaggtggtggaggattcatggttgcagttgcacatgttgcagcattcttattagaagatcgtgcagtttgtgttggaggaggaggagcagaaaacgttgaacaagttttagttacatctattcaaggtgcagttgattatccagatccaggtggaggtggatctttcaaaaaaaaattcgaagaatgtcaacataacattattaaattacaaaacggtcatacaggtggaggaggttctgcattaattgaatctttaaaccaaaaaacacaatctacaggtgatcatccacaaccaacaggaggtggtggaaaagcatatttaccagttaacgaatctttcgcattcacagcagatttacgttctaacacaggtggaggaggtggtcatacattattagaaattacagaagaatctggtgcagttttagttgataaatctgattctgatggaggtggaggttctgttatgtgtacatattctccaccattagataaattattctgtcaattagcaaaaacatgtggaggaggaggtgaatctggtaaacataaatatcgtcaaacagcaatgttcacagcaacaatgccaccagcagttggaggaggtggagcagcaccatctgcagcatcttctccagcagatgttcaatctttaaaaaaagcaatgtcttctggaggtggaggatctcaattattctctttaaacccacgtggtcgttctttagttacagcaggtcgtattgatcgtggtggaggaggatctttagcacgtggtccattatctgaagcaggtttagcattattcgatccatattctaaagaaggaggaggaggacaaaaaaaattatgtcatttatcttctacaggtttaccacgtgaaacaattgcatctttaccaggtggtggaggattaacagcatctaacatggaaggtaaatcttggccatctgaagttttagtttgtacaacatctggtggaggtggatatgcagcacaacaacatgaaacattcttaacaaacggtgatcgtgcaggtttcttaattggtggtggaggaggtcaagcaaaagttccattctctgaagaaacacaaaacttaattttaccatatatttctgatatgggaggtggtggatgtaaccgtgcaggtgaaaaacattgtttctcttctaacgaagcagcacgtgatttcggtggtggaggaggtggtcgtaacccacaattcttagatccagttttagcatatttaatgaaaggtttatgtgaaaaaccaggaggtggaggtttagaatgtgaacgtggtaaacaagaagcaaaattattagcagaacgttctcgtttcgaagatggaggaggaggtgcaccattagaatggttacgttatttcgataaaaaagaattagaattaatgttatgtggtatg

SEQ ID NO: 120 - DNA, 1884 bp, Artificial Sequence
  Plasmid DNA
ccatctttccaagaattcgttgattgggaaaacgtttctccagaattaaactctacagatcaagcagaaaacgttgaacaagttttagttacatctattcaaggtgcagttgattatccagatccatctttcaaaaaaaaattcgaagaatgtcaacataacattattaaattacaaaacggtcatacatctgcattaattgaatctttaaaccaaaaaacacaatctacaggtgatcatccacaaccaacaaaagcatatttaccagttaacgaatctttcgcattcacagcagatttacgttctaacacaggtcatacattattagaaattacagaagaatctggtgcagttttagttgataaatctgattctgattctgttatgtgtacatattctccaccattagataaattattctgtcaattagcaaaaacatgtgaatctggtaaacataaatatcgtcaaacagcaatgttcacagcaacaatgccaccagcagttgcagcaccatctgcagcatcttctccagcagatgttcaatctttaaaaaaagcaatgtcttcttctcaattattctctttaaacccacgtggtcgttctttagttacagcaggtcgtattgatcgttctttagcacgtggtccattatctgaagcaggtttagcattattcgatccatattctaaagaacaaaaaaaattatgtcatttatcttctacaggtttaccacgtgaaacaattgcatctttaccattaacagcatctaacatggaaggtaaatcttggccatctgaagttttagtttgtacaacatcttatgcagcacaacaacatgaaacattcttaacaaacggtgatcgtgcaggtttcttaattggtcaagcaaaagttccattctctgaagaaacacaaaacttaattttaccatatatttctgatatgtgtaaccgtgcaggtgaaaaacattgtttctcttctaacgaagcagcacgtgatttcggtggtcgtaacccacaattcttagatccagttttagcatatttaatgaaaggtttatgtgaaaaaccattagaatgtgaacgtggtaaacaagaagcaaaattattagcagaacgttctcgtttcgaagatgcaccattagaatggttacgttatttcgataaaaaagaattagaattaatgttatgtggtatgccagcattagttgaagaatatttagaacgtggtaacttcgttgcaaacgatttagattggttagaattaaaagcatgtaaaccaaacggtaaacgtaacccatattgtgaagtttctatgggttcttctccattcccagcagcagttattttacgtgatgcattacatatggcacgtggtttaaaatatcaacaattagatacatatattttaaaaaacgttgttgcattctctcgtacagataaatatcgttctttcgttggtcaaacacgtgttttaatgattaacggtgaagaagttgaagaaacagaattagcattcttcattaacttcattgcaatttatcatcatgcatctcgtgcaattccattcggtacaggtttagcattaccaaacaactattgtgatgtttgtttaggtgattctaaaattaacaaaaaagaaggtcaaatttctattgcaaaatatgaaaactgtccaaaagataacccaatgtattattgtaacttcaaacgtaaacgtgttgcagcattccaaaaaaacttaattgaaatgtctgaattagaaaaaatgaaaggtgaattaggtatgatgttaattttacaaaacgttattcaaaaaacaacaacagcacgttctattattaacttcgaaaaattatctcatcatcatcatcatcattaataa